(19) United States
(12) Patent Application Publication
(10) Pub. No.: US 2026/0193623 A1
(43) Pub. Date: Jul. 9, 2026

(54) RETROTRANSPOSON COMPOSITIONS AND METHODS OF USE

(71) Applicant: Metagenomi, Inc., Emeryville, CA (US)

(72) Inventors: Brian C. THOMAS, Berkeley, CA (US); Lisa ALEXANDER, Albany, CA (US); Christopher BROWN, Albany, CA (US); Cindy CASTELLE, San Francisco, CA (US); Daniela S.A. GOLTSMAN, Oakland, CA (US); Sarah LAPERRIERE, Berkeley, CA (US); Morayma TEMOCHE-DIAZ, Emeryville, CA (US); Anu THOMAS, Emeryville, CA (US); Mary Kaitlyn CHIU (née TSAI), San Francisco, CA (US)

(73) Assignee: Metagenomi, Inc., Emeryville, CA (US)

(21) Appl. No.: 19/131,137

(22) PCT Filed: Dec. 8, 2023
(86) PCT No.: PCT/US2023/083224

§ 371 Date: May 19, 2025

Related U.S. Application Data
(60) Provisional application No. 63/386,865, filed on Dec. 9, 2022.
(60) Provisional application No. 63/489,154, filed on Mar. 8, 2023.
(60) Provisional application No. 63/491,939, filed on Mar. 23, 2023.
(60) Provisional application No. 63/501,373, filed on May 10, 2023.

(51) Int. Cl. C12N 9/12; C07K 14/005; C12N 1/16; C12N 5/04; C12N 5/07; C12N 5/071; C12N 5/10; C12N 9/22; C12N 15/10; C12N 15/52; C12N 15/864; C12N 15/867

(52) U.S. Cl. CPC C12N 9/1276; C07K 14/005; C12N 1/16; C12N 5/04; C12N 5/0601; C12N 5/0602; C12N 5/10; C12N 9/22; C12N 15/1096; C12N 15/52; C12N 15/8645; C12N 15/867; C12Y 207/07049; C07K 2319/09; C12N 2510/04; C12N 2740/15043; C12N 2750/14143; C12N 2800/10; C12N 2800/90

(57) ABSTRACT

The present disclosure provides systems and methods for transposing a cargo nucleotide sequence to a target nucleic acid sequence. These systems and methods can comprise a nucleic acid comprising the cargo nucleotide sequence, wherein the cargo nucleotide sequence is configured to interact with a retrotransposase, and the retrotransposase, wherein the retrotransposase is configured to transpose the cargo nucleotide sequence to the target nucleic acid sequence. The systems and methods can also involve use of functional fragments of retrotransposases.

### CROSS-REFERENCE

This application claims the benefit of and priority to U.S. Provisional Patent Application No. 63/386,865, filed Dec. 9, 2022, U.S. Provisional Patent Application No. 63/489,154 filed Mar. 8, 2023, U.S. Provisional Patent Application No. 63/491,939 filed Mar. 23, 2023, and U.S. Provisional Patent Application No. 63/501,373 filed May 10, 2023, each of which is incorporated by reference in its entirety herein.

### BACKGROUND

Transposable elements are movable DNA sequences and play a crucial role in gene function and evolution. While transposable elements are found in nearly all forms of life, their prevalence varies among organisms, with a large proportion of the eukaryotic genome encoding for transposable elements.

### SUMMARY

While the foundational research on transposable elements was conducted in the 1940s, their potential utility in DNA manipulation and gene editing applications has only been recognized in recent years.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 1-29, 393-735, 799-895, 1020-1476, 1544-1554, 1850-2160, 2165-2210, and 2258-2266. In some embodiments, the retrotransposase comprises an amino acid sequence having at least 80% sequence identity to any one of SEQ ID NOs: 1-29, 393-735, 799-895, 1020-1476, 1544-1554, 1850-2160, 2165-2210, and 2258-2266. In some embodiments, the retrotransposase comprises an amino acid sequence having at least 90% sequence identity to any one of SEQ ID NOs: 1-29, 393-735, 799-895, 1020-1476, 1544-1554, 1850-2160, 2165-2210, and 2258-2266. In some embodiments, the retrotransposase comprises an amino acid sequence having at least 95% sequence identity to any one of SEQ ID NOs: 1-29, 393-735, 799-895, 1020-1476, 1544-1554, 1850-2160, 2165-2210, and 2258-2266. In some embodiments, the retrotransposase is encoded by a nucleic acid having at least 75% sequence identity to any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806. In some embodiments, the retrotransposase is encoded by a nucleic acid sequence having at least 80% sequence identity to any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806. In some embodiments, retrotransposase is encoded by a nucleic acid sequence having at least 90% sequence identity to any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806. In some embodiments, retrotransposase is encoded by a nucleic acid sequence having at least 95% sequence identity to any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806. In some embodiments, the double-stranded nucleic acid comprises a 5′ recognition sequence comprising a GG nucleotide sequence and a 3′ recognition sequence comprising a TGAC nucleotide sequence. In some embodiments, the 5′ recognition sequence and the 3′ recognition sequence are configured to interact with the retrotransposase. In some embodiments, the double-stranded nucleic acid comprising a cargo nucleotide sequence is RNA. In some embodiments, the RNA is an in vitro transcribed RNA. In some embodiments, the RNA comprises a sequence 5′ to said cargo sequence or a sequence 3′ to said cargo sequence that has at least 80% sequence identity to an RNA cognate of any one of SEQ ID NOs: 761-798, 2161-2164, and 2211-2257, a complement thereof, or a reverse complement thereof.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 1-29, 393-401, 799-894, 1476, 1850-1926, and 2165-2210. In some embodiments, the retrotransposase is encoded by a nucleic acid sequence having at least 80% sequence identity to any one of SEQ ID NOs: SEQ ID NOs: 1535-1536, 1542-1543, 1611-1623, 1663-1691, and 1786-1806.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to SEQ ID NO: 402 or SEQ ID NO: 895.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to SEQ ID NO: 388.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 403-426. In some embodiments, the retrotransposase is encoded by a nucleic acid sequence having at least 80% sequence identity to any one of SEQ ID NOs: 389-392 and 1504-1507.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 427-439.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 440-554 and 1020-1037. In some embodiments, the retrotransposase is encoded by a nucleic acid sequence having at least 80% sequence identity to any one of SEQ ID NOs: 356-373, 964-981, and 1003-1019.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 555-608 and 1927-2010. In some embodiments, the retrotransposase is encoded by a nucleic acid sequence having at least 80% sequence identity to any one of SEQ ID NOs: 66-173, 740-756, 1521-1534, 1539-1541, 1624-1637, 1645-1662, and 1701-1782.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 609-610 and 1555. In some embodiments, the retrotransposase is encoded by a nucleic acid sequence having at least 80% sequence identity to any one of SEQ ID NOs: 308-309 and 324-325.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 611-615 and 1544-1545. In some embodiments, the retrotransposase is encoded by a nucleic acid sequence having at least 80% sequence identity to any one of SEQ ID NOs: 310-312, 326-328, 1556-1557, and 1569-1570.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to SEQ ID NO: 616 or SEQ ID NO: 617. In some embodiments, the retrotransposase is encoded by a nucleic acid sequence having at least 80% sequence identity to any one of SEQ ID NOs: 313-314 and 329-330.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 618-622 and 2258-2266. In some embodiments, the retrotransposase is encoded by a nucleic acid sequence having at least 80% sequence identity to any one of SEQ ID NOs: 315-319 and 331-335.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to SEQ ID NO: 623. In some embodiments, the retrotransposase is encoded by a nucleic acid sequence having at least 80% sequence identity to SEQ ID NO: 320 or SEQ ID NO: 336.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 624-626. In some embodiments, the retrotransposase is encoded by a nucleic acid sequence having at least 80% sequence identity to any one of SEQ ID NOs: 321-323, 337-339, and 1785.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 624-626. In some embodiments, the retrotransposase is encoded by a nucleic acid sequence having at least 80% sequence identity to any one of SEQ ID NOs: 321-323, 337-339, and 1785.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 627-673, 1039-1475, and 2011-2026. In some embodiments, the retrotransposase is encoded by a nucleic acid sequence having at least 80% sequence identity to any one of SEQ ID NOs: 174-187 and 1508-1520.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 674-678. In some embodiments, the retrotransposase is encoded by a nucleic acid sequence having at least 80% sequence identity to any one of SEQ ID NOs: 188-197.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 679-683. In some embodiments, the retrotransposase is encoded by a nucleic acid sequence having at least 80% sequence identity to any one of SEQ ID NOs: 198-207.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 684-692 and 2027-2046. In some embodiments, the retrotransposase is encoded by a nucleic acid sequence having at least 80% sequence identity to any one of SEQ ID NOs: 208-225 and 757-759.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 693-697 and 2047-2090. In some embodiments, the retrotransposase is encoded by a nucleic acid sequence having at least 80% sequence identity to any one of SEQ ID NOs: 226-235.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 698-702 and 2091-2119. In some embodiments, the retrotransposase is encoded by a nucleic acid sequence having at least 80% sequence identity to any one of SEQ ID NOs: 236-245 and 759-760.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 703-707. In some embodiments, the retrotransposase is encoded by a nucleic acid sequence having at least 80% sequence identity to any one of SEQ ID NOs: 246-255.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 708-718 and 2121-2159. In some embodiments, the retrotransposase is encoded by a nucleic acid sequence having at least 80% sequence identity to any one of SEQ ID NOs: 256-277, 1638-1644, and 1693-1700.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 719-728. In some embodiments, the retrotransposase is encoded by a nucleic acid sequence having at least 80% sequence identity to any one of SEQ ID NOs: 278-297.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 729-733. In some embodiments, the retrotransposase is encoded by a nucleic acid sequence having at least 80% sequence identity to any one of SEQ ID NOs: 298-307.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 734-735 and 1546-1553. In some embodiments, the retrotransposase is encoded by a nucleic acid sequence having at least 80% sequence identity to any one of SEQ ID NOs: 1558-1567, 1571-1580, and 1783-1784.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to SEQ ID NO: 1038 or SEQ ID NO: 2160. In some embodiments, the retrotransposase is encoded by a nucleic acid sequence having at least 80% sequence identity to SEQ ID NO: 1692.

Described herein, in certain embodiments, are engineered retrotransposase systems, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to SEQ ID NO: 1554. In some embodiments, the retrotransposase is encoded by a nucleic acid sequence having at least 80% sequence identity to SEQ ID NO: 1568 or SEQ ID NO: 1594. In some embodiments, the retrotransposase comprises one or more nuclear localization sequences (NLSs) proximal to an N- or C-terminus of the retrotransposase. In some embodiments, the NLS comprises a sequence at least 80% identical to a sequence from the group consisting of SEQ ID NO: 1477-1492. In some embodiments, the NLS comprises SEQ ID NO: 1478. In some embodiments, the NLS is proximal to the N-terminus of the retrotransposase. In some embodiments, the NLS comprises SEQ ID NO: 1477. In some embodiments, the NLS is proximal to the C-terminus of the retrotransposase.

Described herein, in certain embodiments, are polypeptides comprising a reverse transcriptase comprising an amino acid sequence having at least 75% sequence identity to any one of SEQ ID NOs: 1-29, 393-735, 799-895, 1020-1476, 1544-1554, 1850-2160, 2165-2210, and 2258-2266 fused N- or C-terminally to a non-retrotransposase domain or an affinity tag. In some embodiments, the non-retrotransposase domain is an RNA-binding protein domain. In some embodiments, the RNA binding protein domain comprises a bacteriophage MS2 coat protein (MCP) domain.

Described herein, in certain embodiments, are nucleic acids encoding the engineered retrotransposase system described herein or the polypeptide described herein.

Described herein, in certain embodiments, are methods for modifying a target nucleic acid sequence comprising contacting the target nucleic acid sequence using the engineered nuclease system described herein. In some embodiments, modifying the target nucleic acid sequence comprises binding, nicking, or cleaving, the target nucleic acid sequence. In some embodiments, the target nucleic acid sequence comprises genomic DNA, viral DNA, viral RNA, or bacterial DNA. In some embodiments, the target nucleic acid sequence comprises deoxyribonucleic acid (DNA). In some embodiments, the modification is in vitro. In some embodiments, the modification is in vivo. In some embodiments, the modification is ex vivo.

Described herein, in certain embodiments, are methods of modifying a target nucleic acid sequence in a mammalian cell comprising contacting the mammalian cell using the engineered nuclease system described herein.

Described herein, in certain embodiments, are methods for synthesizing complementary DNA (cDNA), comprising: (a) providing an RNA molecule as a template for cDNA synthesis, (b) providing a primer oligonucleotide to initiate cDNA synthesis from the RNA molecule; and (c) synthesizing cDNA initiated by the primer oligonucleotide from the template using a reverse transcriptase comprising a sequence having at least 80% sequence identity to a reverse transcriptase domain of any one of SEQ ID NOs: 1-29, 393-735, 799-895, 1020-1476, 1544-1554, 1850-2160, 2165-2210, and 2258-2266. In some embodiments, the primer oligonucleotide comprises an oligo (dT) sequence or a degenerate sequence of at least six oligonucleotides.

Described herein, in certain embodiments, are vectors comprising the nucleic acid described herein. In some embodiments, the vector is a plasmid, a minicircle, a CELiD, an adeno-associated virus (AAV) derived virion, or a lentivirus.

Described herein, in certain embodiments, are cells comprising the engineered nuclease system described herein or the polypeptide described herein. In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell. In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, primary cell, or a derivative thereof. In some embodiments, the cell is an engineered cell. In some embodiments, the cell is a stable cell.

In some aspects, the present disclosure provides for an engineered retrotransposase system, comprising: (a) an RNA comprising a heterologous engineered cargo nucleotide sequence, wherein the cargo nucleotide sequence is configured to interact with a retrotransposase; and (b) a retrotransposase, wherein: (i) the retrotransposase is configured to transpose the cargo nucleotide sequence to a target nucleic acid locus; and (ii) the retrotransposase comprises a reverse transcriptase (RT) domain, an endonuclease domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RT or endonuclease domain of any one of SEQ ID NOs: 1-29, 393-735, 799-895, 1020-1476, and 1546-1553. In some embodiments, the retrotransposase further comprises any of the Zn-binding ribbon motifs of any one of SEQ ID NOs: 1-29, 393-735, 799-895, 1020-1476, 1544-1554, 1850-2160, 2165-2210, and 2258-2266. In some embodiments, the retrotransposase further comprises a sequence having at least 80% sequence identity to any one of SEQ ID NOs: 1-29, 393-735, 799-895, 1020-1476, 1544-1554, 1850-2160, 2165-2210, and 2258-2266. In some embodiments, the retrotransposase further comprises a conserved catalytic D, QG, [Y/F]XDD, or LG motif. In some embodiments, the retrotransposase further comprises a conserved CX_{[2-3]}C Zn finger motif. In some embodiments, the retrotransposase comprises a sequence having at least 80% sequence identity to any one of SEQ ID NOs: 3, 6, 7, 8, 14, and 402. In some embodiments, the system further comprises: (c) a double-stranded DNA sequence comprising the target nucleic acid locus. In some embodiments, the double-stranded DNA sequence comprises a 5′ recognition sequence and a 3′ recognition sequence configured to interact with the retrotransposase, wherein the 5′ recognition sequence comprises a GG nucleotide sequence and the 3′ recognition sequence comprises a TGAC nucleotide sequence. In some embodiments, the RNA is an in vitro transcribed RNA. In some embodiments, the RNA comprises a sequence 5′ to the cargo sequence or a sequence 3′ to the cargo sequence that has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RNA cognate of any one of SEQ ID NOs: 761-798, 2161-2164, and 2211-2257, a complement thereof, or a reverse complement thereof. In some embodiments, the RNA comprises a sequence encoding the retrotransposase. In some embodiments, the heterologous engineered cargo nucleotide sequence comprises an expression cassette.

In some embodiments, the present disclosure provides for an engineered DNA sequence, comprising: (a) a 5′ sequence capable of encoding an RNA sequence configured to interact with a retrotransposase; (b) a heterologous cargo sequence; (c) a sequence encoding a retrotransposase configured to interact with an RNA cognate of the 5′ sequence, wherein the retrotransposase comprises a reverse transcriptase (RT) domain or an endonuclease domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a RT or endonuclease domain of any one of SEQ ID NOs: 1-29, 393-735, 799-895, 1020-1476, 1544-1554, 1850-2160, 2165-2210, and 2258-2266; and (d) a 3′ sequence capable of encoding an RNA sequence configured to interact with the retrotransposase. In some embodiments, the retrotransposase further comprises any of the Zn-binding ribbon motifs of any one of SEQ ID NOs: 1-29, 393-735, 799-895, 1020-1476, 1544-1554, 1850-2160, 2165-2210, and 2258-2266. In some embodiments, the retrotransposase further comprises a sequence having at least 80% sequence identity to any one of SEQ ID NOs: 1-29, 393-735, 799-895, 1020-1476, 1544-1554, 1850-2160, 2165-2210, and 2258-2266. In some embodiments, the retrotransposase further comprises a conserved catalytic D, QG, [Y/F]XDD or LG motif. In some embodiments, the retrotransposase further comprises a conserved CX [2-3]C Zn finger motif. In some embodiments, the retrotransposase comprises a sequence having at least 80% sequence identity to any one of SEQ ID NOs: 3, 6, 7, 8, 14, and 402. In some embodiments, the 5′ sequence or the 3′ sequence comprises a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RNA cognate of any one of SEQ ID NOs: 761-798, 2161-2164, and 2211-2257, a complement thereof, or a reverse complement thereof.

In some aspects, the present disclosure provides for a method for synthesizing complementary DNA (cDNA), comprising: (a) providing an RNA molecule as a template for cDNA synthesis, (b) providing a primer oligonucleotide to initiate cDNA synthesis from the RNA molecule; and (c) synthesizing cDNA initiated by the primer oligonucleotide from the template using a reverse transcriptase comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 1-29, 393-735, 799-895, 1020-1476, 1544-1554, 1850-2160, 2165-2210, and 2258-2266. In some embodiments, the reverse transcriptase comprises a sequence having at least 80% sequence identity to any one of SEQ ID NOs: 1-29, 393-735, 799-895, 1020-1476, 1544-1554, 1850-2160, 2165-2210, and 2258-2266. In some embodiments, the primer oligonucleotide comprises an oligo (dT) sequence or a degenerate sequence of at least six oligonucleotides. In some embodiments, the synthesizing cDNA comprises incubating the template RNA molecule, the primer oligonucleotide, and the reverse transcriptase in a reaction mixture under conditions suitable for extension of a DNA sequence from the RNA template. In some embodiments, the reaction mixture further comprises dNTPs, a reaction buffer, divalent metal ions, Mg²⁺, or Mn²⁺.

In some aspects, the present disclosure provides for a polypeptide comprising a reverse transcriptase domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 1-29, 393-735, 799-895, 1020-1476, 1544-1554, 1850-2160, 2165-2210, and 2258-2266, wherein the sequence is fused N- or C-terminally to a non-retrotransposase domain or an affinity tag. In some embodiments, the reverse transcriptase domain comprises a sequence having at least 80% sequence identity to any one of SEQ ID NOs: 1-29, 393-735, 799-895, 1020-1476, 1544-1554, 1850-2160, 2165-2210, and 2258-2266. In some embodiments, the non-retrotransposase domain is an RNA-binding protein domain. In some embodiments, the RNA binding protein domain comprises a bacteriophage MS2 coat protein (MCP) domain.

In some aspects, the present disclosure provides for a nucleic acid encoding any of the polypeptides described herein.

In some aspects, the present disclosure provides for a nucleic acid encoding an open reading frame, wherein the open reading frame encodes an RT or endonuclease domain having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RT or endonuclease domain of any one of SEQ ID NOs: 1-29, 393-735, 799-895, 1020-1476, 1544-1554, 1850-2160, 2165-2210, and 2258-2266, wherein: (a) the open reading frame is optimized for expression in an organism and the organism is different to the origin of the RT or endonuclease domain; or (b) the ORF comprises a sequence encoding an affinity tag. In some embodiments, the nucleic acid further encodes a retrotransposase comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RT or endonuclease domain of any one of SEQ ID NOs: 1-29, 393-735, 799-895, 1020-1476, 1544-1554, 1850-2160, 2165-2210, and 2258-2266.

In some embodiments, the present disclosure provides for an engineered retrotransposase system, comprising: (a) an RNA comprising a heterologous engineered cargo nucleotide sequence, wherein the cargo nucleotide sequence is configured to interact with a retrotransposase; and (b) a retrotransposase, wherein: (i) the retrotransposase is configured to transpose the cargo nucleotide sequence to a target nucleic acid locus; and (ii) the retrotransposase comprises a reverse transcriptase (RT) domain or an endonuclease domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a RT or endonuclease domain of SEQ ID NO: 402 or 895. In some embodiments, the retrotransposase further comprises any of the Zn-binding ribbon motifs of SEQ ID NO: 402 or 895. In some embodiments, the retrotransposase further comprises a sequence having at least 80% sequence identity to SEQ ID NO: 402 or 895. In some embodiments, the retrotransposase further comprises a conserved catalytic D, QG, [Y/F]XDD, or LG motif of SEQ ID NO: 402 or 895. In some embodiments, the retrotransposase further comprises a conserved CX_{[2-3]}C Zn finger motif of SEQ ID NO: 402 or 895. In some embodiments, the system further comprises: (c) a double-stranded DNA sequence comprising the target locus. In some embodiments, the RNA is an in vitro transcribed RNA. In some embodiments, the RNA comprises a sequence encoding the retrotransposase.

In some aspects, the present disclosure provides for an engineered DNA sequence, comprising: (a) a 5′ sequence capable of encoding an RNA sequence configured to interact with a retrotransposase; (b) a heterologous cargo sequence; (c) a sequence encoding a retrotransposase configured to interact with an RNA cognate of the 5′ sequence, wherein the retrotransposase comprises a reverse transcriptase (RT) domain, an endonuclease domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a RT or endonuclease domain of SEQ ID NO: 402 or 895; and (d) a 3′ sequence capable of encoding an RNA sequence configured to interact with the retrotransposase. In some embodiments, the retrotransposase further comprises any of the Zn-binding ribbon motifs of SEQ ID NO: 402 or 895. In some embodiments, the retrotransposase further comprises a sequence having at least 80% sequence identity to SEQ ID NO: 402 or 895. In some embodiments, the retrotransposase further comprises a conserved catalytic D, QG, [Y/F]XDD or LG motif of SEQ ID NO: 402 or 895. In some embodiments, the retrotransposase further comprises a conserved CX_{[2-3]}C Zn finger motif of SEQ ID NO: 402 or 895.

In some aspects, the present disclosure provides for a method for synthesizing complementary DNA (cDNA), comprising: (a) providing an RNA molecule as a template for cDNA synthesis, (b) providing a primer oligonucleotide to initiate cDNA synthesis from the RNA molecule; and (c) synthesizing cDNA initiated by the primer oligonucleotide from the template using a reverse transcriptase comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of SEQ ID NO: 402 or 895. In some embodiments, the reverse transcriptase comprises a sequence having at least 80% sequence identity to SEQ ID NO: 402 or 895. In some embodiments, the primer oligonucleotide comprises an oligo (dT) sequence or a degenerate sequence of at least six oligonucleotides. In some embodiments, the synthesizing cDNA comprises incubating the template RNA molecule, the primer oligonucleotide, and the reverse transcriptase in a reaction mixture under conditions suitable for extension of a DNA sequence from the RNA template. In some embodiments, the reaction mixture further comprises dNTPs, a reaction buffer, divalent metal ions, Mg²⁺, or Mn²⁺.

In some aspects, the present disclosure provides for a polypeptide comprising a reverse transcriptase domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of SEQ ID NO: 402 or 895, wherein the sequence is fused N- or C-terminally to a non-retrotransposase domain or an affinity tag. In some embodiments, the reverse transcriptase domain comprises a sequence having at least 80% sequence identity to SEQ ID NO: 402 or 895. In some embodiments, the non-retrotransposase domain is an RNA-binding protein domain. In some embodiments, the RNA binding protein domain comprises a bacteriophage MS2 coat protein (MCP) domain.

In some aspects, the present disclosure provides for a nucleic acid encoding an open reading frame, wherein the open reading frame encodes an RT or endonuclease domain having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RT or endonuclease domain of SEQ ID NO: 402 or 895, wherein: (a) the open reading frame is optimized for expression in an organism and the organism is different to the origin of the RT or endonuclease domain; or (b) the ORF comprises a sequence encoding an affinity tag. In some embodiments, the nucleic acid further encodes a retrotransposase comprising a sequence having at least 80% sequence identity to SEQ ID NO: 402 or 895.

In some aspects, the present disclosure provides for a method for synthesizing complementary DNA (cDNA), comprising: (a) providing an RNA molecule as a template for cDNA synthesis, (b) providing a primer oligonucleotide to initiate cDNA synthesis from the RNA molecule; and (c) synthesizing cDNA initiated by the primer oligonucleotide from the template using a reverse transcriptase comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 555-728. In some embodiments, the reverse transcriptase comprises a sequence having at least 80% sequence identity to any one of SEQ ID NOs: 555-560, 563, 564, 566, 567, 569, 572, 574, 580-582, 584-588, 592, 593, 596, 602, 604, 605, 608, 561, 562, 564, 565, 568, 571, 573, 576-579, 583, 590, 591, 594, 598, 601, 606, and 607. In some embodiments, the reverse transcriptase comprises a sequence having at least 80% sequence identity to any one of SEQ ID NOs: 555-560, 563, 564, 566, 567, 569, 572, 574, 580-582, 584-588, 592, 593, 596, 602, 604, 605, and 608. In some embodiments, the primer oligonucleotide comprises an oligo (dT) sequence or a degenerate sequence of at least six oligonucleotides. In some embodiments, the primer oligonucleotide comprises at least one phosphorothioate linkage. In some embodiments, the synthesizing cDNA comprises incubating the template RNA molecule, the primer oligonucleotide, and the reverse transcriptase in a reaction mixture under conditions suitable for extension of a DNA sequence from the RNA template. In some embodiments, the reaction mixture further comprises dNTPs, a reaction buffer, divalent metal ions, Mg²⁺, or Mn²⁺.

In some aspects, the present disclosure provides for a polypeptide comprising a reverse transcriptase domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 555-728, wherein the sequence is fused N- or C-terminally to a non-retrotransposase domain or an affinity tag. In some embodiments, the reverse transcriptase domain comprises a sequence having at least 80% sequence identity to any one of SEQ ID NOs: 555-560, 563, 564, 566, 567, 569, 572, 574, 580-582, 584-588, 592, 593, 596, 602, 604, 605, 608, 561, 562, 564, 565, 568, 571, 573, 576-579, 583, 590, 591, 594, 598, 601, 606, and 607. In some embodiments, the reverse transcriptase comprises a sequence having at least 80% sequence identity to any one of SEQ ID NOs: 555-560, 563, 564, 566, 567, 569, 572, 574, 580-582, 584-588, 592, 593, 596, 602, 604, 605, and 608. In some embodiments, the non-retrotransposase domain is an RNA-binding protein domain. In some embodiments, the RNA binding protein domain comprises a bacteriophage MS2 coat protein (MCP) domain. In some embodiments, the protein comprises a sequence having at least 80% sequence identity to any one of SEQ ID NOs: 30-32, 40-50, 740-756, and 757-760. In some embodiments, the reverse transcriptase domain comprises a sequence having at least 80% sequence identity to any one of SEQ ID NOs: 555-558, 561-567, 569, 570, and 575.

In some aspects, the present disclosure provides for a nucleic acid encoding an open reading frame, wherein the open reading frame encodes an RT or endonuclease domain having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RT or endonuclease domain of any one of SEQ ID NOs: 555-728, wherein: (a) the open reading frame is optimized for expression in an organism and the organism is different to the origin of the RT or endonuclease domain; or (b) the ORF comprises a sequence encoding an affinity tag. In some embodiments, the nucleic acid further encodes a retrotransposase comprising a sequence having at least 80% sequence identity to an RT or endonuclease domain of any one of SEQ ID NOs: 555-560, 563, 564, 566, 567, 569, 572, 574, 580-582, 584-588, 592, 593, 596, 602, 604, 605, 608, 561, 562, 564, 565, 568, 571, 573, 576-579, 583, 590, 591, 594, 598, 601, 606, and 607. In some embodiments, the reverse transcriptase comprises a sequence having at least 80% sequence identity to any one of SEQ ID NOs: 555-560, 563, 564, 566, 567, 569, 572, 574, 580-582, 584-588, 592, 593, 596, 602, 604, 605, and 608.

In some aspects, the present disclosure provides for a nucleic acid comprising a sequence comprising an open reading frame (ORF) comprising a sequence encoding a reverse transcriptase domain or a maturase domain having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain or a maturase domain of any one of SEQ ID NOs: 729-733, wherein: (a) the open reading frame is optimized for expression in an organism and the organism is different to the origin of the RT or endonuclease domain; or (b) the ORF comprises a sequence encoding an affinity tag. In some embodiments, the ORF encodes a protein having at least 80% sequence identity to any one of SEQ ID NOs: 729-733. In some embodiments, the ORF is optimized for expression in the bacterial organism or wherein the organism is E. coli. In some embodiments, the ORF is optimized for expression in a mammalian organism or wherein the organism is a primate organism. In some embodiments, the primate organism is H. sapiens. In some embodiments, the ORF comprises an affinity tag operably linked to the sequence encoding the reverse transcriptase domain or the maturase domain, wherein the ORF has at least 80% sequence identity to any one of SEQ ID NOs: 298-302. In some embodiments, the ORF comprises a sequence having at least 80% sequence identity to any one of SEQ ID NOs: 303-307. In some embodiments, the reverse transcriptase domain or the maturase domain comprises a conserved Y[I/L]DD active site motif of any one of SEQ ID NOs: 729-733.

In some aspects, the present disclosure provides for a method for synthesizing complementary DNA (cDNA), comprising: (a) providing an RNA molecule as a template for cDNA synthesis; (b) providing a primer oligonucleotide to initiate cDNA synthesis from the RNA molecule; and (c) synthesizing cDNA initiated by the primer oligonucleotide from the template using a reverse transcriptase comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 440-554. In some embodiments, the reverse transcriptase comprises a sequence having at least 80% sequence identity to any one of SEQ ID NOs: 518-522, 524-527, and 529-532. In some embodiments, the reverse transcriptase comprises a sequence having at least 80% sequence identity to any one of SEQ ID NOs: 526. In some embodiments, the primer oligonucleotide comprises an oligo (dT) sequence or a degenerate sequence of at least six oligonucleotides. In some embodiments, the synthesizing cDNA comprises incubating the template RNA molecule, the primer oligonucleotide, and the reverse transcriptase in a reaction mixture under conditions suitable for extension of a DNA sequence from the RNA template. In some embodiments, the reaction mixture further comprises dNTPs, a reaction buffer, divalent metal ions, Mg²⁺, or Mn²⁺.

In some aspects, the present disclosure provides for a polypeptide comprising a reverse transcriptase domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 440-554, wherein the sequence is fused N- or C-terminally to a non-retrotransposase domain or an affinity tag. In some embodiments, the reverse transcriptase domain comprises a sequence having at least 80% sequence identity to any one of SEQ ID NOs: 518-522, 524-527, and 529-532. In some embodiments, the reverse transcriptase comprises a sequence having at least 80% sequence identity to SEQ ID NO: 526. In some embodiments, the non-retrotransposase domain is an RNA-binding protein domain. In some embodiments, the RNA binding protein domain comprises a bacteriophage MS2 coat protein (MCP) domain. In some embodiments, the sequence is fused N- or C-terminally to an affinity tag.

In some aspects, the present disclosure provides for a nucleic acid encoding an open reading frame, wherein the open reading frame encodes an RT domain having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RT domain of any one of SEQ ID NOs: 440-554, wherein: (a) the open reading frame is optimized for expression in an organism and the organism is different to the origin of the RT or endonuclease domain; or (b) the ORF comprises a sequence encoding an affinity tag. In some embodiments, the nucleic acid further encodes an RT having at least 80% sequence identity to any one of SEQ ID NOs: 518-522, 524-527, and 529-532. In some embodiments, the reverse transcriptase comprises a sequence having at least 80% sequence identity to SEQ ID NOs: 526. In some embodiments, the open reading frame comprises a sequence having at least 80% sequence identity to any one of SEQ ID NOs: 356-373.

In some aspects, the present disclosure provides for a method for synthesizing complementary DNA (cDNA), comprising: (a) providing an RNA molecule as a template for cDNA synthesis; (b) providing a primer oligonucleotide to initiate cDNA synthesis from the RNA molecule; and (c) synthesizing cDNA initiated by the primer oligonucleotide from the template using a reverse transcriptase comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 609-610, 611-615, 616-617, 618-622, 623, 624-626, 627-673, 1544-1545, and 1555. In some embodiments, the reverse transcriptase domain comprises a conserved xxDD, [F/Y]XDD, NAxxH, or VTG motif of any one of SEQ ID NOs: 609-610, 611-615, 616-617, 618-622, 623, 624-626, 627-673, 1544-1545, and 1555. In some embodiments, the reverse transcriptase comprises a sequence having at least 80% sequence identity to any one of SEQ ID NOs: 612-613, 616-619, 622, 624, 627-630, and 633. In some embodiments, the primer oligonucleotide comprises an oligo (dT) sequence or a degenerate sequence of at least six oligonucleotides. In some embodiments, the primer oligonucleotide comprises at least six consecutive nucleotides having at least 80% sequence identity to any one of SEQ ID NOs: 340-355, 1582-1594, and 1842-1849. In some embodiments, the synthesizing cDNA comprises incubating the template RNA molecule, the primer oligonucleotide, and the reverse transcriptase in a reaction mixture under conditions suitable for extension of a DNA sequence from the RNA template. In some embodiments, the reaction mixture further comprises dNTPs, a reaction buffer, divalent metal ions, Mg²⁺, or Mn²⁺.

In some aspects, the present disclosure provides for a polypeptide comprising a reverse transcriptase domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 609-610, 611-615, 616-617, 618-622, 623, 624-626, 627-673, 1544-1545, 1555, wherein the sequence is fused N- or C-terminally to a non-retrotransposase domain or affinity tag. In some embodiments, the reverse transcriptase domain comprises a conserved xxDD, [F/Y]XDD, NAxxH, or VTG motif of any one of SEQ ID NOs: 609-610, 611-615, 616-617, 618-622, 623, 624-626, 627-673, 1544-1545, and 1555. In some embodiments, the reverse transcriptase domain comprises a sequence having at least 80% sequence identity to any one of SEQ ID NOs: 612-613, 616-619, 622, 624, 627-630, and 633. In some embodiments, the non-retrotransposase domain is an RNA-binding protein domain. In some embodiments, the RNA binding protein domain comprises a bacteriophage MS2 coat protein (MCP) domain. In some embodiments, the sequence is fused N- or C-terminally to an affinity tag.

In some aspects, the present disclosure provides for a nucleic acid encoding an open reading frame (ORF) optimized for expression in an organism, wherein the open reading frame encodes an RT domain having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RT domain of any one of SEQ ID NOs: 609-610, 611-615, 616-617, 618-622, 623, 624-626, 627-673, 1544-1545, and 1555, wherein: (a) the open reading frame is optimized for expression in an organism and the organism is different to the origin of the RT or endonuclease domain; or (b) the ORF comprises a sequence encoding an affinity tag. In some embodiments, the reverse transcriptase domain comprises a conserved xxDD, [F/Y]XDD, NAxxH, or VTG motif of any one of SEQ ID NOs: 609-610, 611-615, 616-617, 618-622, 623, 624-626, 627-673, 1544-1545, or 1555. In some embodiments, the nucleic acid further encodes an RT having at least 80% sequence identity to any one of SEQ ID NOs: 612-613, 616-619, 622, 624, 627-630, and 633. In some embodiments, the ORF comprises a sequence encoding an affinity tag. In some embodiments, the open reading frame comprises a sequence having at least 80% sequence identity to any one of SEQ ID NOs: 66-119, 174-180, 188-192, 198-202, 208-216, 226-230, 236-240, 246-250, 308-309, 310-312, 313-314, 315-319, 320, 321-323, 363-373, 1569-1570, 1571-1580, and 1581. In some embodiments, the organism is different to the origin of the RT domain. In some embodiments, the ORF comprises a sequence having at least 80% sequence identity to any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806.

In some aspects, the present disclosure provides for a synthetic oligonucleotide comprising at least six consecutive nucleotides having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 340-355, 1582-1594, and 1842-1849. In some embodiments, the synthetic oligonucleotide comprises DNA nucleotides. In some embodiments, the oligonucleotide further comprises at least one phosphorothioate linkage.

In some aspects, the present disclosure provides for a vector comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 340-355, 1582-1594, and 1842-1849.

In some aspects, the present disclosure provides for a vector comprising any of the nucleic acids described herein.

In some aspects, the present disclosure provides for a host cell comprising any of the nucleic acids described herein. In some embodiments, the host cell is an E. coli cell. In some embodiments, the E. coli cell is a λDE3 lysogen or the E. coli cell is a BL21 (DE3) strain. In some embodiments, the E. coli cell has an ompT lon genotype. In some embodiments, the nucleic acid comprises an open reading from (ORF) encoding a retrotransposase, a fragment thereof, or a reverse transcriptase domain, wherein the open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araP_{BAD} promoter, a strong leftward promoter from phage lambda (pL promoter), or any combination thereof. In some embodiments, the open reading frame comprises a sequence encoding an affinity tag linked in-frame to a sequence encoding the retrotransposase, the fragment thereof, or the reverse transcriptase domain.

In some aspects, the present disclosure provides for a culture comprising any of the host cells described herein in compatible liquid medium.

In some aspects, the present disclosure provides for a method of producing a retrotransposase, a fragment thereof, or a reverse transcriptase domain comprising cultivating any of the host cells described herein in compatible liquid medium. In some embodiments, the method further comprises inducing expression of the retrotransposase, the fragment thereof, or the reverse transcriptase domain by addition of an additional chemical agent or an increased amount of a nutrient. In some embodiments, the additional chemical agent or increased amount of a nutrient comprises Isopropyl β-D-1-thiogalactopyranoside (IPTG) or additional amounts of lactose. In some embodiments, the method further comprises isolating the host cell after the cultivation and lysing the host cell to produce a protein extract. In some embodiments, the method further comprises subjecting the protein extract to affinity chromatography specific to an affinity tag or ion-affinity chromatography.

In some aspects, the present disclosure provides for an in vitro transcribed mRNA comprising an RNA cognate of any the nucleic acids described herein.

In some aspects, the present disclosure provides for an engineered retrotransposase system, comprising: (a) a double-stranded nucleic acid comprising a cargo nucleotide sequence, wherein the cargo nucleotide sequence is configured to interact with a retrotransposase; and (b) a retrotransposase, wherein: (i) the retrotransposase is configured to transpose the cargo nucleotide sequence to a target nucleic acid locus; and (ii) the retrotransposase is derived from an uncultivated microorganism. In some embodiments, the cargo nucleotide sequence is engineered. In some embodiments, the cargo nucleotide sequence is heterologous. In some embodiments, the cargo nucleotide sequence does not have the sequence of a wild-type genome sequence present in an organism. In some embodiments, the retrotransposase comprises a sequence having at least 75% sequence identity to any one of SEQ ID NOs: 1-29, 393-735, 799-895, 1020-1476, 1544-1554, 1850-2160, 2165-2210, and 2258-2266. In some embodiments, the retrotransposase comprises a reverse transcriptase domain. In some embodiments, the retrotransposase further comprises one or more zinc finger domains. In some embodiments, the retrotransposase further comprises an endonuclease domain. In some embodiments, the retrotransposase has less than 80% sequence identity to a documented retrotransposase. In some embodiments, the cargo nucleotide sequence is flanked by a 3′ untranslated region (UTR) and a 5′ untranslated region (UTR). In some embodiments, the retrotransposase is configured to transpose the cargo nucleotide sequence via a ribonucleic acid polynucleotide intermediate. In some embodiments, the retrotransposase comprises one or more nuclear localization sequences (NLSs) proximal to an N- or C-terminus of the retrotransposase. In some embodiments, the NLS comprises a sequence at least 80% identical to a sequence selected from the group consisting of SEQ ID NO: 1477-1492. In some embodiments, the sequence identity is determined by a BLASTP, CLUSTALW, MUSCLE, MAFFT, or CLUSTALW with the parameters of the Smith-Waterman homology search algorithm. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides for an engineered retrotransposase system, comprising: (a) a double-stranded nucleic acid comprising a cargo nucleotide sequence, wherein the cargo nucleotide sequence is configured to interact with a retrotransposase; and (b) a retrotransposase, wherein: (i) the retrotransposase is configured to transpose the cargo nucleotide sequence to a target nucleic acid locus; and (ii) the retrotransposase comprises a sequence having at least 75% sequence identity to any one of SEQ ID NOs: 1-29, 393-735, 799-895, 1020-1476, 1544-1554, 1850-2160, 2165-2210, and 2258-2266. In some embodiments, the retrotransposase is derived from an uncultivated microorganism. In some embodiments, the retrotransposase comprises a reverse transcriptase domain. In some embodiments, the retrotransposase further comprises one or more zinc finger domains. In some embodiments, the retrotransposase further comprises an endonuclease domain. In some embodiments, the retrotransposase has less than 80% sequence identity to a documented retrotransposase. In some embodiments, the cargo nucleotide sequence is flanked by a 3′ untranslated region (UTR) and a 5′ untranslated region (UTR). In some embodiments, the retrotransposase is configured to transpose the cargo nucleotide sequence via a ribonucleic acid polynucleotide intermediate. In some embodiments, the sequence identity is determined by a BLASTP, CLUSTALW, MUSCLE, MAFFT, or CLUSTALW with the parameters of the Smith-Waterman homology search algorithm. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides for a deoxyribonucleic acid polynucleotide encoding the engineered retrotransposase system of any one of the aspects or embodiments described herein.

In some aspects, the present disclosure provides for a nucleic acid comprising an engineered nucleic acid sequence optimized for expression in an organism, wherein the nucleic acid encodes a retrotransposase, and wherein the retrotransposase is derived from an uncultivated microorganism, wherein the organism is not the uncultivated microorganism. In some embodiments, the retrotransposase comprises at least 75% sequence identity to any one of SEQ ID NOs: 1-29, 393-735, 799-895, 1020-1476 and 1546-1553. In some embodiments, the retrotransposase comprises a sequence encoding one or more nuclear localization sequences (NLSs) proximal to an N- or C-terminus of the retrotransposase. In some embodiments, the NLS comprises a sequence selected from SEQ ID NOs: 1477-1492. In some embodiments, the NLS comprises SEQ ID NO: 1478. In some embodiments, the NLS is proximal to the N-terminus of the retrotransposase. In some embodiments, the NLS comprises SEQ ID NO: 1477. In some embodiments, the NLS is proximal to the C-terminus of the retrotransposase. In some embodiments, the organism is prokaryotic, bacterial, eukaryotic, fungal, plant, mammalian, rodent, or human

In some aspects, the present disclosure provides for a vector comprising the nucleic acid of any one of the aspects or embodiments described herein. In some embodiments, the vector further comprises a nucleic acid encoding a cargo nucleotide sequence configured to form a complex with the retrotransposase. In some embodiments, the vector is a plasmid, a minicircle, a CELiD, an adeno-associated virus (AAV) derived virion, or a lentivirus.

In some aspects, the present disclosure provides for a cell comprising the vector of any one of any one of the aspects or embodiments described herein.

In some aspects, the present disclosure provides for a method of manufacturing a retrotransposase, comprising cultivating the cell of any of the aspects or embodiments described herein.

In some aspects, the present disclosure provides for a method for binding, nicking, cleaving, marking, modifying, or transposing a double-stranded deoxyribonucleic acid polynucleotide, comprising: (a) contacting the double-stranded deoxyribonucleic acid polynucleotide with a retrotransposase configured to transpose the cargo nucleotide sequence to a target nucleic acid locus; wherein the retrotransposase comprises a sequence having at least 75% sequence identity to any one of SEQ ID NOs: 1-29, 393-735, 799-895, 1020-1476, 1544-1554, 1850-2160, 2165-2210, and 2258-2266. In some embodiments, the retrotransposase is derived from an uncultivated microorganism. In some embodiments, the retrotransposase comprises a reverse transcriptase domain. In some embodiments, the retrotransposase further comprises one or more zinc finger domains. In some embodiments, the retrotransposase further comprises an endonuclease domain. In some embodiments, the retrotransposase has less than 80% sequence identity to a documented retrotransposase. In some embodiments, the cargo nucleotide sequence is flanked by a 3′ untranslated region (UTR) and a 5′ untranslated region (UTR). In some embodiments, the double-stranded deoxyribonucleic acid polynucleotide is transposed via a ribonucleic acid polynucleotide intermediate. In some embodiments, the double-stranded deoxyribonucleic acid polynucleotide is a eukaryotic, plant, fungal, mammalian, rodent, or human double-stranded deoxyribonucleic acid polynucleotide.

In some aspects, the present disclosure provides for a method of modifying a target nucleic acid locus, the method comprising delivering to the target nucleic acid locus the engineered retrotransposase system of any one of the aspects or embodiments described herein, wherein the retrotransposase is configured to transpose the cargo nucleotide sequence to the target nucleic acid locus, and wherein the complex is configured such that upon binding of the complex to the target nucleic acid locus, the complex modifies the target nucleic acid locus In some embodiments, modifying the target nucleic acid locus comprises binding, nicking, cleaving, marking, modifying, or transposing the target nucleic acid locus. In some embodiments, the target nucleic acid locus comprises deoxyribonucleic acid (DNA). In some embodiments, the target nucleic acid locus comprises genomic DNA, viral DNA, or bacterial DNA. In some embodiments, the target nucleic acid locus is in vitro. In some embodiments, the target nucleic acid locus is within a cell. In some embodiments, the cell is a prokaryotic cell, a bacterial cell, a eukaryotic cell, a fungal cell, a plant cell, an animal cell, a mammalian cell, a rodent cell, a primate cell, a human cell, or a primary cell. In some embodiments, the cell is a primary cell. In some embodiments, the primary cell is a T cell. In some embodiments, the primary cell is a hematopoietic stem cell (HSC).

In some aspects, the present disclosure provides for a method of any one of the aspects or embodiments described herein, wherein delivering the engineered retrotransposase system to the target nucleic acid locus comprises delivering the nucleic acid of any one of the aspects or embodiments described herein or the vector of any of the aspects or embodiments described herein. In some embodiments, delivering the engineered retrotransposase system to the target nucleic acid locus comprises delivering a nucleic acid comprising an open reading frame encoding the retrotransposase. In some embodiments, the nucleic acid comprises a promoter to which the open reading frame encoding the retrotransposase is operably linked. In some embodiments, delivering the engineered retrotransposase system to the target nucleic acid locus comprises delivering a capped mRNA containing the open reading frame encoding the retrotransposase. In some embodiments, delivering the engineered retrotransposase system to the target nucleic acid locus comprises delivering a translated polypeptide. In some embodiments, the retrotransposase does not induce a break at or proximal to the target nucleic acid locus.

In some aspects, the present disclosure provides for a host cell comprising an open reading frame encoding a heterologous retrotransposase having at least 75% sequence identity to any one of SEQ ID NOs: 1-29, 393-735, 799-895, 1020-1476, 1544-1554, 1850-2160, 2165-2210, and 2258-2266. In some embodiments, the host cell is an E. coli cell. In some embodiments, the E. coli cell is a λDE3 lysogen or the E. coli cell is a BL21 (DE3) strain. In some embodiments, the E. coli cell has an ompT lon genotype. In some embodiments, the open reading frame is operably linked to a T7 promoter sequence, a T7-lac promoter sequence, a lac promoter sequence, a tac promoter sequence, a tre promoter sequence, a ParaBAD promoter sequence, a PrhaBAD promoter sequence, a T5 promoter sequence, a cspA promoter sequence, an araP_{BAD} promoter, a strong leftward promoter from phage lambda (pL promoter), or any combination thereof. In some embodiments, the open reading frame comprises a sequence encoding an affinity tag linked in-frame to a sequence encoding the retrotransposase. In some embodiments, the affinity tag is an immobilized metal affinity chromatography (IMAC) tag. In some embodiments, the IMAC tag is a polyhistidine tag. In some embodiments, the affinity tag is a myc tag, a human influenza hemagglutinin (HA) tag, a maltose binding protein (MBP) tag, a glutathione S-transferase (GST) tag, a streptavidin tag, a FLAG tag, or any combination thereof. In some embodiments, the affinity tag is linked in-frame to the sequence encoding the retrotransposase via a linker sequence encoding a protease cleavage site. In some embodiments, the protease cleavage site is a tobacco etch virus (TEV) protease cleavage site, a PreScission® protease cleavage site, a Thrombin cleavage site, a Factor Xa cleavage site, an enterokinase cleavage site, or any combination thereof. In some embodiments, the open reading frame is codon-optimized for expression in the host cell. In some embodiments, the open reading frame is provided on a vector. In some embodiments, the open reading frame is integrated into a genome of the host cell

In some aspects, the present disclosure provides for a culture comprising the host cell of any one of the aspects or embodiments described herein in compatible liquid medium.

In some aspects, the present disclosure provides for a method of producing a retrotransposase, comprising cultivating the host cell of any one of the aspects or embodiments described herein in compatible growth medium. In some embodiments, the method further comprises inducing expression of the retrotransposase by addition of an additional chemical agent or an increased amount of a nutrient. In some embodiments, the additional chemical agent or increased amount of a nutrient comprises Isopropyl β-D-1-thiogalactopyranoside (IPTG) or additional amounts of lactose. In some embodiments, the method further comprising isolating the host cell after the cultivation and lysing the host cell to produce a protein extract. In some embodiments, the method further comprises subjecting the protein extract to IMAC, or ion-affinity chromatography. In some embodiments, the open reading frame comprises a sequence encoding an IMAC affinity tag linked in-frame to a sequence encoding the retrotransposase. In some embodiments, the IMAC affinity tag is linked in-frame to the sequence encoding the retrotransposase via a linker sequence encoding protease cleavage site. In some embodiments, the protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof. In some embodiments, the IMAC affinity tag by contacting a protease corresponding to the protease cleavage site to the retrotransposase. In some embodiments, the method further comprises performing subtractive IMAC affinity chromatography to remove the affinity tag from a composition comprising the retrotransposase.

In some aspects, the present disclosure provides for a method of disrupting a locus in a cell, comprising contacting to the cell a composition comprising: (a) a double-stranded nucleic acid comprising a cargo nucleotide sequence, wherein the cargo nucleotide sequence is configured to interact with a retrotransposase; and (b) a retrotransposase, wherein: (i) the retrotransposase is configured to transpose the cargo nucleotide sequence to a target nucleic acid locus; (ii) the retrotransposase comprises a sequence having at least 75% sequence identity to any one of SEQ ID NOs: 1-29, 393-735, 799-895, 1020-1476, 1544-1554, 1850-2160, 2165-2210, and 2258-2266; and (iii) the retrotransposase has at least equivalent transposition activity to a documented retrotransposase in a cell. In some embodiments, the transposition activity is measured in vitro by introducing the retrotransposase to cells comprising the target nucleic acid locus and detecting transposition of the target nucleic acid locus in the cells. In some embodiments, the composition comprises 20 pmoles or less of the retrotransposase. In some embodiments, the composition comprises 1 μmol or less of the retrotransposase.

In some aspects, the present disclosure provides for a host cell comprising an open reading frame encoding any of the proteins or polypeptides described herein. In some embodiments, the host cell is an E. coli cell or a mammalian cell. In some embodiments, the host cell is an E. coli cell, wherein the E. coli cell is a λDE3 lysogen or the E. coli cell is a BL21 (DE3) strain. In some embodiments, the E. coli cell has an ompT lon genotype. In some embodiments, the open reading frame is operably linked to a T7 promoter sequence, a T7-lac promoter sequence, a lac promoter sequence, a tac promoter sequence, a tre promoter sequence, a ParaBAD promoter sequence, a PrhaBAD promoter sequence, a T5 promoter sequence, a cspA promoter sequence, an araP_{BAD} promoter, a strong leftward promoter from phage lambda (pL promoter), or any combination thereof. In some embodiments, the open reading frame comprises a sequence encoding an affinity tag linked in-frame to a sequence encoding the protein. In some embodiments, the affinity tag is an immobilized metal affinity chromatography (IMAC) tag. In some embodiments, the IMAC tag is a polyhistidine tag. In some embodiments, the affinity tag is a myc tag, a human influenza hemagglutinin (HA) tag, a maltose binding protein (MBP) tag, a glutathione S-transferase (GST) tag, a streptavidin tag, a strep tag, a FLAG tag, or any combination thereof. In some embodiments, the affinity tag is linked in-frame to the sequence encoding the protein via a linker sequence encoding a protease cleavage site. In some embodiments, the protease cleavage site is a tobacco etch virus (TEV) protease cleavage site, a PreScission® protease cleavage site, a Thrombin cleavage site, a Factor Xa cleavage site, an enterokinase cleavage site, or any combination thereof. In some embodiments, the open reading frame is codon-optimized for expression in the host cell. In some embodiments, the open reading frame is provided on a vector. In some embodiments, the open reading frame is integrated into a genome of the host cell.

In some aspects, the present disclosure provides for a culture comprising any of the host cells described herein in compatible liquid medium.

In some aspects, the present disclosure provides for a method of producing any of the proteins described herein, comprising cultivating any of the host cells described herein encoding any of the proteins described herein in compatible growth medium. In some embodiments, the method further comprises inducing expression of the protein. In some embodiments, the inducing expression of the nuclease is by addition of an additional chemical agent or an increased amount of a nutrient, or by temperature increase or decrease. In some embodiments, an additional chemical agent or an increased amount of a nutrient comprises Isopropyl β-D-1-thiogalactopyranoside (IPTG) or additional amounts of lactose. In some embodiments, the method further comprises isolating the host cell after the cultivation and lysing the host cell to produce a protein extract comprising the protein. In some embodiments, the method further comprises isolating the protein. In some embodiments, the isolating comprises subjecting the protein extract to IMAC, ion-exchange chromatography, anion exchange chromatography, or cation exchange chromatography. In some embodiments, the host cell comprises a nucleic acid comprising an open reading frame comprising a sequence encoding an affinity tag linked in-frame to a sequence encoding the protein. In some embodiments, the affinity tag is linked in-frame to the sequence encoding the protein via a linker sequence encoding a protease cleavage site. In some embodiments, the protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof. In some embodiments, the method further comprises cleaving the affinity tag by contacting a protease corresponding to the protease cleavage site to the protein. In some embodiments, the affinity tag is an IMAC affinity tag. In some embodiments, the method further comprises performing subtractive IMAC affinity chromatography to remove the affinity tag from a composition comprising the protein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts the genomic context of a bacterial retrotransposon. MG140-1 is a predicted retrotransposase (arrow) encoding a Zn-finger DNA binding domain and a reverse transcriptase domain. Regions flanking the retrotransposase display secondary structure that possibly represent binding sites for the retrotransposase (Secondary structure boxes and zoomed images). Regions of similarity with other homologs indicate putative target sites at which the retrotransposon integrated.

FIG. 2 depicts microbial MG retrotransposases (thick black branches on clade 4) are more closely related to Eukaryotic than viral retrotransposases (thin black branches on clade 6). Clade 1: Telomerase reverse transcriptases; clade 2: Group II intron reverse transcriptases; clade 3: Eukaryotic R1 type retrotransposases; clade 4: microbial and Eukaryotic R2 retrotransposases; clade 5: Eukaryotic retrovirus-related reverse transcriptases; and clade 6: viral reverse transcriptases.

FIG. 3 depicts Clades 3 and 4 from the phylogenetic gene tree from FIG. 2. Some microbial MG retrotransposases contain multiple Zn-finger motifs (vertical rectangles), the conserved RVT_1 reverse transcriptase domain, and APE/RLE or other endonuclease domains (top and bottom panel). Some microbial MG retrotransposases lack an endonuclease domain (mid-panel).

FIG. 4 depicts a phylogenetic tree inferred from a multiple sequence alignment of the reverse transcriptase domain from diverse enzymes. RT sequences were derived from DNA, as well as RNA assemblies. Reference RTs were included in the tree for classification purposes.

FIG. 5A depicts a phylogenetic tree inferred from a multiple sequence alignment of RT domains identified from families of non-LTR retrotransposases (MG140, MG146 and MG147) and related RTs (MG148).

FIG. 5B depicts data demonstrating that non-LTR retrotransposases (MG140, MG146 and MG147) contain an RT domain, an endonuclease domain (Endo), and multiple zinc-binding ribbon motifs, while family MG148 RTs lack an endonuclease domain.

FIG. 6A depicts data demonstrating that MG140 R2 retrotransposases contain RT and endonuclease (EN) domains, as well as multiple zinc-fingers, and share between 24% and 26% average amino acid identity (AAI) with the reference Danio rerio R2 retrotransposase (R2Dr).

FIG. 6B depicts data demonstrating that the MG140-47 R2 retrotransposon integrates into 28S rRNA gene. Alignment of the MG140-47 contig to a reference (GQ398061) ribosomal RNA operon shows a large gap in the reference 28S rDNA gene due to integration of the R2 element into the MG140-47 28S rDNA gene (dotted box).

FIG. 7 depicts genomic context of the MG145-45 retrotransposon. The enzyme contains RT and Zinc-finger domains. A partial 18S rDNA gene hit at the 5′ end and poly-A tail at the 3′ end likely delineate the boundaries of the transposon.

FIG. 8A depicts the contig encoding the MG146-1 retrotransposase with RT and endonuclease domains.

FIG. 8B depicts the MG140-17-R2 retrotransposon encoding three genes predicted to be involved in mobilization: RNA recognition motif gene (RRM); endonuclease enzyme; and reverse transcriptase with RT and RNAse H domains.

FIG. 9A depicts genomic context of two members of the MG148 family of RTs. Predicted genes not associated with the RT are displayed as white arrows.

FIG. 9B depicts nucleotide sequence alignment of five members of the MG148 family indicating conserved regions (boxes underneath the sequence) upstream of the RT (arrow annotated over the consensus sequence).

FIG. 10 depicts screening of in vitro activity of RTns family of enzymes by qPCR (MG140). Activity was detected by qPCR using primers that amplify the full-length cDNA product derived from a primer extension reaction containing the respective RT. Samples are derived from RT reactions containing 100 nM substrate. Negative control: no-template water control in the in vitro expression reaction; positive control 1: R2Tg (Taeniopygia guttata); positive control 2: R2Bm (Bombyx mori). The two positive controls are documented R2 retrotransposons. Active candidates, defined as at least 10-fold signal above the negative control, are marked by hatched bars while candidates inactive in these conditions are white bars.

FIG. 11 depicts screening of in vitro activity of RTns family of enzymes by qPCR (MG146, MG147, MG148). Activity was detected by qPCR using primers that amplify the full-length cDNA product derived from a primer extension reaction containing the respective RT. Samples are derived from RT reactions containing 100 nM substrate. Negative control: no-template water control in the in vitro expression reaction; positive control 1: R2Tg (Taeniopygia guttata), a documented R2 retrotransposon. Active candidates, defined as at least 10-fold signal above the negative control, are marked by hatched bars while candidates inactive in these conditions are white bars.

FIG. 12 depicts an assay to assess the fidelity of R2 and R2-like candidates by next generation sequencing. The resulting cDNA product from a primer extension reaction was PCR-amplified and library prepped for NGS. Trimmed reads were aligned to the reference sequence and the frequency of misincorporation was calculated. Background: no-template water control in the in vitro expression reaction; positive control 1: R2Tg (Taeniopygia guttata).

FIG. 13A depicts a phylogenetic tree inferred from a multiple sequence alignment of full-length Group II intron RTs identified from families from diverse classes.

FIG. 13B depicts a summary table of MG families of Group II introns. AAI: average pairwise amino acid identity of MG families to reference Group II intron sequences.

FIGS. 14A-14D depict screening of in vitro activity of GII intron Class C candidates MG153-1 through MG153-21 and MG153-25 through MG153-27 by primer extension assay. For FIG. 14A through FIG. 14C, lane numbers correspond to the following: 1-PURExpress (in vitro expression) no template control, 2-MMLV control RT, 3-TGIRT-III control RT, 4-MarathonRT control RT. Numbering in bold corresponds to gel lanes with active candidates. Results are representative of two independent experiments. FIG. 14A lane numbers 5-14 correspond to candidates MG153-1 through MG153-10. FIG. 14B lane numbers 5-14 correspond to candidates MG153-11 through MG153-20. FIG. 14C lane numbers 5-8 correspond to candidates MG153-21, MG153-25, MG153-26, and MG153-27, respectively. FIG. 14D depicts detection of full-length cDNA production by qPCR. Hatched bars correspond to RTs that generate product at least 10-fold above background. Results were determined from two technical replicates. Arrows in FIG. 14A through FIG. 14C indicate full-length cDNA product (arrow near the top of the gel) and examples of cDNA drop off (lower arrows).

FIGS. 15A-15D depict screening of in vitro activity of GII intron Class C candidates MG153-28 through MG153-37 and MG153-39 through MG153-57 by primer extension assay. For FIG. 15A through FIG. 15C, lane numbers correspond to the following: 1-PURExpress (in vitro expression) no template control, 2-MMLV control RT, 3-TGIRT-III control RT. Numbering in bold corresponds to gel lanes. FIG. 15A lane numbers 4-13 correspond to candidates MG153-28 through MG153-37. FIG. 15B lane numbers 4-13 correspond to candidates MG153-39 through MG153-48. FIG. 15C lane numbers 4-13 correspond to candidates MG153-49 through MG153-57. FIG. 15D depicts detection of full-length cDNA production by qPCR. Hatched bars correspond to RTs that generate product at least 10-fold above background. Results were determined from two technical replicates. Arrows in FIG. 15A through FIG. 15C indicate full-length cDNA product (arrow near the top of the gel) and examples of cDNA drop off (lower arrows).

FIGS. 16A-16B depict screening of in vitro activity of GII intron Class D MG165 family of reverse transcriptases by primer extension assay. For FIG. 16A, lane numbers correspond to the following: 1-PURExpress (in vitro expression) no template control, 2-MMLV control RT, 3-TGIRT-III control RT, 4 through 12-candidates MG165-1 through 9. Numbering in bold corresponds to gel lanes with active candidates. FIG. 16B depicts quantification of full-length cDNA production by qPCR. Hatched bars correspond to RTs that generate product at least 10-fold above background. Results were determined from two technical replicates. Arrows in FIG. 16A indicate full-length cDNA product (arrow near the top of the gel) and examples of cDNA drop off (lower arrows).

FIGS. 17A-17B depict screening of in vitro activity of GII intron Class F MG167 family of reverse transcriptases by primer extension assay. For FIG. 17A, lane numbers correspond to the following: 1-PURExpress (in vitro expression) no template control, 2-MMLV control RT, 3-TGIRT-III control RT, 4 through 8 MG167-1 candidates. Numbering in bold corresponds to gel lanes with active candidates. FIG. 17B depicts quantification of full-length cDNA production by qPCR. Hatched bars correspond to RTs that generate product at least 10-fold above background. Results were determined from two technical replicates. Arrows in FIG. 17A indicate full-length cDNA product (arrow near the top of the gel) and examples of cDNA drop off (lower arrows).

FIG. 18 depicts an assay to assess the fidelity of GII intron Class C RT candidates from the MG153 family by next generation sequencing. The resulting cDNA product from a primer extension reaction was PCR-amplified and library prepped for NGS. Trimmed reads were aligned to the reference sequence and the frequency of misincorporation was calculated. Results were determined from two independent experiments.

FIGS. 19A-19C depict screening to assess the ability of indicated control RTs and GII intron Class C candidates to synthesize cDNA in mammalian cells. FIG. 19A depicts detection of 542 bp (top) and 100 bp (bottom) PCR products by agarose gel analysis. FIG. 19B depicts detection of 542 bp (top) and 100 bp (bottom) PCR products by D1000 TapeStation. FIG. 19C depicts detection of 542 bp PCR products by D1000 TapeStation for additional candidates. Lanes not relevant for the described experiment in FIG. 19A and FIG. 19B are covered by white boxes.

FIG. 20A depicts a phylogenetic tree of full-length G2L4-like RTs. Reference G2L4 sequences and MG172 candidates (dots) are highlighted.

FIG. 20B depicts data demonstrating that columns 277 to 280 of reference and MG172 RTs represent the catalytic residues responsible for reverse transcriptase function.

FIG. 21A depicts a phylogenetic tree of full-length LTR RTs. Reference LTR RT sequences and MG151 candidates (dots) are highlighted.

FIG. 21B depicts genomic context of MG151-82 RT (labeled ORF 7). Predicted domains are shown as labeled boxes and long terminal repeats (LTR) are shown as arrows flanking the LTR transposon.

FIG. 21C depicts 3D structure prediction of MG151-82 showing the protease, RT, RNAse H and integrase domains.

FIG. 22 depicts multiple sequence alignment of full-length pol protein sequences to highlight the protease, RT-RNAse H, and integrase domains. Catalytic residues for the RT, RNAse H, and integrase domains of the MMLV RT are shown by bars under each domain. The protease domain of the MMLV reference sequence is not shown in the alignment.

FIGS. 23A-23C depict screening of in vitro activity of viral candidates MG151-80 through MG151-97 by primer extension assay. For FIG. 23A, lane numbers correspond to the following: 1-RNA template annealed to primer; 2-MMLV control RT; 3-Ty3 control RT; 4 through 9 candidates MG151-80 through 85; 10-RT control. For FIG. 23B, lane numbers correspond to the following: 1-RNA template annealed to primer, 2 through 12 candidates MG151-87 through 97, 13-MMLV control RT. FIG. 23C depicts testing of in vitro activity of Ty3 control RT in different buffer conditions. Lane numbers correspond to the following: 1-PURExpress (in vitro expression) no template control; 2-Buffer A (40 mM Tris-HCl pH 7.5, 0.2 M NaCl, 10 mM MgCl₂, 1 mM TCEP); 3-Buffer B (20 mM Tris pH 7.5, 150 mM KCl, 5 mM MgCl₂, 1 mM TCEP, 2% PEG-8000); 4-Buffer C (10 mm Tris-HCl pH 7.5, 80 mm NaCl, 9 mm MgCl₂, 1 mM TCEP, 0.01% (v/v) Triton X-100); 5-Buffer D (10 mM Tris pH 7.5, 130 mM NaCl, 9 mM MgCl₂, 1 mM TCEP, 10% glycerol). Arrows in FIG. 23A through FIG. 23C indicate full-length cDNA product (arrow near the top of the gel) and examples of cDNA drop off (lower arrows).

FIGS. 24A-24B depict testing of in vitro RT processivity and priming parameters of candidates MG151-89, MG151-92, and MG151-97 on a structured RNA template. For FIG. 24A and FIG. 24B, lane 1: 6,10, and 16 nucleotide oligo markers (arrows); lane 2: 8, 13, and 20 nucleotide oligo marker; lane 3: 43 and 55 nucleotide oligo marker; lanes 4 and 10: 6 nucleotide primer; lanes 5 and 11: 8 nucleotide primer; lanes 6 and 12: 10 nucleotide primer; lanes 7 and 13: 13 nucleotide primer; lanes 8 and 14: 16 nucleotide primer; lanes 9 and 15: 20 nucleotide primer. FIG. 24A lanes 4-9 correspond to reverse transcription reactions containing MMLV with varying primer lengths. MMLV reverse transcribes through the structured RNA hairpin. Lanes 10-15 correspond to reverse transcription reactions containing MG151-89 with varying primer lengths. MG151-89 prefers primer lengths of 16 and 20 nucleotides and appears to stop reverse transcription at the structured RNA hairpin. FIG. 24B lanes 4-9 correspond to reverse transcription reactions containing MG151-92 with varying primer lengths. Lanes 10-15 correspond to reverse transcription reactions containing MG151-97 with varying primer lengths. Neither MG151-92 or MG151-97 appear active under these experimental conditions.

FIG. 25 depicts phylogenetic analysis of 2407 Retron RTs, with the first candidates selected for downstream characterization in vitro highlighted. 9 of 16 experimentally validated retrons in the literature were added and highlighted in the tree. Stars represent candidate MG154-MG159 and MG173 family members.

FIG. 26 depicts genomic context of the MG157-1 retron (arrow labeled RT on a line). Retron non-coding RNA (ncRNA) is highlighted with a dotted box.

FIG. 27A depicts an inset showing the MG157-1 retron ncRNA with its flanking inverted repeats.

FIG. 27B depicts the predicted structure of the MG157-1 retron ncRNA.

FIG. 28A depicts genomic context of the MG160-3 retron-like single-domain RT. The region upstream from the RT (dotted box) is conserved across MG160 members.

FIG. 28B depicts 3D structure prediction of MG160-3 showing the RT domain aligned to a group II intron cryo-EM structure.

FIG. 28C depicts predicted structures of the 5′ UTR of five MG160 members.

FIGS. 29A-29B depict screening of in vitro activity of retron-like candidates MG160-1 through MG160-6 and MG160-8 by primer extension assay. FIG. 29A lane numbers correspond to the following samples: 1-PURExpress (in vitro expression) no template control, 2-MMLV control RT, 3-TGIRT-III control RT, 4 through 10 candidates MG160-1 through MG160-6 and MG160-8. Numbering in bold corresponds to gel lanes with active candidates. FIG. 29B depicts quantification of full-length cDNA production by qPCR. Hatched bars correspond to RTs that generate product at least 10-fold above background. Results were determined from two technical replicates. Arrows in FIG. 29A indicate full-length cDNA product (arrow near the top of the gel) and examples of cDNA drop off (lower arrows).

FIGS. 30A-30C depict cell-free expression of retron RT candidates and generation of retron ncRNAs by in vitro transcription. FIG. 30A depicts confirmation of retron RT protein production in a cell-free expression system. Lanes correspond to the following: 1: ladder, 2: no template control, 3: MG156-1 (39 kDa), 4: MG156-2 (40 kDa), 5: MG157-1 (38 kDa). FIG. 30B depicts confirmation of retron RT protein production in a cell-free expression system. Lanes correspond to the following-1: ladder, 2: no template control, 3: MG157-2 (37 kDa), 4: MG157-5 (43 kDa), 5: MG159-1 (53 kDa), 6: Ec86 (38 kDa, positive control retron RT). FIG. 30C depicts generation of retron ncRNA templates by in vitro transcription. Lanes correspond to the following ncRNAs corresponding to the following retrons-1: MG154-1, 2: MG154-2, 3: MG155-1, 4: MG155-2, 5: MG155-3, 6: MG156-1, 7: MG156-2, 8: MG157-1, 9: MG157-2, 10: MG157-5, 11: MG158-1, 12: MG159-1, 13: Ec86, 14: MG155-4, 15: MG173-1, 16: MG155-5.

FIG. 31 depicts domain architecture demonstrating that the MG140-1 R2 retrotransposon integrates into 28S rRNA gene. The R2 retrotransposase (less dense hatched bar) contains multiple Zn-fingers, as well as RT and endonuclease domains. MG140-1 is flanked by 5′ and 3′ UTRs, which define the transposon boundaries. MG140-1 integrates precisely between the G and T nucleotides in the target site motif GGTAGC.

FIG. 32 depicts the testing of RT activity by primer extension with DNA oligo containing phosphorothioate bond modifications. Lane numbers correspond to the following, 1: PURExpress (in vitro expression) no template control with PS-modified Primer 1, 2: PURExpress (in vitro expression) no template control with PS-modified Primer 2, 3: PURExpress (in vitro expression) no template control with PS-modified Primer 3, 4: MMLV RT with unmodified primer, 5: MMLV RT with PS-modified primer 1, 6: MMLV RT with PS-modified primer 2, 7: MMLV RT with PS-modified primer 3, 8: TGIRT-III with unmodified primer, 9: TGIRT-III with PS-modified primer 1, 10: TGIRT-III with PS-modified primer 2, 11: TGIRT-III with PS-modified primer 3, 12: MG153-9 with unmodified primer, 13: MG153-9 with PS-modified primer 1, 14: MG153-9 with PS-modified primer 2, 15 MG153-9 with PS-modified primer 3. MMLV RT and TGIRT-III are control RTs.

FIG. 33 depicts the screening of activity of retron RTs on an RNA template by primer extension assay. Lane numbers correspond to the following, 1: PURExpress (in vitro expression) no template control, 2: MMLV control RT, 3: MG154-1, 4: MG155-1, 5: MG155-2, 6: MG155-3, 7: MG156-2, 8: MG157-1, 9: MG157-2, 10: MG157-5, 11: MG158-1, 12: MG159-1, 13: Ec86 control retron RT, 14: Sa163 control retron RT, 15: St85 control retron RT. Lanes in bold correspond to retron RTs that exhibit primer extension activity on the tested substrate.

FIG. 34 depicts the screening of the ability of MG153 GII derived RTs to synthesize cDNA in mammalian cells. Detection of 542 bp cDNA synthesis PCR products were assayed by Taqman qPCR. cDNA activity was normalized to the activity TGIRT control where TGIRT represents a value of 1. Y axis is shown in log 10 scale.

FIGS. 35A-35C depict protein expression of MG153 GII derived RTs by immunoblots. Cells were transfected with plasmids containing the candidate RTs and protein expression was evaluated by immunoblot, detecting the HA peptide fused to the N termini of the RTs. All lanes were normalized to total protein concentration. White arrows point to bands at 2× the expected molecular size of the protein, which indicate protein dimers. Lanes not relevant for the described experiment in FIGS. 35A and 35B are covered by white boxes. FIG. 35C: Multiple sequence alignment of GII derived RT. The region shown corresponds to positions 196 through 201 of the alignment. The dimerization motif CAQQ (SEQ ID NO: 2267) is highlighted.

FIG. 36 depicts relative activity of GII derived RTs normalized to protein expression. cDNA synthesis was detected by Taqman qPCR, protein expression was detected by immunoblots. Activity relative to TGIRT was normalized per total protein concentration. Y axis is shown in a linear scale.

FIGS. 37A-37E depict retroviral RTs for cDNA synthesis. FIG. 37A depicts a phylogenetic tree of full-length LTR RTs. MG151 candidates (grey branches) and a new group of RTs belonging to betaretrovirus (star) are highlighted. FIG. 37B depicts structural alignment of an MG RT domain (dark grey) to reference RT domains from a simian retrovirus and mouse mammary tumor virus (light grey). FIG. 37C depicts a screen of in vitro cDNA synthesis activity of the MG151 family of Retroviral RTs. Lane numbers correspond to the following samples: lane 1: PURExpress (in vitro expression) no template control; lane 2: MMLV control RT; lane 3: MG151-98; lane 4: MG151-99; lane 5: MG151-100; lane 6: MG151-101; lane 7: MG151-102; lane 8: MG151-103; lane 9: MG151-104; lane 10: MG151-105. Lane numbers in bold corresponds to gel lanes with active candidates. Arrows indicate full-length cDNA product (arrow near the top of the gel) and lines indicate examples of cDNA drop off. FIG. 37D depicts a screen of in vitro cDNA synthesis activity of the MG151 family of Retroviral RTs. Lane numbers correspond to the following samples: lane 1: PURExpress (in vitro expression) no template control; lane 2: MMLV control RT; lane 3: MG151-106; lane 4: MG151-107; lane 5: MG151-108; lane 6: MG151-109; lane 7: MG151-110; lane 8: MG151-111; lane 9: MG151-112; lane 10: MG151-113; lane 11: MG151-114; lane 12: MG151-115; lane 13: MG151-116; lane 14: MG151-117. Lane numbers in bold corresponds to gel lanes with active candidates. Arrows indicate full-length cDNA product (arrow near the top of the gel) and lines indicate examples of cDNA drop off. FIG. 37E depicts a screen of in vitro cDNA synthesis activity of the MG151 family of Retroviral RTs with unmodified and modified RNA substrate. Lane numbers correspond to the following samples: lane 1: PURExpress (in vitro expression) no template control using uridine-containing RNA (U-RNA) substrate; lane 2: PURExpress (in vitro expression) no template control using N1-methylpsuedouridine-containing RNA (m1Ψ-RNA) substrate; lane 3: MMLV control RT using U-RNA substrate; lane 4: MMLV control RT using m1Ψ-RNA substrate; lane 5: MG151-118 using U-RNA substrate; lane 6: MG151-118 using m1Ψ-RNA substrate; lane 7: MG151-119 using U-RNA substrate; lane 8: MG151-119 using m1Ψ-RNA substrate; lane 9: MG151-120 using U-RNA substrate; lane 10: MG151-120 using m1Ψ-RNA substrate; lane 11: MG151-121 using U-RNA substrate; lane 12: MG151-121 using m1Ψ-RNA substrate; lane 13: MG151-122 using U-RNA substrate; lane 14: MG151-122 using m1Ψ-RNA substrate; lane 15: MG151-123 using U-RNA substrate; lane 16: MG151-123 using m1Ψ-RNA substrate; lane 17: MG151-124 using U-RNA substrate; lane 18: MG151-124 using m1Ψ-RNA substrate; lane 19: MG151-125 using U-RNA substrate; lane 20: MG151-125 using m1Ψ-RNA substrate; lane 21: MG151-126 using U-RNA substrate; lane 22: MG151-126 using m1Ψ-RNA substrate; lane 23: MG151-127 using U-RNA substrate; lane 24: MG151-127 using m1Ψ-RNA substrate; lane 25: MG151-128 using U-RNA substrate; lane 26: MG151-128 using m1Ψ-RNA substrate. Lane numbers in bold corresponds to gel lanes with active candidates. Arrows indicate full-length cDNA product (arrow near the top of the gel) and lines indicate examples of cDNA drop off.

FIG. 38A depicts a phylogenetic tree of full-length retron and MG160 RTs. MG160 candidates (grey dots) are highlighted within a long divergent branch within the retron clade.

FIG. 38B depicts a structural alignment of MG160 RT (dark grey) to a reference retron RT from E. coli (Ec86, light grey). The additional N-terminus end in Ec86 is boxed.

FIG. 38C depicts multiple sequence alignment of full-length MG160 RTs to the reference Ec86 retron RT. The N-terminus region, RT domain, and C-Terminus regions are shown as bars under the reference sequence, and catalytic residues are highlighted with boxes.

FIG. 38D depicts multiple sequence alignment regions of active MG160 RTs vs. group II intron and retron reference sequences. Enzyme-specific motifs are highlighted with boxes underneath the sequence as follows: MG160-specific motifs AXXXH and GX(3)Y[V/L]XXVN (SEQ ID NO: 2268); retron-specific motifs NAXXH and VTG; group II intron-specific motifs GXXXY (partially shared with MG160 enzymes) and FLG. A conserved histidine residue and motif [N/S]XXK found in most RTs is also highlighted.

FIG. 38E depicts a screen of in vitro cDNA synthesis activity of the MG154, MG155, MG156, MG157, MG158, MG159, and MG160 families of retron and retron-like RTs. Lane numbers correspond to the following samples: lane 1: PURExpress (in vitro expression) no template control; lane 2: MMLV control RT; lane 3: MG160-28; lane 4: MG160-31; lane 5: MG160-37; lane 6: MG160-40; lane 7: MG160-51; lane 8: MG160-52; lane 9: MG160-53; lane 10: MG160-54; lane 11: MG160-55; lane 12: MG160-56; lane 13: MG160-57; lane 14: MG160-58; lane 15: MG160-59; lane 16: MG160-60; lane 17: MG160-61; lane 18: not relevant lane; lane 19: MG160-63; lane 20: MG160-64; lane 21: MG160-65; lane 22: MG160-66; lane 23: MG160-67; lane 24: MG155-4; lane 25: MG155-5; lane 26: MG173-1. Lanes 3-23 correspond to the retron-like MG160 family of RTs. Lanes 24-26 correspond to retron RTs. Lane numbers in bold corresponds to gel lanes with active candidates. Arrows indicate full-length cDNA product (arrow near the top of the gel) and examples of cDNA drop off (lower arrows).

FIG. 38F depicts a screen of in vitro cDNA synthesis activity of the MG154, MG155, MG156, MG157, MG158, and MG159 families of retron RTs. Lane numbers correspond to the following samples: lane 1: PURExpress (in vitro expression) no template control; lane 2: MMLV control RT; lane 3: MG154-1; lane 4: MG155-1; lane 5: MG155-2; lane 6: MG155-3; lane 7: MG156-2; lane 8: MG157-1; lane 9: MG157-2; lane 10: MG157-5; lane 11: MG158-1; lane 12: MG159-1; lane 13: Ec86 control retron RT; lane 14: Sa163 control retron RT; lane 15: St85 control retron RT. Lane numbers in bold corresponds to gel lanes with active candidates. Arrows indicate full-length cDNA product (arrow near the top of the gel) and examples of cDNA drop off (lower lines).

FIG. 38G depicts a screen of in vitro cDNA synthesis activity of the MG154, MG155, MG156, MG157, MG158, MG159, MG160, and MG173 families of retron and retron-like RTs. Lane numbers correspond to the following samples: lane 1: PURExpress (in vitro expression) no template control; lane 2: MMLV control RT; lane 3: TGIRT-III control RT; lanes 4-7: unrelated MG RTs; lane 8: MG160-17; lane 9: MG154-2; lane 10: MG156-1; lane 11: MG157-3; lane 12: MG157-4; lane 13: MG159-2; lane 14: MG159-3; lane 15: MG173-2. Lane 8 corresponds to a retron-like MG160 family of RTs. Lanes 9-15 correspond to MG retron RTs. Lane numbers in bold corresponds to gel lanes with active candidates. Arrows indicate full-length cDNA product (arrow near the top of the gel) and examples of cDNA drop off (lower arrows or vertical line).

FIGS. 39A-39D depict a screen of in vitro cDNA synthesis activity of GII intron RTs. For FIG. 39A, lane numbers correspond to the following samples: lane 1: PURExpress (in vitro expression) no template control; lane 2: MMLV control RT; lane 3: TGIRT-III control RT; lane 4: MG153-38; lanes 5-9: MG163-1 through MG163-5; lanes 10-13: MG166-2 through MG166-5. For FIG. 39B, lane numbers correspond to the following samples: lane 1: PURExpress (in vitro expression) no template control; lane 2: MMLV control RT; lane 3: TGIRT-III control RT; lanes 4-14: MG169-1 through MG169-11. For both panels, lane numbers in bold corresponds to gel lanes with active candidates. Arrows indicate full-length cDNA product (arrow near the top of the gel) and examples of cDNA drop off (lower arrows). FIG. 39C depicts a screen of in vitro activity of GII intron Class C, A, B, E, G, ML, and CL (MG153, MG163, MG164, MG166, MG168, MG169, and MG170). Quantification of full-length cDNA production by qPCR. Loosely hatched bars correspond to RTs that produce sufficient cDNA for gel detection. Tightly hatched bars correspond to RTs that have detectable activity only by qPCR and generate product at least 10-fold above background. FIG. 39D depicts a summary of GII intron Class A-G, Class ML, and Class CL cDNA synthesis activity in vitro. RT activity normalized to TGIRT was determined from quantification of full-length cDNA product after performing primer extension using a 202 nt RNA template.

FIG. 40 depicts screen of in vitro activity of R2 MG140 and MG146 families by primer extension assay with quantification of full-length cDNA production by qPCR. Active RTs are those that generated product at least 10-fold above background (Purex) (dotted line). Results were determined from two technical replicates. Purex is PURExpress (in vitro expression) no-template control; MMLV and Tg R2 are control RTs.

FIGS. 41A-41B depict primer extension activity of GII intron RTs in vitro on a 4.1 kb RNA template. FIG. 41A depicts a schematic of primer extension assay and detection of cDNA products by Taqman qPCR. The RNA template contains MS2 loops located 3′ of the DNA priming oligo. The resulting full-length cDNA product from the RNA template is 4.1 kb. Taqman probes and primers are designed to quantify amplification of the first (FAM) and last (HEX) 100 bp amplicons of the cDNA. FIG. 41B depicts the percentage of products corresponding to the end of the cDNA (HEX) versus beginning (FAM), which was quantified for MG RTs. TGIRT is a GII Class C control RT, and MMLV is a retroviral control RT.

FIG. 42 depicts a cartoon showing the methodology used to detect cDNA synthesis in mammalian cells. The first (FAM) and last (HEX) 100 bps of a 4.1 kb RNA template are detected using Taqman based qPCR.

FIGS. 43A-43I depict a screen of the ability of indicated control RTs and GII intron candidates to synthesize cDNA in mammalian cells. Taqman qPCR was used to detect the first (FAM probe) and last (HEX probe) 100 bp PCR products amplified from cDNA synthesized from an RNA template by the following GII intron RTs: Class A MG163 candidates (FIG. 43A); Class B MG164 candidates (FIG. 43B); Class C MG153 candidates (FIG. 43C); Class D MG165 candidates (FIG. 43D); Class E MG166 candidates (FIG. 43E); Class F MG167 candidates (FIG. 43F); Class G MG168 candidates (FIG. 43G); Class ML MG169 candidates (FIG. 43H); and Class CL MG170 candidates (FIG. 43I).

FIG. 44 depicts a screen of the ability of indicated control RTs and R2 RT candidates to synthesize cDNA in mammalian cells. Taqman qPCR was used to detect the first (FAM probe) and last (HEX probe) 100 bp PCR products amplified from cDNA synthesized from an RNA template by the indicated R2 RT candidates.

FIGS. 45A-45B depict a screen of the ability of the indicated group II intron and R2 RT candidates to synthesize cDNA in mammalian cells, with and without an MCP tag. Taqman qPCR was used to detect the first (FAM probe) and last (HEX probe) 100 bp PCR products amplified from cDNA synthesized from an RNA template by the indicated group II intron and R2 RT candidates, as well as control TGIRT group II intron and R2Tg R2 RTs.

FIG. 46A depicts primer conversion activity of the MG151 family of RTs on standard (U) vs. modified (m1Ψ) RNA template. RT primer extension activity is normalized to MMLV, a control retroviral RT.

FIG. 46B depicts primer extension activity of diverse RTs on standard and m1Ψ-modified RNA template. Lane numbers correspond to the following samples: lane 1: PURExpress (in vitro expression) NTC with standard RNA template; lane 2: PURExpress (in vitro expression) NTC with m1Ψ-modified RNA template; lane 3: MMLV control RT with standard RNA template; lane 4: MMLV control RT with m1Ψ-modified RNA template; lane 5: TGIRT control RT with standard RNA template; lane 6: TGIRT control RT with m1Ψ-modified RNA template; lane 7: MG153-18 with standard RNA template; lane 8: MG153-18 with m1Ψ-modified RNA template; lane 9: MG153-20 with standard RNA template; lane 10: MG153-20 with m1Ψ-modified RNA template; lane 11: MG153-51 with standard RNA template; lane 12: MG153-51 with m1Ψ-modified RNA template; lane 13: MG153-56 with standard RNA template; lane 14: MG153-56 with m1Ψ-modified RNA template; lane 15: MG170-1 with standard RNA template; lane 16: MG170-1 with m1Ψ-modified RNA template; lane 17: MG140-3 with standard RNA template; lane 18: MG140-3 with m1Ψ-modified RNA template; lane 19: MG140-8 with standard RNA template; lane 20: MG140-8 with m1Ψ-modified RNA template; lane 21: MG140-46 with standard RNA template; lane 22: MG140-46 with m1Ψ-modified RNA template; lane 23: Tg R2 control RT with standard RNA template; lane 24: Tg R2 control RT with m1Ψ-modified RNA template; lane 25: MG160-4 with standard RNA template; lane 26: MG160-4 with m1Ψ-modified RNA template. Arrows indicate full-length cDNA product (arrow near the top of the gel) and examples of cDNA drop off (lower vertical line).

FIGS. 46C-46D depict quantification of RT activity on standard vs. modified template for diverse RTs. FIG. 46C depicts quantification of primer conversion by gel analysis. Results were determined from two independent experiments. FIG. 46D depicts quantification of full-length cDNA production by qPCR performed for candidates with little or no detectable primer conversion on denaturing gel. Results were determined from two technical qPCR replicates.

FIGS. 47A-47C depict a screen of the ability of indicated control RTs and candidates RTs to synthesize cDNA in mammalian cells. FIG. 47A depicts a schematic illustration of the methodology used to detect cDNA synthesis in mammalian cells. The first (FAM) and last (HEX) 100 bps of a 4.1 kb RNA template are detected using Taqman based qPCR. Taqman qPCR was used to detect the first (FAM probe) and last (HEX probe) 100 bp PCR products amplified from cDNA synthesized from an RNA template by MG148 family of non-LTR retrotransposon derived RTs (FIG. 47B) and MG160 family of retron-like RTs (FIG. 47C).

FIG. 48 depicts a screen of rationally engineered mutants of optimal RT candidates MG153-18 and MG153-20 for their ability to synthesize cDNA in mammalian cells. Taqman qPCR detection of first (FAM probe) and last (HEX probe) 100 bp PCR products amplified from cDNA synthesized from an RNA template by indicated control and selected RT candidates. MG153-18 variants showed increased activity by 5 fold compared to its WT counterpart while MG153-20 variants did not improve activity.

FIG. 49 depicts a screen of putative inactivating mutants of indicated control RTs and optimal group II intron-derived and R2 RT candidates for their ability to synthesize cDNA in mammalian cells. Taqman qPCR detection of first (FAM probe) and last (HEX probe) 100 bp PCR products amplified from cDNA synthesized from an RNA template by indicated control and selected RT candidates.

FIG. 50 depicts a schematic overview of the mechanism of Retron that produces multiple copy single stranded DNA (msDNA).

FIG. 51 depicts SDS-PAGE analysis of expression of MG173 and MG192 family from PURExpress. Protein expression marked with an arrow. Lane numbers correspond to the following: Lane 1: Protein ladder; Lane 2: No template control (NTC); Lane 3: MG173-3; Lane 4: MG173-4; Lane 5: MG173-5; Lane 6: Skip; Lane 7: MG173-6; Lane 8: MG173-7; Lane 9: Protein Ladder; Lane 10: No template control (NTC); Lane 11: MG173-8; Lane 12: MG173-9; Lane 13: MG173-10; Lane 14: MG192-1.

FIG. 52 depicts a screen of generic in vitro cDNA synthesis activity of MG173 and MG192 family of retron RTs. Lane numbers correspond to the following samples: Lane 1: PURExpress no template control, RT reaction does not contain a reverse transcriptase; Lane 2: positive control retroviral RT MMLV; Lane 3: positive control retron RT Ec86; Lanes 4-11: MG173-3 through MG173-10; Lane 12: MG192-1. Lane numbers in bold corresponds to gel lanes with active candidates. Arrows indicate full-length cDNA product (arrow near the top of the gel) and examples of cDNA drop off (lower arrows or vertical line).

FIGS. 53A and 53B depict in vitro primer extension activity of retron RTs on a 4.1 kb RNA template. FIG. 53A depicts a schematic of primer extension assay and detection of cDNA products by Taqman qPCR. RNA template is annealed to a priming oligo prior to initiation of the cDNA synthesis reaction. The resulting full-length cDNA product from the RNA template is 4.1 kb. Taqman probes and primers are designed to quantify amplification of the first (FAM) and last (HEX) 100 bp amplicons of the cDNA. FIG. 53B depicts the percentage of products corresponding to the end of the cDNA (HEX) versus beginning (FAM) quantified for MG RTs. TGIRT is GII Class C control RT, MMLV is a retroviral control RT, and Ec86 is a retron control RT.

FIG. 54 depicts the RT error substitution rates of GII intron positive control RT TGIRT and MG GII intron RTs MG153-5, MG153-18, MG153-20, MG153-51, and MG153-53 on standard and modified (N1-methyl pseudouridine, m1′) RNA templates.

FIGS. 55A-55D depict a screen of the ability of indicated control RTs and engineered candidate RTs to synthesize cDNA in mammalian cells. FIG. 55A shows a cartoon depicting methodology used to detect cDNA synthesis in mammalian cells. The first (FAM) and last (HEX) 100 bps of a 4.1 kb RNA template are detected using Taqman based qPCR. FIGS. 55B-55D show Taqman qPCR detection of first (FAM probe) and last (HEX probe) 100 bp per products amplified from cDNA synthesized from an RNA template by MG140-3 and MG140-8 variants of non-LTR retrotransposon derived RTs (FIG. 55B), MG153-5, MG153-51, and MG169-1 variants of GII intron RTs (FIG. 55C), and MG153-18 and MG153-20 variants of GII intron RTs (FIG. 55D).

FIGS. 56A-56D depict a screen of the ability of indicated control RTs and candidate RTs to synthesize cDNA in mammalian cells. FIGS. 56A-56D show Taqman qPCR detection of first (FAM probe) and last (HEX probe) 100 bp per products amplified from cDNA synthesized from an RNA template by MG140 family of non-LTR retrotransposon derived RTs (FIG. 56A), MG169 family of GII intron derived RTs (FIG. 56B), MG153 family of GII intron derived RTs (FIG. 56C), and retron RTs (FIG. 56D).

FIGS. 57A-57B depict analysis of protein expression of selected RT candidates by Western blot. Western blot analysis of MG153-18 and MG153-20 variants of GII intron RTs (FIG. 57A) and selected candidates of GII intron Rts and R2 RTs with high cDNA synthesis activity and processivity (FIG. 57B). Blot showing anti-HA (top) and anti-cyclophilin (bottom). *indicates a non-specific band detected in the anti-HA blot.

FIGS. 58A-58B depict a screen of the ability of indicated control RTs and trimmed candidate RTs to synthesize cDNA in mammalian cells. Taqman qPCR detection of first (FAM probe) and last (HEX probe) 100 bp PCR products amplified from cDNA synthesized from an RNA template by trimmed variants of MG140-3 and MG140-8 family of non-LTR retrotransposon derived RTs (FIG. 58A) and MG140-74 and MG140-88 family of non-LTR retrotransposon derived RTs (FIG. 58B) in comparison to the activity of endonuclease domain (ED) inactivated and/or reverse transcriptase (RT) domain inactivated RT versions.

FIGS. 59A-59B depict RT substitution error rates. FIG. 59A shows substitution error rate of RTs calculated from consensable UMI sequences as mismatches/(matches+mismatches) for standard (U) and modified (m1Ψ) RNA templates. Bar graph displays the mean and upper and lower bars indicate the 95% CI determined by Bayesian analysis. Data are derived from two independent experiments each of which were performed in technical triplicate. FIG. 59B shows theoretical length of substitution-free cDNA molecule for each RT calculated as 1/(substitution error rate) for standard and modified RNA templates. MMLV, TGIRT, and MarathonRT are referred to in the text as Control 1, Control 2, and Control 3 respectively.

FIG. 60 depicts RT error type (mismatch, insertion, or deletion) by position along the standard RNA template. Arrows indicate substitution (or mismatch) hotspot shared between RTs at position 78. The inset image shows a portion of the predicted RNA template fold and that position 78 is located within a putative hairpin. MMLV, TGIRT, and MarathonRT are referred to as Control 1, Control 2, and Control 3 respectively.

FIG. 61 depicts RT error type (mismatch, insertion, or deletion) by position along the modified RNA template. MMLV, TGIRT, and MarathonRT are referred to as Control 1, Control 2, and Control respectively.

FIG. 62 depicts RT substitution preference on standard or modified (RNA templates displayed as a confusion matrix comparing the reference nucleotide to the observed nucleotide identity. MMLV, TGIRT, and MarathonRT are referred to as Control 1, Control 2, and Control 3 respectively.

FIG. 63 depicts RT indel analysis on the standard RNA template, displaying frequency and size of each observed insertion (positive number) or deletion (negative number). MMLV, TGIRT, and MarathonRT are referred to as Control 1, Control 2, and Control 3 respectively.

FIG. 64 depicts RT indel analysis on the modified RNA template, displaying frequency and size of each observed insertion (positive number) or deletion (negative number). MMLV, TGIRT, and MarathonRT are referred to as Control 1, Control 2, and Control 3 respectively.

FIG. 65 depicts RT distribution of cDNA length on standard template, showing cDNA drop-off products, full-length, and non-templated additions (NTA). MMLV, TGIRT, and MarathonRT are referred to as Control 1, Control 2, and Control 3 respectively.

FIG. 66 depicts RT distribution of cDNA length on modified template, showing cDNA drop-off products, full-length, and non-templated additions (NTA). MMLV, TGIRT, and MarathonRT are referred to as Control 1, Control 2, and Control 3 respectively.

FIG. 67 depicts analysis of RT non-templated addition (NTA) nucleotide incorporation preference on standard RNA template. MMLV, TGIRT, and MarathonRT are referred to as Control 1, Control 2, and Control 3 respectively.

FIG. 68 depicts analysis of RT non-templated addition (NTA) nucleotide incorporation preference on modified RNA template. MMLV, TGIRT, and MarathonRT are referred to as Control 1, Control 2, and Control 3 respectively.

FIG. 69 depicts expression screen of MG140-8c5. Medium throughput heterologous expression screen of MG140-8c5 in E. coli. Constructs are expressed in small-scale culture flasks and induced at various temperatures in different growth media. Purification is performed in a 24 deep-well plate, and eluates are run on a gel for analysis. The data show that MG140-8c5 can be purified from the pMGE expression vector with a SUMO fusion, while expression in the pMGD vector with an MBP fusion does not yield full-length protein.

FIGS. 70A-70D depict large-scale MG140-8c5 expression and purification. MG140-8c5 induced at either 16° C. overnight (FIGS. 70A-70B) or at 23.5° C. for 5 hrs (FIGS. 70C-70D) purified over a 5 mL HisTrap was eluted with an imidazole gradient. Elution profiles monitoring A280 and A260 show significantly higher A260 levels in the 23.5° C. purification (presumably more nucleic acid contamination, FIG. 70C) than in the 16° C. purification (FIG. 70A). Sample run on a gel revealed elution of the protein of interest (~116 kDa) at relatively high imidazole concentrations.

FIG. 71 depicts primer extension activity of MG140-8c5 on standard and modified RNA template. Gel lanes correspond to the following samples: Lane 1: no RT control, standard template; Lane 2: no RT control, modified template; Lane 3: MMLV control enzyme 1, standard template, replicate 1; Lane 4: MMLV control enzyme 1, modified template, replicate 1; Lane 5:140-8c5, standard template, replicate 1; Lane 6:140-8c5, modified template, replicate 1; Lane 7: AccuScript control enzyme 4, standard template, replicate 1; Lane 8: AccuScript control enzyme 4, modified template, replicate 1; Lane 9: MMLV control enzyme 1, standard template, replicate 2; Lane 10: MMLV control enzyme 1, modified template, replicate 2; Lane 11:140-8c5, standard template, replicate 2; Lane 12:140-8c5, modified template, replicate 2; Lane 13: AccuScript control enzyme 4, standard template, replicate 2; and Lane 14: AccuScript control enzyme 4, modified template, replicate 2.

FIGS. 72A-72B depict the use of fluorescence anisotropy to detect strand displacement during cDNA synthesis. FIG. 72A: A substrate template RNA is annealed to a priming oligo and a displacement oligo conjugated to a FAM fluorophore. In the annealed state, the fluorophore tumbles slowly and emitted light is not depolarized. Upon strand displacement, the much-smaller oligo-conjugated FAM tumbles in solution much faster and depolarizes light after emission. FIG. 72B: Reactions containing annealed substrate template and purified MG140-8c5 enzyme are initiated by the addition of dNTPs, allowing the enzyme to polymerize cDNA and displace the FAM-labeled oligo, which is detectable by a depolarization of emitted light. By comparison, substrate template without dNTPs maintains emits polarized light due to lack of displacement, and FAM-oligo-only controls depolarize emitted light to a high degree.

FIGS. 73A-73B depict the use of fluorescence unquenching to detect strand displacement during second-strand synthesis. FIG. 73A: A substrate template ssDNA, synthesized with a 5′ FAM fluorophore conjugation, is annealed to a priming oligo and a displacement oligo with a 3′ quencher moiety. In the annealed state, the fluorophore is quenched by the displacement oligo's quencher molecule, and fluorescence is low. Upon strand displacement, the template FAM is no longer quenched and an increase in fluorescence is observed. FIG. 73B: Reactions containing annealed substrate template and purified MG140-8c5 enzyme are initiated by the addition of dNTPs, allowing the enzyme to polymerize second-strand DNA and displace the quenching oligo thus producing an increase in fluorescence. By contrast, reactions without dNTPs added do not depict the same gradual increase in fluorescence over time

FIGS. 74A-74B depict the use of strand displacement to measure enzyme activity from PURExpress. FIG. 74A: 1004 nt ssDNA template was produced by PCR amplification followed by Lambda Exonuclease digestion. FIG. 74B: Fluorescence-unquenching assays were set up using template annealed to a priming oligo and a displacement oligo. The data show a rapid increase in fluorescence when PURExpress products MG153-5 and MG153-51 were added, but not when a non-templated control (NTC) was added.

FIG. 75 depicts a schematic of Template Switching Assay. RT initiates production of cDNA at 3′ end of Donor RNA template. The Acceptor RNA template was used as an equal molar mixture of templates with different 3′ terminal nucleotides (NN-UU, AA, CC, GG), unless otherwise specified. The cDNA products resulting from initiation (FAM probe) and template switch (HEX probe) is quantified by multiplexed Taqman qPCR. Template switching efficiency (% TS) is calculated as the percentage of cDNA detected by HEX divided by FAM

FIGS. 76A-76B depict template switching of GII intron RTs using Acceptor RNA with terminal 3′UU nucleotides. FIG. 76A: The amount of cDNA produced (nM) determined by the FAM and HEX signal quantified by Taqman qPCR. RTs are derived from a cell-free expression system. NTC is a Non Templated Control, where no RT expression template is provided to the cell-free expression system. Full is the control template, where the Acceptor and Donor sequences are concatenated and the FAM and HEX signals are expected to be equivalent. 10× A:D denotes that the Acceptor RNA template (in this experiment contains 3′ terminal UU nucleotides) was used in 10-fold molar excess to the Donor RNA template. FIG. 76B: The template switching efficiency for each RT, calculated as described in FIG. 75. In both figure panels, TGIRT (GII intron), MMLV (retroviral), and MarathonRT (GII intron) are referred to as Control 1, 2, and 3 respectively.

FIGS. 77A-77B depict template switching of GII intron RTs using Acceptor RNA with mixed 3′ terminal nucleotides. FIG. 77A: The amount of cDNA produced (nM) determined by the FAM and HEX signal quantified by Taqman qPCR. RTs are derived from a cell-free expression system. NTC is a Non Templated Control, where no RT expression template is provided to the cell-free expression system. Full is the control template, where the Acceptor and Donor sequences are concatenated and the FAM and HEX signals are expected to be equivalent. 10× A:D denotes that the Acceptor RNA template (in this experiment contains mixed 3′ terminal nucleotides described whose preparation is described in the text) was used in 10-fold molar excess to the Donor RNA template. FIG. 77B: The template switching efficiency for each RT, calculated as described in FIG. 75. In both figure panels, TGIRT (GII intron), MMLV (retroviral), and MarathonRT (GII intron) are referred to as Control 1, 2, and 3 respectively.

FIGS. 78A-78B depict template switching of R2 MG140-8c5 with Acceptor titration. FIG. 78A: The amount of cDNA produced (nM) determined by the FAM and HEX signal quantified by Taqman qPCR. Two buffers were tested to evaluate if buffer composition impacts template switching activity. Buffer 1 composition is specified in methods, as it is the primary buffer used for the template switching reactions. Buffer 2 is composed of 40 mM Tris-HCl (pH 7.5), 0.2 M NaCl, 10 mM MgCl₂, 1 mM TCEP, RNase inhibitor, and 0.5 mM dNTPs. No RT control reactions were performed for each buffer to establish signal background. MG140-8c5 was tested as purified protein. Full is the control template, where the Acceptor and Donor sequences are concatenated and the FAM and HEX signals are expected to be equivalent. 10×, 5×, and 1×A:D denotes that the Acceptor RNA template (in this experiment contains mixed 3′ terminal nucleotides described whose preparation is described in the text) was used in 10-fold, 5-fold, or 1-fold molar excess to the Donor RNA template. FIG. 78B: The template switching efficiency for 140-8c5 with 10-fold, 5-fold, or 1-fold molar excess of Acceptor to Donor in Buffer 1 or Buffer 2, calculated as described in FIG. 75.

FIG. 79 depicts primed vs. unprimed cDNA synthesis of RTs quantified by qPCR. RNA template designs are indicated at the top of the figure. The first two templates contain a 22-nt poly A sequence on the 3′ end, and is referred to as “A” in the bar graph below. The last two templates have an MS2 hairpin instead of the polyA and are referred to as “MS2” in the bar graph below. The polyA and MS2 templates were tested either with the free 3′ hydroxyl (denoted as 3′OH) or with the free 3′OH blocked (denoted as 3′B, IDT 3′ C3 Spacer/3SpC3/). Each of the templates was also tested primed (P), meaning that is was annealed to a 20-nt priming DNA oligo, or unprimed (UP). The dashed line represents cDNA quantities 10-fold above the highest background negative control, which is PURExpress with a no RT expression template (PUREx NTC). MMLV and TGIRT are referred to as Control 1 and Control 2, respectively.

FIG. 80 depicts primed cDNA synthesis activity divided by the unprimed activity for each template described in FIG. 79, where A-3OH refers to the polyA sequence with free 3′ hydroxyl, A-3B refers to the polyA sequence with the 3′OH blocked, MS2-3OH refers to the MS2 sequence with a free 3′ hydroxyl, and MS2-3B refers to the MS2 sequence with the 3′OH blocked. MMLV and TGIRT are referred to as Control 1 and Control 2, respectively.

FIG. 81 depicts primed cDNA synthesis activity divided by the unprimed activity averaged across all 4 templates as described in FIG. 79. RTs ordered by their preference for primed RNA templates. MMLV and TGIRT are referred to as Control 1 and Control 2, respectively.

FIGS. 82A-82B depict the evaluation of primed and unprimed activity in MG140-8c5. FIG. 82A: 5′-labeled 100 nt RNA template annealed to quenching displacement oligo either in the presence or absence of priming oligo was used as substrate in a reaction containing purified MG140-8c5 and initiated with the addition of dNTPs. The data show an increase in fluorescence—and by extension both cDNA synthesis and strand displacement—for both primed and unprimed substrates. FIG. 82B: 5′-labeled 100 nt ssDNA template annealed to quenching displacement oligo either in the presence or absence of priming oligo was used as substrate in a reaction containing purified MG140-8c5 and initiated with the addition of dNTPs. The data show an increase in fluorescence—and by extension both second-strand synthesis and strand displacement—for both primed and unprimed substrates.

FIG. 83 depicts a cladogram of the reconstructed ancestral variants of the MG160 family of retron-like RTs. A phylogenetic tree was generated.

FIG. 84 depicts generic in vitro cDNA synthesis activity of MG157 retron RTs. Lane numbers correspond to the following samples-1: PURExpress no template control, RT reaction does not contain a reverse transcriptase. 2: positive control group II intron RT TGIRT. Lanes 3-9 correspond to MG157 family candidates. Lane numbers in bold corresponds to gel lanes with active candidates. Arrows indicate full-length cDNA product (arrow near the top of the gel) and examples of cDNA drop off (lower arrows or vertical line).

FIGS. 85A-85B depict graphs showing screening results of the ability of indicated RTs and engineered candidate RTs to synthesize cDNA in mammalian cells. FIG. 85A: Taqman qPCR detection of first (FAM probe) and last (HEX probe) 100 bp PCR products amplified from cDNA synthesized from an RNA template by group II intron RTs of the MG165, MG166, MG167, and MG169 families. FIG. 85B: Taqman qPCR detection of first (FAM probe) and last (HEX probe) 100 bp PCR products amplified from cDNA synthesized from a 4.1 kb RNA template by rationally engineered variants of non-LTR retrotransposon RTs MG140-74 and MG140-88. Bottom dotted line indicates background (no RT control), while top dotted line represents the maximum cDNA synthesis activity of positive control RT TGIRT. Additional positive control RTs include MMLV WT and engineered RT, and R2Tg. Variants of the MG140 family of RTs that display the highest cDNA synthesis activity levels are highlighted with a star.

FIGS. 86A-86C depict schematic and results of the Template Switching Assay. FIG. 86A shows a schematic of the Template Switching Assay. RT initiates production of cDNA at 3′ end of Donor RNA template. The Acceptor RNA template was used as an equal molar mixture of templates with different 3′ terminal nucleotides (NN-UU, AA, CC, GG), unless otherwise specified. The cDNA products resulting from initiation (FAM probe) and template switch (HEX probe) is quantified by multiplexed Taqman qPCR. Template switching efficiency (% TS) is calculated as the percentage of cDNA detected by HEX divided by FAM. FIG. 86B depicts a graph showing assay results as % Template Switch. The template switching efficiency was quantified for the non-LTR retrotransposase variants MG140-8c5, MG140-8 with a dead endonuclease domain (Endodead), and MG140-8 with a dead endonuclease domain with a D451A mutation, as well as for group II intron RTs MG153-18 and MG153-51. Enzymes were purified prior to template switching experiments. FIG. 86C depicts a graph showing assay results as % Template Switch. The template switching efficiency was quantified for the group II intron RTs MG153-18 and MG153-51, and for the non-LTR retrotransposase variants MG140-3 with a dead endonuclease domain (Endodead), and MG140-3 with the dead endonuclease domain and D451A, F702A and L698A mutations. Enzymes were expressed in a cell-free expression system.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

### MG140

SEQ ID NOs: 1-29, 393-401, 1476, 1850-1926, and 2165-2210 show the full-length peptide sequences of MG140 transposition proteins.

SEQ ID NOs: 374-386 show the nucleotide sequences of genes encoding HA-His-tagged MG140 reverse transcriptase proteins.

SEQ ID NOs: 761-798, 2161-2164, and 2211-2232 show the nucleotide sequences of MG140 UTRs.

SEQ ID NOs: 799-894 show the full-length peptide sequences of MG140 reverse transcriptase proteins.

SEQ ID NOs: 1535-1536, 1611-1623, 1663-1691, and 1786-1806 show the nucleotide sequences of genes encoding MG140 reverse transcriptase proteins optimized for expression in mammalian cells.

SEQ ID NOs: 1542-1543 show the nucleotide sequences of genes encoding dead mutant MG140 reverse transcriptase proteins optimized for expression in mammalian cells.

### MG146

SEQ ID NOs: 402 and 895 show the full-length peptide sequences of MG146 transposition proteins.

SEQ ID NO: 387 shows the nucleotide sequence of a gene encoding an HA-His-tagged MG146 reverse transcriptase protein.

### MG147

SEQ ID NO: 388 shows the nucleotide sequence of a gene encoding an HA-His-tagged MG147 reverse transcriptase protein.

### MG148

SEQ ID NOs: 403-426 show the full-length peptide sequences of MG148 reverse transcriptase proteins.

SEQ ID NOs: 389-392 show the nucleotide sequences of genes encoding HA-His-tagged MG148 reverse transcriptase proteins.

SEQ ID NOs: 1504-1507 show the nucleotide sequences of genes encoding MG148 reverse transcriptase proteins optimized for expression in mammalian cells.

### MG149

SEQ ID NOs: 427-439 show the full-length peptide sequences of MG149 reverse transcriptase proteins.

### MG151

SEQ ID NOs: 440-554 and 1020-1037 show the full-length peptide sequences of MG151 reverse transcriptase proteins.

SEQ ID NOs: 356-362 show the nucleotide sequences of genes encoding TwinStrep-tagged MG151 reverse transcriptase proteins.

SEQ ID NOs: 363-373 show the nucleotide sequences of genes encoding strep-tagged MG151 reverse transcriptase proteins.

SEQ ID NOs: 964-981 and 1003-1019 show the nucleotide sequences of genes encoding MG151 reverse transcriptase proteins optimized for expression in mammalian cells and cloned into an untethered plasmid.

### MG153

SEQ ID NOs: 555-608 and 1927-2010 show the full-length peptide sequences of MG153 reverse transcriptase proteins.

SEQ ID NOs: 30-32 and 40-50 show the nucleotide sequences of fusion proteins comprising MG153 reverse transcriptase proteins and MS2 coat proteins (MCP).

SEQ ID NOs: 66-119 show the nucleotide sequences of genes encoding strep-tagged MG153 reverse transcriptase proteins.

SEQ ID NOs: 120-173 show the nucleotide sequences of E. coli codon optimized genes encoding MG153 reverse transcriptase proteins.

SEQ ID NOs: 740-756 show the nucleotide sequences of genes encoding MCP-tagged MG153 reverse transcriptase proteins.

SEQ ID NOs: 1521-1534, 1624-1637, 1645-1662, and 1701-1782 show the nucleotide sequences of genes encoding MG153 reverse transcriptase proteins optimized for expression in mammalian cells.

SEQ ID NOs: 1539-1541 show the nucleotide sequences of genes encoding dead mutant MG153 reverse transcriptase proteins optimized for expression in mammalian cells.

SEQ ID NOs: 2233-2257 show the nucleotide sequences of MG153 UTRs.

### MG154

SEQ ID NOs: 609-610 and 1555 show the full-length peptide sequences of MG154 reverse transcriptase proteins.

SEQ ID NOs: 308-309 show the nucleotide sequences of genes encoding strep-tagged MG154 reverse transcriptase proteins.

SEQ ID NOs: 324-325 show the nucleotide sequences of E. coli codon optimized genes encoding MG154 reverse transcriptase proteins.

SEQ ID NOs: 340-341 show the nucleotide sequences of ncRNAs compatible with MG154 nucleases.

### MG155

SEQ ID NOs: 611-615 and 1544-1545 show the full-length peptide sequences of MG155 reverse transcriptase proteins.

SEQ ID NOs: 310-312 and 1569-1570 show the nucleotide sequences of genes encoding strep-tagged MG155 reverse transcriptase proteins.

SEQ ID NOs: 326-328 and 1556-1557 show the nucleotide sequences of E. coli codon optimized genes encoding MG155 reverse transcriptase proteins.

SEQ ID NOs: 342-344 and 1582-1583 show the nucleotide sequences of ncRNAs compatible with MG155 nucleases.

### MG156

SEQ ID NOs: 616-617 show the full-length peptide sequences of MG156 reverse transcriptase proteins.

SEQ ID NOs: 313-314 show the nucleotide sequences of genes encoding strep-tagged MG156 reverse transcriptase proteins.

SEQ ID NOs: 329-330 show the nucleotide sequences of E. coli codon optimized genes encoding MG156 reverse transcriptase proteins.

SEQ ID NOs: 345-346 show the nucleotide sequences of ncRNAs compatible with MG156 nucleases.

### MG157

SEQ ID NOs: 618-622 and 2258-2266 show the full-length peptide sequences of MG157 reverse transcriptase proteins.

SEQ ID NOs: 315-319 show the nucleotide sequences of genes encoding strep-tagged MG157 reverse transcriptase proteins.

SEQ ID NOs: 331-335 show the nucleotide sequences of E. coli codon optimized genes encoding MG157 reverse transcriptase proteins.

SEQ ID NOs: 347-351 and 1842-1849 show the nucleotide sequences of ncRNAs compatible with MG157 nucleases.

### MG158

SEQ ID NO: 623 shows the full-length peptide sequence of an MG158 reverse transcriptase protein.

SEQ ID NO: 320 shows the nucleotide sequence of a gene encoding a strep-tagged MG158 reverse transcriptase protein.

SEQ ID NO: 336 shows the nucleotide sequence of an E. coli codon optimized gene encoding an MG158 reverse transcriptase protein.

SEQ ID NO: 352 shows the nucleotide sequence of an ncRNA compatible with MG158 nucleases.

### MG159

SEQ ID NOs: 624-626 show the full-length peptide sequences of MG159 reverse transcriptase proteins.

SEQ ID NOs: 321-323 show the nucleotide sequences of genes encoding strep-tagged MG159 reverse transcriptase proteins.

SEQ ID NOs: 337-339 show the nucleotide sequences of E. coli codon optimized genes encoding MG159 reverse transcriptase proteins.

SEQ ID NOs: 353-355 show the nucleotide sequences of ncRNAs compatible with MG159 nucleases.

SEQ ID NO: 1785 shows the nucleotide sequence of a gene encoding a MG159 reverse transcriptase protein optimized for expression in mammalian cells.

### MG160

SEQ ID NOs: 627-673, 1039-1475, and 2011-2026 show the full-length peptide sequences of MG160 reverse transcriptase proteins.

SEQ ID NOs: 174-180 show the nucleotide sequences of genes encoding strep-tagged MG160 reverse transcriptase proteins.

SEQ ID NOs: 181-187 show the nucleotide sequences of E. coli codon genes encoding optimized MG160 reverse transcriptase proteins.

SEQ ID NOs: 982-1002 show the nucleotide sequences of genes encoding MG160 reverse transcriptase proteins optimized for expression in mammalian cells and cloned into a tethered spCas9 (H840A) plasmid.

SEQ ID NOs: 1508-1520 show the nucleotide sequences of genes encoding MG160 reverse transcriptase proteins optimized for expression in mammalian cells.

### MG163

SEQ ID NOs: 674-678 show the full-length peptide sequences of MG163 reverse transcriptase proteins.

SEQ ID NOs: 188-192 show the nucleotide sequences of genes encoding strep-tagged MG163 reverse transcriptase proteins.

SEQ ID NOs: 193-197 show the nucleotide sequences of E. coli codon genes encoding optimized MG163 reverse transcriptase proteins.

### MG164

SEQ ID NOs: 679-683 show the full-length peptide sequences of MG164 reverse transcriptase proteins.

SEQ ID NOs: 198-202 show the nucleotide sequences of genes encoding strep-tagged MG164 reverse transcriptase proteins.

SEQ ID NOs: 203-207 show the nucleotide sequences of E. coli codon genes encoding optimized MG164 reverse transcriptase proteins.

### MG165

SEQ ID NOs: 684-692 and 2027-2046 show the full-length peptide sequences of MG165 reverse transcriptase proteins.

SEQ ID NOs: 208-216 show the nucleotide sequences of genes encoding strep-tagged MG165 reverse transcriptase proteins.

SEQ ID NOs: 217-225 show the nucleotide sequences of E. coli codon genes encoding optimized MG165 reverse transcriptase proteins.

SEQ ID NOs: 757-759 show the nucleotide sequences of genes encoding MCP-tagged MG165 reverse transcriptase proteins.

### MG166

SEQ ID NOs: 693-697 and 2047-2090 show the full-length peptide sequences of MG166 reverse transcriptase proteins.

SEQ ID NOs: 226-230 show the nucleotide sequences of genes encoding strep-tagged MG166 reverse transcriptase proteins.

SEQ ID NOs: 231-235 show the nucleotide sequences of E. coli codon genes encoding optimized MG166 reverse transcriptase proteins.

### MG167

SEQ ID NOs: 698-702 and 2091-2120 show the full-length peptide sequences of MG167 reverse transcriptase proteins.

SEQ ID NOs: 236-240 show the nucleotide sequences of genes encoding strep-tagged MG167 reverse transcriptase proteins.

SEQ ID NOs: 241-245 show the nucleotide sequences of E. coli codon genes encoding optimized MG167 reverse transcriptase proteins.

SEQ ID NOs: 759-760 show the nucleotide sequences of genes encoding MCP-tagged MG167 reverse transcriptase proteins.

### MG168

SEQ ID NOs: 703-707 show the full-length peptide sequences of MG168 reverse transcriptase proteins.

SEQ ID NOs: 246-250 show the nucleotide sequences of genes encoding strep-tagged MG168 reverse transcriptase proteins.

SEQ ID NOs: 251-255 show the nucleotide sequences of E. coli codon genes encoding optimized MG168 reverse transcriptase proteins.

### MG169

SEQ ID NOs: 708-718 and 2121-2159 show the full-length peptide sequences of MG169 reverse transcriptase proteins.

SEQ ID NOs: 256-266 show the nucleotide sequences of genes encoding strep-tagged MG169 reverse transcriptase proteins.

SEQ ID NOs: 267-277 show the nucleotide sequences of E. coli codon genes encoding optimized MG169 reverse transcriptase proteins.

SEQ ID NOs: 1638-1644 and 1693-1700 show the nucleotide sequences of genes encoding MG169 reverse transcriptase proteins optimized for expression in mammalian cells.

### MG170

SEQ ID NOs: 719-728 show the full-length peptide sequences of MG170 reverse transcriptase proteins.

SEQ ID NOs: 278-287 show the nucleotide sequences of genes encoding strep-tagged MG170 reverse transcriptase proteins.

SEQ ID NOs: 288-297 show the nucleotide sequences of E. coli codon genes encoding optimized MG170 reverse transcriptase proteins.

### MG172

SEQ ID NOs: 729-733 show the full-length peptide sequences of MG172 reverse transcriptase proteins.

SEQ ID NOs: 298-302 show the nucleotide sequences of genes encoding strep-tagged MG172 reverse transcriptase proteins.

SEQ ID NOs: 303-307 show the nucleotide sequences of E. coli codon genes encoding optimized MG172 reverse transcriptase proteins.

### MG173

SEQ ID NOs: 734-735 and 1546-1553 show the full-length peptide sequences of MG173 reverse transcriptase proteins.

SEQ ID NOs: 1571-1580 show the nucleotide sequences of genes encoding strep-tagged MG173 reverse transcriptase proteins.

SEQ ID NOs: 1558-1567 show the nucleotide sequences of E. coli codon optimized genes encoding MG173 reverse transcriptase proteins.

SEQ ID NOs: 1584-1593 show the nucleotide sequences of ncRNAs compatible with MG173 nucleases.

SEQ ID NOs: 1783-1784 show the nucleotide sequences of genes encoding MG173 reverse transcriptase proteins optimized for expression in mammalian cells.

### MG176

SEQ ID NOs: 1038 and 2160 show the full-length peptide sequences of MG176 retrotransposition proteins.

SEQ ID NO: 1692 shows the nucleotide sequence of a gene encoding a MG176 reverse transcriptase protein optimized for expression in mammalian cells.

### MG192

SEQ ID NO: 1554 shows the full-length peptide sequence of an MG192 reverse transcriptase protein.

SEQ ID NO: 1581 shows the nucleotide sequence of a gene encoding a strep-tagged MG192 reverse transcriptase protein.

SEQ ID NO: 1568 shows the nucleotide sequence of an E. coli codon optimized gene encoding an MG192 reverse transcriptase protein.

SEQ ID NO: 1594 shows the nucleotide sequence of an ncRNA compatible with MG192 nucleases.

### Other Sequences

SEQ ID NOs: 736-738, 897-900, 927-928, 952-955, 1494-1497, 1595-1599, 1601-1604, 1809-1810, 1812-1815, 1818-1819 show the nucleotide sequences of primers.

SEQ ID NOs: 739, 901-902, 1498-1499, and 1605-1606 show the nucleotide sequences of Taqman probes for qPCR.

SEQ ID NOs: 896, 1493, and 1600 show the nucleotide sequence of an RNA template for cDNA synthesis.

SEQ ID NOs: 903-926 and 934-951 show the full-length sequences of chemically modified guide RNAs.

SEQ ID NOs: 929 and 932-933 shows the nucleotide sequences of cDNAs encoding gene targets.

SEQ ID NO: 930 shows the nucleotide sequence of an RT-nickase linker.

SEQ ID NO: 931 shows the nucleotide sequence of MG3-6 (H586A).

SEQ ID NOs: 956-963 show the nucleotide sequences of reverse transcriptases cloned into a tethered MG3-6 (H586A) plasmid.

SEQ ID NOs: 1500-1502 and 1607-1610 show the nucleotide sequences of genes encoding control reverse transcriptase proteins optimized for expression in mammalian cells.

SEQ ID NOs: 1537-1538 show the nucleotide sequences of genes encoding dead mutant control reverse transcriptase proteins optimized for expression in mammalian cells.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

The practice of some methods disclosed herein employ, unless otherwise indicated, techniques of immunology, biochemistry, chemistry, molecular biology, microbiology, cell biology, genomics, and recombinant DNA.

As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, e.g., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

The term “nucleotide,” as used herein, refers to a base-sugar-phosphate combination. Contemplated nucleotides include naturally occurring nucleotides and synthetic nucleotides. Nucleotides are monomeric units of a nucleic acid sequence (e.g., deoxyribonucleic acid (DNA) and ribonucleic acid (RNA)). The term nucleotide includes ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein encompasses dideoxyribonucleoside triphosphates (ddNTPs) and their derivatives. Illustrative examples of ddNTPs include, but are not limited to, ddATP, ddCTP, ddGTP, ddITP, and ddTTP. A nucleotide may be unlabeled or detectably labeled, such as using moieties comprising optically detectable moieties (e.g., fluorophores) or quantum dots. Detectable labels include, for example, radioactive isotopes, fluorescent labels, chemiluminescent labels, bioluminescent labels, and enzyme labels. Fluorescent labels of nucleotides include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, IL; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. The term nucleotide encompasses chemically modified nucleotides. An exemplary chemically-modified nucleotide is biotin-dNTP. Non-limiting examples of biotinylated dNTPs include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. Contemplated polynucleotides include a gene or fragment thereof. Exemplary polynucleotides include, but are not limited to, DNA, RNA, coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, cell-free polynucleotides including cell-free DNA (cfDNA) and cell-free RNA (cfRNA), nucleic acid probes, and primers. In a polynucleotide when referring to a T, a T means U (Uracil) in RNA and T (Thymine) in DNA. A polynucleotide can be exogenous or endogenous to a cell and/or exist in a cell-free environment. The term polynucleotide encompasses modified polynucleotides (e.g., altered backbone, sugar, or nucleobase). If present, modifications to the nucleotide structure are imparted before or after assembly of the polymer. Non-limiting examples of modifications include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. The sequence of nucleotides may be interrupted by non-nucleotide components.

The terms “transfection” or “transfected” refer to introduction of a nucleic acid into a cell by non-viral or viral-based methods. The nucleic acid molecules may be gene sequences encoding complete proteins or functional portions thereof.

As used herein, the “non-native” refers to a nucleic acid or polypeptide sequence that is non-naturally occurring. Non-native refers to a non-naturally occurring nucleic acid or polypeptide sequence that comprises modifications such as mutations, insertions, or deletions. The term non-native encompasses fusion nucleic acids or polypeptides that encodes or exhibits an activity (e.g., enzymatic activity, methyltransferase activity, acetyltransferase activity, kinase activity, ubiquitinating activity, etc.) of the nucleic acid or polypeptide sequence to which the non-native sequence is fused. A non-native nucleic acid or polypeptide sequence includes those linked to a naturally-occurring nucleic acid or polypeptide sequence (or a variant thereof) by genetic engineering to generate a chimeric nucleic acid or polypeptide sequence encoding a chimeric nucleic acid or polypeptide.

As used herein, the “non-native” can also refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions, or deletions. A non-native sequence may exhibit or encode for an activity (e.g., enzymatic activity, methyltransferase activity, acetyltransferase activity, kinase activity, ubiquitinating activity, etc.) that may also be exhibited by the nucleic acid or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid or polypeptide sequence encoding a chimeric nucleic acid or polypeptide.

The term “promoter”, as used herein, refers to the regulatory DNA region which controls transcription or expression of a polynucleotide (e.g., a gene) and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. Eukaryotic basal promoters typically, though not necessarily, contain a TATA-box and/or a CAAT box.

The term “expression,” as used herein, refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof refer to an arrangement of genetic elements, e.g., a promoter, an enhancer, a polyadenylation sequence, etc., wherein an operation (e.g., movement or activation) of a first genetic element has some effect on the second genetic element. The effect on the second genetic element can be, but need not be, of the same type as operation of the first genetic element. For example, two genetic elements are operably linked if movement of the first element causes an activation of the second element. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, refers to a macromolecule or association of macromolecules that comprises or associates with a polynucleotide and which mediates delivery of the polynucleotide to a cell. Examples of vectors include nucleic-based vectors (e.g., plasmids and viral vectors) and liposomes. An exemplary nucleic-acid based vector comprises genetic elements, e.g., regulatory elements, operatively linked to a gene to facilitate expression of the gene in a target.

As used herein, “expression cassette” and “nucleic acid cassette” are used interchangeably to refer to a component of a vector comprising a combination of nucleic acid sequences or elements (e.g., therapeutic gene, promoter, and a terminator) that are expressed together or are operably linked for expression. The terms encompass an expression cassette including a combination of regulatory elements and a gene or genes to which they are operably linked for expression.

A “functional fragment” of a DNA or protein sequence refers to a fragment that retains a biological activity (either functional or structural) that is substantially similar to a biological activity of the full-length DNA or protein sequence. A biological activity of a DNA sequence includes its ability to influence expression in a manner attributed to the full-length sequence.

The terms “engineered,” “synthetic,” and “artificial” are used interchangeably herein to refer to an object that has been modified by human intervention. For example, the terms refer to a polynucleotide or polypeptide that is non-naturally occurring. An engineered peptide has, but does not require, low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains. Non-limiting examples include the following: a nucleic acid modified by changing its sequence to a sequence that does not occur in nature; a nucleic acid modified by ligating it to a nucleic acid that it does not associate with in nature such that the ligated product possesses a function not present in the original nucleic acid; an engineered nucleic acid synthesized in vitro with a sequence that does not exist in nature; a protein modified by changing its amino acid sequence to a sequence that does not exist in nature; an engineered protein acquiring a new function or property. An “engineered” system comprises at least one engineered component.

As used herein, the term “transposable element” refers to a DNA sequence that can move from one location in the genome to another (i.e., it can be “transposed”). Transposable elements can be generally divided into two classes. Class I transposable elements, or “retrotransposons”, are transposed via transcription and translation of an RNA intermediate which is subsequently reincorporated into its new location into the genome via reverse transcription (a process mediated by a reverse transcriptase). Class II transposable elements, or “DNA transposons”, are transposed via a complex of single- or double-stranded DNA flanked on either side by a transposase.

As used herein, the term “retrotransposons” refers to Class I transposable elements that function according to a two-part “copy and paste” mechanism involving an RNA intermediate. “Retrotransposase” refers to an enzyme responsible for transposition of a retrotransposon. The retrotransposase can comprise a reverse transcriptase domain, one or more zinc finger domains, an endonuclease domain, or combinations thereof.

As used herein, the terms “gene editing” and “genome editing” can be used interchangeably. Gene editing or genome editing means to change the nucleic acid sequence of a gene or a genome. Genome editing can include, for example, insertions, deletions, and mutations. Genome editing can be performed by a gene editing system, for example a retrotransposase.

As used herein, the term “complex” refers to a joining of at least two components. The two components may each retain the properties/activities they had prior to forming the complex or gain properties as a result of forming the complex. The joining includes, but is not limited to, covalent bonding, non-covalent bonding (i.e., hydrogen bonding, ionic interactions, Van der Waals interactions, and hydrophobic bond), use of a linker, fusion, or any other suitable method. Contemplated components of the complex include polynucleotides, polypeptides, or combinations thereof. For example, a complex comprises an endonuclease and a guide polynucleotide.

The term “sequence identity” or “percent identity” in the context of two or more nucleic acids or polypeptide sequences, refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with the Smith-Waterman homology search algorithm parameters with a match of 2, a mismatch of −1, and a gap of −1; MUSCLE with default parameters; MAFFT with parameters of a retree of 2 and max iterations of 1000; Novafold with default parameters; HMMER hmmalign with default parameters.

The term “optimally aligned” in the context of two or more nucleic acids or polypeptide sequences, refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

The term “open reading frame” or “ORF” refers to a nucleotide sequence that can encode a protein, or a portion of a protein. An open reading frame can begin with a start codon (represented as, e.g., AUG for an RNA molecule and ATG in a DNA molecule in the standard code) and can be read in codon-triplets until the frame ends with a STOP codon (represented as, e.g., UAA, UGA, or UAG for an RNA molecule and TAA, TGA, or TAG in a DNA molecule in the standard code).

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of the retrotransposase protein sequences described herein (e.g., MG140 family retrotransposases described herein, or any other family retrotransposase described herein). In some embodiments, such conservatively substituted variants are functional variants. Such functional variants can encompass sequences with substitutions such that the activity of one or more critical active site residues of the retrotransposase are not disrupted. In some embodiments, a functional variant of any of the proteins described herein lacks substitution of at least one of the conserved or functional residues. In some embodiments, a functional variant of any of the proteins described herein lacks substitution of all of the conserved or functional residues.

Also included in the current disclosure are variants of any of the enzymes described herein with substitution of one or more catalytic residues to decrease or eliminate activity of the enzyme (e.g., decreased-activity variants). In some embodiments, a decreased activity variant as a protein described herein comprises a disrupting substitution of at least one, at least two, or all three catalytic residues.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M).

Also included in the current disclosure are variants of any of the nucleic acid sequences described herein with one or more substitutions, deletions, or insertions. In some embodiments, such a variant has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of the nucleic acid sequences described herein.

Some of the protein sequences described herein involve the determination of a particular domain (e.g., a reverse transcriptase or RT domain) from the sequence of a selected larger protein (e.g., a retrotransposase). In such cases, multiple sequence alignments (MSA) with a reference larger protein (e.g., a retrotransposase) where the domains have been validated (e.g., with 3D structures) is used to identify domain boundaries by aligning the selected protein to the larger protein with validated domains. When MSAs are inconclusive because the sequences are so divergent, 3D structures of the larger proteins are determined and the structural domains are compared with known domains to define the boundaries. These boundaries can be further verified by ensuring the presence of important catalytic residues for the domain within the domain boundaries.

As used herein, the term “LINE retrotransposase” refers to a class of autonomous non-LTR retrotransposons (Long INterspersed Element). As used herein, the term “R2 retrotransposase” or “R4 retrotransposase” refer to subclasses of LINE retrotransposases that share similar domain architecture but differ in that R2 retrotransposases can be site specific (e.g., integrating at specific sites of an rRNA gene) while R4 retrotransposons can integrate both at an rRNA gene as well as other non-specific sites containing repeats.

### Overview

The discovery of new transposable elements with unique functionality and structure may offer the potential to further disrupt deoxyribonucleic acid (DNA) editing technologies, improving speed, specificity, functionality, and ease of use. Relative to the predicted prevalence of transposable elements in microbes and the sheer diversity of microbial species, relatively few functionally characterized transposable elements exist in the literature. This is partly because a huge number of microbial species may not be readily cultivated in laboratory conditions. Metagenomic sequencing from natural environmental niches containing large numbers of microbial species can offer the potential to drastically increase the number of new transposable elements documented and speed the discovery of new oligonucleotide editing functionalities.

Transposable elements are deoxyribonucleic acid sequences that can change position within a genome, often resulting in the generation or amelioration of mutations. In eukaryotes, a great proportion of the genome, and a large share of the mass of cellular DNA, is attributable to transposable elements. Although transposable elements are “selfish genes” which propagate themselves at the expense of other genes, they have been found to serve various important functions and to be crucial to genome evolution. Based on their mechanism, transposable elements are classified as either Class I “retrotransposons” or Class II “DNA transposons”.

Class I transposable elements, also referred to as retrotransposons, function according to a two-part “copy and paste” mechanism involving an RNA intermediate. First, the retrotransposon is transcribed. The resulting RNA is subsequently converted back to DNA by reverse transcriptase (generally encoded by the retrotransposon itself), and the reverse transcribed retrotransposon is integrated into its new position in the genome by integrase. Retrotransposons are further classified into three orders. Retrotransposons with long terminal repeats (“LTRs”) encode reverse transcriptase and are flanked by long strands of repeating DNA. Retrotransposons with long interspersed nuclear elements (“LINEs”) encode reverse transcriptase, lack LTRs, and are transcribed by RNA polymerase II. Retrotransposons with short interspersed nuclear elements (“SINEs”) are transcribed by RNA polymerase III but lack reverse transcriptase, instead relying on the reverse transcription machinery of other transposable elements (e.g., LINEs).

Class II transposable elements, also referred to as DNA transposons, function according to mechanisms that do not involve an RNA intermediate. Many DNA transposons display a “cut and paste” mechanism in which transposase binds terminal inverted repeats (“TIRs”) flanking the transposon, cleaves the transposon from the donor region, and inserts it into the target region of the genome. Others, referred to as “helitrons,” display a “rolling circle” mechanism involving a single-stranded DNA intermediate and mediated by an undocumented protein understood to possess HUH endonuclease function and 5′ to 3′ helicase activity. First, a circular strand of DNA is nicked to create two single DNA strands. The protein remains attached to the 5′ phosphate of the nicked strand, leaving the 3′ hydroxyl end of the complementary strand exposed and thus allowing a polymerase to replicate the non-nicked strand. Once replication is complete, the new strand disassociates and is itself replicated along with the original template strand. Still other DNA transposons, “Polintons,” are theorized to undergo a “self-synthesis” mechanism. The transposition is initiated by an integrase's excision of a single-stranded extra-chromosomal Polinton element, which forms a racket-like structure. The Polinton undergoes replication with DNA polymerase B, and the double stranded Polinton is inserted into the genome by the integrase. Additionally, some DNA transposons, such as those in the IS200/IS605 family, proceed via a “peel and paste” mechanism in which TnpA excises a piece of single-stranded DNA (as a circular “transposon joint”) from the lagging strand template of the donor gene and reinserts it into the replication fork of the target gene.

While transposable elements have found some use as biological tools, documented transposable elements do not encompass the full range of possible biodiversity and targetability, and may not represent all possible activities. Here, thousands of genomic fragments were mined from numerous metagenomes for transposable elements. The documented diversity of transposable elements may have been expanded and systems may have been developed into highly targetable, compact, and precise gene editing agents.

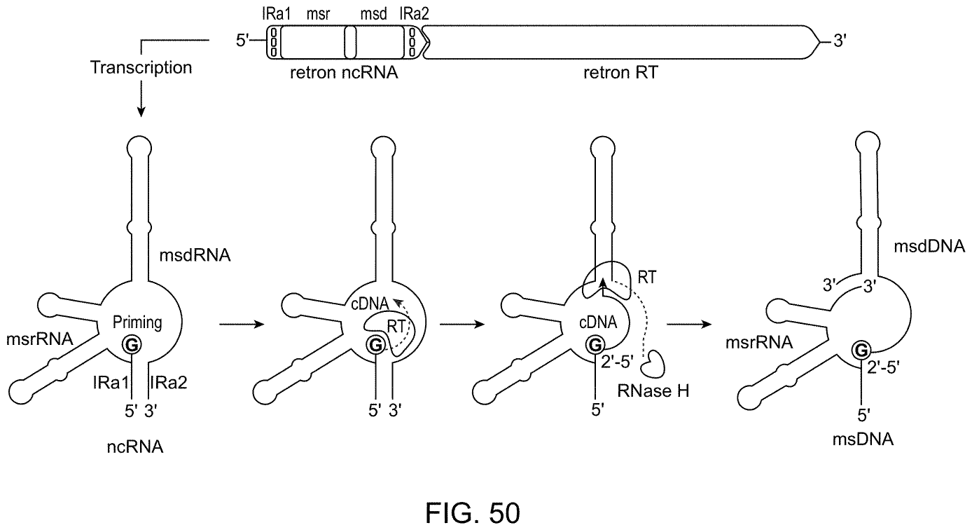

Retrons are bacterial retroelements that produce single-stranded, reverse-transcribed DNA (RT-DNA) that is a critical part of a newly discovered phage defense system. Retrons have the unique ability to produce multicopy single stranded DNAs (msDNAs) that are comprised of one strand of structured RNA, the ‘msr,’ connected to one strand of DNA, the ‘msd’ and flanked by two inverted and complementary repeats (5′ IRa1 and 3′ IRa2; FIG. 50). The msr and msd are encoded in a compact, contiguous transcriptional cassette that also includes a specialized reverse transcriptase (RT; ~300-400 amino acids); this cassette is referred to as a whole retron (FIG. 50). The RT initiates reverse transcription using as primers the base-paired 5′ and 3′ stem (IRa1 and IRa2) of the msr-msd and the conserved priming guanosine within a conserved AGC sequence in the msr at the 3′ end. The msr and msd molecules are joined by a 2′-5′ phosphodiester bond between a priming guanosine and the phosphate of the 5′ end of the msd that covalently links the RNA and DNA strands into a single branched molecule (FIG. 50). The mechanism for precise termination is not yet understood. The RT extends the reverse transcript until a defined position at which reverse transcription stops via an unknown mechanism. It has been observed that the RT terminates at similar sites in vitro and in cells, strongly suggesting that RNA structure may direct RT termination (Shimamoto T. et al., 1995; Simon A. et al., 2019). Interestingly, retron reverse transcriptases (RTs) typically lack an RNase H domain and, therefore, depend on endogenous RNase H1 to remove RNA templates from RT-DNA. Concurrently with reverse transcription, cellular RNAse H1 activity degrades the template of the msd, excluding ~5-10 RNA bases at its 5′ end. This segment of the RNA remains hybridized to the complementary reverse transcript and is considered to be part of both the msr and msd in the mature msDNA form (FIG. 50).

Retrons could be harnessed to become powerful tools for genome editing as they are able to produce high copy number intracellular DNA molecules in hosts. Early experiments showed that a Retron from E. coli (Ec67) msr and RT could successfully reverse transcribe another Retron (Ec73) msd. This experiment indicated that while a specific retron's msr and associated RT are always paired and essential to initiate reverse transcription, the msd could be variable and can encode an in-situ DNA with an artificial sequence of interest. This critical finding could enable the repurposing of retrons for biotechnological and therapeutic applications.

### MG Enzymes

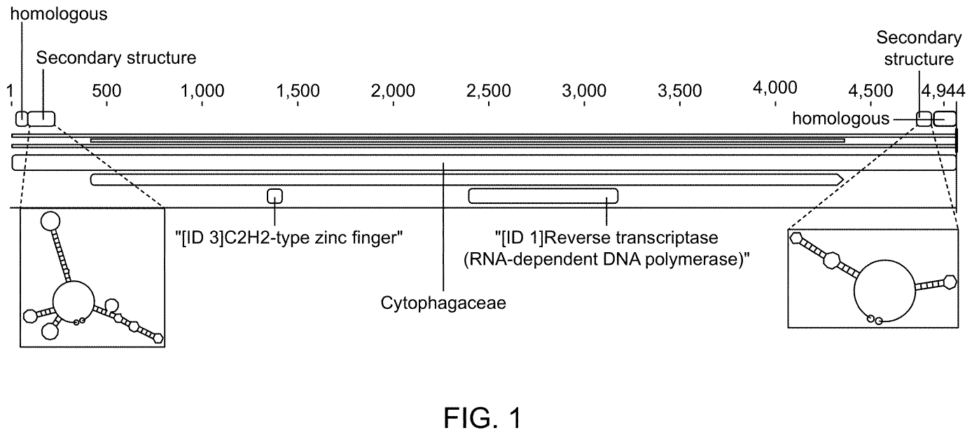

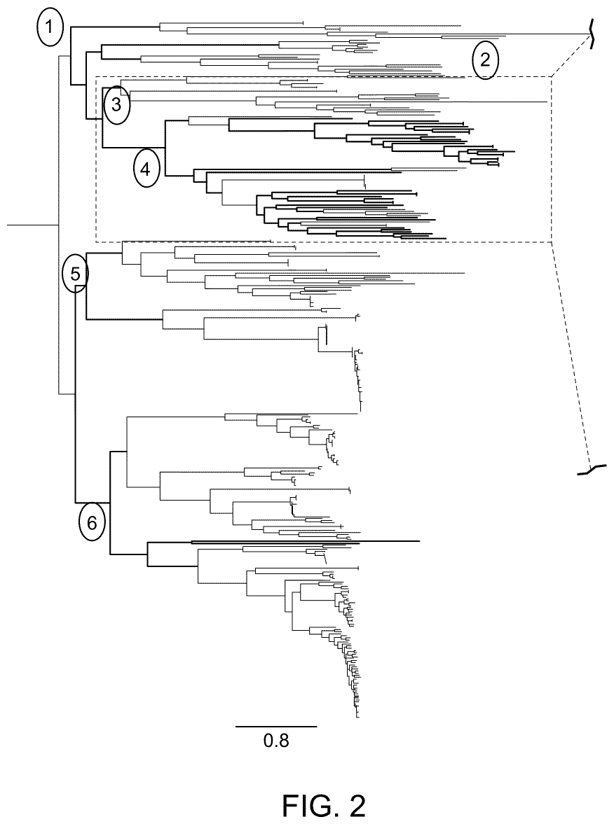

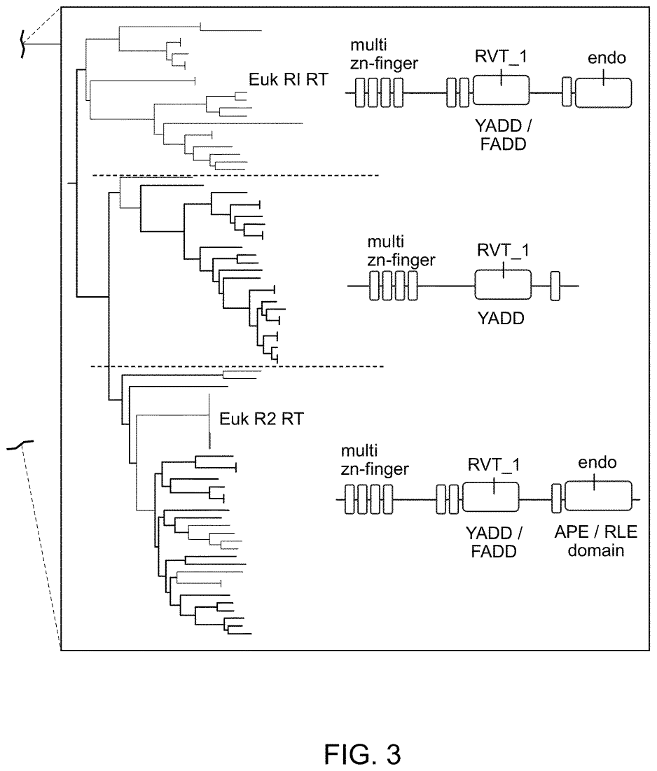

In some aspects, the present disclosure provides for retrotransposases. In some embodiments, the retrotransposase is a MG140, MG146, MG147, MG148, MG149, MG151, MG153, MG154, MG155, MG156, MG157, MG158, MG159, MG160, MG163, MG164, MG165, MG166, MG167, MG168, MG169, MG170, MG172, MG173, or MG176 retrotransposase. (see FIG. 1). In some embodiments, the retrotransposases are less than about 1,400 amino acids in length. In some embodiments, the retrotransposases simplify delivery and extend therapeutic applications.

In some embodiments, the present disclosure provides for an engineered retrotransposase system discovered through metagenomic sequencing. In some embodiments, the metagenomic sequencing is conducted on samples. In some embodiments, the samples are collected from a variety of environments. In some embodiments, the environment is a human microbiome, an animal microbiome, environments with high temperatures, environments with low temperatures. In some embodiments, the environment includes sediment.

In some embodiments, the present disclosure provides for an engineered retrotransposase system comprising a retrotransposase derived from an uncultivated microorganism. In some embodiments, the retrotransposase is configured to bind a 3′ untranslated region (UTR). In some embodiments, the retrotransposase binds a 5′ untranslated region (UTR).

In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-29, 393-735, 799-895, 1020-1476, 1544-1554, 1850-2160, 2165-2210, and 2258-2266. In some embodiments, the retrotransposase comprises a sequence having at least about 70% identity to any one of SEQ ID NOs: 1-29, 393-735, 799-895, 1020-1476, 1544-1554, 1850-2160, 2165-2210, and 2258-2266. In some embodiments, the retrotransposase comprises a sequence having at least about 75% identity to any one of SEQ ID NOs: 1-29, 393-735, 799-895, 1020-1476, 1544-1554, 1850-2160, 2165-2210, and 2258-2266. In some embodiments, the retrotransposase comprises a sequence having at least about 80% identity to any one of SEQ ID NOs: 1-29, 393-735, 799-895, 1020-1476, 1544-1554, 1850-2160, 2165-2210, and 2258-2266. In some embodiments, the retrotransposase comprises a sequence having at least about 85% identity to any one of SEQ ID NOs: 1-29, 393-735, 799-895, 1020-1476, 1544-1554, 1850-2160, 2165-2210, and 2258-2266. In some embodiments, the retrotransposase comprises a sequence having at least about 90% identity to any one of SEQ ID NOs: 1-29, 393-735, 799-895, 1020-1476, 1544-1554, 1850-2160, 2165-2210, and 2258-2266. In some embodiments, the retrotransposase comprises a sequence having at least about 95% identity to any one of SEQ ID NOs: 1-29, 393-735, 799-895, 1020-1476, 1544-1554, 1850-2160, 2165-2210, and 2258-2266. In some embodiments, the retrotransposase comprises a sequence having at least about 96% identity to any one of SEQ ID NOs: 1-29, 393-735, 799-895, 1020-1476, 1544-1554, 1850-2160, 2165-2210, and 2258-2266. In some embodiments, the retrotransposase comprises a sequence having at least about 97% identity to any one of SEQ ID NOs: 1-29, 393-735, 799-895, 1020-1476, 1544-1554, 1850-2160, 2165-2210, and 2258-2266. In some embodiments, the retrotransposase comprises a sequence having at least about 98% identity to any one of SEQ ID NOs: 1-29, 393-735, 799-895, 1020-1476, 1544-1554, 1850-2160, 2165-2210, and 2258-2266. In some embodiments, the retrotransposase comprises a sequence having at least about 99% identity to any one of SEQ ID NOs: 1-29, 393-735, 799-895, 1020-1476, 1544-1554, 1850-2160, 2165-2210, and 2258-2266. In some embodiments, the retrotransposase comprises a sequence having 100% identity to any one of SEQ ID NOs: 1-29, 393-735, 799-895, 1020-1476, 1544-1554, 1850-2160, 2165-2210, and 2258-2266.

In some embodiments, the retrotransposase is a MG140 retrotransposase (i.e., SEQ ID NOs: 1-29, 393-401, 799-894, 1476, 1850-1926, and 2165-2210). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-29, 393-401, 799-894, 1476, 1850-1926, and 2165-2210. In some embodiments, the retrotransposase comprises a sequence having at least about 70% identity to any one of SEQ ID NOs: 1-29, 393-401, 799-894, 1476, 1850-1926, and 2165-2210. In some embodiments, the retrotransposase comprises a sequence having at least about 75% identity to any one of SEQ ID NOs: 1-29, 393-401, 799-894, 1476, 1850-1926, and 2165-2210. In some embodiments, the retrotransposase comprises a sequence having at least about 80% identity to any one of SEQ ID NOs: 1-29, 393-401, 799-894, 1476, 1850-1926, and 2165-2210. In some embodiments, the retrotransposase comprises a sequence having at least about 85% identity to any one of SEQ ID NOs: 1-29, 393-401, 799-894, 1476, 1850-1926, and 2165-2210. In some embodiments, the retrotransposase comprises a sequence having at least about 90% identity to any one of SEQ ID NOs: 1-29, 393-401, 799-894, 1476, 1850-1926, and 2165-2210. In some embodiments, the retrotransposase comprises a sequence having at least about 95% identity to any one of SEQ ID NOs: 1-29, 393-401, 799-894, 1476, 1850-1926, and 2165-2210. In some embodiments, the retrotransposase comprises a sequence having at least about 96% identity to any one of SEQ ID NOs: 1-29, 393-401, 799-894, 1476, 1850-1926, and 2165-2210. In some embodiments, the retrotransposase comprises a sequence having at least about 97% identity to any one of SEQ ID NOs: 1-29, 393-401, 799-894, 1476, 1850-1926, and 2165-2210. In some embodiments, the retrotransposase comprises a sequence having at least about 98% identity to any one of SEQ ID NOs: 1-29, 393-401, 799-894, 1476, 1850-1926, and 2165-2210. In some embodiments, the retrotransposase comprises a sequence having at least about 99% identity to any one of SEQ ID NOs: 1-29, 393-401, 799-894, 1476, 1850-1926, and 2165-2210. In some embodiments, the retrotransposase comprises a sequence having 100% identity to any one of SEQ ID NOs: 1-29, 393-401, 799-894, 1476, 1850-1926, and 2165-2210.

In some embodiments, the retrotransposase is a MG146 retrotransposase (i.e., SEQ ID NO: 402 or SEQ ID NO: 895). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 402 or SEQ ID NO: 895. In some embodiments, the retrotransposase comprises a sequence having at least about 70% identity to SEQ ID NO: 402 or SEQ ID NO: 895. In some embodiments, the retrotransposase comprises a sequence having at least about 75% identity to SEQ ID NO: 402 or SEQ ID NO: 895. In some embodiments, the retrotransposase comprises a sequence having at least about 80% identity to SEQ ID NO: 402 or SEQ ID NO: 895. In some embodiments, the retrotransposase comprises a sequence having at least about 85% identity to SEQ ID NO: 402 or SEQ ID NO: 895. In some embodiments, the retrotransposase comprises a sequence having at least about 90% identity to SEQ ID NO: 402 or SEQ ID NO: 895. In some embodiments, the retrotransposase comprises a sequence having at least about 95% identity to SEQ ID NO: 402 or SEQ ID NO: 895. In some embodiments, the retrotransposase comprises a sequence having at least about 96% identity to SEQ ID NO: 402 or SEQ ID NO: 895. In some embodiments, the retrotransposase comprises a sequence having at least about 97% identity to SEQ ID NO: 402 or SEQ ID NO: 895. In some embodiments, the retrotransposase comprises a sequence having at least about 98% identity to SEQ ID NO: 402 or SEQ ID NO: 895. In some embodiments, the retrotransposase comprises a sequence having at least about 99% identity to SEQ ID NO: 402 or SEQ ID NO: 895. In some embodiments, the retrotransposase comprises a sequence having 100% identity to SEQ ID NO: 402 or SEQ ID NO: 895.

In some embodiments, the retrotransposase is a MG148 retrotransposase (i.e., SEQ ID NOs: 403-426). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 403-426. In some embodiments, the retrotransposase comprises a sequence having at least about 70% identity to any one of SEQ ID NOs: 403-426. In some embodiments, the retrotransposase comprises a sequence having at least about 75% identity to any one of SEQ ID NOs: 403-426. In some embodiments, the retrotransposase comprises a sequence having at least about 80% identity to any one of SEQ ID NOs: 403-426. In some embodiments, the retrotransposase comprises a sequence having at least about 85% identity to any one of SEQ ID NOs: 403-426. In some embodiments, the retrotransposase comprises a sequence having at least about 90% identity to any one of SEQ ID NOs: 403-426. In some embodiments, the retrotransposase comprises a sequence having at least about 95% identity to any one of SEQ ID NOs: 403-426. In some embodiments, the retrotransposase comprises a sequence having at least about 96% identity to any one of SEQ ID NOs: 403-426. In some embodiments, the retrotransposase comprises a sequence having at least about 97% identity to any one of SEQ ID NOs: 403-426. In some embodiments, the retrotransposase comprises a sequence having at least about 98% identity to any one of SEQ ID NOs: 403-426. In some embodiments, the retrotransposase comprises a sequence having at least about 99% identity to any one of SEQ ID NOs: 403-426. In some embodiments, the retrotransposase comprises a sequence having 100% identity to any one of SEQ ID NOs: 403-426.

In some embodiments, the retrotransposase is a MG149 retrotransposase (i.e., SEQ ID NOs: 427-439). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 427-439. In some embodiments, the retrotransposase comprises a sequence having at least about 70% identity to any one of SEQ ID NOs: 427-439. In some embodiments, the retrotransposase comprises a sequence having at least about 75% identity to any one of SEQ ID NOs: 427-439. In some embodiments, the retrotransposase comprises a sequence having at least about 80% identity to any one of SEQ ID NOs: 427-439. In some embodiments, the retrotransposase comprises a sequence having at least about 85% identity to any one of SEQ ID NOs: 427-439. In some embodiments, the retrotransposase comprises a sequence having at least about 90% identity to any one of SEQ ID NOs: 427-439. In some embodiments, the retrotransposase comprises a sequence having at least about 95% identity to any one of SEQ ID NOs: 427-439. In some embodiments, the retrotransposase comprises a sequence having at least about 96% identity to any one of SEQ ID NOs: 427-439. In some embodiments, the retrotransposase comprises a sequence having at least about 97% identity to any one of SEQ ID NOs: 427-439. In some embodiments, the retrotransposase comprises a sequence having at least about 98% identity to any one of SEQ ID NOs: 427-439. In some embodiments, the retrotransposase comprises a sequence having at least about 99% identity to any one of SEQ ID NOs: 427-439. In some embodiments, the retrotransposase comprises a sequence having 100% identity to any one of SEQ ID NOs: 427-439.

In some embodiments, the retrotransposase is a MG151 retrotransposase (i.e., SEQ ID NOs: 440-554 and 1020-1037). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 440-554 and 1020-1037. In some embodiments, the retrotransposase comprises a sequence having at least about 70% identity to any one of SEQ ID NOs: 440-554 and 1020-1037. In some embodiments, the retrotransposase comprises a sequence having at least about 75% identity to any one of SEQ ID NOs: 440-554 and 1020-1037. In some embodiments, the retrotransposase comprises a sequence having at least about 80% identity to any one of SEQ ID NOs: 440-554 and 1020-1037. In some embodiments, the retrotransposase comprises a sequence having at least about 85% identity to any one of SEQ ID NOs: 440-554 and 1020-1037. In some embodiments, the retrotransposase comprises a sequence having at least about 90% identity to any one of SEQ ID NOs: 440-554 and 1020-1037. In some embodiments, the retrotransposase comprises a sequence having at least about 95% identity to any one of SEQ ID NOs: 440-554 and 1020-1037. In some embodiments, the retrotransposase comprises a sequence having at least about 96% identity to any one of SEQ ID NOs: 440-554 and 1020-1037. In some embodiments, the retrotransposase comprises a sequence having at least about 97% identity to any one of SEQ ID NOs: 440-554 and 1020-1037. In some embodiments, the retrotransposase comprises a sequence having at least about 98% identity to any one of SEQ ID NOs: 440-554 and 1020-1037. In some embodiments, the retrotransposase comprises a sequence having at least about 99% identity to any one of SEQ ID NOs: 440-554 and 1020-1037. In some embodiments, the retrotransposase comprises a sequence having 100% identity to any one of SEQ ID NOs: 440-554 and 1020-1037.

In some embodiments, the retrotransposase is a MG153 retrotransposase (i.e., SEQ ID NOs: 555-608 and 1927-2010). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 555-608 and 1927-2010. In some embodiments, the retrotransposase comprises a sequence having at least about 70% identity to any one of SEQ ID NOs: 555-608 and 1927-2010. In some embodiments, the retrotransposase comprises a sequence having at least about 75% identity to any one of SEQ ID NOs: 555-608 and 1927-2010. In some embodiments, the retrotransposase comprises a sequence having at least about 80% identity to any one of SEQ ID NOs: 555-608 and 1927-2010. In some embodiments, the retrotransposase comprises a sequence having at least about 85% identity to any one of SEQ ID NOs: 555-608 and 1927-2010. In some embodiments, the retrotransposase comprises a sequence having at least about 90% identity to any one of SEQ ID NOs: 555-608 and 1927-2010. In some embodiments, the retrotransposase comprises a sequence having at least about 95% identity to any one of SEQ ID NOs: 555-608 and 1927-2010. In some embodiments, the retrotransposase comprises a sequence having at least about 96% identity to any one of SEQ ID NOs: 555-608 and 1927-2010. In some embodiments, the retrotransposase comprises a sequence having at least about 97% identity to any one of SEQ ID NOs: 555-608 and 1927-2010. In some embodiments, the retrotransposase comprises a sequence having at least about 98% identity to any one of SEQ ID NOs: 555-608 and 1927-2010. In some embodiments, the retrotransposase comprises a sequence having at least about 99% identity to any one of SEQ ID NOs: 555-608 and 1927-2010. In some embodiments, the retrotransposase comprises a sequence having 100% identity to any one of SEQ ID NOs: 555-608 and 1927-2010.

In some embodiments, the retrotransposase is a MG154 retrotransposase (i.e., SEQ ID NOs: 609-610 and 1555). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 609-610 and 1555. In some embodiments, the retrotransposase comprises a sequence having at least about 70% identity to any one of SEQ ID NOs: 609-610 and 1555. In some embodiments, the retrotransposase comprises a sequence having at least about 75% identity to any one of SEQ ID NOs: 609-610 and 1555. In some embodiments, the retrotransposase comprises a sequence having at least about 80% identity to any one of SEQ ID NOs: 609-610 and 1555. In some embodiments, the retrotransposase comprises a sequence having at least about 85% identity to any one of SEQ ID NOs: 609-610 and 1555. In some embodiments, the retrotransposase comprises a sequence having at least about 90% identity to any one of SEQ ID NOs: 609-610 and 1555. In some embodiments, the retrotransposase comprises a sequence having at least about 95% identity to any one of SEQ ID NOs: 609-610 and 1555. In some embodiments, the retrotransposase comprises a sequence having at least about 96% identity to any one of SEQ ID NOs: 609-610 and 1555. In some embodiments, the retrotransposase comprises a sequence having at least about 97% identity to any one of SEQ ID NOs: 609-610 and 1555. In some embodiments, the retrotransposase comprises a sequence having at least about 98% identity to any one of SEQ ID NOs: 609-610 and 1555. In some embodiments, the retrotransposase comprises a sequence having at least about 99% identity to any one of SEQ ID NOs: 609-610 and 1555. In some embodiments, the retrotransposase comprises a sequence having 100% identity to any one of SEQ ID NOs: 609-610 and 1555.

In some embodiments, the retrotransposase is a MG155 retrotransposase (i.e., SEQ ID NOs: 611-615 and 1544-1545). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 611-615 and 1544-1545. In some embodiments, the retrotransposase comprises a sequence having at least about 70% identity to any one of SEQ ID NOs: 611-615 and 1544-1545. In some embodiments, the retrotransposase comprises a sequence having at least about 75% identity to any one of SEQ ID NOs: 611-615 and 1544-1545. In some embodiments, the retrotransposase comprises a sequence having at least about 80% identity to any one of SEQ ID NOs: 611-615 and 1544-1545. In some embodiments, the retrotransposase comprises a sequence having at least about 85% identity to any one of SEQ ID NOs: 611-615 and 1544-1545. In some embodiments, the retrotransposase comprises a sequence having at least about 90% identity to any one of SEQ ID NOs: 611-615 and 1544-1545. In some embodiments, the retrotransposase comprises a sequence having at least about 95% identity to any one of SEQ ID NOs: 611-615 and 1544-1545. In some embodiments, the retrotransposase comprises a sequence having at least about 96% identity to any one of SEQ ID NOs: 611-615 and 1544-1545. In some embodiments, the retrotransposase comprises a sequence having at least about 97% identity to any one of SEQ ID NOs: 611-615 and 1544-1545. In some embodiments, the retrotransposase comprises a sequence having at least about 98% identity to any one of SEQ ID NOs: 611-615 and 1544-1545. In some embodiments, the retrotransposase comprises a sequence having at least about 99% identity to any one of SEQ ID NOs: 611-615 and 1544-1545. In some embodiments, the retrotransposase comprises a sequence having 100% identity to any one of SEQ ID NOs: 611-615 and 1544-1545.

In some embodiments, the retrotransposase is a MG156 retrotransposase (i.e., SEQ ID NO: 616 or SEQ ID NO: 617). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 616 or SEQ ID NO: 617. In some embodiments, the retrotransposase comprises a sequence having at least about 70% identity to SEQ ID NO: 616 or SEQ ID NO: 617. In some embodiments, the retrotransposase comprises a sequence having at least about 75% identity to SEQ ID NO: 616 or SEQ ID NO: 617. In some embodiments, the retrotransposase comprises a sequence having at least about 80% identity to SEQ ID NO: 616 or SEQ ID NO: 617. In some embodiments, the retrotransposase comprises a sequence having at least about 85% identity to SEQ ID NO: 616 or SEQ ID NO: 617. In some embodiments, the retrotransposase comprises a sequence having at least about 90% identity to SEQ ID NO: 616 or SEQ ID NO: 617. In some embodiments, the retrotransposase comprises a sequence having at least about 95% identity to SEQ ID NO: 616 or SEQ ID NO: 617. In some embodiments, the retrotransposase comprises a sequence having at least about 96% identity to SEQ ID NO: 616 or SEQ ID NO: 617. In some embodiments, the retrotransposase comprises a sequence having at least about 97% identity to SEQ ID NO: 616 or SEQ ID NO: 617. In some embodiments, the retrotransposase comprises a sequence having at least about 98% identity to SEQ ID NO: 616 or SEQ ID NO: 617. In some embodiments, the retrotransposase comprises a sequence having at least about 99% identity to SEQ ID NO: 616 or SEQ ID NO: 617. In some embodiments, the retrotransposase comprises a sequence having 100% identity to SEQ ID NO: 616 or SEQ ID NO: 617.

In some embodiments, the retrotransposase is a MG157 retrotransposase (i.e., SEQ ID NOs: 618-622 and 2258-2266). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 618-622 and 2258-2266. In some embodiments, the retrotransposase comprises a sequence having at least about 70% identity to any one of SEQ ID NOs: 618-622 and 2258-2266. In some embodiments, the retrotransposase comprises a sequence having at least about 75% identity to any one of SEQ ID NOs: 618-622 and 2258-2266. In some embodiments, the retrotransposase comprises a sequence having at least about 80% identity to any one of SEQ ID NOs: 618-622 and 2258-2266. In some embodiments, the retrotransposase comprises a sequence having at least about 85% identity to any one of SEQ ID NOs: 618-622 and 2258-2266. In some embodiments, the retrotransposase comprises a sequence having at least about 90% identity to any one of SEQ ID NOs: 618-622 and 2258-2266. In some embodiments, the retrotransposase comprises a sequence having at least about 95% identity to any one of SEQ ID NOs: 618-622 and 2258-2266. In some embodiments, the retrotransposase comprises a sequence having at least about 96% identity to any one of SEQ ID NOs: 618-622 and 2258-2266. In some embodiments, the retrotransposase comprises a sequence having at least about 97% identity to any one of SEQ ID NOs: 618-622 and 2258-2266. In some embodiments, the retrotransposase comprises a sequence having at least about 98% identity to any one of SEQ ID NOs: 618-622 and 2258-2266. In some embodiments, the retrotransposase comprises a sequence having at least about 99% identity to any one of SEQ ID NOs: 618-622 and 2258-2266. In some embodiments, the retrotransposase comprises a sequence having 100% identity to any one of SEQ ID NOs: 618-622 and 2258-2266.

In some embodiments, the retrotransposase is a MG158 retrotransposase (i.e., SEQ ID NO: 623). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 623. In some embodiments, the retrotransposase comprises a sequence having at least about 70% identity to SEQ ID NO: 623. In some embodiments, the retrotransposase comprises a sequence having at least about 75% identity to SEQ ID NO: 623. In some embodiments, the retrotransposase comprises a sequence having at least about 80% identity to SEQ ID NO: 623. In some embodiments, the retrotransposase comprises a sequence having at least about 85% identity to SEQ ID NO: 623. In some embodiments, the retrotransposase comprises a sequence having at least about 90% identity to SEQ ID NO: 623. In some embodiments, the retrotransposase comprises a sequence having at least about 95% identity to SEQ ID NO: 623. In some embodiments, the retrotransposase comprises a sequence having at least about 96% identity to SEQ ID NO: 623. In some embodiments, the retrotransposase comprises a sequence having at least about 97% identity to SEQ ID NO: 623. In some embodiments, the retrotransposase comprises a sequence having at least about 98% identity to SEQ ID NO: 623. In some embodiments, the retrotransposase comprises a sequence having at least about 99% identity to SEQ ID NO: 623. In some embodiments, the retrotransposase comprises a sequence having 100% identity to SEQ ID NO: 623.

In some embodiments, the retrotransposase is a MG159 retrotransposase (i.e., SEQ ID NOs: 624-626). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 624-626. In some embodiments, the retrotransposase comprises a sequence having at least about 70% identity to any one of SEQ ID NOs: 624-626. In some embodiments, the retrotransposase comprises a sequence having at least about 75% identity to any one of SEQ ID NOs: 624-626. In some embodiments, the retrotransposase comprises a sequence having at least about 80% identity to any one of SEQ ID NOs: 624-626. In some embodiments, the retrotransposase comprises a sequence having at least about 85% identity to any one of SEQ ID NOs: 624-626. In some embodiments, the retrotransposase comprises a sequence having at least about 90% identity to any one of SEQ ID NOs: 624-626. In some embodiments, the retrotransposase comprises a sequence having at least about 95% identity to any one of SEQ ID NOs: 624-626. In some embodiments, the retrotransposase comprises a sequence having at least about 96% identity to any one of SEQ ID NOs: 624-626. In some embodiments, the retrotransposase comprises a sequence having at least about 97% identity to any one of SEQ ID NOs: 624-626. In some embodiments, the retrotransposase comprises a sequence having at least about 98% identity to any one of SEQ ID NOs: 624-626. In some embodiments, the retrotransposase comprises a sequence having at least about 99% identity to any one of SEQ ID NOs: 624-626. In some embodiments, the retrotransposase comprises a sequence having 100% identity to any one of SEQ ID NOs: 624-626.

In some embodiments, the retrotransposase is a MG160 retrotransposase (i.e., SEQ ID NOs: 627-673, and 1039-1475, and 2011-2026). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 627-673, and 1039-1475, and 2011-2026. In some embodiments, the retrotransposase comprises a sequence having at least about 70% identity to any one of SEQ ID NOs: 627-673, and 1039-1475, and 2011-2026. In some embodiments, the retrotransposase comprises a sequence having at least about 75% identity to any one of SEQ ID NOs: 627-673, and 1039-1475, and 2011-2026. In some embodiments, the retrotransposase comprises a sequence having at least about 80% identity to any one of SEQ ID NOs: 627-673, and 1039-1475, and 2011-2026. In some embodiments, the retrotransposase comprises a sequence having at least about 85% identity to any one of SEQ ID NOs: 627-673, and 1039-1475, and 2011-2026. In some embodiments, the retrotransposase comprises a sequence having at least about 90% identity to any one of SEQ ID NOs: 627-673, and 1039-1475, and 2011-2026. In some embodiments, the retrotransposase comprises a sequence having at least about 95% identity to any one of SEQ ID NOs: 627-673, and 1039-1475, and 2011-2026. In some embodiments, the retrotransposase comprises a sequence having at least about 96% identity to any one of SEQ ID NOs: 627-673, and 1039-1475, and 2011-2026. In some embodiments, the retrotransposase comprises a sequence having at least about 97% identity to any one of SEQ ID NOs: 627-673, and 1039-1475, and 2011-2026. In some embodiments, the retrotransposase comprises a sequence having at least about 98% identity to any one of SEQ ID NOs: 627-673, and 1039-1475, and 2011-2026. In some embodiments, the retrotransposase comprises a sequence having at least about 99% identity to any one of SEQ ID NOs: 627-673, and 1039-1475, and 2011-2026. In some embodiments, the retrotransposase comprises a sequence having 100% identity to any one of SEQ ID NOs: 627-673, and 1039-1475, and 2011-2026.

In some embodiments, the retrotransposase is a MG163 retrotransposase (i.e., SEQ ID NOs: 674-678). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 674-678. In some embodiments, the retrotransposase comprises a sequence having at least about 70% identity to any one of SEQ ID NOs: 674-678. In some embodiments, the retrotransposase comprises a sequence having at least about 75% identity to any one of SEQ ID NOs: 674-678. In some embodiments, the retrotransposase comprises a sequence having at least about 80% identity to any one of SEQ ID NOs: 674-678. In some embodiments, the retrotransposase comprises a sequence having at least about 85% identity to any one of SEQ ID NOs: 674-678. In some embodiments, the retrotransposase comprises a sequence having at least about 90% identity to any one of SEQ ID NOs: 674-678. In some embodiments, the retrotransposase comprises a sequence having at least about 95% identity to any one of SEQ ID NOs: 674-678. In some embodiments, the retrotransposase comprises a sequence having at least about 96% identity to any one of SEQ ID NOs: 674-678. In some embodiments, the retrotransposase comprises a sequence having at least about 97% identity to any one of SEQ ID NOs: 674-678. In some embodiments, the retrotransposase comprises a sequence having at least about 98% identity to any one of SEQ ID NOs: 674-678. In some embodiments, the retrotransposase comprises a sequence having at least about 99% identity to any one of SEQ ID NOs: 674-678. In some embodiments, the retrotransposase comprises a sequence having 100% identity to any one of SEQ ID NOs: 674-678.

In some embodiments, the retrotransposase is a MG164 retrotransposase (i.e., SEQ ID NOs: 679-683). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 679-683. In some embodiments, the retrotransposase comprises a sequence having at least about 70% identity to any one of SEQ ID NOs: 679-683. In some embodiments, the retrotransposase comprises a sequence having at least about 75% identity to any one of SEQ ID NOs: 679-683. In some embodiments, the retrotransposase comprises a sequence having at least about 80% identity to any one of SEQ ID NOs: 679-683. In some embodiments, the retrotransposase comprises a sequence having at least about 85% identity to any one of SEQ ID NOs: 679-683. In some embodiments, the retrotransposase comprises a sequence having at least about 90% identity to any one of SEQ ID NOs: 679-683. In some embodiments, the retrotransposase comprises a sequence having at least about 95% identity to any one of SEQ ID NOs: 679-683. In some embodiments, the retrotransposase comprises a sequence having at least about 96% identity to any one of SEQ ID NOs: 679-683. In some embodiments, the retrotransposase comprises a sequence having at least about 97% identity to any one of SEQ ID NOs: 679-683. In some embodiments, the retrotransposase comprises a sequence having at least about 98% identity to any one of SEQ ID NOs: 679-683. In some embodiments, the retrotransposase comprises a sequence having at least about 99% identity to any one of SEQ ID NOs: 679-683. In some embodiments, the retrotransposase comprises a sequence having 100% identity to any one of SEQ ID NOs: 679-683.

In some embodiments, the retrotransposase is a MG165 retrotransposase (i.e., SEQ ID NOs: 684-692 and 2027-2046). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 684-692 and 2027-2046. In some embodiments, the retrotransposase comprises a sequence having at least about 70% identity to any one of SEQ ID NOs: 684-692 and 2027-2046. In some embodiments, the retrotransposase comprises a sequence having at least about 75% identity to any one of SEQ ID NOs: 684-692 and 2027-2046. In some embodiments, the retrotransposase comprises a sequence having at least about 80% identity to any one of SEQ ID NOs: 684-692 and 2027-2046. In some embodiments, the retrotransposase comprises a sequence having at least about 85% identity to any one of SEQ ID NOs: 684-692 and 2027-2046. In some embodiments, the retrotransposase comprises a sequence having at least about 90% identity to any one of SEQ ID NOs: 684-692 and 2027-2046. In some embodiments, the retrotransposase comprises a sequence having at least about 95% identity to any one of SEQ ID NOs: 684-692 and 2027-2046. In some embodiments, the retrotransposase comprises a sequence having at least about 96% identity to any one of SEQ ID NOs: 684-692 and 2027-2046. In some embodiments, the retrotransposase comprises a sequence having at least about 97% identity to any one of SEQ ID NOs: 684-692 and 2027-2046. In some embodiments, the retrotransposase comprises a sequence having at least about 98% identity to any one of SEQ ID NOs: 684-692 and 2027-2046. In some embodiments, the retrotransposase comprises a sequence having at least about 99% identity to any one of SEQ ID NOs: 684-692 and 2027-2046. In some embodiments, the retrotransposase comprises a sequence having 100% identity to any one of SEQ ID NOs: 684-692 and 2027-2046.

In some embodiments, the retrotransposase is a MG166 retrotransposase (i.e., SEQ ID NOs: 693-697 and 2047-2090). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 693-697 and 2047-2090. In some embodiments, the retrotransposase comprises a sequence having at least about 70% identity to any one of SEQ ID NOs: 693-697 and 2047-2090. In some embodiments, the retrotransposase comprises a sequence having at least about 75% identity to any one of SEQ ID NOs: 693-697 and 2047-2090. In some embodiments, the retrotransposase comprises a sequence having at least about 80% identity to any one of SEQ ID NOs: 693-697 and 2047-2090. In some embodiments, the retrotransposase comprises a sequence having at least about 85% identity to any one of SEQ ID NOs: 693-697 and 2047-2090. In some embodiments, the retrotransposase comprises a sequence having at least about 90% identity to any one of SEQ ID NOs: 693-697 and 2047-2090. In some embodiments, the retrotransposase comprises a sequence having at least about 95% identity to any one of SEQ ID NOs: 693-697 and 2047-2090. In some embodiments, the retrotransposase comprises a sequence having at least about 96% identity to any one of SEQ ID NOs: 693-697 and 2047-2090. In some embodiments, the retrotransposase comprises a sequence having at least about 97% identity to any one of SEQ ID NOs: 693-697 and 2047-2090. In some embodiments, the retrotransposase comprises a sequence having at least about 98% identity to any one of SEQ ID NOs: 693-697 and 2047-2090. In some embodiments, the retrotransposase comprises a sequence having at least about 99% identity to any one of SEQ ID NOs: 693-697 and 2047-2090. In some embodiments, the retrotransposase comprises a sequence having 100% identity to any one of SEQ ID NOs: 693-697 and 2047-2090.

In some embodiments, the retrotransposase is a MG167 retrotransposase (i.e., SEQ ID NOs: 698-702 and 2091-2119). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 698-702 and 2091-2119. In some embodiments, the retrotransposase comprises a sequence having at least about 70% identity to any one of SEQ ID NOs: 698-702 and 2091-2119. In some embodiments, the retrotransposase comprises a sequence having at least about 75% identity to any one of SEQ ID NOs: 698-702 and 2091-2119. In some embodiments, the retrotransposase comprises a sequence having at least about 80% identity to any one of SEQ ID NOs: 698-702 and 2091-2119. In some embodiments, the retrotransposase comprises a sequence having at least about 85% identity to any one of SEQ ID NOs: 698-702 and 2091-2119. In some embodiments, the retrotransposase comprises a sequence having at least about 90% identity to any one of SEQ ID NOs: 698-702 and 2091-2119. In some embodiments, the retrotransposase comprises a sequence having at least about 95% identity to any one of SEQ ID NOs: 698-702 and 2091-2119. In some embodiments, the retrotransposase comprises a sequence having at least about 96% identity to any one of SEQ ID NOs: 698-702 and 2091-2119. In some embodiments, the retrotransposase comprises a sequence having at least about 97% identity to any one of SEQ ID NOs: 698-702 and 2091-2119. In some embodiments, the retrotransposase comprises a sequence having at least about 98% identity to any one of SEQ ID NOs: 698-702 and 2091-2119. In some embodiments, the retrotransposase comprises a sequence having at least about 99% identity to any one of SEQ ID NOs: 698-702 and 2091-2119. In some embodiments, the retrotransposase comprises a sequence having 100% identity to any one of SEQ ID NOs: 698-702 and 2091-2119.

In some embodiments, the retrotransposase is a MG168 retrotransposase (i.e., SEQ ID NOs: 703-707). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 703-707. In some embodiments, the retrotransposase comprises a sequence having at least about 70% identity to any one of SEQ ID NOs: 703-707. In some embodiments, the retrotransposase comprises a sequence having at least about 75% identity to any one of SEQ ID NOs: 703-707. In some embodiments, the retrotransposase comprises a sequence having at least about 80% identity to any one of SEQ ID NOs: 703-707. In some embodiments, the retrotransposase comprises a sequence having at least about 85% identity to any one of SEQ ID NOs: 703-707. In some embodiments, the retrotransposase comprises a sequence having at least about 90% identity to any one of SEQ ID NOs: 703-707. In some embodiments, the retrotransposase comprises a sequence having at least about 95% identity to any one of SEQ ID NOs: 703-707. In some embodiments, the retrotransposase comprises a sequence having at least about 96% identity to any one of SEQ ID NOs: 703-707. In some embodiments, the retrotransposase comprises a sequence having at least about 97% identity to any one of SEQ ID NOs: 703-707. In some embodiments, the retrotransposase comprises a sequence having at least about 98% identity to any one of SEQ ID NOs: 703-707. In some embodiments, the retrotransposase comprises a sequence having at least about 99% identity to any one of SEQ ID NOs: 703-707. In some embodiments, the retrotransposase comprises a sequence having 100% identity to any one of SEQ ID NOs: 703-707.

In some embodiments, the retrotransposase is a MG169 retrotransposase (i.e., SEQ ID NOs: 708-718 and 2121-2159). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 708-718 and 2121-2159. In some embodiments, the retrotransposase comprises a sequence having at least about 70% identity to any one of SEQ ID NOs: 708-718 and 2121-2159. In some embodiments, the retrotransposase comprises a sequence having at least about 75% identity to any one of SEQ ID NOs: 708-718 and 2121-2159. In some embodiments, the retrotransposase comprises a sequence having at least about 80% identity to any one of SEQ ID NOs: 708-718 and 2121-2159. In some embodiments, the retrotransposase comprises a sequence having at least about 85% identity to any one of SEQ ID NOs: 708-718 and 2121-2159. In some embodiments, the retrotransposase comprises a sequence having at least about 90% identity to any one of SEQ ID NOs: 708-718 and 2121-2159. In some embodiments, the retrotransposase comprises a sequence having at least about 95% identity to any one of SEQ ID NOs: 708-718 and 2121-2159. In some embodiments, the retrotransposase comprises a sequence having at least about 96% identity to any one of SEQ ID NOs: 708-718 and 2121-2159. In some embodiments, the retrotransposase comprises a sequence having at least about 97% identity to any one of SEQ ID NOs: 708-718 and 2121-2159. In some embodiments, the retrotransposase comprises a sequence having at least about 98% identity to any one of SEQ ID NOs: 708-718 and 2121-2159. In some embodiments, the retrotransposase comprises a sequence having at least about 99% identity to any one of SEQ ID NOs: 708-718 and 2121-2159. In some embodiments, the retrotransposase comprises a sequence having 100% identity to any one of SEQ ID NOs: 708-718 and 2121-2159.

In some embodiments, the retrotransposase is a MG170 retrotransposase (i.e., SEQ ID NOs: 719-728). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 719-728. In some embodiments, the retrotransposase comprises a sequence having at least about 70% identity to any one of SEQ ID NOs: 719-728. In some embodiments, the retrotransposase comprises a sequence having at least about 75% identity to any one of SEQ ID NOs: 719-728. In some embodiments, the retrotransposase comprises a sequence having at least about 80% identity to any one of SEQ ID NOs: 719-728. In some embodiments, the retrotransposase comprises a sequence having at least about 85% identity to any one of SEQ ID NOs: 719-728. In some embodiments, the retrotransposase comprises a sequence having at least about 90% identity to any one of SEQ ID NOs: 719-728. In some embodiments, the retrotransposase comprises a sequence having at least about 95% identity to any one of SEQ ID NOs: 719-728. In some embodiments, the retrotransposase comprises a sequence having at least about 96% identity to any one of SEQ ID NOs: 719-728. In some embodiments, the retrotransposase comprises a sequence having at least about 97% identity to any one of SEQ ID NOs: 719-728. In some embodiments, the retrotransposase comprises a sequence having at least about 98% identity to any one of SEQ ID NOs: 719-728. In some embodiments, the retrotransposase comprises a sequence having at least about 99% identity to any one of SEQ ID NOs: 719-728. In some embodiments, the retrotransposase comprises a sequence having 100% identity to any one of SEQ ID NOs: 719-728.

In some embodiments, the retrotransposase is a MG172 retrotransposase (i.e., SEQ ID NOs: 729-733). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 729-733. In some embodiments, the retrotransposase comprises a sequence having at least about 70% identity to any one of SEQ ID NOs: 729-733. In some embodiments, the retrotransposase comprises a sequence having at least about 75% identity to any one of SEQ ID NOs: 729-733. In some embodiments, the retrotransposase comprises a sequence having at least about 80% identity to any one of SEQ ID NOs: 729-733. In some embodiments, the retrotransposase comprises a sequence having at least about 85% identity to any one of SEQ ID NOs: 729-733. In some embodiments, the retrotransposase comprises a sequence having at least about 90% identity to any one of SEQ ID NOs: 729-733. In some embodiments, the retrotransposase comprises a sequence having at least about 95% identity to any one of SEQ ID NOs: 729-733. In some embodiments, the retrotransposase comprises a sequence having at least about 96% identity to any one of SEQ ID NOs: 729-733. In some embodiments, the retrotransposase comprises a sequence having at least about 97% identity to any one of SEQ ID NOs: 729-733. In some embodiments, the retrotransposase comprises a sequence having at least about 98% identity to any one of SEQ ID NOs: 729-733. In some embodiments, the retrotransposase comprises a sequence having at least about 99% identity to any one of SEQ ID NOs: 729-733. In some embodiments, the retrotransposase comprises a sequence having 100% identity to any one of SEQ ID NOs: 729-733.

In some embodiments, the retrotransposase is a MG173 retrotransposase (i.e., SEQ ID NOs: 734-735 and 1546-1553). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 734-735 and 1546-1553. In some embodiments, the retrotransposase comprises a sequence having at least about 70% identity to any one of SEQ ID NOs: 734-735 and 1546-1553. In some embodiments, the retrotransposase comprises a sequence having at least about 75% identity to any one of SEQ ID NOs: 734-735 and 1546-1553. In some embodiments, the retrotransposase comprises a sequence having at least about 80% identity to any one of SEQ ID NOs: 734-735 and 1546-1553. In some embodiments, the retrotransposase comprises a sequence having at least about 85% identity to any one of SEQ ID NOs: 734-735 and 1546-1553. In some embodiments, the retrotransposase comprises a sequence having at least about 90% identity to any one of SEQ ID NOs: 734-735 and 1546-1553. In some embodiments, the retrotransposase comprises a sequence having at least about 95% identity to any one of SEQ ID NOs: 734-735 and 1546-1553. In some embodiments, the retrotransposase comprises a sequence having at least about 96% identity to any one of SEQ ID NOs: 734-735 and 1546-1553. In some embodiments, the retrotransposase comprises a sequence having at least about 97% identity to any one of SEQ ID NOs: 734-735 and 1546-1553. In some embodiments, the retrotransposase comprises a sequence having at least about 98% identity to any one of SEQ ID NOs: 734-735 and 1546-1553. In some embodiments, the retrotransposase comprises a sequence having at least about 99% identity to any one of SEQ ID NOs: 734-735 and 1546-1553. In some embodiments, the retrotransposase comprises a sequence having 100% identity to any one of SEQ ID NOs: 734-735 and 1546-1553.

In some embodiments, the retrotransposase is a MG176 retrotransposase (i.e., SEQ ID NO: 1038 or SEQ ID NO: 2160). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 1038 or SEQ ID NO: 2160. In some embodiments, the retrotransposase comprises a sequence having at least about 70% identity to SEQ ID NO: 1038 or SEQ ID NO: 2160. In some embodiments, the retrotransposase comprises a sequence having at least about 75% identity to SEQ ID NO: 1038 or SEQ ID NO: 2160. In some embodiments, the retrotransposase comprises a sequence having at least about 80% identity to SEQ ID NO: 1038 or SEQ ID NO: 2160. In some embodiments, the retrotransposase comprises a sequence having at least about 85% identity to SEQ ID NO: 1038 or SEQ ID NO: 2160. In some embodiments, the retrotransposase comprises a sequence having at least about 90% identity to SEQ ID NO: 1038 or SEQ ID NO: 2160. In some embodiments, the retrotransposase comprises a sequence having at least about 95% identity to SEQ ID NO: 1038 or SEQ ID NO: 2160. In some embodiments, the retrotransposase comprises a sequence having at least about 96% identity to SEQ ID NO: 1038 or SEQ ID NO: 2160. In some embodiments, the retrotransposase comprises a sequence having at least about 97% identity to SEQ ID NO: 1038 or SEQ ID NO: 2160. In some embodiments, the retrotransposase comprises a sequence having at least about 98% identity to SEQ ID NO: 1038 or SEQ ID NO: 2160. In some embodiments, the retrotransposase comprises a sequence having at least about 99% identity to SEQ ID NO: 1038 or SEQ ID NO: 2160. In some embodiments, the retrotransposase comprises a sequence having 100% identity to SEQ ID NO: 1038 or SEQ ID NO: 2160.

In some embodiments, the retrotransposase is a MG192 retrotransposase (i.e., SEQ ID NO: 1554). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO: 1554. In some embodiments, the retrotransposase comprises a sequence having at least about 70% identity to SEQ ID NO: 1554. In some embodiments, the retrotransposase comprises a sequence having at least about 75% identity to SEQ ID NO: 1554. In some embodiments, the retrotransposase comprises a sequence having at least about 80% identity to SEQ ID NO: 1554. In some embodiments, the retrotransposase comprises a sequence having at least about 85% identity to SEQ ID NO: 1554. In some embodiments, the retrotransposase comprises a sequence having at least about 90% identity to SEQ ID NO: 1554. In some embodiments, the retrotransposase comprises a sequence having at least about 95% identity to SEQ ID NO: 1554. In some embodiments, the retrotransposase comprises a sequence having at least about 96% identity to SEQ ID NO: 1554. In some embodiments, the retrotransposase comprises a sequence having at least about 97% identity to SEQ ID NO: 1554. In some embodiments, the retrotransposase comprises a sequence having at least about 98% identity to SEQ ID NO: 1554. In some embodiments, the retrotransposase comprises a sequence having at least about 99% identity to SEQ ID NO: 1554. In some embodiments, the retrotransposase comprises a sequence having 100% identity to SEQ ID NO: 1554.

In some embodiments, the retrotransposase is encoded by a nucleic acid sequence that is codon optimized. In some embodiments, the retrotransposase is encoded by a nucleic acid sequence that is codon optimized for expression in a mammalian cell. In some embodiments, the retrotransposase is encoded by a nucleic acid sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity with the nucleic acid sequence of any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806. In some embodiments, the retrotransposase is encoded by a nucleic acid sequence having at least 70% sequence identity with the nucleic acid sequence of any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806. In some embodiments, the retrotransposase is encoded by a nucleic acid sequence having at least 75% sequence identity with the nucleic acid sequence of any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806. In some embodiments, the retrotransposase is encoded by a nucleic acid sequence having at least 80% sequence identity with the nucleic acid sequence of any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806. In some embodiments, the retrotransposase is encoded by a nucleic acid sequence having at least 85% sequence identity with the nucleic acid sequence of any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806. In some embodiments, the retrotransposase is encoded by a nucleic acid sequence having at least 90% sequence identity with the nucleic acid sequence of any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806. In some embodiments, the retrotransposase is encoded by a nucleic acid sequence having at least 95% sequence identity with the nucleic acid sequence of any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568. In some embodiments, the retrotransposase is encoded by a nucleic acid sequence having at least 96% sequence identity with the nucleic acid sequence of any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568. In some embodiments, the retrotransposase is encoded by a nucleic acid sequence having at least 97% sequence identity with the nucleic acid sequence of any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806. In some embodiments, the retrotransposase is encoded by a nucleic acid sequence having at least 98% sequence identity with the nucleic acid sequence of any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536,1539-1543, 1556-1568, and 1611-1806. In some embodiments, the retrotransposase is encoded by a nucleic acid sequence having at least 99% sequence identity with the nucleic acid sequence of any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806. In some embodiments, the retrotransposase is encoded by a nucleic acid sequence of any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806.

In some embodiments, the retrotransposase comprises a reverse transcriptase domain. In some embodiments, the retrotransposase further comprises one or more zinc finger domains. In some embodiments, the retrotransposase further comprises an endonuclease finger domain. In some embodiments, the retrotransposase comprises a conserved catalytic D, QG, [Y/F]XDD, or LG motif. In some embodiments, the retrotransposase comprises a conserved CX_{[2-3]}C Zn finger motif.

In some embodiments, the retrotransposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a documented retrotransposase.

In some embodiments, the cargo nucleotide sequence is flanked by a 3′ untranslated region (UTR) and a 5′ untranslated region (UTR).

In some embodiments, the retrotransposase is configured to transpose the cargo nucleotide sequence as single-stranded deoxyribonucleic acid polynucleotide. In some embodiments, the retrotransposase is configured to transpose the cargo nucleotide sequence as double-stranded deoxyribonucleic acid polynucleotide. In some embodiments, the retrotransposase is configured to transpose the cargo nucleotide sequence via a ribonucleic acid polynucleotide intermediate.

In some embodiments, the retrotransposase comprises one or more nuclear localization sequences (NLSs). In some embodiments, the NLS is proximal to the N- or C-terminus of the retrotransposase. In some embodiments, the NLS is appended N-terminal or C-terminal of the retrotransposase and comprise any one of SEQ ID NOs: 1477-1492, or having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1477-1492. In some cases, the NLS comprises a sequence having at least about 80% identity to SEQ ID NOs: 1477-1492. In some cases, the NLS comprises a sequence having at least about 85% identity to SEQ ID NOs: 1477-1492. In some cases, the NLS comprises a sequence having at least about 90% identity to SEQ ID NOs: 1477-1492. In some cases, the NLS comprises a sequence having at least about 91% identity to SEQ ID NOs: 1477-1492. In some cases, the NLS comprises a sequence having at least about 92% identity to SEQ ID NOs: 1477-1492. In some cases, the NLS comprises a sequence having at least about 93% identity to SEQ ID NOs: 1477-1492. In some cases, the NLS comprises a sequence having at least about 94% identity to SEQ ID NOs: 1477-1492. In some cases, the NLS comprises a sequence having at least about 95% identity to SEQ ID NOs: 1477-1492. In some cases, the NLS comprises a sequence having at least about 96% identity to SEQ ID NOs: 1477-1492. In some cases, the NLS comprises a sequence having at least about 97% identity to SEQ ID NOs: 1477-1492. In some cases, the NLS comprises a sequence having at least about 98% identity to SEQ ID NOs: 1477-1492. In some cases, the NLS comprises a sequence having at least about 99% identity to SEQ ID NOs: 1477-1492. In some cases, the NLS comprises a sequence having 100% identity to SEQ ID NOs: 1477-1492. In some cases, the NLS comprises a sequence having 100% identity to SEQ ID NO: 1477. In some cases, the NLS comprises a sequence having 100% identity to SEQ ID NOs: 1478.

TABLE 1
Example NLS Sequences that may be used with
retrotransposases according to the disclosure

| | NLS amino | SEQ ID |
|---|---|---|
| Source | acid sequence | NO: |
| SV40 | PKKKRKV | 1477 |
| nucleoplasmin | KRPAATKKAGQAKKKK | 1478 |
| bipartite NLS | | |
| c-myc NLS | PAAKRVKLD | 1479 |
| c-myc NLS | RQRRNELKRSP | 1480 |
| hRNPA1 M9 NLS | NQSSNFGPMKGGNFGGRSSGP | 1481 |
| | YGGGGQYFAKPRNQGGY | |
| Importin-alpha | RMRIZFKNKGKDTAELRRRRV | 1482 |
| IBB domain | EVSVELRKAKKDEQILKRRNV | |
| Myoma T protein | VSRKRPRP | 1483 |
| Myoma T protein | PPKKARED | 1484 |
| p53 | PQPKKKPL | 1485 |
| mouse c-abl IV | SALIKKKKKMAP | 1486 |
| influenza virus | DRLRR | 1487 |
| NS1 | | |
| influenza virus | PKQKKRK | 1488 |
| NS1 | | |
| Hepatitis virus | RKLKKKIKKL | 1489 |
| delta antigen | | |
| mouse Mx1 protein | REKKKFLKRR | 1490 |
| human poly(ADP- | KRKGDEVDGVDEVAKKKSKK | 1491 |
| ribose) polymerase | | |
| steroid hormone | RKCLQAGMNLEARKTKK | 1492 |
| receptors (human) | | |
| glucocorticoid | | |

In some embodiments, the retrotransposase comprises a tag. In some embodiments, the tag is an affinity tag. Exemplary affinity tags include, but are not limited to, a His-tag, a Flag tag, a Myc-tag, an MBP-tag, and a GST-tag.

In some embodiments, the retrotransposase comprises a protease cleavage site. Exemplary protease cleavage sites include, but are not limited to, a TEV site, a C3 site, a Factor Xa site, and an Enterokinase site.

In some embodiments, the retrotransposase is tethered to a site directed nuclease. In some embodiments, the retrotransposase is fused to a site directed nuclease. In some embodiments, the retrotransposase is recruited to a site directed nuclease. In some embodiments, the site directed nuclease is an endonuclease. In some embodiments, the site directed nuclease is a Cas nuclease. In some embodiments, the Cas nuclease is an RNA guided CRISPR Cas9 nuclease. In some embodiments, the site directed nuclease is a dead nuclease or a nickase. In some embodiments, the site directed nuclease brings the retrotransposase into close proximity of a target site that is to be modified.

### Guide Nucleic Acids

In some embodiments, the retrotransposase system further comprises a site directed nuclease and a guide RNA (e.g., gRNA). In a polynucleotide when referring to a T, a T means U (Uracil) in RNA and T (Thymine) in DNA. In some embodiments, the retrotransposase systems described herein comprise a means for directing the site directed nuclease to a particular location in the target nucleic acid.

In some embodiments, the guide RNA comprises synthetic nucleotides or modified nucleotides. In some embodiments, the guide RNA comprises one or more inter-nucleoside linkers modified from the natural phosphodiester. In some embodiments, all of the inter-nucleoside linkers of the guide RNA, or contiguous nucleotide sequence thereof, are modified. For example, in some embodiments, the inter nucleoside linkage comprises Sulphur(S), such as a phosphorothioate inter-nucleoside linkage.

In some embodiments, the guide RNA comprises modifications to a ribose sugar or nucleobase. In some embodiments, the guide RNA comprises one or more nucleosides comprising a modified sugar moiety, wherein the modified sugar moiety is a modification of the sugar moiety when compared to the ribose sugar moiety found in deoxyribose nucleic acid (DNA) and RNA. In some embodiments, the modification is within the ribose ring structure. Exemplary modifications include, but are not limited to, replacement with a hexose ring (HNA), a bicyclic ring having a biradical bridge between the C2 and C4 carbons on the ribose ring (e.g., locked nucleic acids (LNA)), or an unlinked ribose ring which typically lacks a bond between the C2 and C3 carbons (e.g., UNA). In some embodiments, the sugar-modified nucleosides comprise bicyclohexose nucleic acids or tricyclic nucleic acids. In some embodiments, the modified nucleosides comprise nucleosides where the sugar moiety is replaced with a non-sugar moiety, for example peptide nucleic acids (PNA) or morpholino nucleic acids.

In some embodiments, the guide RNA comprises one or more modified sugars. In some embodiments, the sugar modifications comprise modifications made by altering the substituent groups on the ribose ring to groups other than hydrogen, or the 2′-OH group naturally found in DNA and RNA nucleosides. In some embodiments, substituents are introduced at the 2′, 3′, 4′, or 5′ positions, or combinations thereof. In some embodiments, nucleosides with modified sugar moieties comprise 2′ modified nucleosides, e.g., 2′ substituted nucleosides. A 2′ sugar modified nucleoside, in some embodiments, is a nucleoside that has a substituent other than —H or —OH at the 2′ position (2′ substituted nucleoside) or comprises a 2′ linked biradical, and comprises 2′ substituted nucleosides and LNA (2′-4′ biradical bridged) nucleosides. Examples of 2′-substituted modified nucleosides comprise, but are not limited to, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the guide RNA comprises one or more modified sugars. In some embodiments, the guide RNA comprises only modified sugars. In certain embodiments, the guide RNA comprises greater than about 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the guide RNA comprises both inter-nucleoside linker modifications and nucleoside modifications.

In some cases, the guide RNA comprises a sequence complementary to a eukaryotic, fungal, plant, mammalian, or human genomic polynucleotide sequence. In some cases, the guide RNA comprises a sequence complementary to a eukaryotic genomic polynucleotide sequence. In some cases, the guide RNA comprises a sequence complementary to a fungal genomic polynucleotide sequence. In some cases, the guide RNA comprises a sequence complementary to a plant genomic polynucleotide sequence. In some cases, the guide RNA comprises a sequence complementary to a mammalian genomic polynucleotide sequence. In some cases, the guide RNA comprises a sequence complementary to a human genomic polynucleotide sequence.

In some cases, the guide RNA is 30-400 nucleotides in length. In some cases, the guide RNA is 85-245 nucleotides in length. In some cases, the guide RNA is more than 90 nucleotides in length. In some cases, the guide RNA is less than 245 nucleotides in length. In some embodiments, the guide RNA is 30, 40, 50, 60, 70, 80, 90, 100, 120, 140, 160, 180, 200, 220, 240, or more than 240 nucleotides in length. In some embodiments, the guide RNA is about 30 to about 40, about 30 to about 50, about 30 to about 60, about 30 to about 70, about 30 to about 80, about 30 to about 90, about 30 to about 100, about 30 to about 120, about 30 to about 140, about 30 to about 160, about 30 to about 180, about 30 to about 200, about 30 to about 220, about 30 to about 240, about 50 to about 60, about 50 to about 70, about 50 to about 80, about 50 to about 90, about 50 to about 100, about 50 to about 120, about 50 to about 140, about 50 to about 160, about 50 to about 180, about 50 to about 200, about 50 to about 220, about 50 to about 240, about 100 to about 120, about 100 to about 140, about 100 to about 160, about 100 to about 180, about 100 to about 200, about 100 to about 220, about 100 to about 240, about 160 to about 180, about 160 to about 200, about 160 to about 220, or about 160 to about 240 nucleotides in length.

In some embodiments, the gRNA is encoded by any one of the nucleic acid sequences of SEQ ID NOs: 903-926 and 934-951, a sequence having at least about 80%, 85%, 90%, 95%, 97%, 98%, or 99% sequence identity to any one of the nucleic acid sequences of SEQ ID NOs: 903-926 and 934-951, or a reverse complement thereof. In some embodiments, the guide RNA is encoded by a sequence having at least about 80% sequence identity to any one of the nucleic acid sequences of SEQ ID NOs: 903-926 and 934-951 or a reverse complement thereof. In some embodiments, the guide RNA is encoded by a sequence having at least about 85% sequence identity to any one of the nucleic acid sequences of SEQ ID NOs: 903-926 and 934-951 or a reverse complement thereof. In some embodiments, the guide RNA is encoded by a sequence having at least about 90% sequence identity to any one of the nucleic acid sequences of SEQ ID NOs: 903-926 and 934-951 or a reverse complement thereof. In some embodiments, the guide RNA is encoded by a sequence having at least about 95% sequence identity to any one of the nucleic acid sequences of SEQ ID NOs: 903-926 and 934-951 or a reverse complement thereof. In some embodiments, the guide RNA is encoded by a sequence having at least about 97% sequence identity to any one of the nucleic acid sequences of SEQ ID NOs: 903-926 and 934-951 or a reverse complement thereof. In some embodiments, the guide RNA is encoded by a sequence having at least about 98% sequence identity to any one of the nucleic acid sequences of SEQ ID NOs: 903-926 and 934-951 or a reverse complement thereof. In some embodiments, the guide RNA is encoded by a sequence having at least about 99% sequence identity to any one of the nucleic acid sequences of SEQ ID NOs: 903-926 and 934-951 or a reverse complement thereof. In some embodiments, the guide RNA is encoded by a sequence according to any one of the nucleic acid sequences of SEQ ID NOs: 903-926 and 934-951 or a reverse complement thereof.

In some embodiments, the sequence is determined by a BLASTP, CLUSTALW, MUSCLE, or MAFFT algorithm, or a CLUSTALW algorithm with the Smith-Waterman homology search algorithm parameters. In some embodiments, the sequence is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

### Cargo Nucleic Acids

In some embodiments, the retrotransposase system comprises a cargo nucleic acid or polynucleotide. In some embodiments, the cargo nucleic acid is comprised in a double-stranded deoxyribonucleic acid. In some embodiments, the cargo nucleic acid is a eukaryotic, plant, fungal, mammalian, rodent, or human double-stranded deoxyribonucleic acid polynucleotide. In some embodiments, the cargo nucleotide sequence is flanked by a 3′ untranslated region (UTR) and a 5′ untranslated region (UTR).

In some embodiments, the cargo nucleic acid comprises synthetic nucleotides or modified nucleotides. In some embodiments, the cargo nucleic acid comprises one or more inter-nucleoside linkers modified from the natural phosphodiester. In some embodiments, all of the inter-nucleoside linkers of the cargo nucleic acid, or contiguous nucleotide sequence thereof, are modified. For example, in some embodiments, the inter-nucleoside linkage comprises Sulphur (S), such as a phosphorothioate inter-nucleoside linkage.

In some embodiments, the cargo nucleic acid comprises modifications to a ribose sugar or nucleobase. In some embodiments, the cargo nucleic acid comprises one or more nucleosides comprising a modified sugar moiety, wherein the modified sugar moiety is a modification of the sugar moiety when compared to the ribose sugar moiety found in deoxyribose nucleic acid (DNA) and RNA. In some embodiments, the modification is within the ribose ring structure. Exemplary modifications include, but are not limited to, replacement with a hexose ring (HNA), a bicyclic ring having a biradical bridge between the C2 and C4 carbons on the ribose ring (e.g., locked nucleic acids (LNA)), or an unlinked ribose ring which typically lacks a bond between the C2 and C3 carbons (e.g., UNA). In some embodiments, the sugar-modified nucleosides comprise bicyclohexose nucleic acids or tricyclic nucleic acids. In some embodiments, the modified nucleosides comprise nucleosides where the sugar moiety is replaced with a non-sugar moiety, for example peptide nucleic acids (PNA) or morpholino nucleic acids.

In some embodiments, the cargo nucleic acid comprises one or more modified sugars. In some embodiments, the sugar modifications comprise modifications made by altering the substituent groups on the ribose ring to groups other than hydrogen, or the 2′-OH group naturally found in DNA and RNA nucleosides. In some embodiments, substituents are introduced at the 2′, 3′, 4′, 5′ positions, or combinations thereof. In some embodiments, nucleosides with modified sugar moieties comprise 2′ modified nucleosides, e.g., 2′ substituted nucleosides. A 2′ sugar modified nucleoside, in some embodiments, is a nucleoside that has a substituent other than —H or —OH at the 2′ position (2′ substituted nucleoside) or comprises a 2′ linked biradical, and comprises 2′ substituted nucleosides and LNA (2′-4′ biradical bridged) nucleosides. Examples of 2′-substituted modified nucleosides comprise, but are not limited to, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the cargo nucleic acid comprises one or more modified sugars. In some embodiments, the cargo nucleic acid comprises only modified sugars. In certain embodiments, the cargo nucleic acid comprises greater than about 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the cargo nucleic acid comprises both inter-nucleoside linker modifications and nucleoside modifications.

### MG Systems

Described herein, in certain embodiments, are engineered retrotransposase system, comprising: (a) a double-stranded nucleic acid comprising a cargo nucleotide sequence configured to form a complex with a retrotransposase; and (b) a retrotransposase configured to transpose the cargo nucleotide sequence to a target nucleic acid sequence. In some embodiments, engineered retrotransposase systems described herein comprise a means for cutting a target nucleic acid sequence.

In some embodiments, the engineered retrotransposase system comprises (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 70% sequence identity to any one of SEQ ID NOs: 1-29, 393-735, 799-895, 1020-1476, 1544-1554, 1850-2160, 2165-2210, and 2258-2266. In some embodiments, the engineered retrotransposase system comprises (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 75% sequence identity to any one of SEQ ID NOs: 1-29, 393-735, 799-895, 1020-1476, 1544-1554, 1850-2160, 2165-2210, and 2258-2266. In some embodiments, the engineered retrotransposase system comprises (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 80% sequence identity to any one of SEQ ID NOs: 1-29, 393-735, 799-895, 1020-1476, 1544-1554, 1850-2160, 2165-2210, and 2258-2266. In some embodiments, the engineered retrotransposase system comprises (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 85% sequence identity to any one of SEQ ID NOs: 1-29, 393-735, 799-895, 1020-1476, 1544-1554, 1850-2160, 2165-2210, and 2258-2266. In some embodiments, the engineered retrotransposase system comprises (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 90% sequence identity to any one of SEQ ID NOs: 1-29, 393-735, 799-895, 1020-1476, 1544-1554, 1850-2160, 2165-2210, and 2258-2266. In some embodiments, the engineered retrotransposase system comprises (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 95% sequence identity to any one of SEQ ID NOs: 1-29, 393-735, 799-895, 1020-1476, 1544-1554, 1850-2160, 2165-2210, and 2258-2266. In some embodiments, the engineered retrotransposase system comprises (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 96% sequence identity to any one of SEQ ID NOs: 1-29, 393-735, 799-895, 1020-1476, 1544-1554, 1850-2160, 2165-2210, and 2258-2266. In some embodiments, the engineered retrotransposase system comprises (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 97% sequence identity to any one of SEQ ID NOs: 1-29, 393-735, 799-895, 1020-1476, 1544-1554, 1850-2160, 2165-2210, and 2258-2266. In some embodiments, the engineered retrotransposase system comprises (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 98% sequence identity to any one of SEQ ID NOs: 1-29, 393-735, 799-895, 1020-1476, 1544-1554, 1850-2160, 2165-2210, and 2258-2266. In some embodiments, the engineered retrotransposase system comprises (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having at least 99% sequence identity to any one of SEQ ID NOs: 1-29, 393-735, 799-895, 1020-1476, 1544-1554, 1850-2160, 2165-2210, and 2258-2266. In some embodiments, the engineered retrotransposase system comprises (a) a double-stranded nucleic acid comprising a cargo nucleotide sequence configured to form a complex with a retrotransposase; and (b) a retrotransposase configured to transpose the cargo nucleotide sequence to a target nucleic acid sequence and comprising an amino acid sequence having 100% sequence identity to any one of SEQ ID NOs: 1-29, 393-735, 799-895, 1020-1476, 1544-1554, 1850-2160, 2165-2210, and 2258-2266.

In some embodiments, the retrotransposase is a MG140 retrotransposase (i.e., SEQ ID NOs: 1-29, 393-401, 799-894, 1476, 1850-1926, and 2165-2210). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 1-29, 393-401, 799-894, 1476, 1850-1926, and 2165-2210.

In some embodiments, the retrotransposase is a MG146 retrotransposase (i.e., SEQ ID NO: 402 or SEQ ID NO: 895). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 402 or SEQ ID NO: 895.

In some embodiments, the retrotransposase is a MG148 retrotransposase (i.e., SEQ ID NOs: 403-426). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 403-426.

In some embodiments, the retrotransposase is a MG149 retrotransposase (i.e., SEQ ID NOs: 427-439). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 427-439.

In some embodiments, the retrotransposase is a MG151 retrotransposase (i.e., SEQ ID NOs: 440-554 and 1020-1037). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity ty to any one of SEQ ID NOs: 440-554 and 1020-1037.

In some embodiments, the retrotransposase is a MG153 retrotransposase (i.e., SEQ ID NOs: 555-608 and 1927-2010). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 555-608 and 1927-2010.

In some embodiments, the retrotransposase is a MG154 retrotransposase (i.e., SEQ ID NOs: 609-610 and 1555). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 609-610 and 1555.

In some embodiments, the retrotransposase is a MG155 retrotransposase (i.e., SEQ ID NOs: 611-615 and 1544-1545). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 611-615 and 1544-1545.

In some embodiments, the retrotransposase is a MG156 retrotransposase (i.e., SEQ ID NO: 616 or SEQ ID NO: 617). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 616 or SEQ ID NO: 617.

In some embodiments, the retrotransposase is a MG157 retrotransposase (i.e., SEQ ID NOs: 618-622 and 2258-2266). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 618-622 and 2258-2266.

In some embodiments, the retrotransposase is a MG158 retrotransposase (i.e., SEQ ID NO: 623). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 623.

In some embodiments, the retrotransposase is a MG159 retrotransposase (i.e., SEQ ID NOs: 624-626). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 624-626.

In some embodiments, the retrotransposase is a MG160 retrotransposase (i.e., SEQ ID NOs: 627-673, and 1039-1475, and 2011-2026). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 627-673, and 1039-1475, and 2011-2026.

In some embodiments, the retrotransposase is a MG163 retrotransposase (i.e., SEQ ID NOs: 674-678). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 674-678.

In some embodiments, the retrotransposase is a MG164 retrotransposase (i.e., SEQ ID NOs: 679-683). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 679-683.

In some embodiments, the retrotransposase is a MG165 retrotransposase (i.e., SEQ ID NOs: 684-692 and 2027-2046). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 684-692 and 2027-2046.

In some embodiments, the retrotransposase is a MG166 retrotransposase (i.e., SEQ ID NOs: 693-697 and 2047-2090). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 693-697 and 2047-2090.

In some embodiments, the retrotransposase is a MG167 retrotransposase (i.e., SEQ ID NOs: 698-702 and 2091-2119). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 698-702 and 2091-2119.

In some embodiments, the retrotransposase is a MG168 retrotransposase (i.e., SEQ ID NOs: 703-707). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 703-707.

In some embodiments, the retrotransposase is a MG169 retrotransposase (i.e., SEQ ID NOs: 708-718 and 2121-2159). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 708-718 and 2121-2159.

In some embodiments, the retrotransposase is a MG170 retrotransposase (i.e., SEQ ID NOs: 719-728). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 719-728.

In some embodiments, the retrotransposase is a MG172 retrotransposase (i.e., SEQ ID NOs: 729-733). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 729-733.

In some embodiments, the retrotransposase is a MG173 retrotransposase (i.e., SEQ ID NOs: 734-735 and 1546-1553). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 734-735 and 1546-1553.

In some embodiments, the retrotransposase is a MG176 retrotransposase (i.e., SEQ ID NO: 1038 or SEQ ID NO: 2160). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 1038 or SEQ ID NO: 2160.

In some embodiments, the retrotransposase is a MG192 retrotransposase (i.e., SEQ ID NO: 1554). In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 1554.

### Cells

Described herein, in certain embodiments, is a cell comprising the systems described herein.

In some embodiments, the cell is a eukaryotic cell (e.g., a plant cell, an animal cell, a protist cell, or a fungi cell), a mammalian cell (a Chinese hamster ovary (CHO) cell, baby hamster kidney (BHK), human embryo kidney (HEK), mouse myeloma (NSO), or human retinal cells), an immortalized cell (e.g., a HeLa cell, a COS cell, a HEK-293T cell, a MDCK cell, a 3T3 cell, a PC12 cell, a Huh7 cell, a HepG2 cell, a K562 cell, a N2a cell, or a SY5Y cell), an insect cell (e.g., a Spodoptera frugiperda cell, a Trichoplusia ni cell, a Drosophila melanogaster cell, a S2 cell, or a Heliothis virescens cell), a yeast cell (e.g., a Saccharomyces cerevisiae cell, a Cryptococcus cell, or a Candida cell), a plant cell (e.g., a parenchyma cell, a collenchyma cell, or a sclerenchyma cell), a fungal cell (e.g., a Saccharomyces cerevisiae cell, a Cryptococcus cell, or a Candida cell), or a prokaryotic cell (e.g., a E. coli cell, a streptococcus bacterium cell, a streptomyces soil bacteria cell, or an archaea cell). In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell.

In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, a primary cell, or derivative thereof. In some embodiments, the cell is an engineered cell. In some embodiments, the cell is a stable cell (i.e., a cell that has constant expression of a specific gene or protein).

### Delivery and Vectors

Disclosed herein, in some embodiments, are nucleic acid sequences encoding the engineered retrotransposase systems described herein.

In some embodiments, the present disclosure provides a nucleic acid comprising an engineered nucleic acid sequence encoding a retrotransposase described herein. In some embodiments, the engineered nucleic acid sequence encoding a retrotransposase is optimized for expression in an organism. In some embodiments, the retrotransposase is derived from an uncultivated microorganism. In some embodiments, the organism is not the uncultivated organism.

In some embodiments, the organism is prokaryotic. In some embodiments, the organism is bacterial. In some embodiments, the organism is eukaryotic. In some embodiments, the organism is fungal. In some embodiments, the organism is a plant. In some embodiments, the organism is mammalian. In some embodiments, the organism is a rodent. In some embodiments, the organism is human.

In some embodiments, the nucleic acid encoding the engineered retrotransposase system is a DNA, for example a linear DNA, a plasmid DNA, or a minicircle DNA. In some embodiments, the nucleic acid encoding the engineered nuclease system is an RNA, for example a mRNA.

In some embodiments, the nucleic acid encoding the engineered retrotransposase systems is delivered by a nucleic acid-based vector. In some embodiments, the nucleic acid-based vector is plasmid (e.g., circular DNA molecules that can autonomously replicate inside a cell), cosmid (e.g., pWE or sCos vectors), artificial chromosome, human artificial chromosome (HAC), yeast artificial chromosomes (YAC), bacterial artificial chromosome (BAC), P1-derived artificial chromosomes (PAC), phagemid, phage derivative, bacmid, or virus. In some embodiments, the vector is selected from the group consisting of: pSF-CMV-NEO-NH2-PPT-3×FLAG, pSF-CMV-NEO-COOH-3×FLAG, pSF-CMV-PURO-NH2-GST-TEV, pSF-OXB20-COOH-TEV-FLAG (R)-6His, pCEP4 pDEST27, pSF-CMV-Ub-KrYFP, pSF-CMV-FMDV-daGFP, pEFla-mCherry-N1 vector, pEFla-tdTomato vector, pSF-CMV-FMDV-Hygro, pSF-CMV-PGK-Puro, pMCP-tag (m), pSF-CMV-PURO-NH2-CMYC, pSF-OXB20-BetaGal, pSF-OXB20-Fluc, pSF-OXB20, pSF-Tac, pRI 101-AN DNA, pCambia2301, pTYB21 pKLAC2, pAc5.1/V5-His A, and pDEST8.

In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the virus is an alphavirus. In some embodiments, the virus is a parvovirus. In some embodiments, the virus is an adenovirus. In some embodiments, the virus is an AAV. In some embodiments, the virus is a baculovirus. In some embodiments, the virus is a Dengue virus. In some embodiments, the virus is a lentivirus. In some embodiments, the virus is a herpesvirus. In some embodiments, the virus is a poxvirus. In some embodiments, the virus is an anellovirus. In some embodiments, the virus is a bocavirus. In some embodiments, the virus is a vaccinia virus. In some embodiments, the virus is a retrovirus.

In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2YF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, AAV-HSC16, or a derivative thereof. In some embodiments, the herpesvirus is HSV type 1, HSV-2, VZV, EBV, CMV, HHV-6, HHV-7, or HHV-8.

In some embodiments, the nucleic acid encoding the engineered retrotransposase system is delivered by a non-nucleic acid-based delivery system (e.g., a non-viral delivery system). In some embodiments, the non-viral delivery system is a liposome. In some embodiments, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the nucleic acid, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, the nucleic acid is comprised in a lipid nanoparticle (LNP).

In some embodiments, the endonuclease or gene editing system (e.g., retrotransposase) is introduced into a cell (e.g., host cell) in any suitable way, either stably or transiently. In some embodiments, the endonuclease or gene editing system is transfected into the cell. In some embodiments, the cell is transduced or transfected with a nucleic acid construct that encodes the endonuclease or gene editing system. For example, a cell is transduced (e.g., with a virus encoding the endonuclease or gene editing system), or transfected (e.g., with a plasmid encoding the endonuclease or gene editing system) with a nucleic acid that encodes the endonuclease or gene editing system. In some embodiments, the transduction is a stable or transient transduction. In some embodiments, cells expressing the endonuclease or gene editing system or containing the endonuclease or gene editing system are transduced or transfected with one or more gRNA molecules, for example when the endonuclease or gene editing system comprises the retrotransposase. In some embodiments, a plasmid expressing the endonuclease or gene editing system is introduced into cells through electroporation, transient (e.g., lipofection) or stable genome integration (e.g., piggybac), or viral transduction (for example lentivirus or AAV), or other methods known to those of skill in the art. In some embodiments, the gene editing system is introduced into the cell as one or more polypeptides. In some embodiments, delivery is achieved through the use of RNP complexes. Delivery methods to cells for polypeptides and/or RNPs are known in the art, for example by electroporation or by cell squeezing.

Exemplary methods of delivery of nucleic acids include lipofection, nucleofection, electroporation, stable genome integration (e.g., piggybac), microinjection, biolistics, virosomes, liposomes, immunoliposomes, polycation or lipidnucleic acid conjugates, naked DNA, artificial virions, and agent-enhanced uptake of DNA. Lipofection is described in e.g., U.S. Pat. Nos. 5,049,386; 4,946,787; and 4,897,355; and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of WO 91/17424 and WO 91/16024. In some embodiments, the delivery is to cells (e.g., in vitro or ex vivo administration) or target tissues (e.g., in vivo administration). In some embodiments, the nucleic acid is comprised in a liposome or a nanoparticle that specifically targets a host cell.

Additional methods for the delivery of nucleic acids to cells are known to those skilled in the art. See, for example, US 2003/0087817.

### Methods of Use

Systems of the present disclosure may be used for various applications, such as, for example, nucleic acid editing (e.g., gene editing), binding to a nucleic acid molecule (e.g., sequence-specific binding). Such systems may be used, for example, for addressing (e.g., removing or replacing) a genetically inherited mutation that may cause a disease in a subject, inactivating a gene in order to ascertain its function in a cell, as a diagnostic tool to detect disease-causing genetic elements (e.g., via cleavage of reverse-transcribed viral RNA or an amplified DNA sequence encoding a disease-causing mutation), as deactivated enzymes in combination with a probe to target and detect a specific nucleotide sequence (e.g., sequence encoding antibiotic resistance int bacteria), to render viruses inactive or incapable of infecting host cells by targeting viral genomes, to add genes or amend metabolic pathways to engineer organisms to produce valuable small molecules, macromolecules, or secondary metabolites, to establish a gene drive element for evolutionary selection, to detect cell perturbations by foreign small molecules and nucleotides as a biosensor.

Described herein, in certain embodiments, are methods for modifying a target nucleic acid comprising providing an engineered retrotransposase system. In some embodiments, the present disclosure provides a method for binding, nicking, cleaving, marking, modifying, or transposing a double-stranded deoxyribonucleic acid polynucleotide. In some embodiments, the method comprises contacting the double-stranded deoxyribonucleic acid polynucleotide with a retrotransposase.

In some embodiments, the double-stranded deoxyribonucleic acid polynucleotide is a eukaryotic, plant, fungal, mammalian, rodent, or human double-stranded deoxyribonucleic acid polynucleotide.

In some embodiments, the retrotransposase is configured to transpose the cargo nucleotide sequence as single-stranded deoxyribonucleic acid polynucleotide. In some embodiments, the retrotransposase is configured to transpose the cargo nucleotide sequence as double-stranded deoxyribonucleic acid polynucleotide. In some embodiments, the retrotransposase is configured to transpose the cargo nucleotide sequence via a ribonucleic acid polynucleotide intermediate. In some embodiments, the cargo nucleotide sequence is flanked by a 3′ untranslated region (UTR) and a 5′ untranslated region (UTR).

In some embodiments, the present disclosure provides a method of modifying a target nucleic acid sequence (e.g., locus). In some embodiments, the method comprises delivering to the target nucleic acid sequence the engineered retrotransposase system described herein. In some embodiments, the complex is configured such that upon binding of the complex to the target nucleic acid sequence, the complex modifies the target nucleic acid sequence.

In some embodiments, modifying the target nucleic acid sequence comprises binding, nicking, cleaving, marking, modifying, or transposing the target nucleic acid sequence. In some embodiments, the target nucleic acid sequence comprises deoxyribonucleic acid (DNA) or ribonucleic acid (RNA). In some embodiments, the target nucleic acid comprises genomic DNA, viral DNA, viral RNA, or bacterial DNA. In some embodiments, the target nucleic acid sequence is in vitro. In some embodiments, the target nucleic acid sequence is within a cell. In some embodiments, the cell is a prokaryotic cell, a bacterial cell, a eukaryotic cell, a fungal cell, a plant cell, an animal cell, a mammalian cell, a rodent cell, a primate cell, or a human cell. In some embodiments, the cell is a primary cell. In some embodiments, the primary cell is a T cell. In some embodiments, the primary cell is a hematopoietic stem cell (HSC). In some embodiments, the cell is a human cell. In some embodiments, the cell is genome edited ex vivo. In some embodiments, the cell is genome edited in vivo.

In some embodiments, delivery of the engineered retrotransposase system to the target nucleic acid sequence comprises delivering the nucleic acid described herein or the vector described herein. In some embodiments, delivery of engineered retrotransposase system to the target nucleic acid sequence comprises delivering a nucleic acid comprising an open reading frame encoding the retrotransposase. In some embodiments, the nucleic acid comprises a promoter. In some embodiments, the open reading frame encoding the retrotransposase is operably linked to the promoter.

In some embodiments, delivery of the engineered retrotransposase system to the target nucleic acid sequence comprises delivering a capped mRNA containing the open reading frame encoding the retrotransposase. In some embodiments, delivery of the engineered retrotransposase system to the target nucleic acid sequence comprises delivering a translated polypeptide. In some embodiments, delivery of the engineered retrotransposase system to the target nucleic acid sequence comprises delivering a deoxyribonucleic acid (DNA) encoding the engineered retrotransposase operably linked to a ribonucleic acid (RNA) pol III promoter.

In some embodiments, the retrotransposase does not induce a break at or proximal to the target nucleic acid sequence.

In some embodiments, the transposition activity is measured in vitro by introducing the retrotransposase to cells comprising the target nucleic acid sequence and detecting transposition of the target nucleic acid sequence in the cells. In some embodiments, the composition comprises 20 pmoles or less of the retrotransposase. In some embodiments, the composition comprises 1 pmol or less of the retrotransposase.

Further described herein, in certain embodiments, are methods of manufacturing a retrotransposase. In some embodiments, the method comprises cultivating a host cell with the engineered retrotransposase system described herein.

In some embodiments, the host cell is a bacterial cell. In some embodiments, the bacterial cell is Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus reuteri, Lactobacillus rhamnosus, Lactobacillus johnsonii, Lactobacillus plantarum, Lactobacillus fermentum, Lactococcus lactis, Streptococcus thermophilus, Lactococcus lactis, Lactococcus diacetylactis, Lactococcus cremoris, Lactobacillus bulgaricus, Lactobacillus helveticus, Lactobacillus delbrueckii, or Escherichia coli. In some embodiments, the host cell is an E. coli cell. In some embodiments, the E. coli cell is a λDE3 lysogen or a BL21 (DE3) strain. In some embodiments, the E. coli cell has an ompT lon genotype.

In some embodiments, the host cell is an E. coli cell. In some embodiments, the E. coli cell is a λDE3 lysogen or the E. coli cell is a BL21 (DE3) strain. In some embodiments, the E. coli cell has an ompT lon genotype.

In some embodiments, the open reading frame is operably linked to a promoter sequence. In some embodiments, the promoter is selected from the group consisting of a mini promoter, an inducible promoter, a constitutive promoter, and derivatives thereof. In some embodiments, the promoter is selected from the group consisting of CMV, CBA, EF1a, CAG, PGK, TRE, U6, UAS, T7, Sp6, lac, araBad, trp, Ptac, p5, p19, p40, Synapsin, CaMKII, GRK1, and derivatives thereof.

In some embodiments, the open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araP_{BAD} promoter, a strong leftward promoter from phage lambda (pL promoter), or any combination thereof.

In some embodiments, the open reading frame comprises a sequence encoding an affinity tag linked in-frame to a sequence encoding the retrotransposase. In some embodiments, the affinity tag is an immobilized metal affinity chromatography (IMAC) tag. In some embodiments, the IMAC tag is a polyhistidine tag. In some embodiments, the affinity tag is a myc tag, a human influenza hemagglutinin (HA) tag, a maltose binding protein (MBP) tag, a glutathione S-transferase (GST) tag, a streptavidin tag, a FLAG tag, or any combination thereof. In some embodiments, the affinity tag is linked in-frame to the sequence encoding the retrotransposase via a linker sequence encoding a protease cleavage site. In some embodiments, the protease cleavage site is a tobacco etch virus (TEV) protease cleavage site, a PreScission® protease cleavage site, a Thrombin cleavage site, a Factor Xa cleavage site, an enterokinase cleavage site, or any combination thereof.

In some embodiments, the open reading frame is codon-optimized for expression in the host cell. In some embodiments, the open reading frame is provided on a vector. In some embodiments, the open reading frame is integrated into a genome of the host cell.

In some embodiments, the present disclosure provides a culture comprising a host cell described herein in compatible liquid medium.

In some embodiments, the present disclosure provides a method of producing a retrotransposase, comprising cultivating a host cell described herein in compatible growth medium. In some embodiments, the method further comprises inducing expression of the retrotransposase by addition of an additional chemical agent or an increased amount of a nutrient. In some embodiments, the additional chemical agent or increased amount of a nutrient comprises Isopropyl β-D-1-thiogalactopyranoside (IPTG) or additional amounts of lactose. In some embodiments, the method further comprises isolating the host cell after the cultivation and lysing the host cell to produce a protein extract. In some embodiments, the method further comprises subjecting the protein extract to IMAC, or ion-affinity chromatography. In some embodiments, the open reading frame comprises a sequence encoding an IMAC affinity tag linked in-frame to a sequence encoding the retrotransposase. In some embodiments, the IMAC affinity tag is linked in-frame to the sequence encoding the retrotransposase via a linker sequence encoding protease cleavage site. In some embodiments, the protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof. In some embodiments, the method further comprises cleaving the IMAC affinity tag by contacting a protease corresponding to the protease cleavage site to the retrotransposase. In some embodiments, the method further comprises performing subtractive IMAC affinity chromatography to remove the affinity tag from a composition comprising the retrotransposase.

### Kits

In some embodiments, this disclosure provides kits comprising one or more nucleic acid constructs encoding the various components of the retrotransposase or gene editing system described herein, e.g., comprising a nucleotide sequence encoding the components of the retrotransposase or gene editing system capable of modifying a target DNA sequence. In some embodiments, the nucleotide sequence comprises a heterologous promoter that drives expression of the gene editing system components.

In some embodiments, any of the retrotransposase or gene editing systems disclosed herein is assembled into a pharmaceutical, diagnostic, or research kit to facilitate its use in therapeutic, diagnostic, or research applications. A kit may include one or more containers housing any of the vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions, in some embodiments, are in a form prescribed by a governmental agency regulating the manufacture, use, or sale of pharmaceuticals or biological products, which instructions can also reflect approval by the agency of manufacture, use, or sale for animal administration.

### EXAMPLES

### Example 1—A Method of Metagenomic Analysis for New Proteins

Samples for metagenomic analysis were collected from sediment, soil, and animals. Samples were collected with consent of property owners. Additional raw sequence data from public sources included animal microbiomes, sediment, soil, hot springs, hydrothermal vents, marine, peat bogs, permafrost, and sewage sequences. Deoxyribonucleic acid (DNA) was extracted with a DNA mini-prep kit and sequenced. Metagenomic sequence data was searched based on documented retrotransposase protein sequences to identify new retrotransposases. Retrotransposase proteins identified by the search were aligned to documented proteins to identify potential active sites. This metagenomic workflow resulted in the delineation of the MG140 family described herein.

### Example 2-Discovery of MG140, MG146, MG147, MG148, MG149, MG151, MG153, MG154, MG155, MG156, MG157, MG158, MG159, MG160, MG163, MG164, MG165, MG166, MG167, MG168, MG169, MG170, MG172, MG173, and MG176 Families of Retrotransposases

Metagenomic data analysis of the retrotransposase proteins identified in Example 1 revealed a new cluster of undescribed putative retrotransposase systems comprising several families (MG140, MG146, MG147, MG148, MG149, MG151, MG153, MG154, MG155, MG156, MG157, MG158, MG159, MG160, MG163, MG164, MG165, MG166, MG167, MG168, MG169, MG170, MG172, MG173, and MG176). The corresponding protein sequences for these new enzymes and their example subdomains are presented as SEQ ID NOs: 1-29, 393-735, 799-895, 1020-1476, 1544-1554, 1850-2160, 2165-2210, and 2258-2266.

### Example 3—Integration of Reverse Transcribed DNA In Vitro Activity (Prophetic)

Integrase activity can be conducted via expression in an E. coli lysate-based expression system. The components used for in vitro testing are three plasmids: an expression plasmid with the retrotransposon gene(s) under a T7 promoter, a target plasmid, and a donor plasmid which contains 5′ and 3′ UTR sequences recognized by the retrotransposase around a selection marker gene (e.g., Tet resistance gene). The lysate-based expression products, target DNA, and donor plasmid are incubated to allow for transposition to occur. Transposition is detected via PCR. In addition, the transposition product will be tagmented with T5 and sequenced via NGS to determine the insertion sites on a population of transposition events. Alternatively, the in vitro transposition products can be transformed into E. coli under antibiotic (e.g., Tet) selection, where growth occurs when the selection marker is stably inserted into a plasmid. Either single colonies or a population of E. coli can be sequenced to determine the insertion sites.

Integration efficiency can be measured via ddPCR or qPCR of the experimental output of target DNA with integrated cargo, normalized to the amount of unmodified target DNA also measured via ddPCR.

This assay may also be conducted with purified protein components rather than from lysate-based expression. In this case, the proteins are expressed in E. coli protease-deficient B strain under T7 inducible promoter, the cells are lysed using sonication, and the His-tagged protein of interest is purified using Ni-NTA affinity chromatography on a FPLC. Purity is determined using densitometry of the protein bands resolved on SDS-PAGE and coomassie stained acrylamide gels. The protein is desalted in storage buffer composed of 50 mM Tris-HCl, 300 mM NaCl, 1 mM TCEP, 5% glycerol; pH 7.5 (or other buffers as determined for maximum stability) and stored at −80° C. After purification the transposon gene(s) are added to the target DNA and donor plasmid as described above in a reaction buffer, for example 26 mM HEPES pH 7.5, 4.2 mM TRIS pH 8, 50 μg/mL BSA, 2 mM ATP, 2.1 mM DTT, 0.05 mM EDTA, 0.2 mM MgCl₂, 30-200 mM NaCl, 21 mM KCl, 1.35% glycerol, (measured pH 7.5) supplemented with 15 mM MgOAc₂.

### Example 4—Retrotransposon End Verification Via Gel Shift (Prophetic)

The retrotransposon ends are tested for retrotransposase binding via an electrophoretic mobility shift assay (EMSA). In this case, a target DNA fragment (100-500 bp) is end-labeled with FAM via PCR with FAM-labeled primers. The 3′ UTR RNA and 5′ UTR RNA are generated in vitro using T7 RNA polymerase and purified. The retrotransposase proteins are synthesized in an in vitro transcription/translation system. After synthesis, 1 μL of protein is added to 50 nM of the labeled DNA and 100 ng of the 3′ or 5′ UTR RNA in a 10 μL reaction in binding buffer (e.g., 20 mM HEPES pH 7.5, 2.5 mM Tris pH 7.5, 10 mM NaCl, 0.0625 mM EDTA, 5 mM TCEP, 0.005% BSA, 1 μg/mL poly(dI-dC), and 5% glycerol). The binding is incubated at 30° for 40 minutes, then 2 μL of 6× loading buffer (60 mM KCl, 10 mM Tris pH 7.6, 50% glycerol) is added. The binding reaction is separated on a 5% TBE gel and visualized. Shifts of the 3′ or 5′ UTR in the presence of retrotransposase protein and target DNA can be attributed to successful binding and are indicative of retrotransposase activity. This assay can also be performed with retrotransposase truncations or mutations, as well as using E. coli extract or purified protein.

### Example 5—Cleavage of Target DNA Verification (Prophetic)

To confirm that the retrotransposase is involved in cleavage of target DNA, short (~140 bp) DNA fragments are labelled at both ends with FAM via PCR with FAM-labeled primers. In vitro transcription/translation retrotransposase products are pre-incubated with 1 μg of Rnase A (negative control), or 3′ UTR, 5′ UTR or non-specific RNA fragments (control), followed by incubating with labeled target DNA at 37° C. The DNA is then analyzed on a denaturing gel. Cleavage of one or both strands of DNA can result in labelled fragments of various sizes, which migrate at different rates on the gel.

### Example 6—Integrase Activity in E. coli (Prophetic)

Engineered E. coli strains are transformed with a plasmid expressing the retrotransposon genes and a plasmid containing a temperature-sensitive origin of replication with a selectable marker flanked by 5′ and 3′ UTR of the retrotransposon involved in integration. Transformants induced for expression of these genes are then screened for transfer of the marker to a genomic target by selection at restrictive temperature for plasmid replication and the marker integration in the genome is confirmed by PCR.

Integrations are screened using an unbiased approach. In brief, purified gDNA is tagmented with Tn5, and DNA of interest is then PCR amplified using primers specific to the Tn5 tagmentation and the selectable marker. The amplicons are then prepared for NGS sequencing. Analysis of the resulting sequences is trimmed of the transposon sequences and flanking sequences are mapped to the genome to determine insertion position, and insertion rates are determined.

### Example 7—Integration of Reverse Transcribed DNA into Mammalian Genomes (Prophetic)

To show targeting and cleavage activity in mammalian cells, the integrase proteins are purified in E. coli or sf9 cells with 2 NLS peptides either in the N, C or both terminus of the protein sequence. In this procedure, a plasmid containing a selectable neomycin resistance marker (NeoR), or a fluorescent marker flanked by the 5′ and 3′ UTR regions involved in transposition and under control of a CMV promoter is synthesized. Cells are be transfected with the plasmid, recovered for 4-6 hours for RNA transcription, and subsequently electroporated with purified integrase proteins. Antibiotic resistance integration into the genome is quantified by G418-resistant colony counts (selection to start 7 days post-transfection), and positive transposition by the fluorescent marker is assayed by fluorescence activated cell cytometry. 7-10 days after the second transfection, genomic DNA is extracted and used for the preparation of an NGS library. Off target frequency is assayed by fragmenting the genome and preparing amplicons of the transposon marker and flanking DNA for NGS library preparation. At least 40 different target sites are chosen for testing each targeting system's activity.

Integration in mammalian cells can also be assessed via RNA delivery. An RNA encoding the retrotransposase with 2 NLS is designed, and cap and polyA tail are added. A second RNA is designed containing a selectable neomycin resistance marker (NeoR) or a fluorescent marker flanked by the 5′ and 3′ UTR regions. The RNA constructs are introduced into mammalian cells via liposome based transfection reagent. 10 days post-transfection, genomic DNA is extracted to measure transposition efficiency using ddPCR and NGS.

### Example 8—Bioinformatic Discovery of RTs

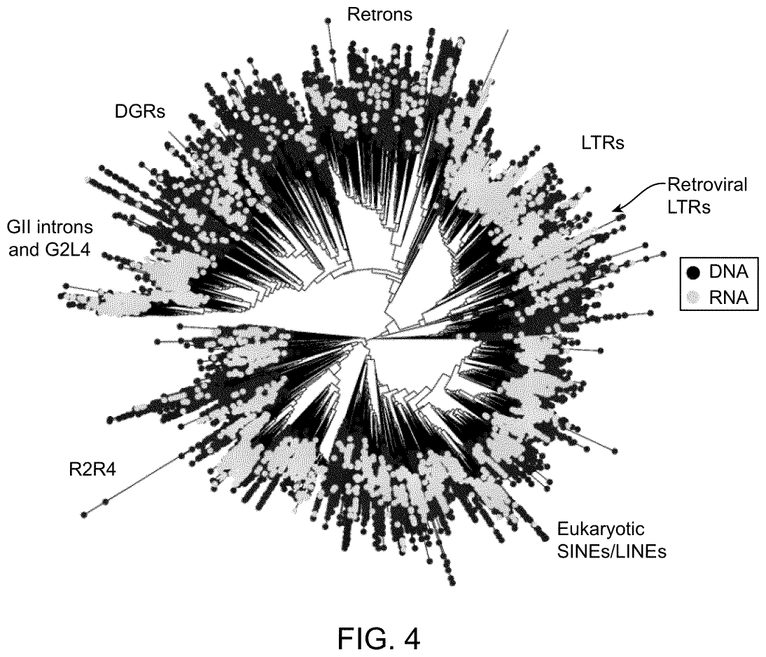

An extensive assembly-driven metagenomic database of microbial, viral, and eukaryotic genomes was mined to retrieve predicted proteins with reverse transcriptase function. Over 4.5 million RT proteins were predicted on the basis of having a hit to the Pfam domains PF00078 and PF07727, of which 3.4 million had a significant e-value (<1×10⁻⁵). After filtering for complete ORFs with an RT (reverse transcriptase) domain coverage of ≥70%, and with predicted catalytic residues ([F/Y]XDD), nearly half a million proteins were retained for further analysis. The RT domains were extracted from this set of proteins, as well as from reference sequences retrieved from public databases. The domain sequences were clustered at 50% identity over 80% coverage with Mmseqs2 easy-cluster, representative sequences (26,824 in total) were aligned, and the domain alignment was used to infer a phylogenetic tree. Phylogenetic analysis of RT domains suggest that many different classes of RTs with high sequence diversity were recovered (FIG. 4).

### Example 9—Example Non-LTR Retrotransposons (MG140, MG146, MG147, MG148, and MG149 Families)

### Retrotransposon Bioinformatic Analysis

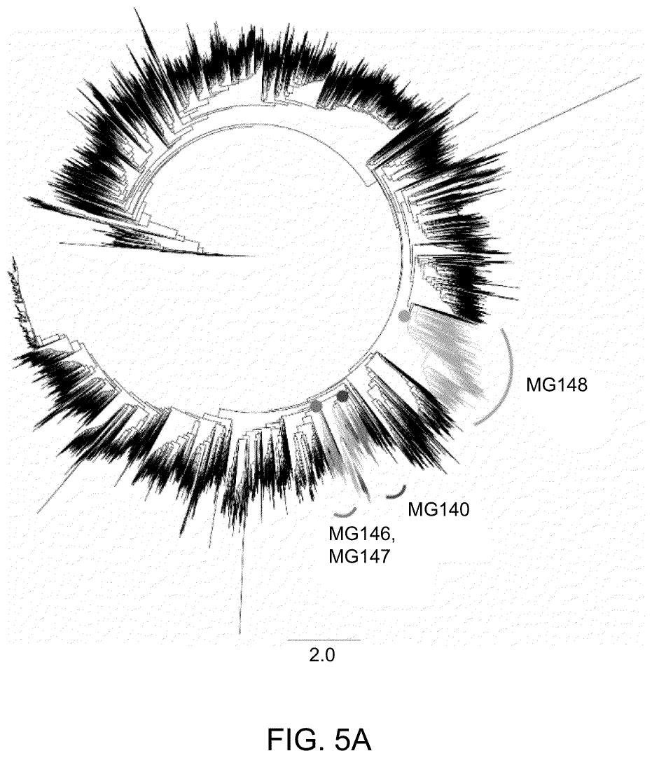

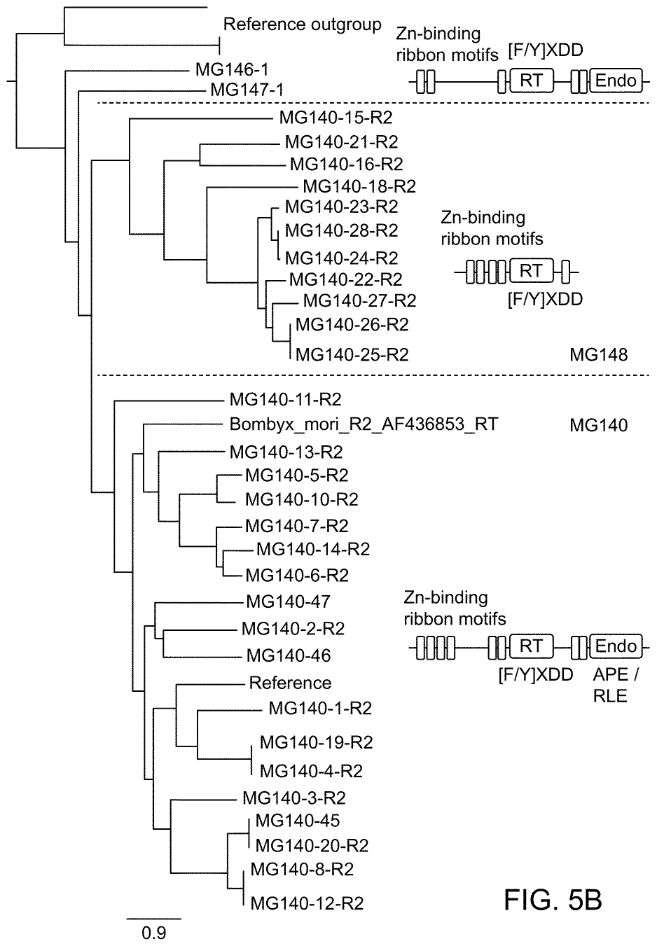

Non long terminal repeat (non-LTR) retrotransposases are capable of integrating large cargo into a target site via reverse transcription of an RNA template. Non-LTR retrotransposases were identified within the R2/R4 and LINE clades from the phylogenetic tree in FIG. 4. Full-length proteins containing RT domains classified as R2, R4, and LINEs were clustered at 99% sequence identity, and representative sequences were aligned. A phylogenetic tree was inferred from this alignment and R2/R4 retrotransposase families, as well as other RT-related families, were delineated (FIG. 5A).

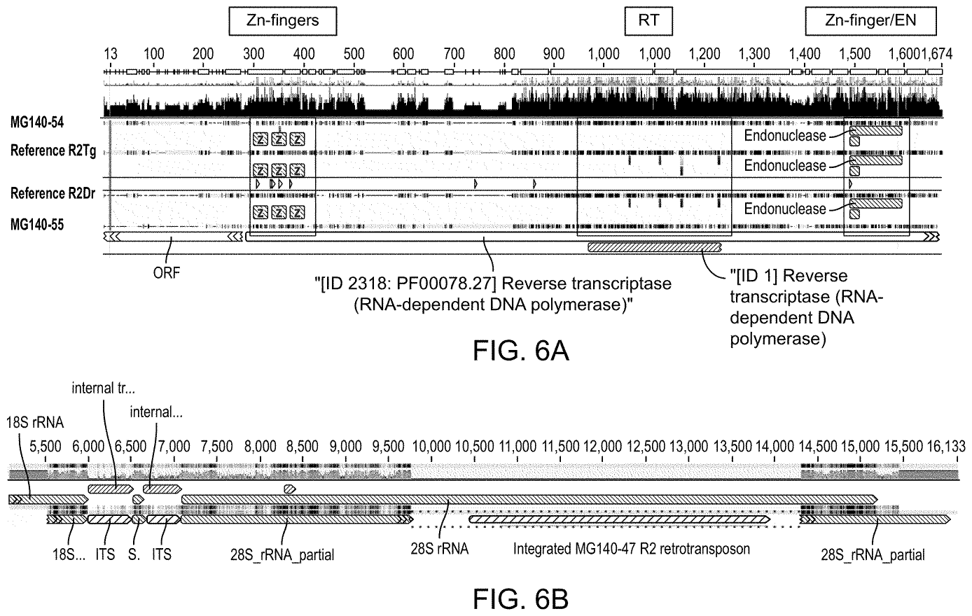

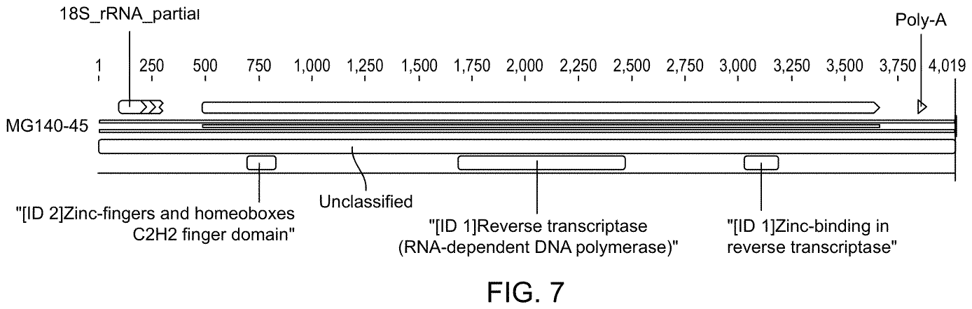

R2s are non-LTR retrotransposons that integrate cargo via target-primed reverse transcription (TPRT). Many R2 enzymes of the MG140 family contain an RT domain, as well as endonuclease domain and multiple Zn-binding ribbon motifs that delineate Zn-Fingers (FIGS. 5B and 6A). Some R2 retrotransposons integrate into the 28S rDNA, as shown by the boundaries of the MG140-47 (SEQ ID NO: 395) R2 retrotransposon flanked by fragments of a 28S rDNA gene (FIG. 6B). Other retrotransposons integrate into the 18S rRNA gene and contain a polyA or polyT tail that defines the 3′ end of the transposon (FIG. 7). It is possible that the exact target binding site, as well as 5′-UTR, 3′-UTR, and poly-T are involved in accurate and specific integration.

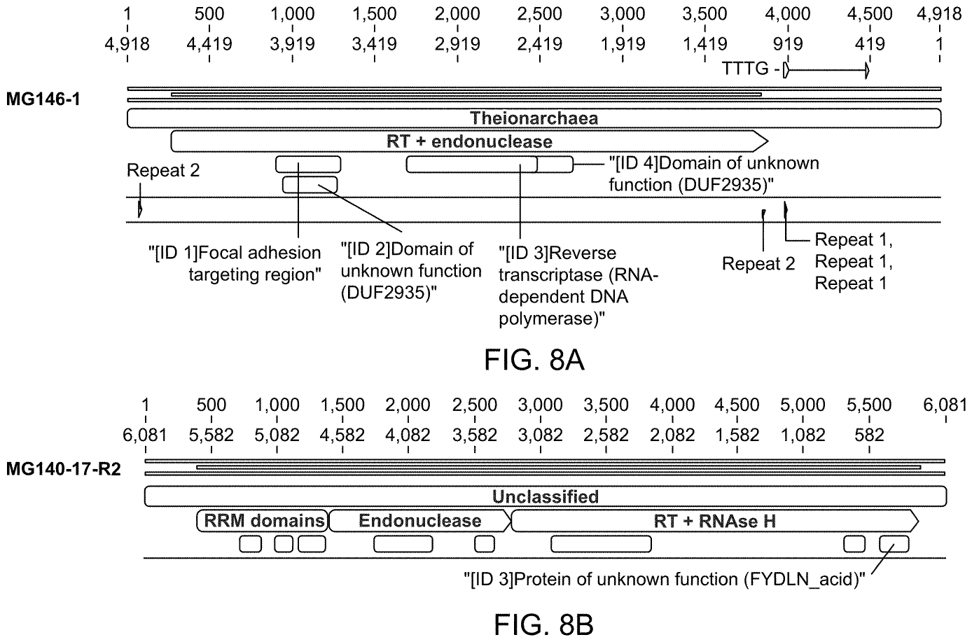

The retrotransposon MG146-1 (SEQ ID NO: 402), which was derived from an Archaeal genome, contains an RT domain, Zn-binding ribbon motifs, and an endonuclease domain, and the domain architecture within the enzyme differs from that of other single ORF non-LTR retrotransposons (FIG. 8A).

MG147 family member MG140-17-R2 (SEQ ID NO: 18) retrotransposon is organized into three ORFs flanked by 5′ and 3′ UTRs (FIG. 8B). The RNA recognition motif (RRM) gene is likely involved in recognition of the RNA template, while the endonuclease gene is likely involved in recognition and nicking of the target site. ORF three is the enzyme responsible for reverse transcription of the template and contains an RT domain, Zn-binding ribbon motifs, and an RNAse-H domain.

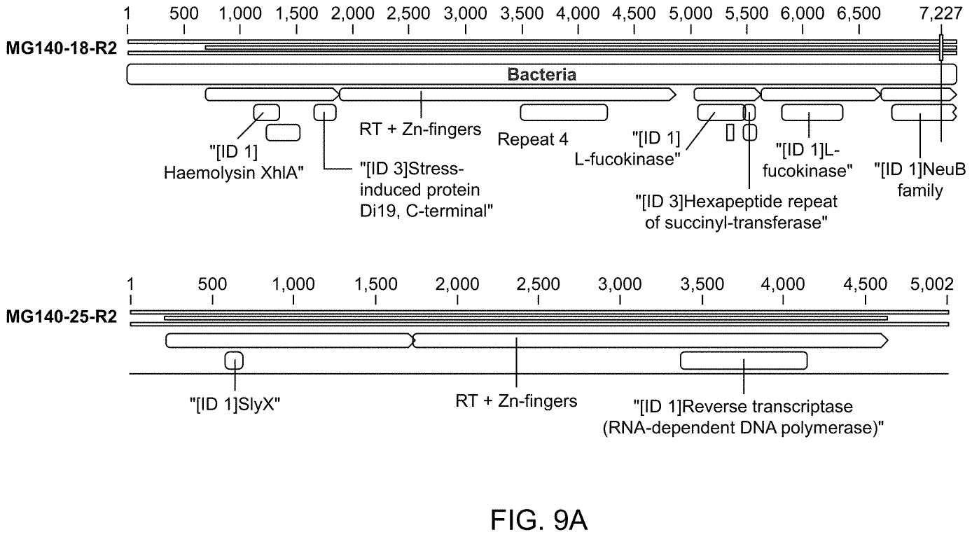

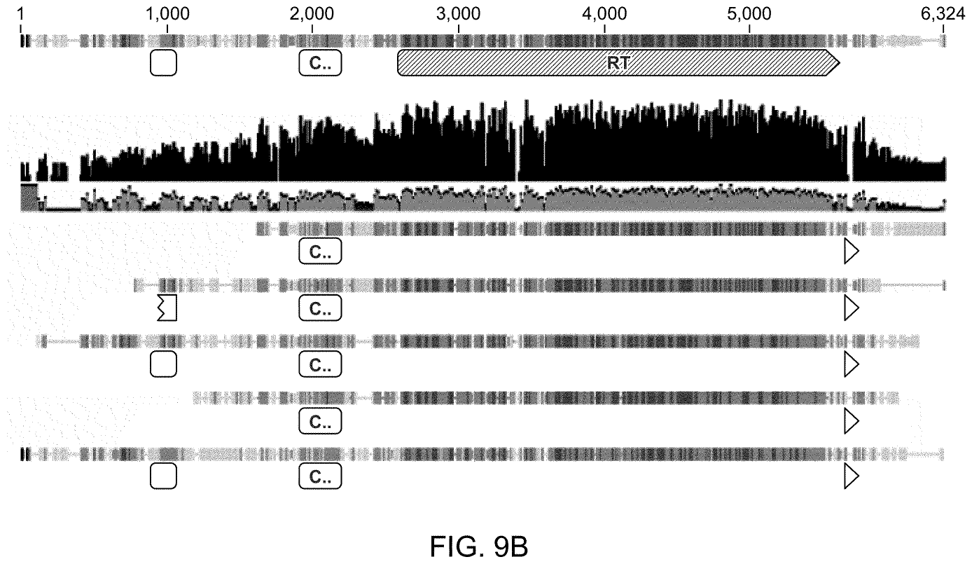

Family MG148 includes extremely divergent RT homologs, predicted to be active by the presence of all expected catalytic residues. Alignment at the nucleotide level for several family members uncovered conserved regions within the 5′ UTR, which are possibly involved in RT function, activity or mobilization (FIG. 9B).

Testing the In Vitro Activity of Retrotransposon RTs (Reverse Transcriptases) by qPCR

The in vitro activity of retrotransposon RTs was assessed by a primer extension reaction containing RT enzyme derived from a cell-free expression system and 100 nM of RNA template (200 nt) annealed to a DNA primer in reaction buffer containing 40 mM Tris-HCl (pH 7.5), 0.2 M NaCl, 10 mM MgCl₂, 1 mM TCEP, and 0.5 mM dNTPs. The resulting full-length cDNA product was quantified by qPCR by extrapolating values from a standard curve generated with the DNA template of specific concentrations.

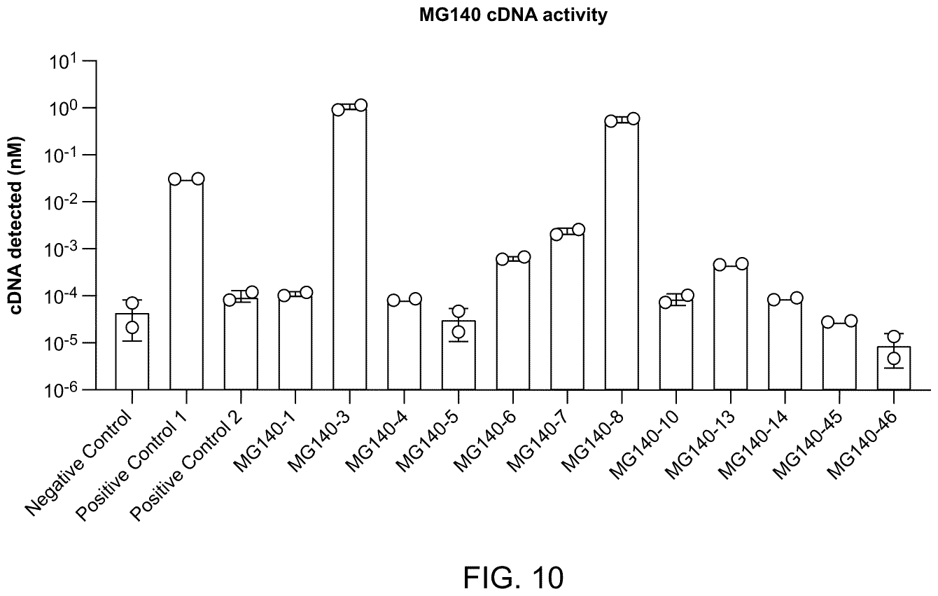

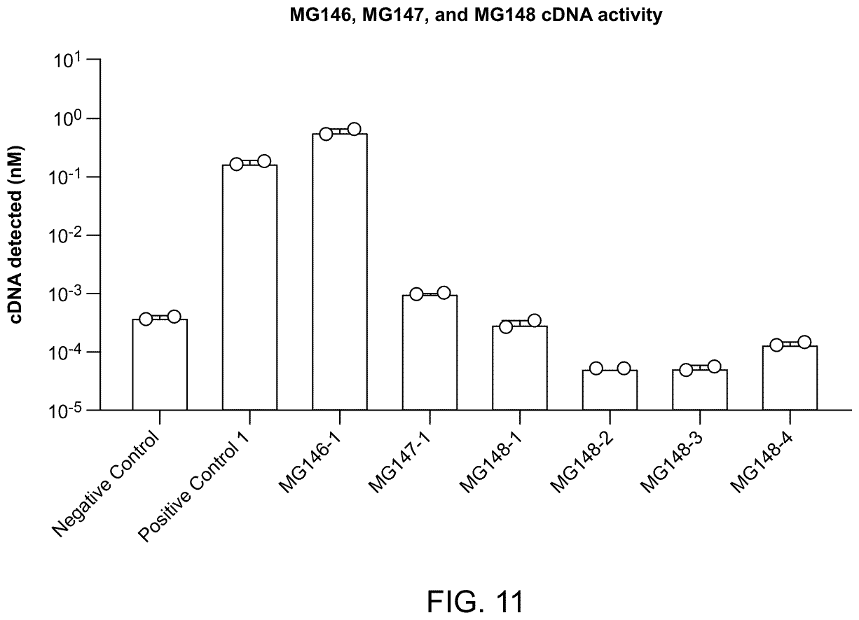

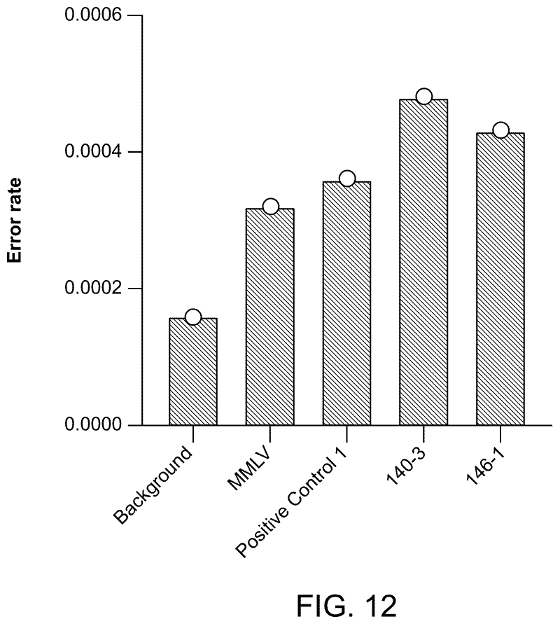

MG140-3 (SEQ ID NO: 3), MG140-6 (SEQ ID NO: 6), MG140-7 (SEQ ID NO: 7), MG140-8 (SEQ ID NO: 8), MG140-13 (SEQ ID NO: 14), and MG146-1 (SEQ ID NO: 402) are active via primer extension (FIGS. 10 and 11). Preliminary assessment of fidelity was performed for MG140-3 and MG146-1, resulting in a relative error rate 1.5 and 1.35-times higher than MMLV, respectively (FIG. 12). For fidelity measurements, the resulting full-length cDNA product generated in the primer extension assay described above was PCR-amplified, library-prepped, and subjected to next generation sequencing. Trimmed reads were aligned to the reference sequence and the frequency of misincorporation was calculated.

### Integration Site

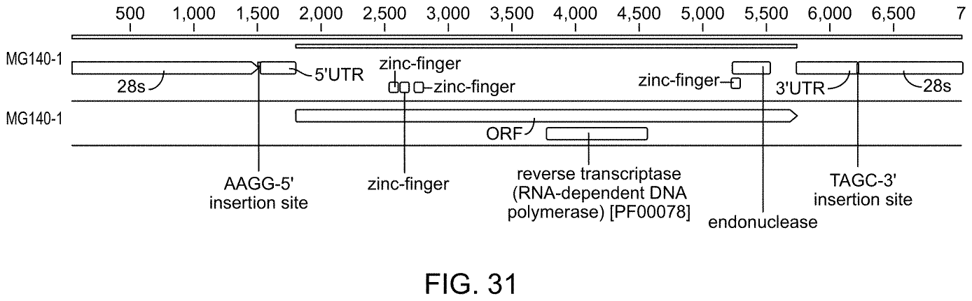

Some non-LTR retrotransposons (e.g., MG140 family such as MG140-1) are predicted to integrate into the 28S rDNA gene by targeting specific GGTGAC motifs, with the insertion site between the second (G) and third (T) positions. The N-terminus of such retrotransposon proteins contains three zinc (Zn) fingers (two of the CCHH type and one of type CCHC), which are followed by the reverse transcriptase (RT) domain with a YADD (SEQ ID NO: 2269) active site. The C-terminus of such retrotransposon proteins includes an endonuclease domain with an additional CCHC Zn-finger. The protein is flanked by 5′ and 3′ UTRs that are 289 and 478 bp long, respectively (FIG. 31).

### Example 10—Group II Intron RTs (MG153, MG163, MG164, MG165, MG166, MG167, MG168, MG169, and MG170 Families)

### Group II Bioinformatic Analysis

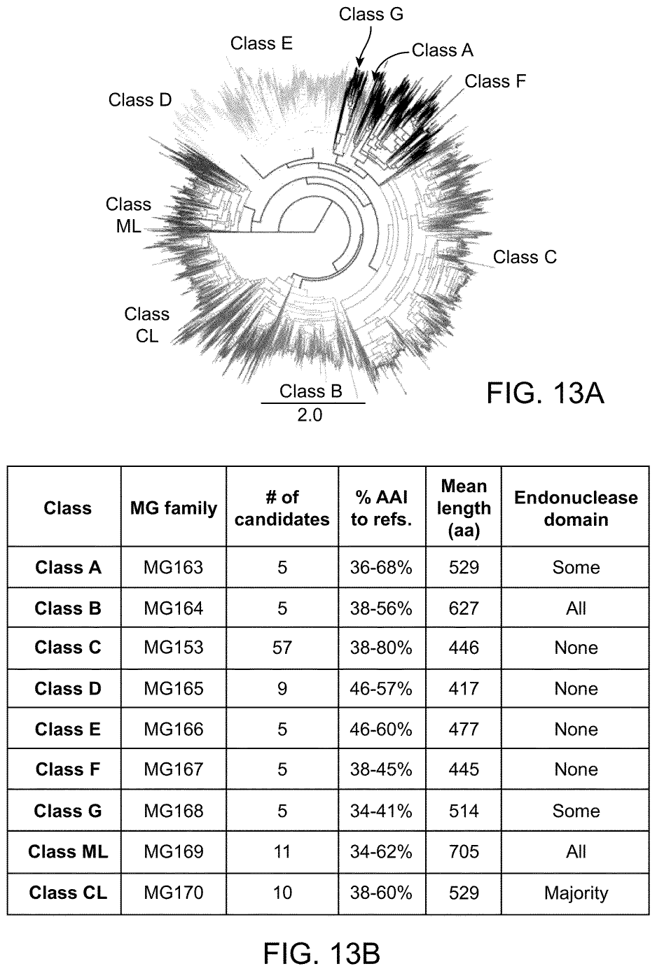

Group II introns are capable of integrating large cargo into a target site via reverse transcription of an RNA template. RT domains from Group II introns were identified and delineated in the phylogenetic tree in FIG. 4. Over 10,000 unique full-length Group II intron proteins containing RT domains from contigs with >2 kb of sequence flanking the RT enzyme were aligned. A phylogenetic tree was inferred from this alignment and Group II intron families were further identified (FIGS. 13A-13B). Group II intron enzymes can be classified into classes A-G, ML, and CL, and their domain architecture includes an RT domain predicted to be active, as well as a maturase domain involved in intron mobilization. Some Group II intron proteins contain an additional endonuclease domain likely involved in target recognition and cleavage. Many candidates from all families identified were nominated for further characterization.

### Testing the In Vitro Activity of Group II Intron RTs Class C, D, and F

The in vitro activity of GII intron Class C (MG153), Class D (MG165), and Class F (MG167) RTs was assessed by a primer extension reaction containing RT enzyme derived from a cell-free expression system. Expression constructs were codon-optimized for E. coli and contained an N-terminal single Strep tag. Expression of the RT was confirmed by SDS-PAGE analysis. The substrate for the reaction was 100 nM of RNA template (200 nt) annealed to a 5′-FAM labeled primer. The reaction buffer contained the following components: 50 mM Tris-HCl (pH 8.0), 75 mM KCl, 3 mM MgCl₂, 10 mM DTT, and 0.5 mM dNTPs. Following incubation at 37° C. for 1 h, the reaction was quenched via incubation with RnaseH, followed by the addition of 2×RNA loading dye. The resulting cDNA product(s) were separated on a 10% denaturing polyacrylamide gel and were visualized using visualization system. RT activity was also assessed by qPCR with primers that amplify the full-length cDNA product. Products from the primer extension assay were diluted to ensure cDNA concentrations were within the linear range of detection. The amount of cDNA was quantified by extrapolating values from a standard curve generated with the DNA template of specific concentrations.

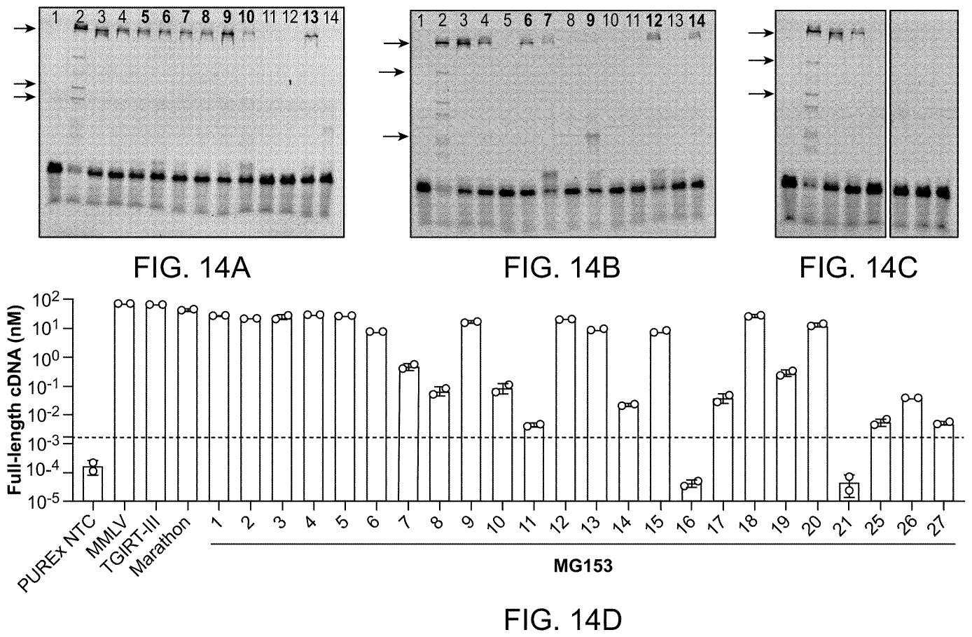

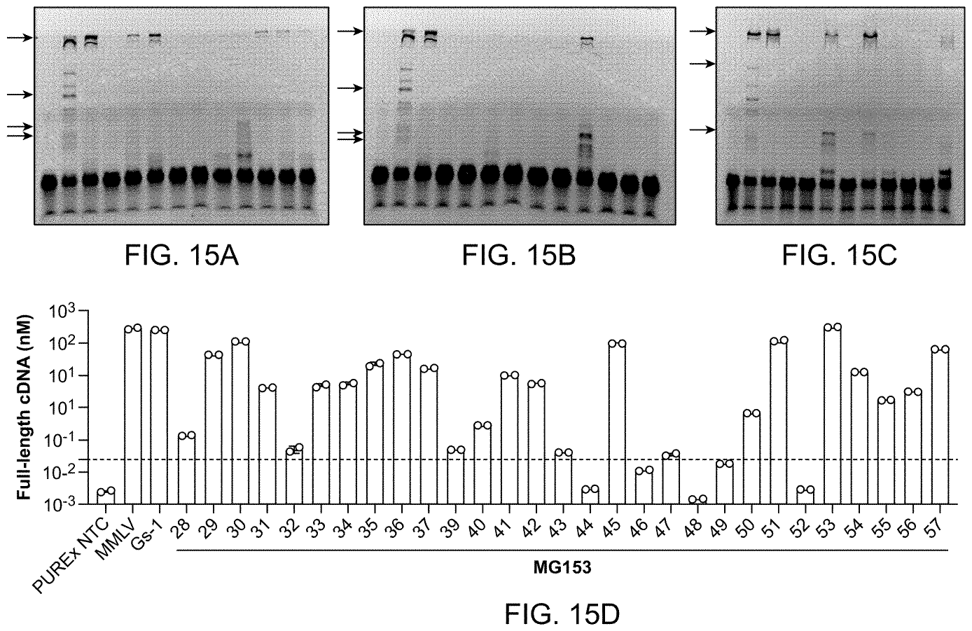

By detection of cDNA products on a denaturing gel, the following GII intron class C candidates were active under these experimental conditions: MG153-1 through MG153-6 (SEQ ID NOs: 555-560), MG153-9 (SEQ ID NO: 563), MG153-10 (SEQ ID NO: 564), MG153-12 (SEQ ID NO: 566), MG153-13 (SEQ ID NO: 567), MG153-15 (SEQ ID NO: 569), MG153-18 (SEQ ID NO: 572), MG153-20 (SEQ ID NO: 574), MG153-29 through MG153-31 (SEQ ID NOs: 580-582), MG153-33 through MG153-37 (SEQ ID NOs: 584-588), MG153-41 (SEQ ID NO: 592), MG153-42 (SEQ ID NO: 593), MG153-45 (SEQ ID NO: 596), MG153-51 (SEQ ID NO: 602), MG153-53 (SEQ ID NO: 604), MG153-54 (SEQ ID NO: 605), and MG153-57 (SEQ ID NO: 608). (FIGS. 14A-14D and 15A-15D). Active candidates exhibit a varying degree of apparent processivity compared to the highly processive control GII Class C RTs GsI-IIC and MarathonRT, indicated by the presence of smaller cDNA drop-off products. By qPCR, the following additional candidates are also active under these experimental conditions (cDNA detected >10-fold above background): MG153-7 (SEQ ID NO: 561), MG153-8 (SEQ ID NO: 562), MG153-10 (SEQ ID NO: 564), MG153-11 (SEQ ID NO: 565), MG153-14 (SEQ ID NO: 568), MG153-17 (SEQ ID NO: 571), MG153-19 (SEQ ID NO: 573), MG153-25 through MG153-28 (SEQ ID NOs: 576-579), MG153-32 (SEQ ID NO: 583), MG153-39 (SEQ ID NO: 590), MG153-40 (SEQ ID NO: 591), MG153-43 (SEQ ID NO: 594), MG153-47 (SEQ ID NO: 598), MG153-50 (SEQ ID NO: 601), MG153-55 (SEQ ID NO: 606) and MG153-56 (SEQ ID NO: 607) (FIGS. 14D and 15D).

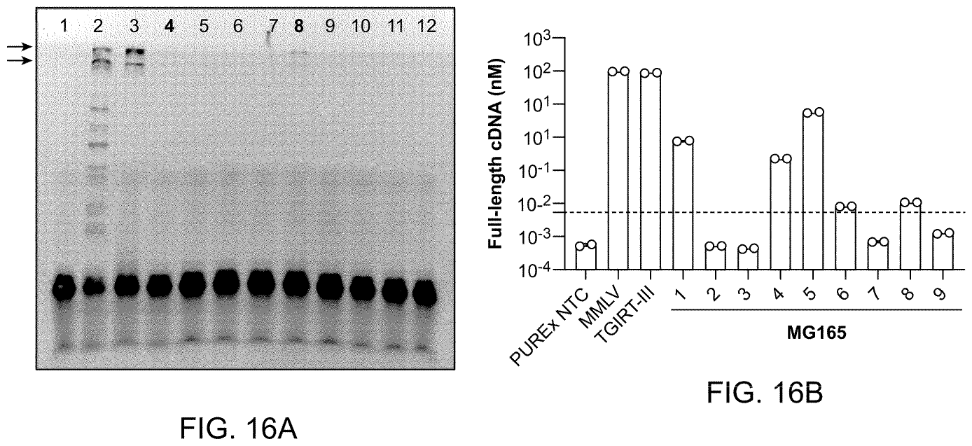

By detection of cDNA products on a denaturing gel, GII intron class D candidates MG165-1 (SEQ ID NO: 684) and MG165-5 (SEQ ID NO: 688) are active under these experimental conditions (FIG. 16A). By qPCR, additional candidates MG165-4 (SEQ ID NO: 687), MG165-6 (SEQ ID NO: 689), and MG165-8 (SEQ ID NO: 691) are also active under these experimental conditions (cDNA detected >10-fold above background) (FIG. 16B).

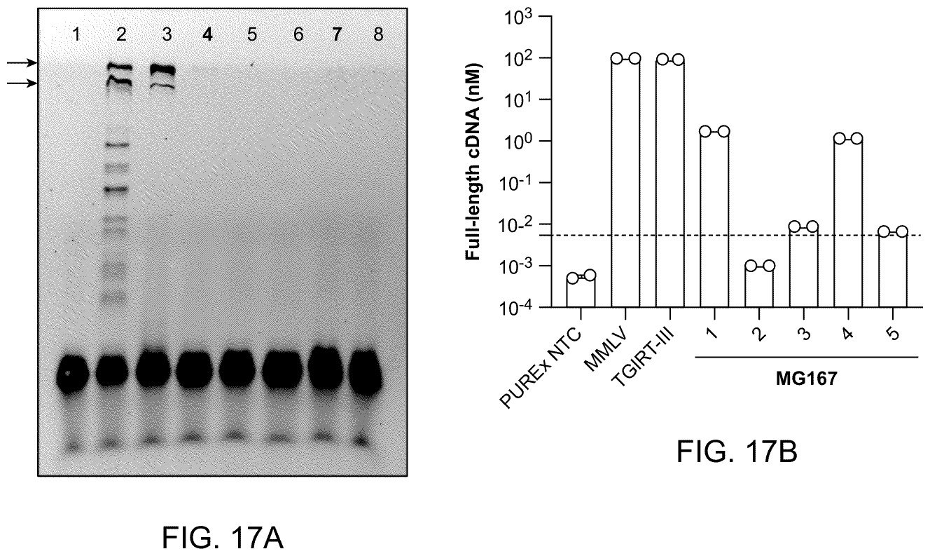

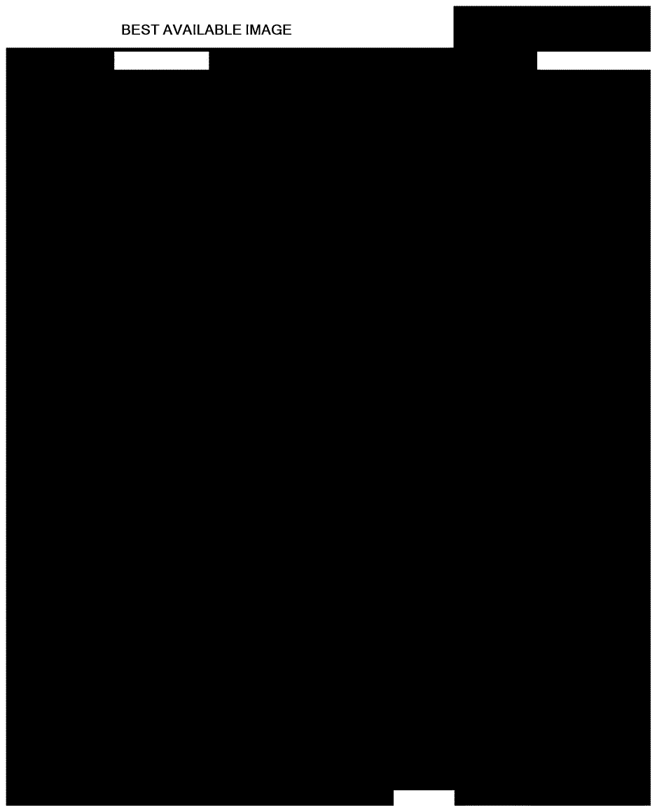

By detection of cDNA products on a denaturing gel, GII intron Class F candidates MG167-1 (SEQ ID NO: 698) and MG167-4 (SEQ ID NO: 701) are active under these experimental conditions (FIG. 17A). By qPCR, additional candidates MG167-3 (SEQ ID NO: 700) and MG167-5 (SEQ ID NO: 702) are also active under these experimental conditions (cDNA detected >10-fold above background) (FIG. 17B).

### Assessment of Relative Fidelity of GII Intron RTs

To assess the relative fidelity of GII Class C MG153 candidates, the resulting full-length cDNA product generated in the primer extension assay described above was PCR-amplified, library-prepped, and subjected to next generation sequencing. Paired reads were merged using bbmerge.sh requiring a perfect overlap and trimming all non-overlapping portions. Merged reads were then aligned to the reference template and the number of mismatches at each position relative to the reference was calculated. Of the GII Class C candidates tested, MG153-6 (SEQ ID NO: 560) and MG153-12 (SEQ ID NO: 566) have reproducibly higher error rates compared to MMLV control RT and other GII intron Class CRTs (FIG. 18).

Human Cells cDNA Synthesis Results

The ability of these enzymes to produce cDNA in a mammalian environment was tested by expressing them in mammalian cells and detecting cDNA synthesis by PCR, followed by agarose electrophoresis and D1000 TapeStation. Reverse transcriptases were cloned in a plasmid for mammalian expression under the CMV promoter as fusion proteins having MS2 coat protein (MCP) at the N terminus, in addition to a flag-HA tag (FH). MCP is a protein derived from the MS2 bacteriophage that recognizes a 20 nucleotide RNA stem loop with high affinity (subnanomolar Kd). By fusing the RTs with MCP and having the MS2 loops in the RNA template, it is ensured that once the RT is translated, it finds the RNA template and starts cDNA synthesis from the DNA primer hybridized to the RNA template.

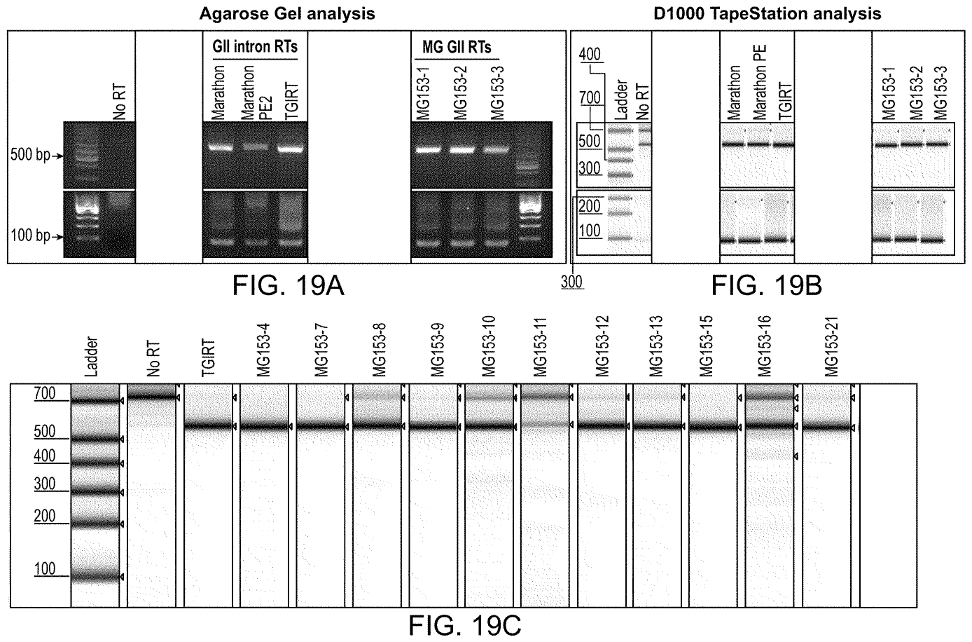

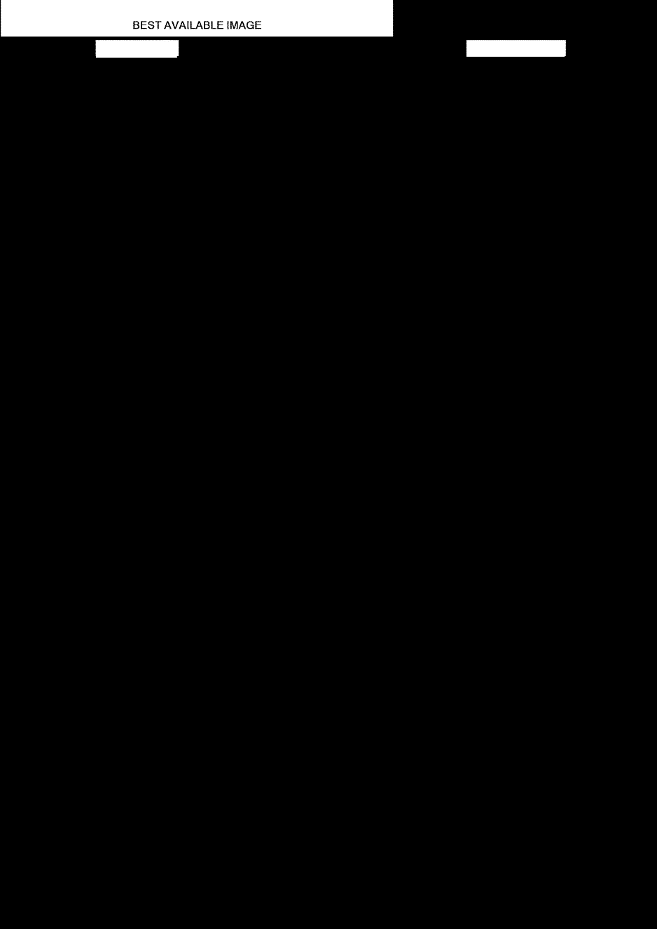

A plasmid containing MCP fused to the RT candidate under CMV promoter was cloned and isolated for transfection in HEK293T cells. Transfection was performed using liposome based system. mRNA codifying nanoluciferase (SEQ ID NO: 33) was produced. In order to degrade any DNA template left in the mRNA preparation, the reaction was treated with Dnase for 1 hour, and the mRNA was cleaned using a transcription Clean-Up kit. The mRNA was hybridized to a complementary DNA primer (SEQ ID NO: 34) in 10 mM Tris pH 7.5, 50 mM NaCl at 95° C. for 2 min and cooled to 4° C. at the rate of 0.1° C./s. The mRNA/DNA hybrid was transfected into HEK293T cells using liposome based technology 6 hours after the plasmid containing the MCP-RT fusion was transfected. 18 hours post mRNA/DNA transfection, cells were lysed using a DNA extraction solution, 100 μL of quick extract was added per 24 well in a 24 well plate. The nanoluciferase is ~500 bp long, primers to amplify products of 100 bp and 542 bp from the newly synthesized cDNA were designed (SEQ ID NOs: 38 and 39). cDNA was amplified using the set of primers mentioned above, and PCR products were detected by agarose gel electrophoresis (FIG. 19A) or DNA Tape Station (FIG. 19B).

Activity for the control GII intron RTs Marathon, Marathon PE2, and TGIRT was detected (FIGS. 19A and 19B), as shown by the presence of a 100 bp and 500 bp DNA product. Moreover, activity for GII intron derived RTs MG153-1 through MG153-4 (SEQ ID NOs: 555-558), MG153-7 through MG153-13 (SEQ ID NOs: 561-567), MG153-15 (SEQ ID NO: 569), MG153-16 (SEQ ID NO: 570) and MG153-21 (SEQ ID NO: 575) was also shown (FIGS. 19A, 19B, and 19C). The signal of the PCR product for the RTs was similar to that of Marathon and TGIRT. Altogether, this shows that these newly discovered RTs are expressed, fold properly, and are active inside living mammalian cells, opening options for their biotechnological applications. Group II intron RTs are capable of synthesizing cDNA using modified primers

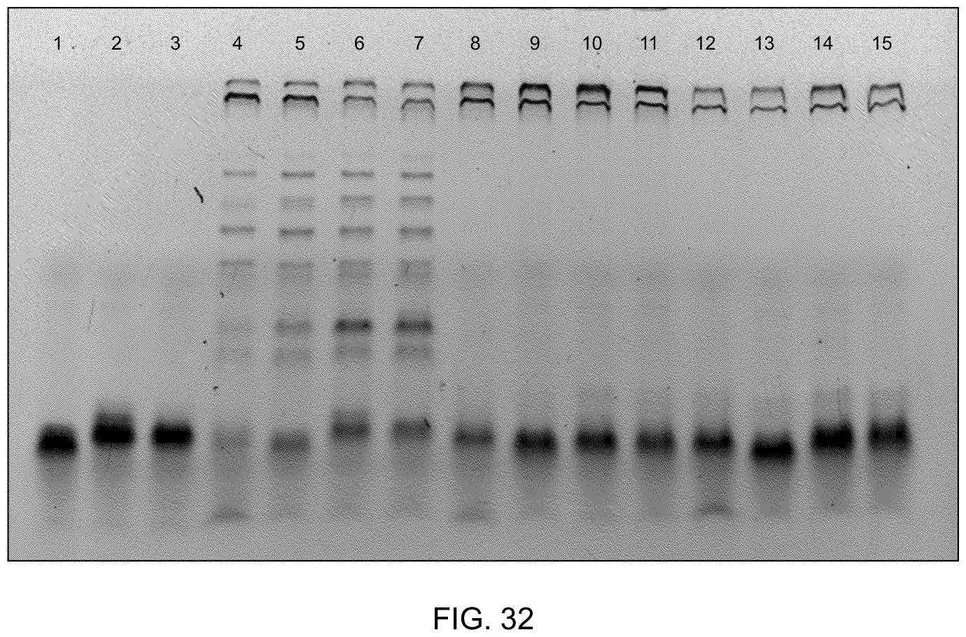

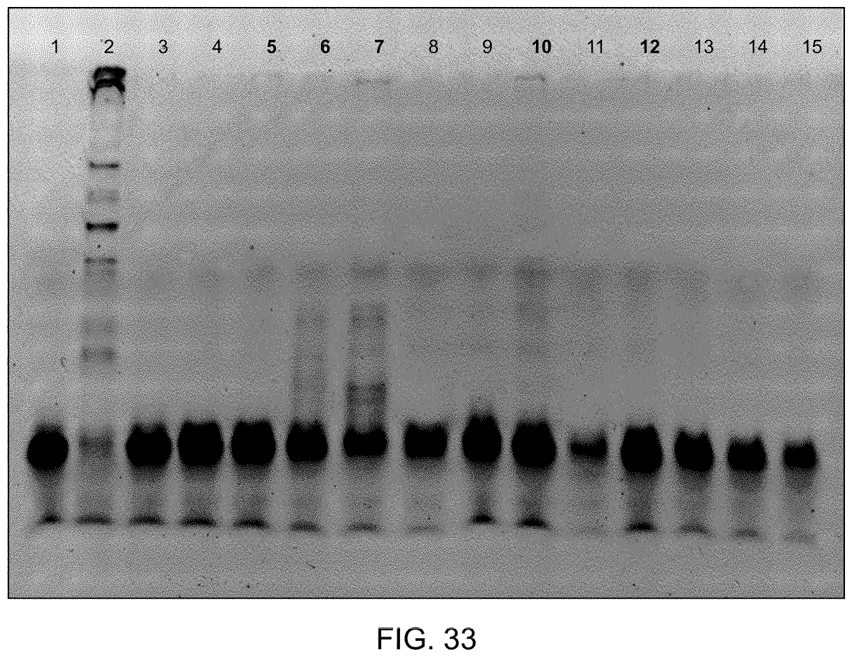

The in vitro activity of RTs was assessed by a primer extension reaction containing RT enzyme derived from a cell-free expression system. Expression constructs were codon-optimized for E. coli and contained an N-terminal single Strep tag. The substrate for the reaction was 100 nM of RNA template (202 nt) annealed to a 5′-FAM labeled DNA primer containing phosphorothioate (PS) bond modifications at various locations within the primer. Primer 1 (SEQ ID NO: 736, comprising a sequence/56-FAM/A*G*A*C*G*GTCACAGCTTGTCTG) contains 5 PS bonds at the 5′ end of the oligo. Primer 2 (SEQ ID NO: 737, comprising a sequence/56-FAM/A*G*A*C*G*GTCACAGCTT*G*T*C*T*G wherein * denotes a phosphorothioate bond) contains 5 PS bonds at both 5′ and 3 ends of the oligo. Primer 3 (SEQ ID NO: 738, comprising a sequence of/56-FAM/A*G*A*C*G*GTCACAGCTT*G*T*C*TG, wherein * denotes a phosphorothioate bond) differs from Primer 2 in that a standard bond is replaced between the two most 3′ terminal nucleotides. The reaction buffer contained the following components: 50 mM Tris-HCl (pH 8.0), 75 mM KCl, 3 mM MgCl₂, 10 mM DTT, and 0.5 mM dNTPs. Following incubation at 37° C. for 1 h, the reaction was quenched via incubation with RnaseH, followed by the addition of 2×RNA loading dye. The resulting cDNA product(s) were separated on a 10% denaturing polyacrylamide gel and were visualized using an imaging system. Based on these results, the control RTs MMLV (viral) and TGIRT-III (GII intron) are both capable of performing primer extension with all modified primers (FIG. 32). The GII intron RT MG153-9 is also capable of extending from all tested PS-modified DNA primers (FIG. 33).

Human Cells RT Expression and cDNA Synthesis Results

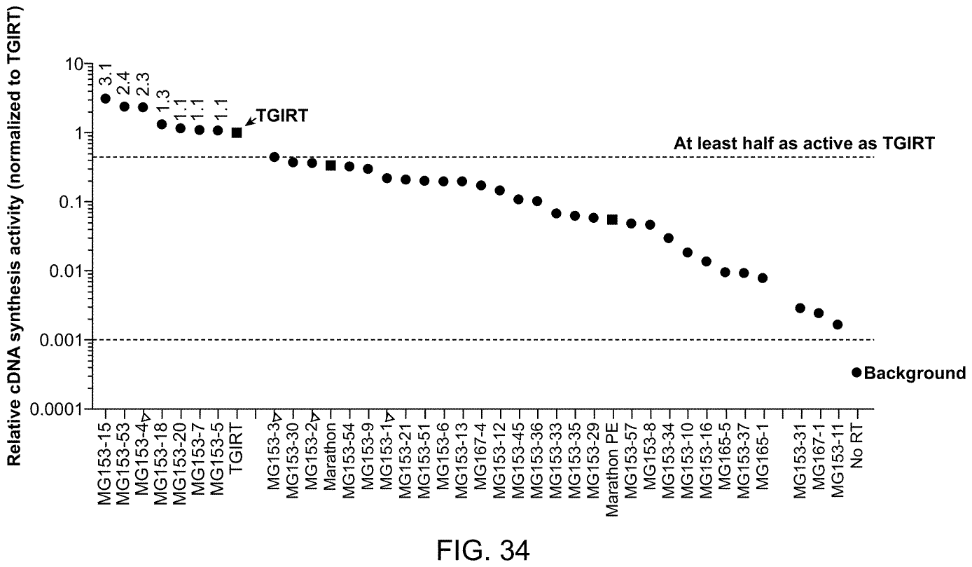

The ability of GII RTs to synthesize cDNA in a mammalian cell environment was tested as previously described with insubstantial modifications. cDNA synthesis was detected using PCR and analyzed by agarose gel electrophoresis or TapeStation. In order to have a quantitative readout, a qPCR assay was developed using qPCR primers already documented with a probe listed as SEQ ID NO: 739. All tested candidates of the MG153 family were active to various degrees, with activity as broad as four orders of magnitude (FIG. 34). RTs of families tested include MG153-1 through MG153-13, MG153-15, MG153-16, MG153-18, MG153-20, MG153-21, MG153-29 through MG153-31, MG153-33 through MG153-37, MG153-45, MG153-51, MG153-53, MG153-54, MG153-57, MG165-1, MG165-5, MG167-1 and MG167-4. Several RTs (MG153-15, MG153-53, MG153-4, MG153-18, MG153-20, MG153-7 and MG153-5) outperformed the TGIRT control (FIG. 34).

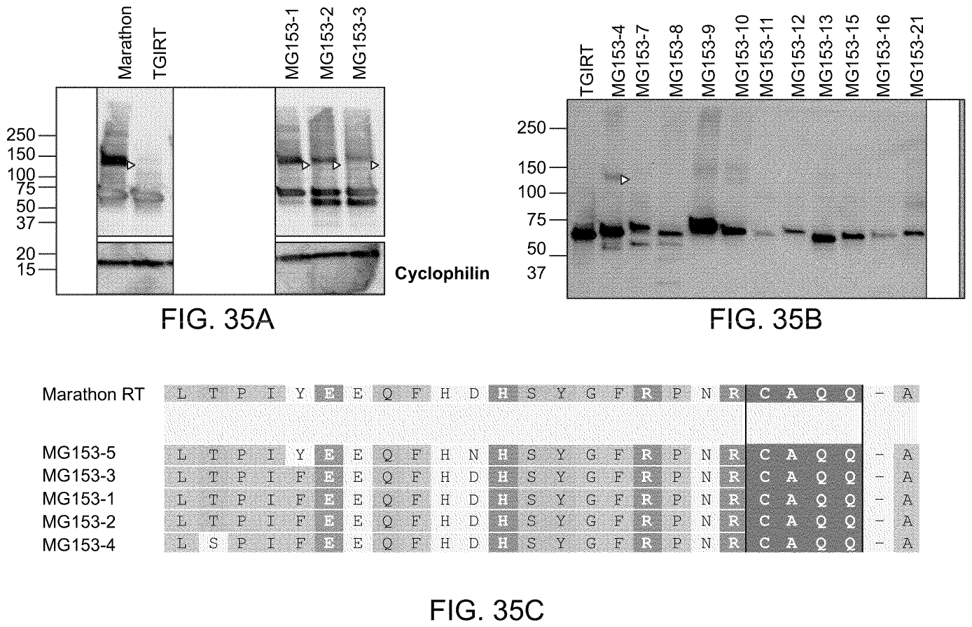

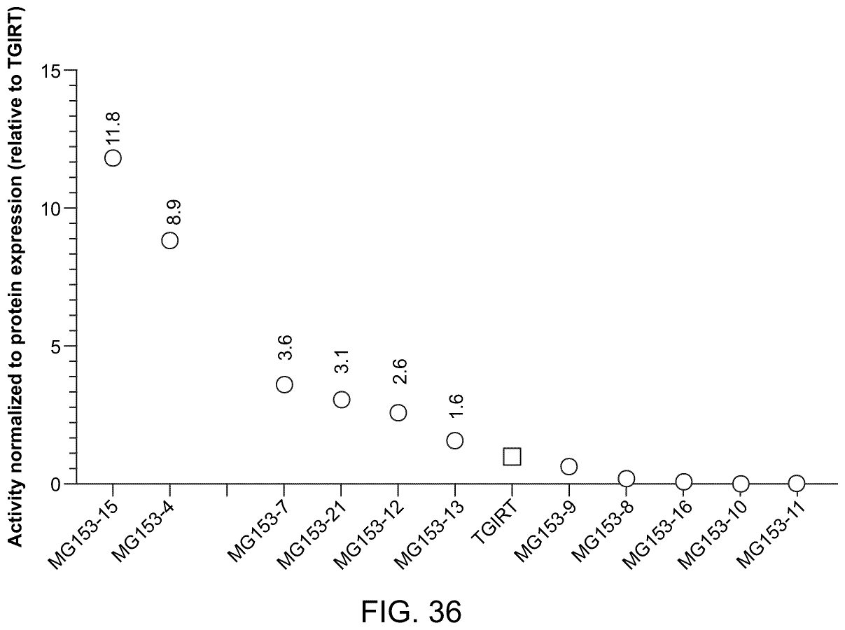

In order to understand protein expression and stability of the GII RTs in mammalian cells, immunoblots were performed. Briefly, transfected cells were lysed with RIPA lysis buffer supplemented with protease inhibitors (80 μL per well in a 24 well format). The lysate was centrifuged at 14,000 g for 10 min at 4° C. in order to remove insoluble aggregates. Proteins were quantified using BCA. 3 or 10 μg of total protein was loaded per lane in a 4-12% polyacrylamide SDS gel. All lanes were normalized to the same amount of protein. Proteins were transferred to a PVDF membrane using the iBlot gel transfer system. Proteins were detected by using a rabbit HA antibody, using an HRP-based detection method. Results suggest varying levels of protein expression or stability, as given by the intensity of the band (FIGS. 35A-35C). The expression of each protein was quantified and cDNA synthesis activity was normalized to total protein expression: seven MG153 RTs outperformed the TGIRT control (FIG. 36). Remarkably MG153-15 shows 10-fold higher cDNA synthesis activity than TGIRT under these conditions.

Some GII derived RTs form very stable dimers, including one of the positive controls, MarathonRT, as well as MG153-1 through MG153-4 and MG153-9 (FIGS. 35A-35C). The “CAQQ” motif (SEQ ID NO: 2267) was documented as responsible for stable dimerization in Marathon RT (Nat Struct Mol Biol. 2016 June; 23 (6): 558-565). RTs that showed stable dimer formation on immunoblots (MG153-1 through MG153-4) also contain the CAQQ (SEQ ID NO: 2267) dimerization amino acid motif (FIG. 35C). Dimerization may be an unfavorable feature due to added complexity, therefore RTs that do not form dimers may be optimal for specific biotechnological applications.

TABLE 2
Expected molecular sizes for tested RT candidates

| RT | Expected Protein Size (kDa)* |
|---|---|
| Marathon | 67.8 |
| TGIRT | 67 |
| MG153-1 | 74 |
| MG153-2 | 74 |
| MG153-3 | 74 |
| MG153-4 | 67.6 |
| MG153-7 | 71.7 |
| MG153-8 | 67.6 |
| MG153-9 | 72 |
| MG153-10 | 72.2 |
| MG153-11 | 70.9 |
| MG153-12 | 72.5 |
| MG153-13 | 67.9 |
| MG153-15 | 68.6 |
| MG153-16 | 71.7 |
| MG153-21 | 70.6 |

*Size includes a Flag-HA-MCP tag

### Example 11—G2L4 (MG172 Family)

G2L4 are RT-containing sequences distantly related to Group II introns (Group II intron-like RTs), which were identified in FIG. 4. Over 600 full-length G2L4 enzymes were aligned and a phylogenetic tree was inferred from this alignment (FIGS. 20A-20B). MG172 family members contain RT and maturase domains, and were predicted to have a conserved Y[I/L]DD active site motif. The motif YIDD (SEQ ID NO: 2270) was recently reported to display increased efficiency with shorter DNA primers in one G2L4 reference. MG172 enzymes have an average length of 425 aa and share 32% AAI, which highlights the improvement of these systems.

### Example 12—LTR Retrotransposons (MG151 Family)

### LTR Retrotransposon Bioinformatic Analysis

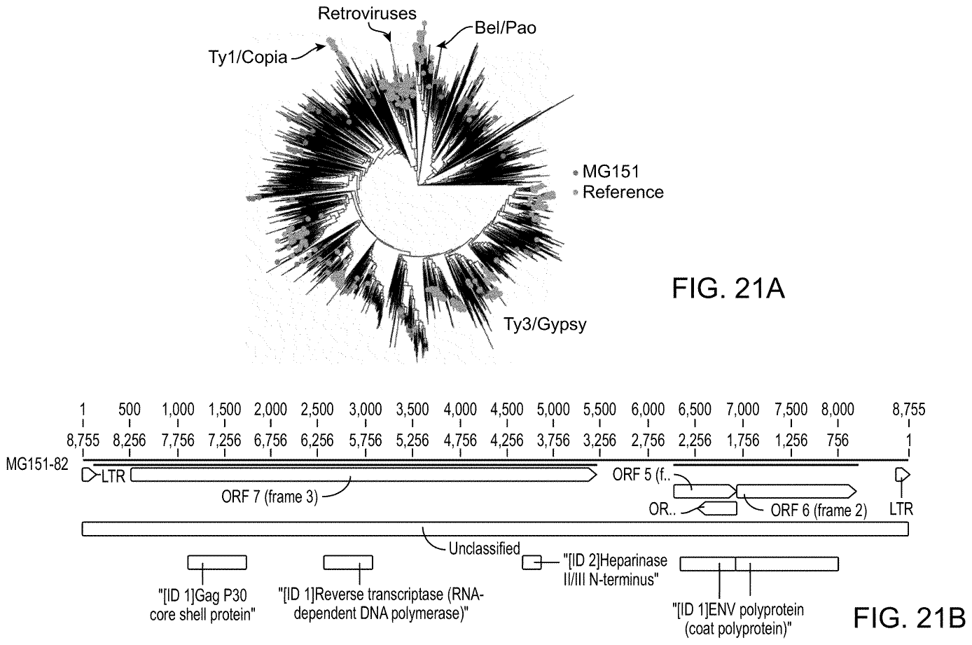

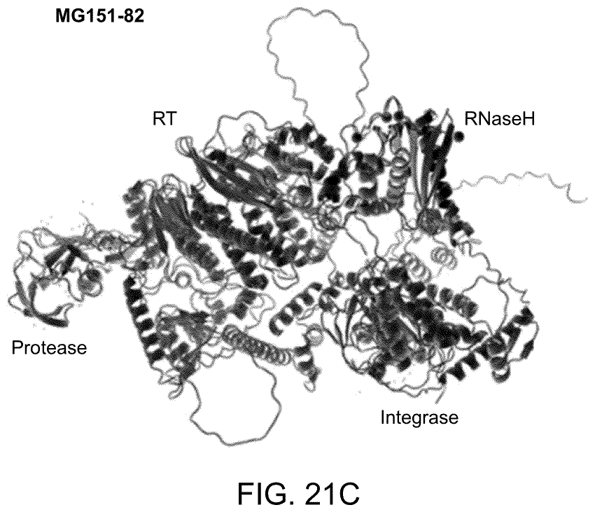

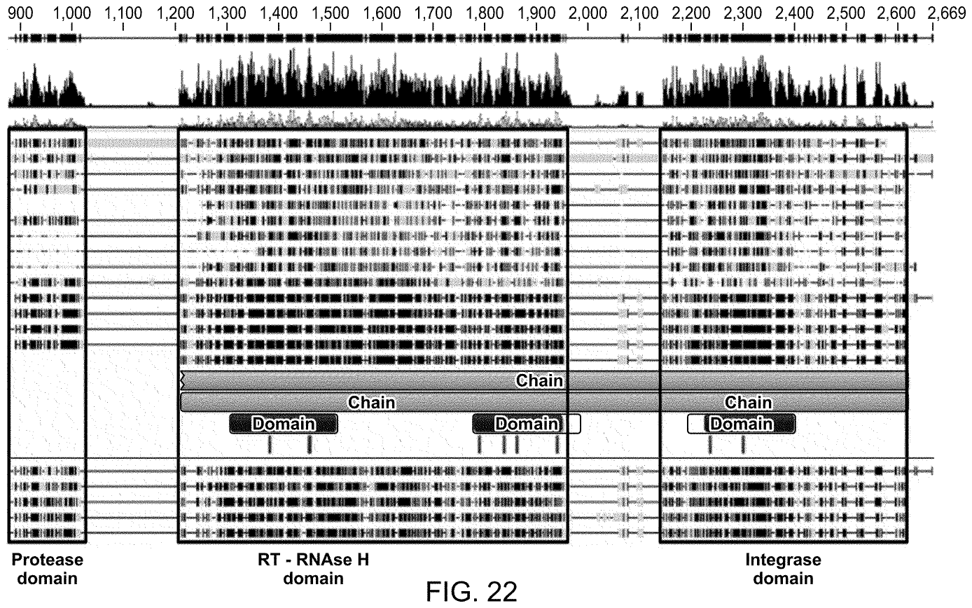

Long terminal repeat (LTR) retrotransposons integrate into their target sites via reverse transcription of an RNA template. The MG151 family of LTR retrotransposons, which include retroviral and non-viral transposons, was identified in the phylogenetic tree in FIG. 4. Full-length proteins containing LTR RT domains were aligned. A phylogenetic tree was inferred from this alignment (FIG. 21A). More than 100 non-viral and retroviral RT enzymes of the MG151 family contain RT and RnaseH domains, and are predicted to be active based on the presence of catalytic residues. The LTR RT polyprotein also encodes protease and integrase domains in a similar architecture seen for HIV and MMLV LTR RTs (FIGS. 21A, 21B, 21C, and 22). The RT and other genes, such as gag or envelope, are flanked by long imperfect long terminal repeats (FIG. 21B). MG151 family members are diverse and new, sharing 30% amino acid identity (FIG. 22).

The polyprotein of LTR retrotransposons is naturally processed into protease, RT and Rnase H, and integrase functional units. Therefore, the MG151 RT-RNAse H functional unit boundaries were determined by a combination of sequence and structural alignments. The 3D structure for MG151 polyproteins was predicted and visualized. For example, for MG151-82 (SEQ ID NO: 457), the predicted 3D structure identified discrete protease, RT, RNAseH, and integrase domains separated by unstructured linker regions (FIG. 21C). Therefore, the RT-RNAse H functional unit was determined as the two relevant structural domains flanked by unstructured loops. Trimmed variants containing RT and RNAse H domains were nominated for synthesis and laboratory characterization.

### Testing the In Vitro Activity of LTR Retrotransposon RTs

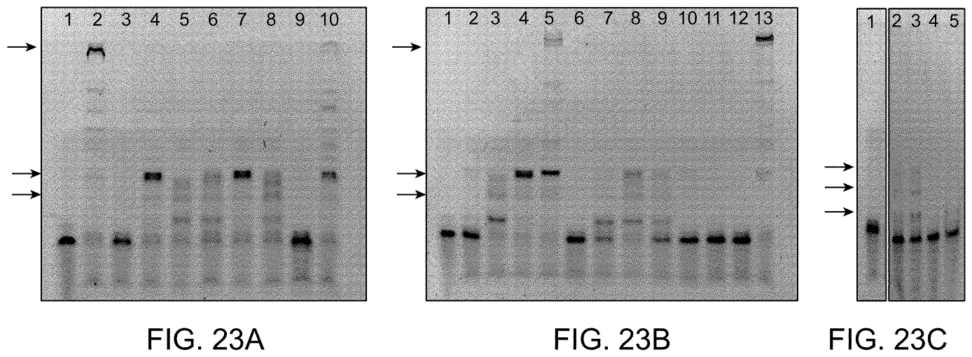

The in vitro activity of LTR retrotransposon RTs (MG151) was assessed by a primer extension reaction containing RT enzyme derived from a cell-free expression system and RNA template annealed to a 5′-FAM labeled primer as described above, in reaction buffer containing 50 mM Tris-HCl pH 8, 75 mM KCl, 3 mM MgCl₂, 1 mM TCEP, and 0.5 mM dNTPs. The resulting cDNA product(s) were separated on a denaturing polyacrylamide gel and visualized using an imaging system. Based on these results, MG151-80 through MG151-84 (FIG. 23A), as well as MG151-87 through MG151-90 (SEQ ID NOs: 524-527), and MG151-92 through MG151-95 (SEQ ID NOs: 529-532) (FIG. 23B) can synthesize cDNA in vitro.

To determine assay conditions under which in vitro activity is observed for Ty3, a control LTR retrotransposon RT, the following four reaction buffers were tested: Buffer A (40 mM Tris-HCl pH 7.5, 0.2 M NaCl, 10 mM MgCl₂, 1 mM TCEP); Buffer B (20 mM Tris pH 7.5, 150 mM KCl, 5 mM MgCl₂, 1 mM TCEP, 2% PEG-8000); Buffer C (10 mm Tris-HCl pH 7.5, 80 mm NaCl, 9 mm MgCl₂, 1 mM TCEP, 0.01% (v/v) Triton X-100); and Buffer D (10 mM Tris pH 7.5, 130 mM NaCl, 9 mM MgCl₂, 1 mM TCEP, 10% glycerol). In vitro activity was observed for Buffers A and B (FIG. 23C).

### Testing Priming Parameters and Processivity on a Structure RNA Template

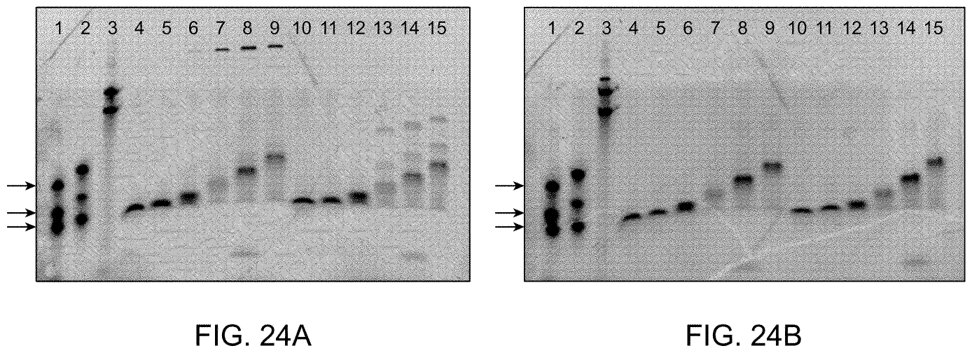

To determine the reverse transcriptase activity of these LTR RTs on a structured RNA template, different primers of length 6, 8, 10, 13, 16, and 20 nt were annealed onto a structured RNA scaffold. These annealed RNA/DNA hybrids were used in a cDNA generation assay equivalent to those used for overall activity. As shown in FIGS. 24A-24B, MMLV is active on a structured RNA with a primer binding site from 10-20 nt and extends the template completely to the 5′ end, opening up all structure in the template. MG151-89 (SEQ ID NO: 526) is active with primer lengths of 13-20 and can extend approximately 18 nt, the length of pegRNA until the sgRNA scaffold hairpin is reached. MG151-92 (SEQ ID NO: 529) and MG151-97 (SEQ ID NO: 534) were not active on this template at our level of detection.

### Example 13—Retron RTs (MG154, MG155, MG156, MG157, MG158, MG159, and MG160 Families)

### Retron Bioinformatic Analysis

Bacterial retrons are DNA elements of approximately 2000 bp in length that encode an RT-coding gene (ret) and a contiguous non-coding RNA containing inverted sequences, the msr and msd. Retrons employ a unique mechanism for RT-DNA synthesis, in which the ncRNA template folds into a conserved secondary structure, insulated between two inverted repeats (a1/a2). The retron RT recognizes the folded ncRNA, and reverse transcription is initiated from a conserved guanosine 2′OH adjacent to the inverted repeats, forming a 2′-5′ linkage between the template RNA and the nascent cDNA strand. In some retrons this 2′-5′ linkage persists into the mature form of processed RT-DNA, while in others an exonuclease cleaves the DNA product resulting in a free 5′ end. Moreover, the RT targets the msr-msd derived from the same retron as its RNA template, providing specificity that may avoid off-target reverse transcription.

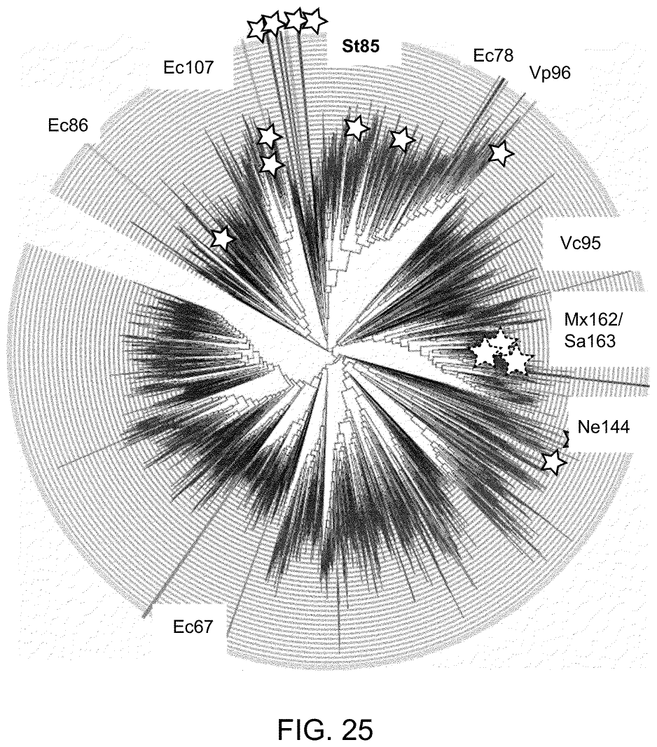

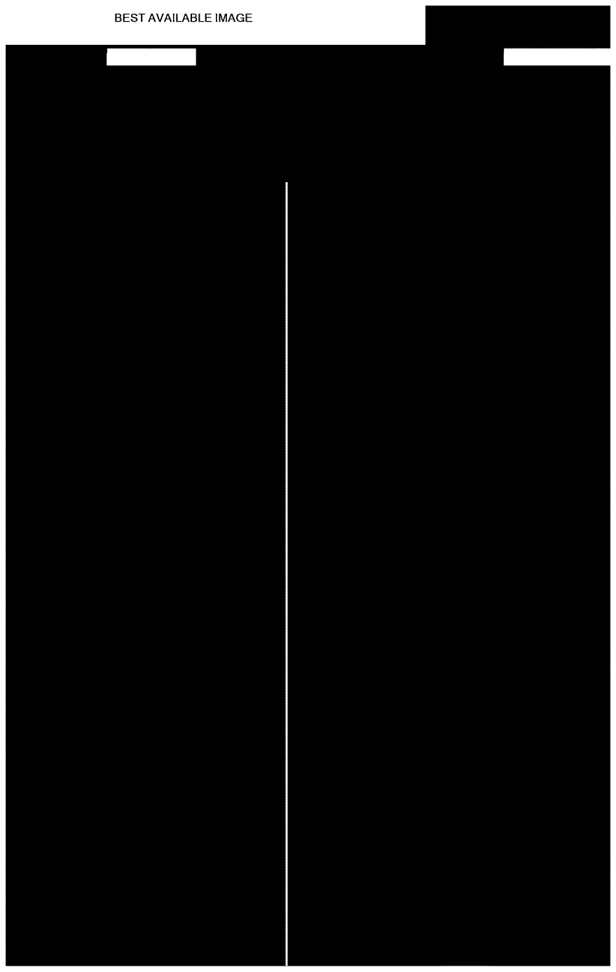

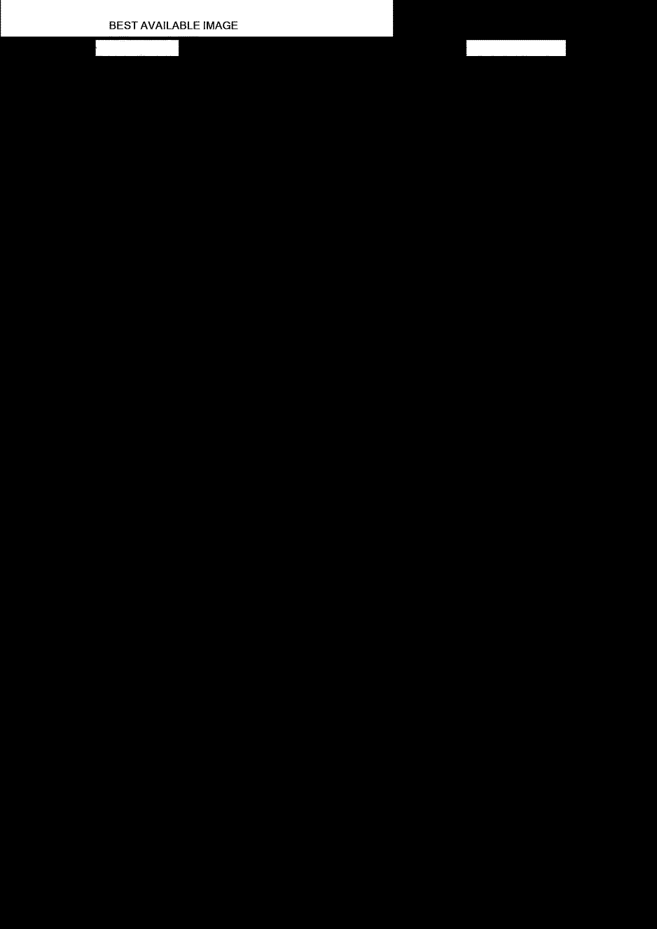

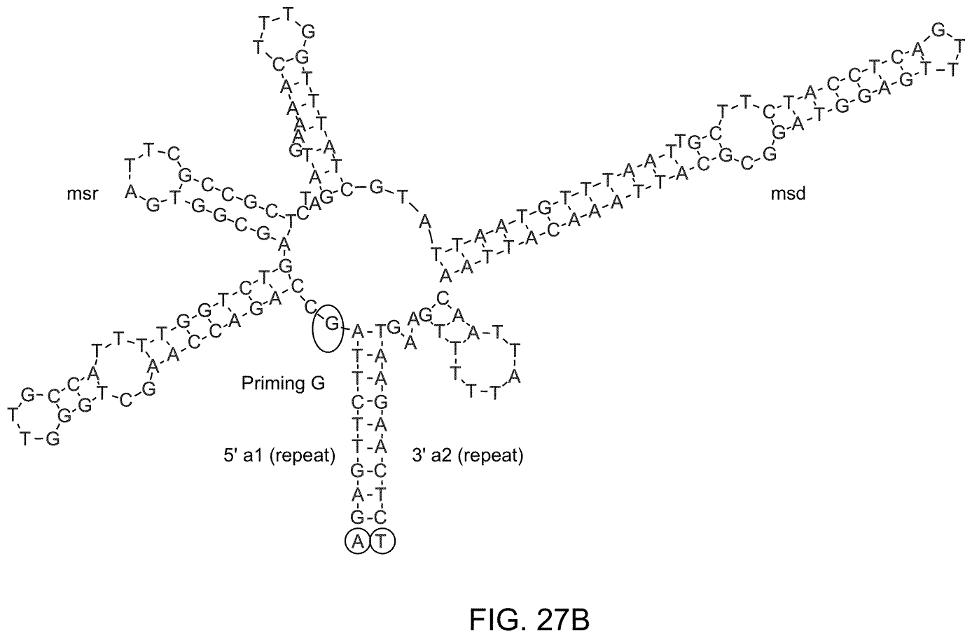

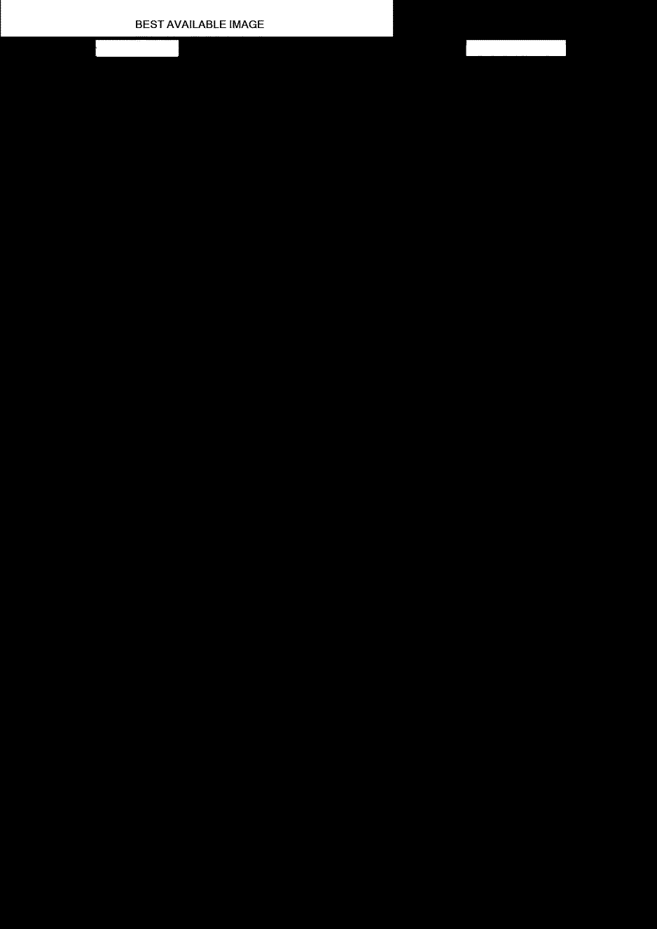

Over 4031 RT domain sequences were identified as retron RTs in the phylogenetic tree in FIG. 4. A subset of 2407 full-length retron protein sequences were selected for further analysis based on the presence of catalytic residues (xxDD) and conserved motifs documented in retron RTs (NaxxH and VTG) (FIGS. 25 and 26). Retrons of families MG154-MG159 and MG173 include members that range between 300 and 650 aa in length, and their 5′ UTR contains predicted ncRNA (msr-msd) trimmed flanked by inverted repeats (FIGS. 27A-27B).

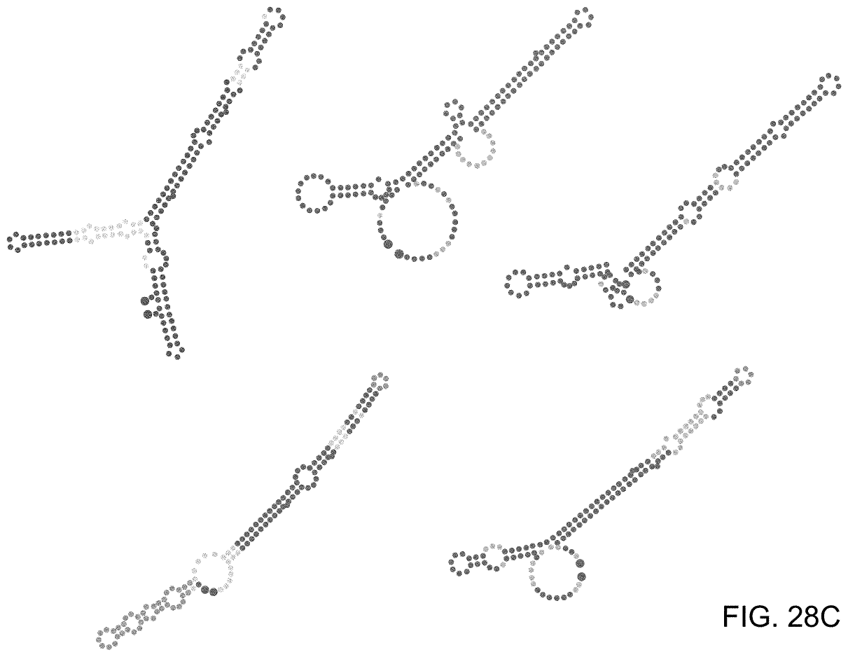

In addition, a divergent group of “retron-like” single-domain RT sequences were identified within the retron clade in FIG. 4. The single-domain RTs of the MG160 family range between 250 and 300 aa and are predicted to be active based on the presence of expected RT catalytic residues [F/Y]XDD. Although there is a lack of retron RT crystal and cryo-EM structures in public databases, 3D structure prediction of MG160-3 (SEQ ID NO: 629) indicates a conserved RT domain that aligns with a Group II intron RT domain (FIGS. 28A and 28B). The 5′ UTR of the MG160 family are conserved among family members and fold into conserved secondary structures (FIG. 28C) that are likely important for element activity or mobilization.

### In Vitro Activity of MG154, MG155, MG156, MG157, MG158, and MG159 Family of Retron-Like RTs

The in vitro activity of retron RTs on a general RNA template was assessed by a primer extension reaction containing RT enzyme derived from a cell-free expression system. Expression constructs were codon-optimized for E. coli and contained an N-terminal single Strep tag. The substrate for the reaction was 100 nM of RNA template (202 nt) annealed to a 5′-FAM labeled primer. The reaction buffer contained the following components: 50 mM Tris-HCl (pH 8.0), 75 mM KCl, 3 mM MgCl₂, 10 mM DTT, and 0.5 mM dNTPs. Following incubation at 37° C. for 1 h, the reaction was quenched via incubation with RnaseH, followed by the addition of 2×RNA loading dye. The resulting cDNA product(s) were separated on a 10% denaturing polyacrylamide gel and were visualized using an imaging system. Based on these results, the following retron RTs are capable of performing primer extension on a general RNA template that is not their own ncRNA: MG155-2 (SEQ ID NO: 612), MG155-3 (SEQ ID NO: 613), MG156-2 (SEQ ID NO: 617), MG157-5 (SEQ ID NO: 622), and MG159-1 (SEQ ID NO: 624).

### In Vitro Activity of MG160 Family of Retron-Like RTs

The in vitro activity of retron-like RTs (MG160 family) was assessed by a primer extension reaction containing RT enzyme derived from a cell-free expression system. Expression constructs were codon-optimized for E. coli and contained an N-terminal single Strep tag. The substrate for the reaction was 100 nM of RNA template (200 nt) annealed to a 5′-FAM labeled primer. The reaction buffer contained the following components: 50 mM Tris-HCl (pH 8.0), 75 mM KCl, 3 mM MgCl₂, 10 mM DTT, and 0.5 mM dNTPs. Following incubation at 37° C. for 1 h, the reaction was quenched via incubation with RnaseH, followed by the addition of 2×RNA loading dye. The resulting cDNA product(s) were separated on a 10% denaturing polyacrylamide gel and were visualized using an imaging system. RT activity was also assessed by qPCR with primers that amplify the full-length cDNA product. Products from the primer extension assay were diluted to ensure cDNA concentrations were within the linear range of detection. The amount of cDNA was quantified by extrapolating values from a standard curve generated with the DNA template of documented concentrations.

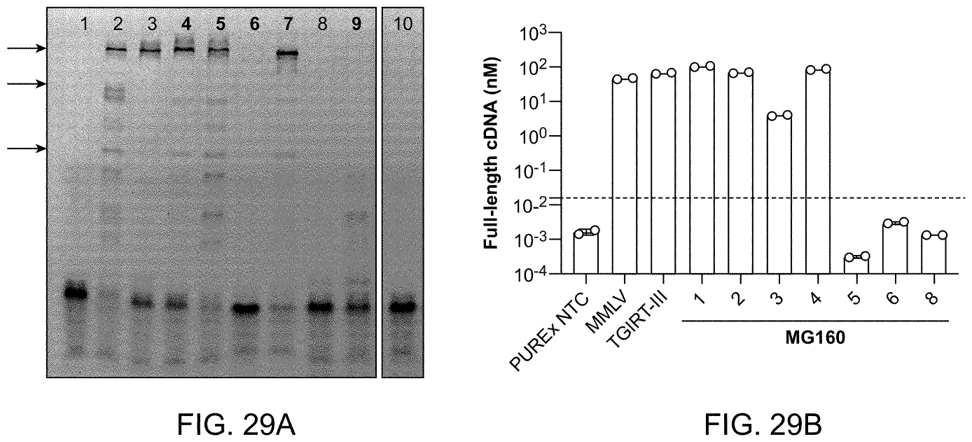

By gel analysis, MG160-1 through MG160-4 (SEQ ID NOs: 627-630) and MG160-6 (SEQ ID NO: 633) are active and had diminished processivity compared to GsI-IIC, a control GII intron Class C RT (FIGS. 29A-29B). Processivity appears more similar to that of MMLV, a retroviral control RT that produces a similar drop-off pattern of cDNA products (FIG. 29A). By qPCR, MG160-1 through MG160-4 (SEQ ID NOs: 627-630) can produce full-length cDNA, while MG160-6 (SEQ ID NO: 633) produced a less than full-length product (FIG. 29B).

Cell-Free Expression of Retron RTs (MG154, MG155, MG156, MG157, MG158, MG159, and MG173 Families) and In Vitro Transcription of Retron ncRNAs

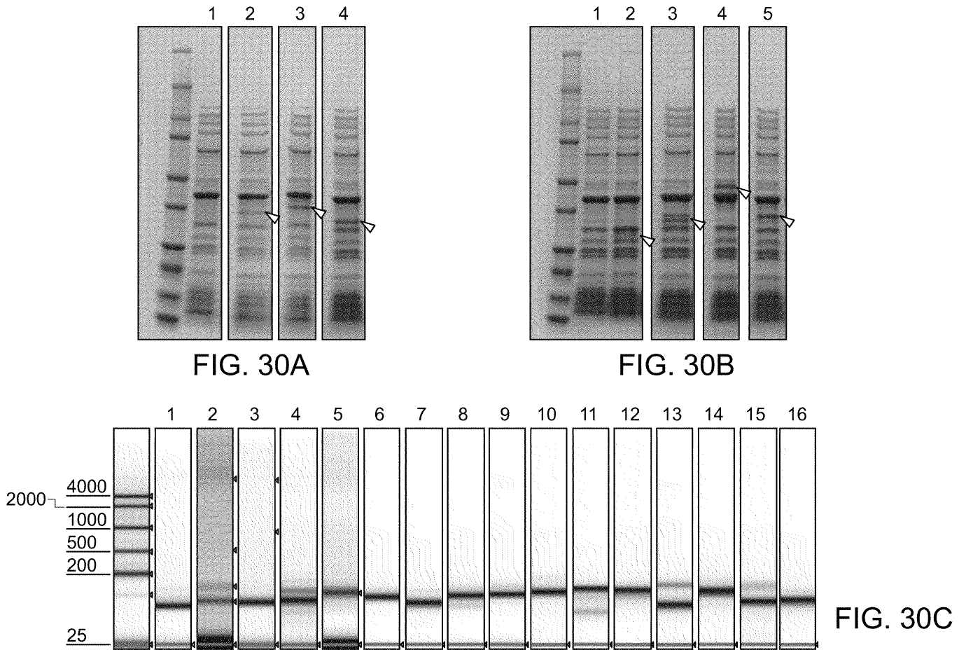

Retron RTs were produced in a cell-free expression system by incubating 10 ng/μL of a DNA template encoding the E. coli-optimized gene with an N-terminal single Strep tag with the in vitro expression components for 2 h at 37° C. All tested retron RTs (MG156-1 (SEQ ID NO: 616), MG156-2 (SEQ ID NO: 617), MG157-1 (SEQ ID NO: 618), MG157-2 (SEQ ID NO: 619), MG157-5 (SEQ ID NO: 622), MG159-1 (SEQ ID NO: 624)) were produced as indicated by SDS-PAGE analysis (FIGS. 30A and 30B).

The retron ncRNAs were generated using the a T7 in vitro transcription kit and a DNA template encoding the respective ncRNA gene following a T7 promoter. The reaction is then incubated with Dnase-I to eliminate the DNA template and then purified by an RNA cleanup kit. Quantity of the ncRNA was determined, and the purity was assessed (FIG. 30C).

### Example 14—Testing Retron RT In Vitro Activity (Prophetic)

The retron RT enzyme is produced in a cell-free expression system using a construct containing an E. coli codon-optimized gene with an N-terminal single Strep tag as described above. Expression of the enzyme is confirmed by SDS-PAGE analysis. Retron RT activity on a general template is determined by primer extension assay as described above, containing a 200 nt RNA annealed to a 5′-FAM labeled DNA primer. The resulting cDNA product(s) are detected on a denaturing polyacrylamide gel or by qPCR with primers specific for the full-length cDNA product.

Retron RT in vitro activity on its own ncRNA is assessed in a reaction containing buffer, dNTPs, the retron RT produced from a cell-free expression system, and the refolded ncRNA. RT activity before and after purification of the RT from the cell-free expression system via the N-terminal single Strep tag is compared. After incubation, half of the reaction is treated with Rnase A/T1. Products before and after Rnase A/T1 treatment are evaluated on a denaturing polyacrylamide gel and visualized. In this procedure, Rnase A/T1 is understood to digest away the RNA template and result in a mass shift towards a smaller product containing the ssDNA. Since Rnase H is expected to improve homogeneity of the 5′ and 3′ ssDNA boundaries, the impact of Rnase H on the distribution of products is also evaluated by gel analysis. The covalent linkage between the ncRNA template and ssDNA is confirmed by incubating the RT product with a 5′ to 3′ ssDNA exonuclease (RecJ) before or after treatment with a debranching enzyme (DBR1). RecJ is expected to be able to degrade the ssDNA after DBR1 has removed the 2′-5′ phosphodiester linkage between the RNA and ssDNA.

### Example 15—Determining Retron msr-msd Boundaries by NGS (Prophetic)

The msr-msd boundaries are determined by unbiased ligation of adapter sequences to the 5′ and 3′ end of the msDNA product after removal of the 2′-5′ phosphodiester linkage by DBR1. The resulting ligated product is PCR-amplified, library prepped, and subjected to next generation sequencing. Sequencing reads are aligned to the reference sequence to determine the 5′ and 3′ boundaries of the msd. The impact of the presence of Rnase H in the RT reaction on the homogeneity of 5′ and 3′ msd boundaries is also evaluated.

### Example 16—Systematic Evaluation of Insertion Sequences into the Msd on RT Activity (Prophetic)

Sequences of distinct length, predicted secondary structure, and GC-content are inserted into the msd at select insertion sites informed by the msd boundaries determined by NGS and secondary structure predictions of the ncRNA. The impact of these insertion sequences on RT activity are assessed by gel analysis or NGS as described above.

### Example 17—Testing the In Vitro Activity of RTs (Prophetic)

RT activity is assessed using a primer extension assay containing the RT derived from a cell-free expression system and an RNA template annealed to a DNA primer as described above. The resulting cDNA product(s) are detected by a denaturing polyacrylamide gel and qPCR as described above. Detection of cDNA drop-off products on the denaturing gel provides a relative assessment of processivity for candidates.

### Example 18—Evaluating the Priming Parameters of RTs (Prophetic)

Optimal primer length is determined by testing the RT's activity on an RNA template annealed to 5′-FAM labeled DNA primers of either 6, 8, 10, 13, 16, or 20 nucleotides in length. The RT is derived from a cell-free expression system as described above. After incubating the reaction, the reaction is quenched via the addition of Rnase H. The size distribution of cDNA products is analyzed on a denaturing polyacrylamide gel as described above. Optimal primer length is determined as the length that enables the RT to convert the most primer into cDNA product. The experimentally determined optimal primer length is then used in subsequent experiments, such as fidelity and processivity assays, to further characterize the RT in vitro.

### Example 19—Evaluating RT Fidelity (Prophetic)

To account for errors introduced during PCR and sequencing, RT fidelity is assessed by a primer extension assay as described above with the exception that a 14-nt unique molecular identifier (UMI) barcode is included in the primer for the reverse transcription reaction. The resulting full-length cDNA product is PCR-amplified, library-prepped, and subjected to next-generation sequencing. Barcodes with >5 reads are analyzed. After aligning to the reference sequence, mutations, insertions, and deletions are counted if the error is present in all sequence reads with the same barcode. Errors present in one but not all sequencing reads are considered to be introduced during PCR or sequencing. Further analysis of substitution, insertion, and deletion profile is performed, in addition to identification of mutation hotspots within the RNA template. The fidelity measurements are also performed with modified bases, e.g., pseudouridine, in the template.

### Example 20—Determining the Processivity Coefficient of RTs (Prophetic)

RT processivity is evaluated using a primer extension assay containing the RT enzyme derived from a cell-free expression system as described above and RNA templates between 1.6 kb-6.6 kb in length annealed to either a 5′-FAM labeled primer (for gel analysis) or unlabeled primer (for sequencing analysis).

Reverse transcription reactions are performed under single cycle conditions to disfavor rebinding of RT enzymes that have dropped off the RNA template during cDNA synthesis. The optimal trap molecule and concentration to achieve single cycle conditions are experimentally determined. The selected conditions are designed to provide sufficient inhibition of cDNA synthesis if incubated before reaction initiation but otherwise are designed to not impact the velocity of the reaction. Optimal trap molecules to test include unrelated RNA templates and unrelated RNA templates annealed to DNA primers of various lengths.

Once single cycle reaction conditions have been optimized, processivity is evaluated by initiating the reaction with the addition of dNTPs and the selected trap molecule after pre-equilibrating the RT with the RNA template annealed to a DNA primer in the reaction buffer. After incubating the reaction, the reaction is quenched by the addition of RnaseH. The size distribution of cDNA products is analyzed on a denaturing polyacrylamide gel as described above or subjected to PCR and library prepped for long-read sequencing. From these experiments, a processivity coefficient is quantified as the template length which yields 50% of the full-length cDNA product. The median length of the cDNA product from the single cycle primer extension reaction is used to estimate the probability that the RT will dissociate on the tested template. From this, the probability that the RT will dissociate at each nucleotide position is calculated, assuming that each dissociation is an independent event and that the probability of dissociation is equal at all nucleotide positions. The processivity coefficient representing the length of template at 50% of RT dissociated is then determined as 1/(2*P_{d}), where P_{d} is the probability of dissociation at each nucleotide.

### Example 21—Systematic Analysis of Challenge Structures on Primer Extension (Prophetic)

To evaluate the impact of challenging templates on RT activity, a primer extension reaction is conducted as stated above, with modifications. The RNA template contains one of the following challenge motifs at fixed distance (100-300 nt) downstream of the primer binding site: homopolymeric stretches, thermodynamically stable GC-rich stem loop, pseudoknot, tRNA, GII intron, and RNA template containing base or backbone modifications (e.g., pseudouridine, phosphothiorate bonds). After quenching the reaction, the size distribution of cDNA products is analyzed by denaturing polyacrylamide gel. An adapter sequence is also unbiasedly ligated to the 3′ ends of the cDNA products using T4 ligase. The ligated product(s) are then PCR-amplified and library prepped for next generation sequencing to identify both sites of RT misincorporation/insertions/deletions and sites of RT drop-off with single nucleotide resolution. Extent of RT drop-off at a given position is quantified by comparing the number of sequencing reads corresponding to the drop-off product to the number of sequencing reads corresponding to the full-length product.

### Example 22—Evaluating Non-Templated Base Additions (Prophetic)

Non-templated addition of bases to the 5′ end of the cDNA product is evaluated by next generation sequencing. Primer extension reactions containing the RT derived from the cell-free expression system and RNA template are conducted as described above. Systematic analysis of different RNA template lengths and sequence motifs at the 5′ end are tested. An adapter sequence is unbiasedly ligated to the 3′ ends of the resulting cDNA products by T4 ligase, resulting in capture of all cDNA products despite the potential heterogeneous nature of their 3′ ends. The ligated product(s) are then PCR-amplified and library prepped for next generation sequencing. Comparison of the expected full-length cDNA reference sequence to experimentally produced cDNA sequences that are longer than full-length enable identification of both the type and number of base additions to the 5′-end that were not templated by the RNA.

### Example 23—Determining 5′ and 3′ UTR Parameters for Activity and Processivity for R2, Non-LTR, and Similar Systems (Prophetic)

Proteins of interest are purified via a Twin-strep tag after IPTG-induced overexpression in E. coli. Purified proteins are tested against 1 kb and 4 kb cargos flanked by the 3′ UTRs identified from their native contexts and the 5′ UTRs plus 400 bp past the start codon. The 5′ and 3′ flanking sequences' effect on activity is assayed via qPCR to sections near the end of the template to determine if cargos with these native features produce superior results.

### Example 24—RT cDNA Synthesis Activity can be Harnessed for Multiple Applications (Prophetic)

Processes dependent on RNA are important in biology, such as expression, processing, modifications, and half-life. Quality control procedures in biotechnology performed on RNA utilize conversion of RNA to cDNA. Therefore, multiple RTs have been used for the production of cDNA libraries over the years. RTs used for these purposes include the MMLV RT, AMV RT, and GsI-IIC RT (TGIRT). The first two represent retroviral RTs, while the latter is a GII intron derived RT. GII intron derived RTs, as well as non-LTR derived RTs, show several advantages compared to their retroviral counterparts. For example, they are more processive, reading through structural and modified RNAs. Structural or modified RNAs may not be optimal substrates for retroviral RTs, as they create early termination products that can be misinterpreted as RNA fragments. In addition, the ability to template switch of some RTs can be harnessed for early adaptor addition, making the adaptor ligation procedures less important during library preparation. Therefore, highly processive RTs are suitable for the generation of libraries with complex RNA. Further, some highly processive RTs are generally smaller than currently used retroviral RTs, making their production and associated downstream processes easier. Several RTs described herein outperform the commercially available TGIRT enzyme, some with over 10-fold its cDNA synthesis activity.

### Example 25—LTR Restrotransposon RTs (MG151 Family)

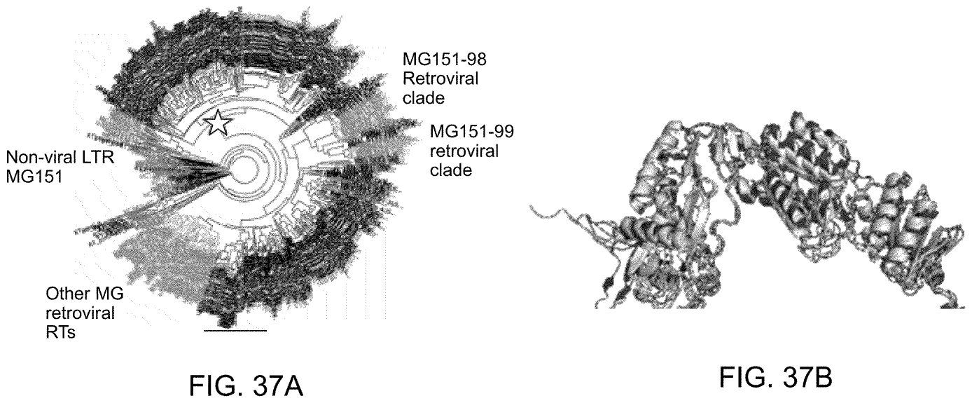

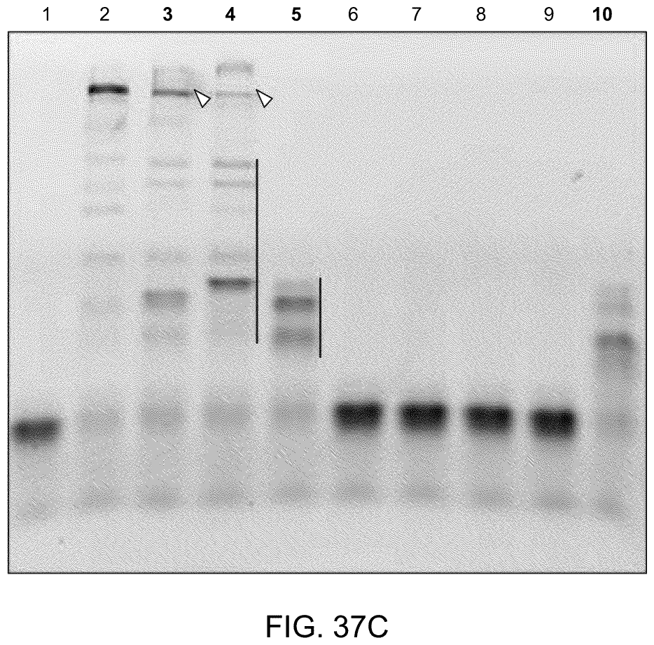

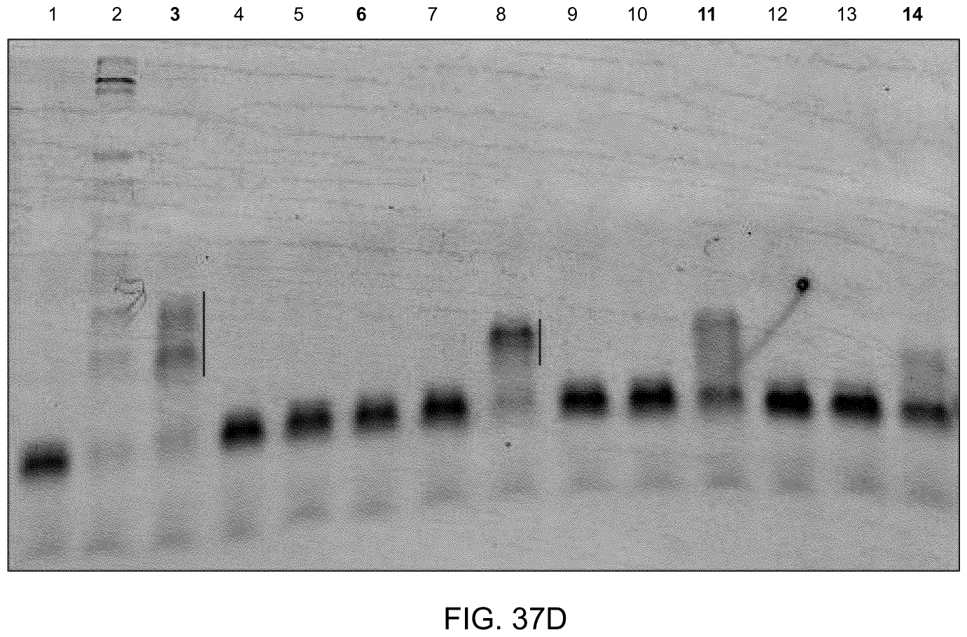

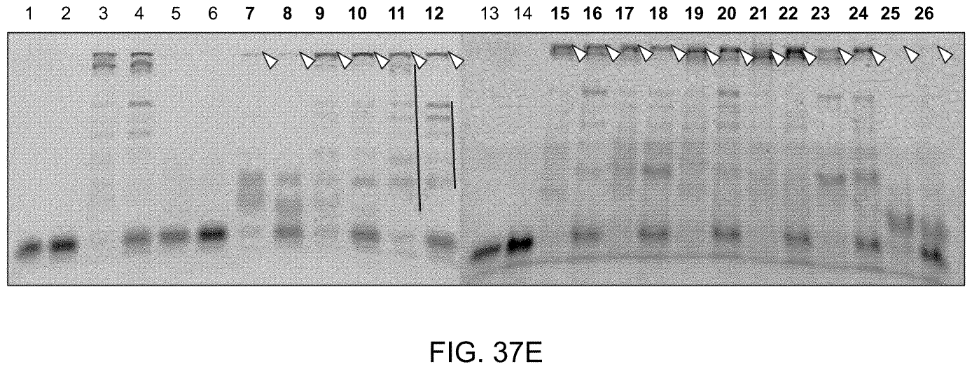

Long terminal repeat (LTR) retrotransposons, endogenous retroviruses, and proviral retroviruses integrate into their target sites via reverse transcription of an RNA template. Retroviral RTs of the MG151 family of LTR retrotransposons were identified from a phylogenetic tree from a multiple sequence alignment of full-length proteins containing LTR RT domains (FIG. 37A). The RT-RNAse H functional unit was determined from 3D structural predictions as the two relevant structural domains flanked by unstructured loops (FIG. 37B). Trimmed variants containing only RT and RNAse H domains were nominated for synthesis and laboratory characterization.

The in vitro activity of the LTR retrotransposon RTs family, which may include LTR retrotransposons, endogenous retroviruses, and proviral retroviruses (MG151 family), was assessed by a primer extension reaction containing RT enzyme derived from a cell-free expression system. Expression constructs were codon-optimized for E. coli. The substrate for the reaction was 100 nM of RNA template (202 nt) annealed to a 5′-FAM-labeled primer. The reaction buffer contained the following components: 50 mM Tris-HCl (pH 8.0), 75 mM KCl, 3 mM MgCl₂, 10 mM DTT, and 0.5 mM dNTPs. Following incubation at 37° C. for 1 h, the reaction was quenched via incubation with RNaseH, followed by the addition of 2×RNA loading dye. The resulting cDNA product(s) were separated on a 10% denaturing polyacrylamide gel and visualized using an imaging system. Based on these results (FIGS. 37C-37E), MG151-98 through MG151-100, MG151-105, MG151-106, MG151-111, MG151-114, MG151-117, MG151-119 through MG151-121, and MG151-123 through MG151-128 can synthesize cDNA from an RNA template in vitro.

### Example 26—Retron-Like RTs (MG154, MG155, MG156, MG157, MG158, MG159, MG160, and MG173 Families)

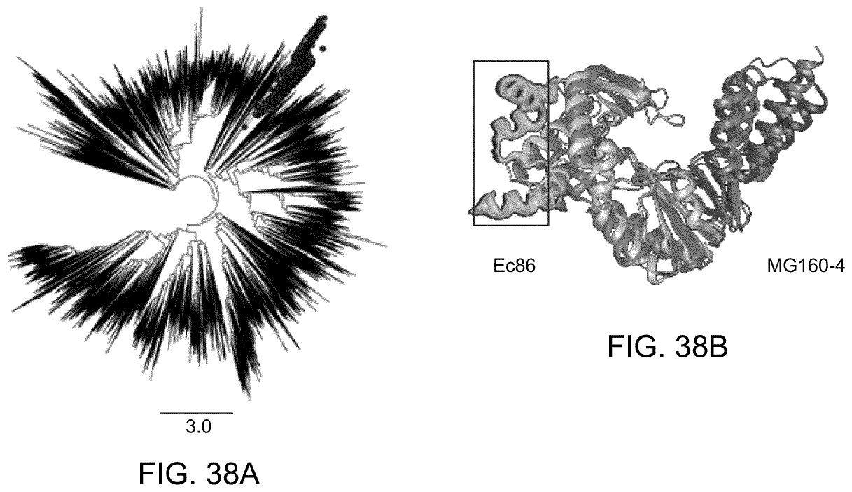

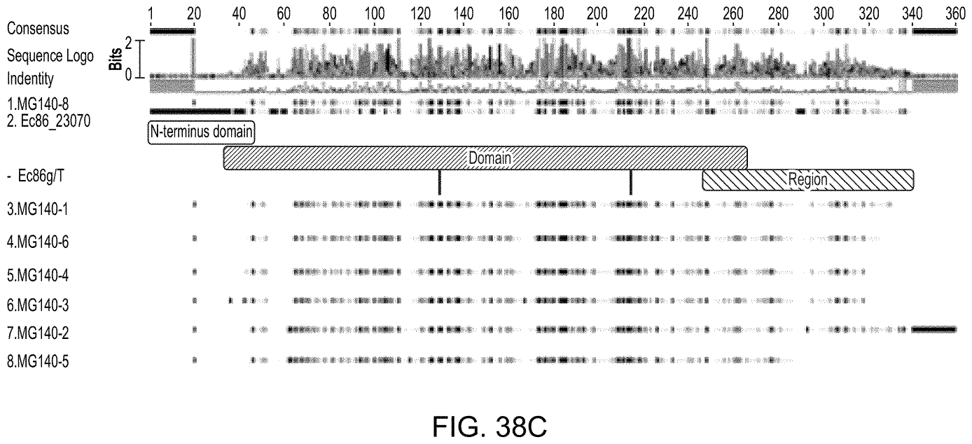

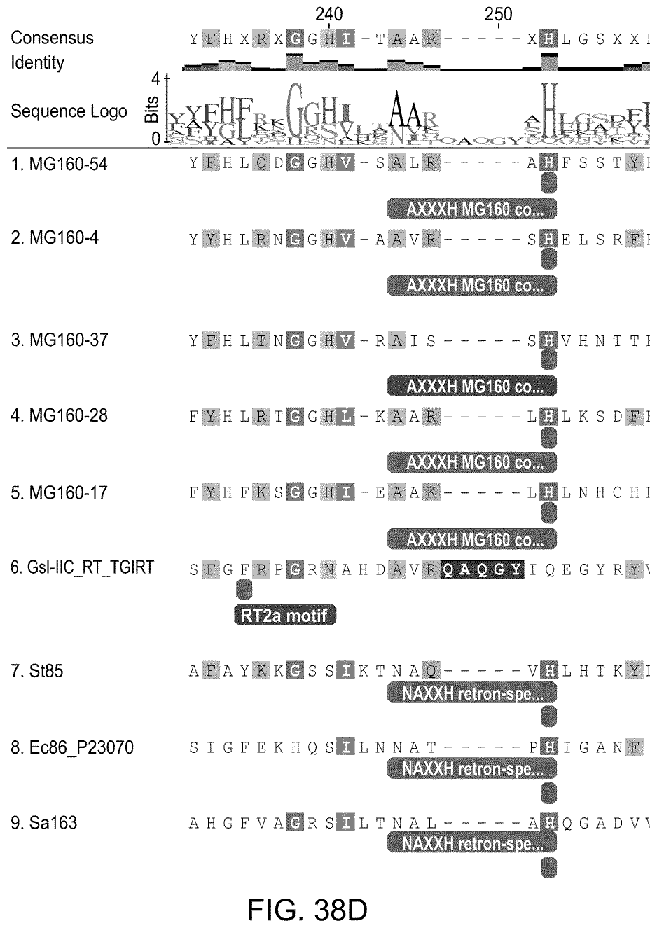

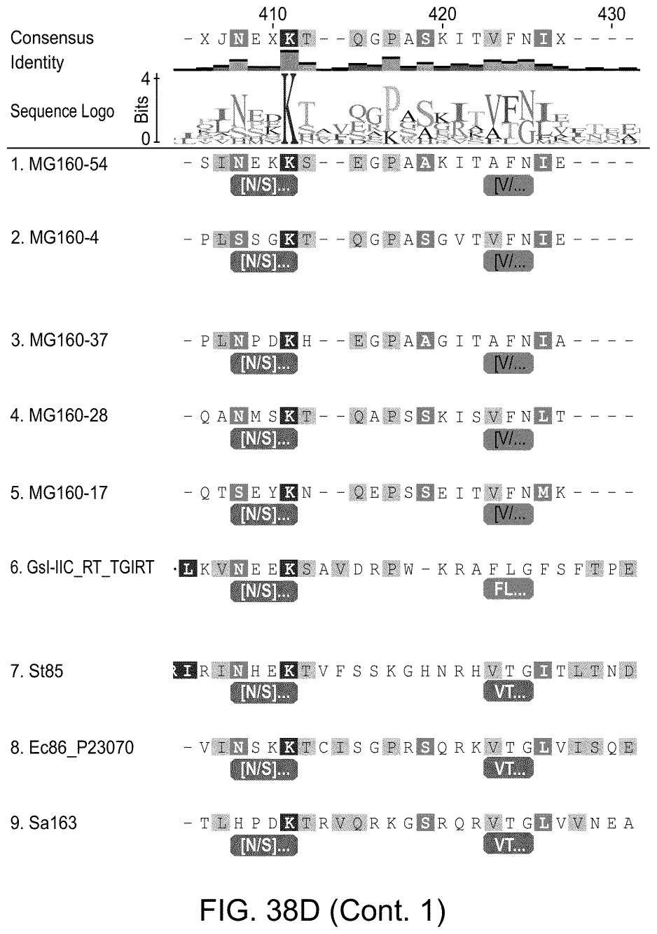

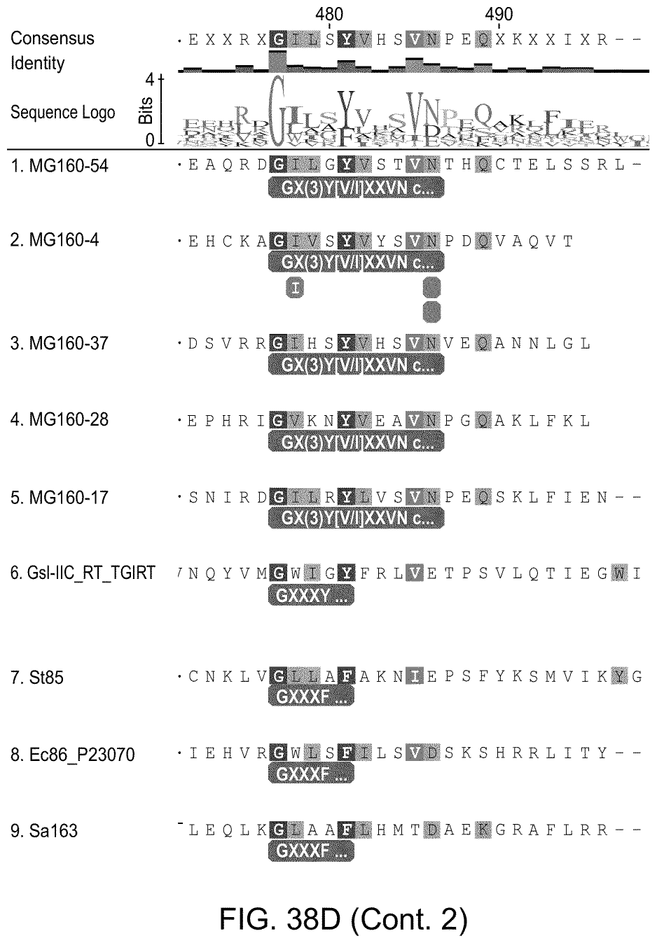

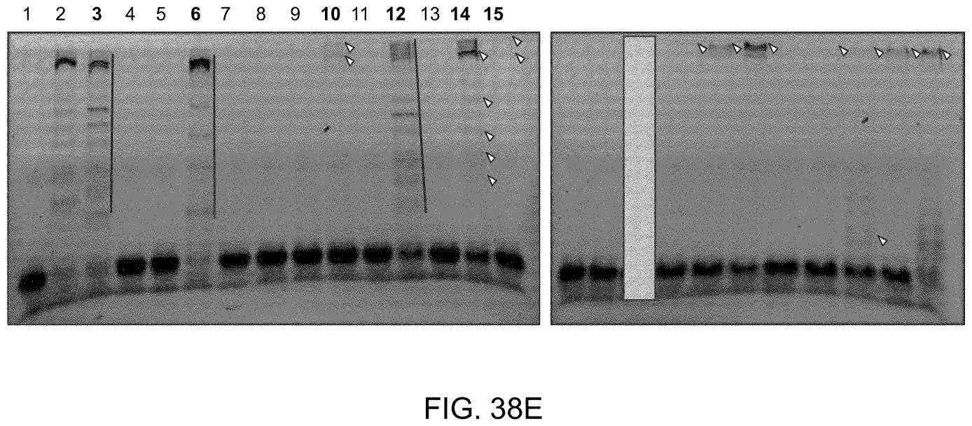

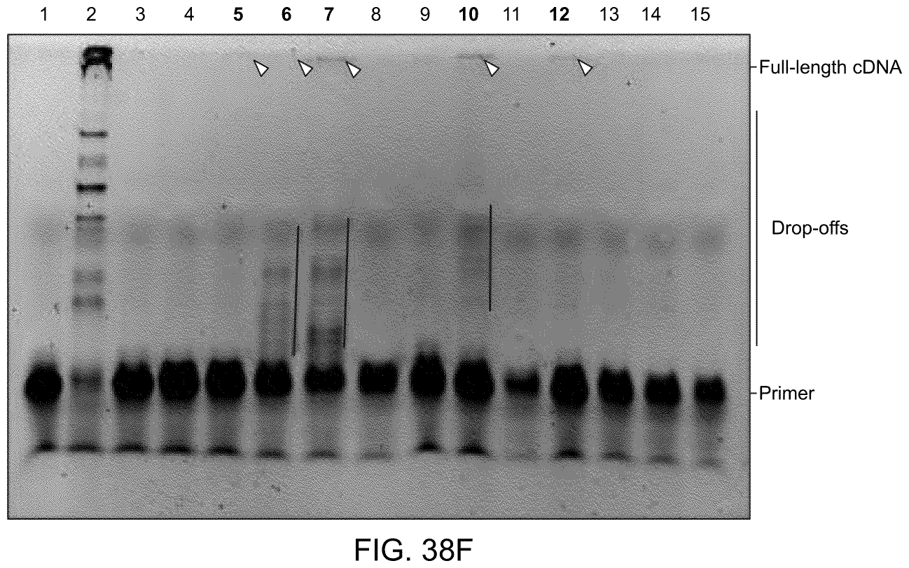

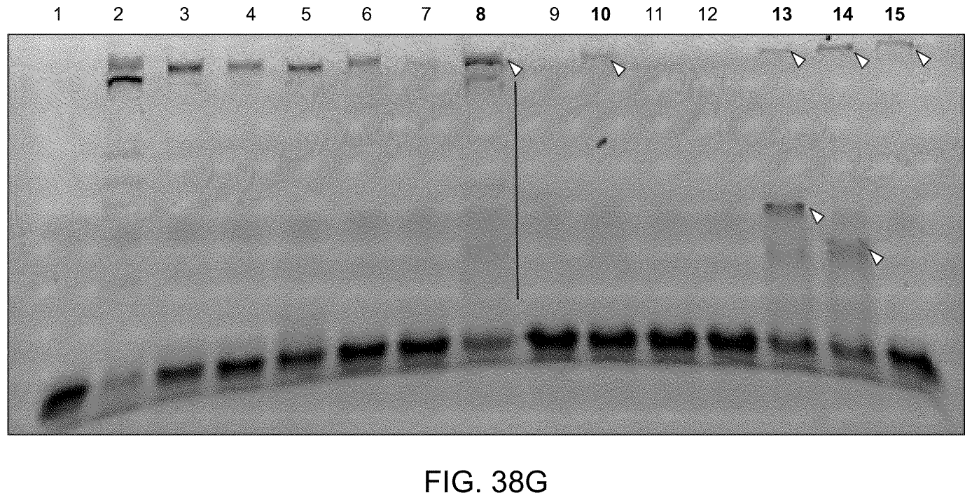

The MG160 family of RTs is a divergent group of “retron-like” single-domain RT enzymes previously identified within the retron RT clade, which form a distantly branching group (FIG. 38A). The enzymes range between 250 and 300 aa and are predicted to be active based on the presence of expected RT catalytic residues [F/Y]XDD. When aligned with a reference retron RT (Ec86 retron RT from E. coli), both structural and sequence alignments indicate that the MG160 enzymes lack an N-terminal region found in retrons and display C-termini of variable lengths (FIGS. 38B-38C). Although they are phylogenetically related to retrons, the MG160 family of RTs contain specific motifs that distinguish them from known RTs. For example, MG160 RTs lack the conserved amino acid motifs NAXXH and VTG present in other retrons. Instead, MG160 enzymes contain family-specific conserved amino acid motifs AXXXH and [V/A]FN. In addition, they share conserved motifs with group II intron RTs, such as the GXXXY motif, although the motif is longer in MG160 enzymes (GX(3)YV(X)xVN) (SEQ ID NO: 2275) (FIG. 38D).

The in vitro activity of retrons and retron-like RTs (MG160 family) was assessed by a primer extension reaction as described above. Expression constructs were codon-optimized for E. coli and contained an N-terminal single Strep tag. The substrate for the reaction was 100 nM of RNA template (202 nt) annealed to a 5′-FAM-labeled primer. The reaction buffer contained the following components: 50 mM Tris-HCl (pH 8.0), 75 mM KCl, 3 mM MgCl₂, 10 mM DTT, and 0.5 mM dNTPs. Following incubation, the reaction was quenched and the resulting cDNA product(s) were visualized as described above.

Based on these results (FIGS. 38E and 38G), the following retron-like MG160 RTs are capable of synthesizing cDNA in vitro: MG160-17, MG160-28, MG160-40, MG160-54, MG160-56, MG160-58, MG160-59, MG160-63, MG160-64, and MG160-65. Notably, MG160-65 has higher apparent processivity than MMLV and other MG160 family RTs on the 202 nt RNA template as shown by the strong full-length cDNA band and lack of apparent drop offs (FIG. 38E, lane 21).

By gel analysis (FIGS. 38E-38G), the following retron-like RTs of families MG154-MG159 and MG173 have non-specific cDNA synthesis activity on a generic, noncognate RNA template in vitro: MG155-2, MG155-3, MG155-4, MG155-5, MG156-1, MG156-2, MG157-5, MG159-1, MG159-2, MG159-3, MG173-1, and MG173-2.

### Example 27—cDNA Synthesis by Group II Intron and Non-LTR R2 Retrotransposase RTs

Group II introns and non-LTR retrotransposases are capable of integrating large cargo into a target site via reverse transcription of an RNA template. These RTs integrate an RNA template via target-primed reverse transcription (TPRT), a mechanism in which cDNA synthesis is primed by the free 3′ hydroxyl group at the target DNA nick.

### Testing the In Vitro Activity of Group II Intron RTs Class a, B, C, E, G, ML and CL (MG163, MG164, MG153, MG166, MG168, MG169, and MG170 Families)

The in vitro activity of GII intron Class A (MG163), Class B (MG164), Class C (MG153), Class E (MG166), Class G (MG168), Class ML (MG169), and Class CL (MG170) RTs was assessed by a primer extension reaction as described above. Expression constructs were codon-optimized for E. coli and contained an N-terminal single Strep tag. The substrate for the reaction was 100 nM of RNA template (202 nt) annealed to a 5′-FAM-labeled primer. The reaction buffer contained the following components: 50 mM Tris-HCl (pH 8.0), 75 mM KCl, 3 mM MgCl₂, 10 mM DTT, and 0.5 mM dNTPs. Following incubation, the reaction was quenched and cDNA products visualized as described above. RT activity was also assessed by qPCR with primers that amplify the full-length cDNA product. Products from the primer extension assay were diluted to ensure cDNA concentrations were within the linear range of detection. The amount of cDNA was quantified by extrapolating values from a standard curve generated with the DNA template of known concentrations.

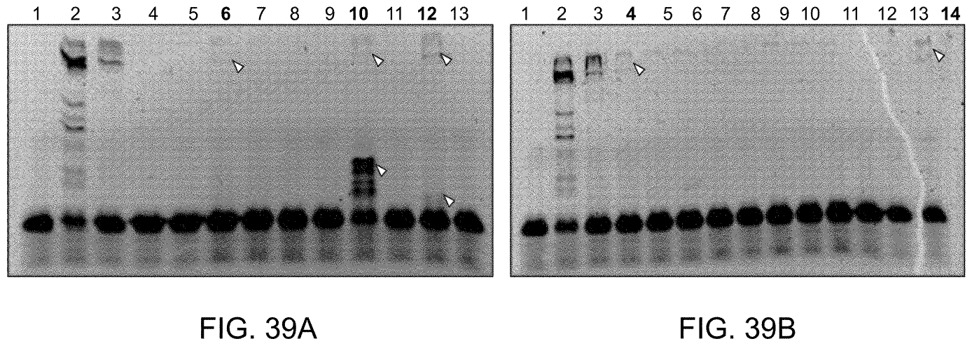

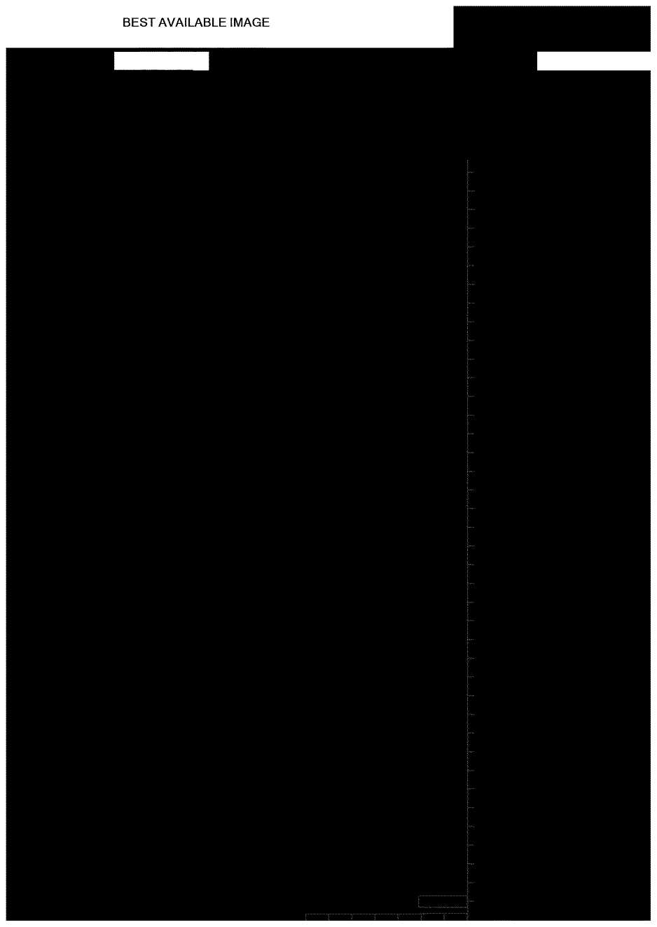

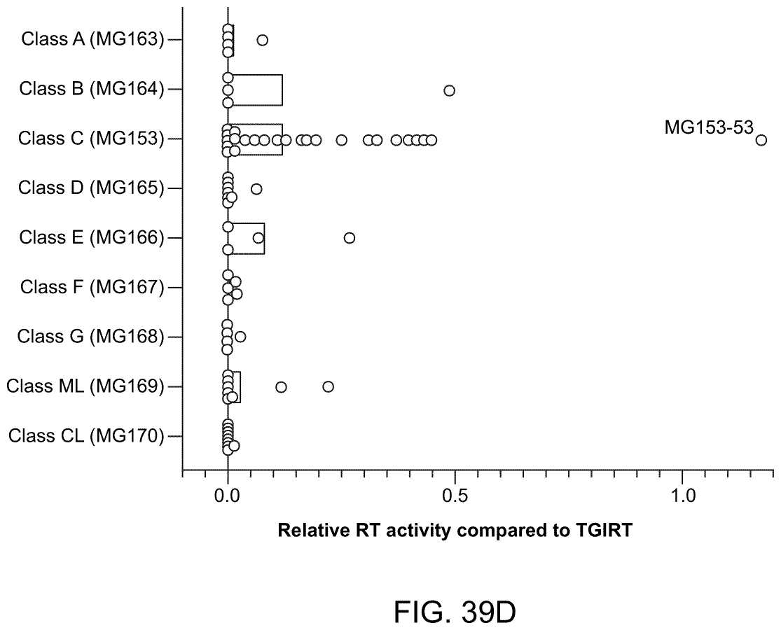

By detection of cDNA products on a denaturing gel (FIGS. 39A-39B), the following GII intron candidates were active under these experimental conditions: MG163-2, MG166-2, MG166-4, MG169-1, and MG169-11. Active candidates exhibited a varying degree of apparent processivity compared to the highly processive control GII Class C RT TGIRT as indicated by the presence of smaller cDNA drop-off products (i.e., MG166-2). By qPCR, the following additional candidates were also active under these experimental conditions (cDNA detected >10-fold above background): MG163-1, MG163-3, MG164-3, MG164-5, MG168-1, MG169-9, MG170-1, MG170-4, and MG170-7 (FIG. 39C).

A summary of in vitro cDNA synthesis activity across all GII intron RTs is shown in FIG. 39D. Enzyme activity relative to the control GII class C RT TGIRT was determined from qPCR quantification of full-length cDNA production as described above.

### Testing the In Vitro Activity of Non-LTR R2 Retrotransposase RTs (MG140, MG146, MG148, and MG176 Families)

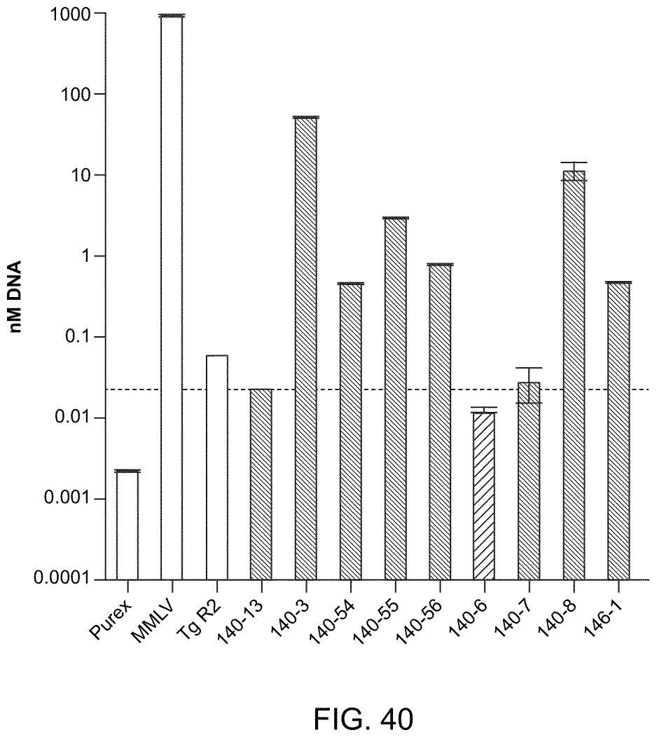

The in vitro activity of non-LTR R2 and other retrotransposon-associated RTs was assessed by a primer extension reaction containing RT enzymes derived from a cell-free expression system. Expression constructs were codon-optimized for E. coli. The substrate for the reaction was 100 nM of RNA template (202 nt) annealed to a 5′-FAM-labeled primer. The reaction buffer contained the following components: 40 mM Tris-HCl (pH 7.5), 0.2 M NaCl, 10 mM MgCl₂, and 1 mM TCEP. Following incubation at 37° C. for 1 h, RT activity was assessed by qPCR with primers that amplify the full-length cDNA product as described above. An RT was considered active in vitro if cDNA product is detectable 10-fold above a cell-free expression system no-template control background. Based on these results (FIG. 40), MG140-54 through MG140-56 can perform cDNA synthesis in vitro.

### Testing the In Vitro Activity of GII Intron RTs on 4.1 kb RNA Template

The ability for GII intron RTs to reverse transcribe a long 4.1 kb RNA template was assessed by a primer extension reaction containing RT enzymes derived from a cell-free expression system. Expression constructs were codon-optimized for E. coli and contained an N-terminal single Strep tag. The substrate for the reaction was 100 nM of RNA template annealed to a DNA priming oligo. The reaction buffer contained the following components: 50 mM Tris-HCl (pH 8.0), 75 mM KCl, 3 mM MgCl₂, 10 mM DTT, and 0.5 mM dNTPs. Following incubation at 37° C. for 1 h, cDNA products were detected by Taqman qPCR using taqman probes and primers that amplify 100 bp amplicons corresponding to the beginning (FAM signal) and end (HEX signal) of the resulting cDNA product (4.1 kb). The cDNA products were quantified by extrapolating against a standard curve. The calculated % HEX/FAM represents the percentage of cDNA that corresponds to a full-length, 4.1 kb product.

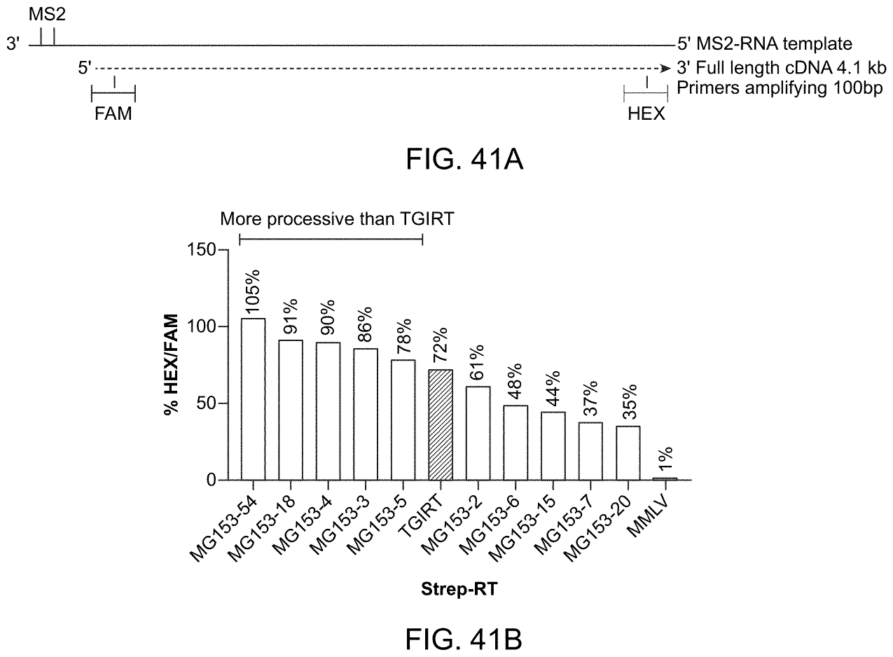

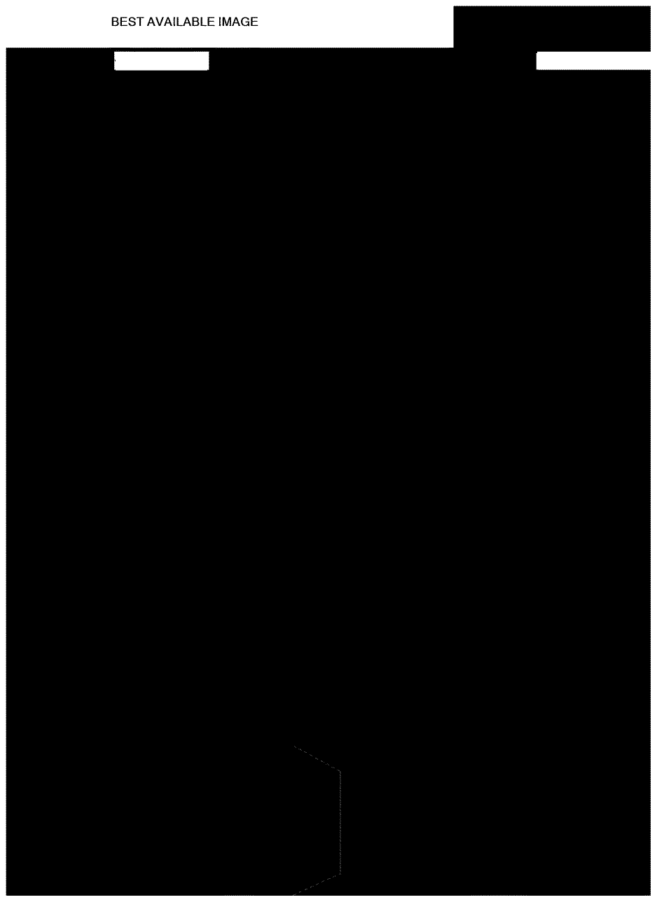

Based on these results (FIGS. 41A-41B), all tested GII Class C RTs (MG153) were capable of producing appreciable amounts of full-length cDNA (at least 35%) compared to the retroviral control RT MMLV (1%). Many GII RTs (including MG153-54, MG153-18, MG153-3 through MG153-5) exhibited higher apparent processivity than the control GII RT (TGIRT) under these experimental conditions.

Human Cells cDNA Synthesis by RTs

The ability of these enzymes to produce cDNA in a mammalian environment was tested by expressing them in mammalian cells and detecting cDNA synthesis by qPCR. Reverse transcriptases were cloned in a plasmid for mammalian expression under the CMV promoter as fusion proteins having MS2 coat protein (MCP) at the N terminus, in addition to a flag-HA tag (FH). MCP is a protein derived from the MS2 bacteriophage that recognizes a 20 nucleotide RNA stem loop with high affinity (subnanomolar Kd). By fusing the RTs with MCP and having the MS2 loops in the RNA template, it is ensured that once the RT is translated, it finds the RNA template and starts cDNA synthesis from the DNA primer hybridized to the RNA template.

A plasmid containing MCP fused to the RT candidate under CMV promoter was cloned and isolated for transfection in HEK293T cells. Transfection was performed using liposome based system. mRNA codifying dCas9 fused to nanoluciferase was generated. In order to degrade any DNA template left in the mRNA preparation, the reaction was treated with Turbo DNase for 1.5 hour, and the mRNA was cleaned. The mRNA was hybridized to a complementary DNA primer in 10 mM Tris pH 7.5 and 50 mM NaCl at 95° C. for 2 min and cooled to 4° C. at a rate of 0.1° C./s. The mRNA/DNA hybrid was transfected into HEK293T cells using a liposome based transfection 6 hours after the plasmid containing the MCP-RT fusion was transfected. 18 hours post mRNA/DNA transfection, cells were lysed using DNA Extraction Solution, and 100 μL of quick extract was added per 24 well in a 24 well plate. The RNA template is ~4247 nt (SEQ ID NO: 896). Primers to amplify first and last 100 bps products from the newly synthesized cDNA (4100 bp) were designed, along with Taqman probes to quantify their amplification (SEQ ID NOs: 897-902) (FIG. 42).

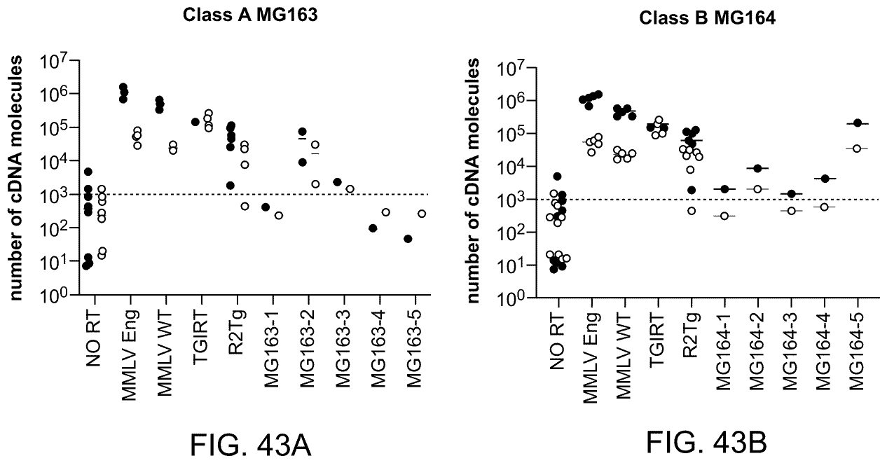

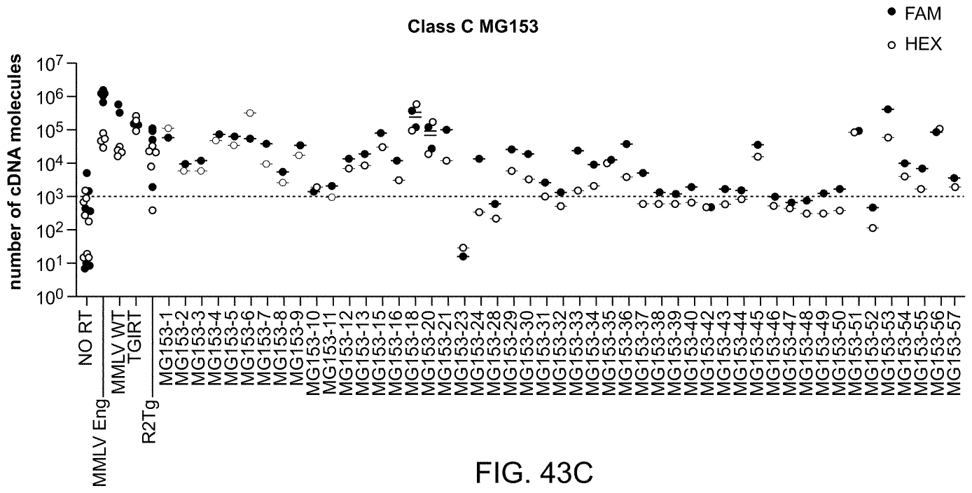

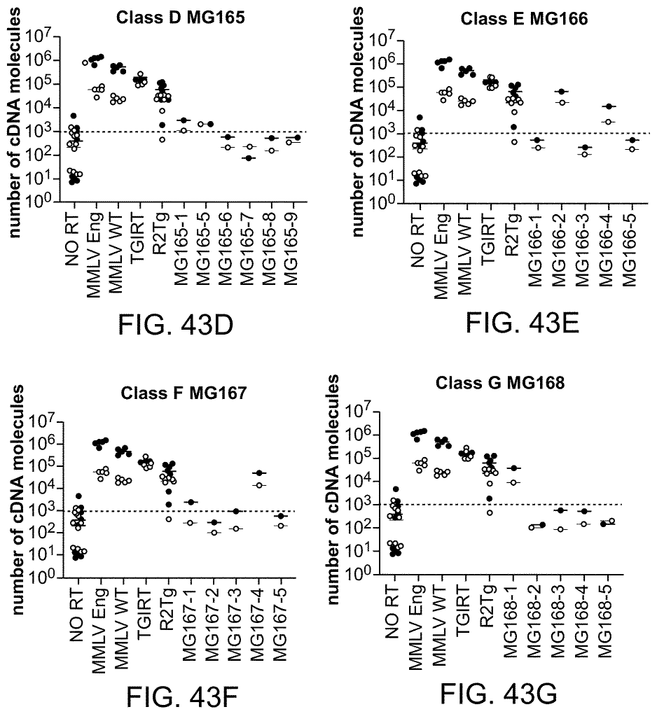

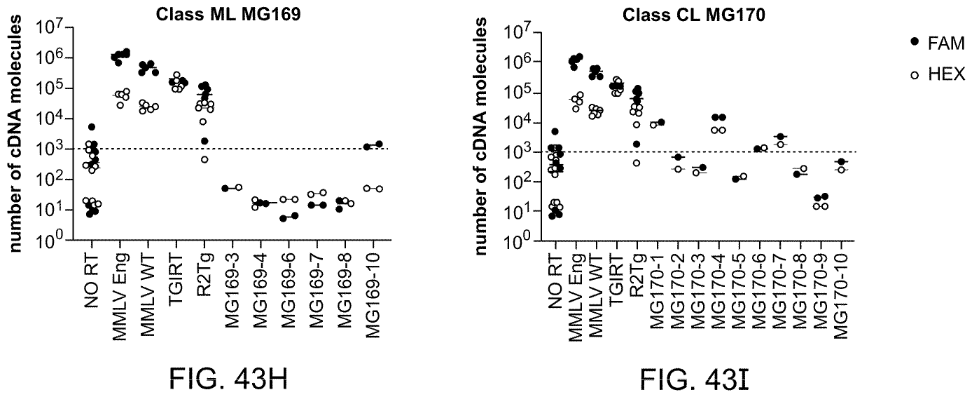

Activity for the control GII intron RT TGIRT, the retroviral MMLV (WT and penta-mutant), as well as a positive control for R2, R2Tg, was detected (FIGS. 43A-431), as shown by an early amplification of the first and last 100 bp products. As expected for a low processivity RT, the retroviral RTs (MMLVs) showed high amplification levels of the first 100 bps (FAM signal), but the levels at which they completed cDNA synthesis (the last 100 bps) was lower (20-fold lower than first 100 bp, as observed by the FAM/HEX ratio signal). Group II intron-derived RTs and R2 non-LTR retrotransposon RTs showed a closer FAM/HEX ratio, demonstrating their high processivity (FIGS. 43A-431 and 44, respectively).

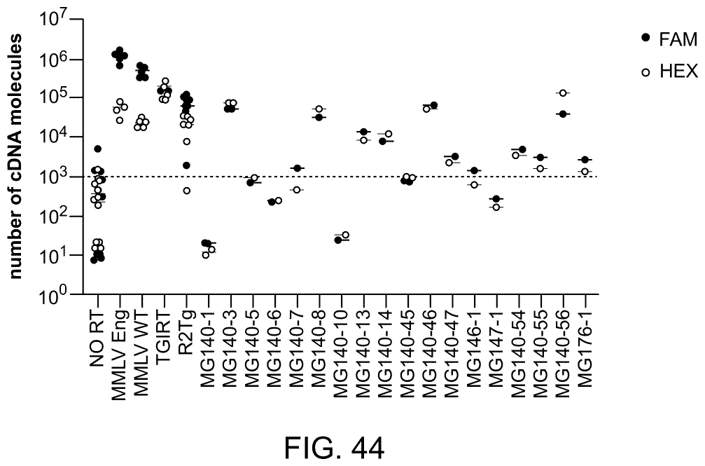

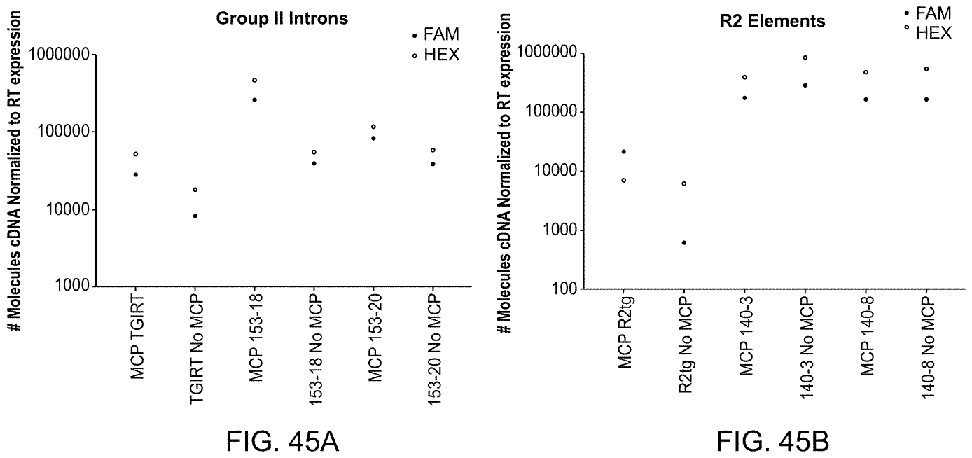

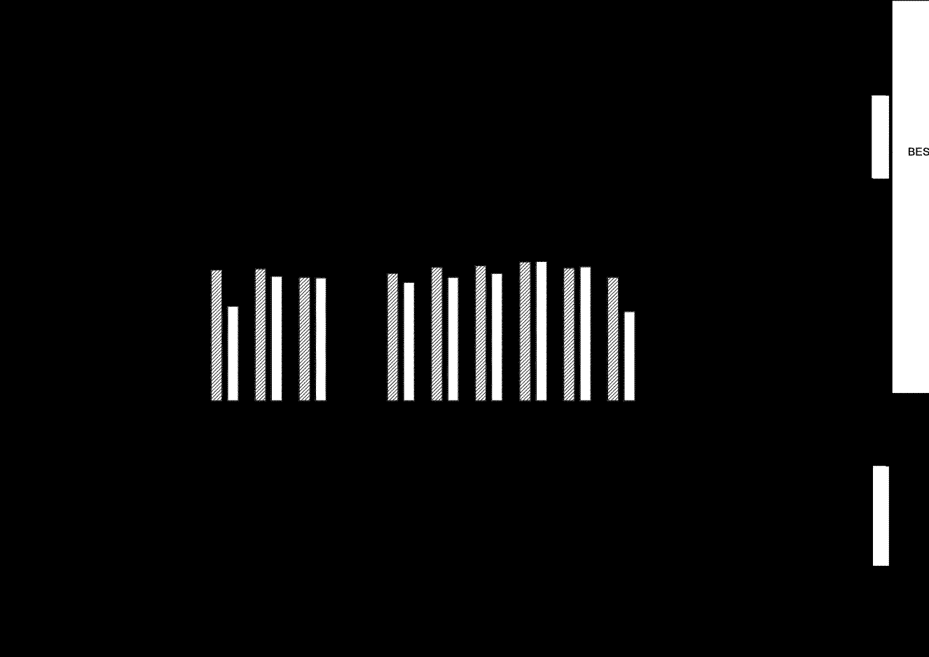

Most of the tested candidates showed a wide range of RT activity in mammalian cells. Candidates with high cDNA synthesis efficiency include Group II intron Class A (MG163-2), Class B (MG164-5), Class C (MG153-18, MG153-20, MG153-21, MG153-51, and MG153-53), Class E (MG166-2), Class F (MG167-4), and Class G (MG168-1). From the R2 non-LTR family, well-performing candidates include MG140-3 and MG140-8 (FIG. 44). Addition of an MCP tag fused to the RT did not affect cDNA synthesis activity and, for some candidates, it increased cDNA synthesis in mammalian cells (FIGS. 45A-45B).

### Example 28—In Vitro cDNA Synthesis of Modified RNA Template by Diverse RTs

In order determine the effect of a modified RNA template on cDNA synthesis activity for some RT candidates, a modified 202 bp RNA template was prepared by performing in vitro transcription of the template with complete replacement of uridine with N1-methyl pseudourine (m1Ψ). In vitro cDNA synthesis activity of RTs was assessed by a primer extension reaction as described above. The substrate for the reaction was 100 nM of standard U or m1Ψ-modified RNA template (202 nt) annealed to a 5′-FAM labeled primer. Following incubation, the reaction was quenched, and cDNA products ere visualized as described above. Reverse transcription activity was quantified from the denaturing gel by determining the percentage of primer converted into cDNA product(s) using imaging software.

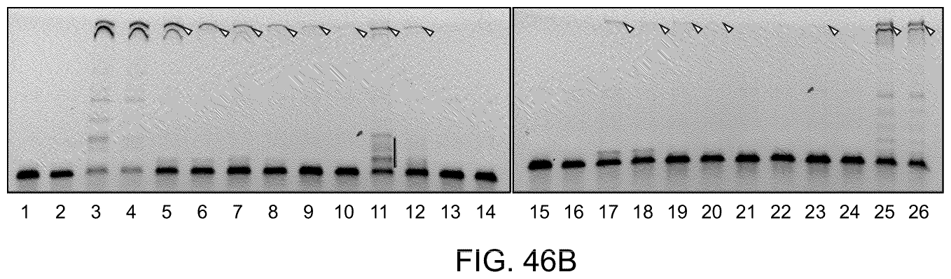

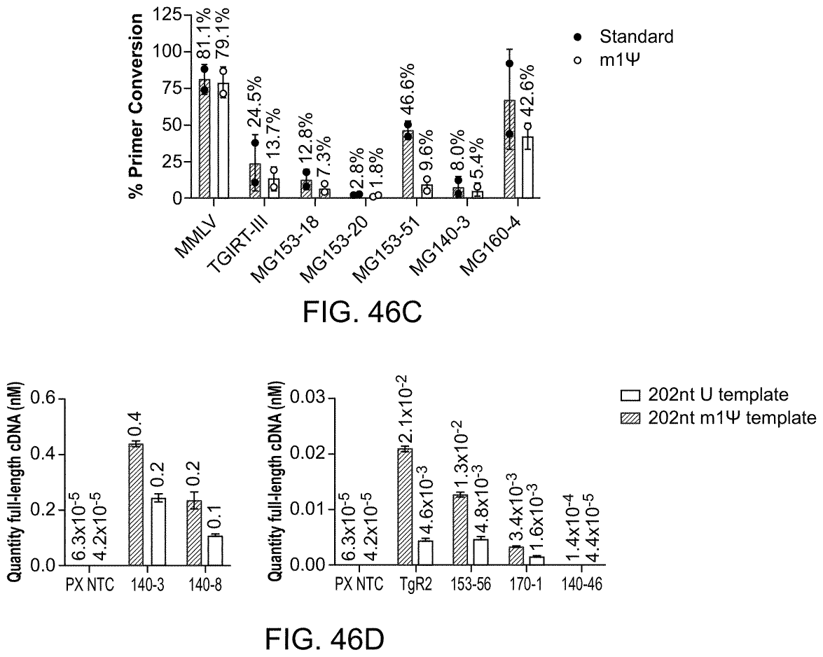

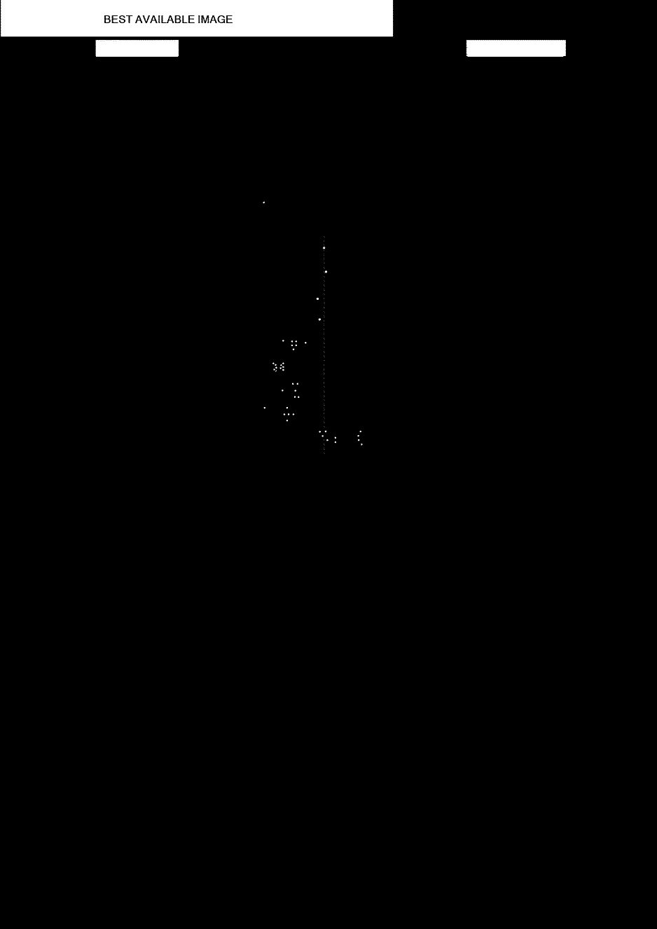

Based on these results, MG151 RTs that demonstrated robust activity on the standard RNA template were also highly active on the m1Ψ-modified RNA template, namely MG151-119 through MG151-121 and MG151-123 through MG151-128 (FIG. 37E). This result was supported by gel quantification of primer conversion normalized to MMLV activity, which indicates that the MG151 family of RTs have similar activity levels with both RNA templates (FIG. 46A). Similarly, analysis of cDNA synthesis products on a denaturing gel (FIG. 46B) and quantification of RT activity by gel analysis or qPCR (FIGS. 46C-46D) for group II intron, R2, and retron-like MG160 candidates indicated that diverse RTs that are active with the standard RNA template maintained appreciable activity on the m1l′-modified RNA template.

### Example 29—cDNA Synthesis by Group II Intron RTs, Non-LTR Retrotransposon RTs, and Retron-Like RTs

Group II introns and non-LTR retrotransposases are capable of integrating large cargo into a target site via reverse transcription of an RNA template. These reverse transcriptases (RTs) integrate an RNA template via target primed reverse transcription (TPRT), a mechanism in which cDNA synthesis is primed by the free 3′ hydroxyl group at the target DNA nick. The MG160 family of RTs are a divergent group of “retron-like” single-domain RT enzymes previously identified within the retron RT clade, which form a distantly branching group. The enzymes are predicted to be active based on the presence of expected RT catalytic residues [F/Y]XDD.

Results: Human Cells cDNA Synthesis by RTs

The ability of RTs to produce cDNA in a mammalian environment was tested by expressing them in mammalian cells and detecting cDNA synthesis by qPCR. Reverse transcriptases were cloned in a plasmid for mammalian expression under the CMV promoter as fusion proteins having MS2 coat protein (MCP) at the N terminus, in addition to a flag-HA tag (FH). MCP is a protein derived from the MS2 bacteriophage that recognizes a 20 nucleotide RNA stem loop with high affinity (subnanomolar Kd). By fusing the RTs with MCP and having the MS2 loops in the RNA template, it is ensured that once the RT is translated it finds the RNA template and starts cDNA synthesis from the DNA primer hybridized to the RNA template.

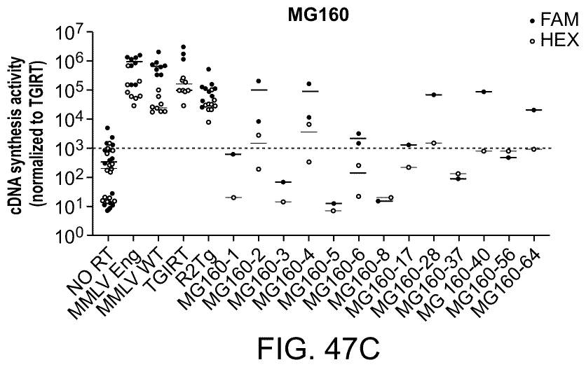

A plasmid containing MCP fused to the RT candidate under CMV promoter was cloned and isolated for transfection in HEK293T cells. Transfection was performed using liposome based system. mRNA codifying dCas9 fused to nanoluciferase was generated. To degrade any DNA template left in the mRNA preparation the reaction was treated with DNase for 1.5 hours, and the mRNA was cleaned up using Transcription Clean-Up kit. The mRNA was hybridized to a complementary DNA primer in 10 mM Tris pH 7.5, 50 mM NaCl at 95° C. for 2 min and cooled to 4° C. at the rate of 0.1° C./s. The mRNA/DNA hybrid was transfected into HEK293T cells using liposome based system 6 hours after the plasmid containing the MCP-RT fusion was transfected. 18 hours post mRNA/DNA transfection, cells were lysed using DNA extraction solution. 100 μl of quick extract was added per 24 well in a 24 well plate. The RNA template was ~4247 nt. Primers to amplify first and last 100 bps products from the newly synthesized cDNA (4100 bp) were designed, along with taqman probes to quantify their amplification (FIG. 47A).

Activity for the control GII intron RT TGIRT, the retroviral MMLV (WT and penta-mutant) as well as a positive control for R2 RTs, R2Tg, was detected (FIGS. 47B and 47C), as shown by an early amplification of the first and last 100 bp products. As expected for a low processivity RT, the retroviral RTs (MMLVs) showed high amplification levels of the first 100 bps (FAM signal) but the levels at which they complete cDNA synthesis (the last 100 bps) was lower (20-fold lower than first 100 bp, as observed by the FAM/HEX ratio signal). Control group II intron RT TGIRT and control R2 non-LTR retrotransposon RT R2Tg showed a closer FAM/HEX ratio, demonstrating their high processivity (FIGS. 47B and 47C). Four candidates of the MG148 family of non-LTR retrotransposon RTs were tested in mammalian cells (FIG. 47B). All tested candidates showed low activity compared to the control RTs. Thirteen candidates of the MG160 family of retron-like RTs were also tested similarly. Although some candidates (namely MG160-2, MG160-4, MG160-28, MG160-40, and MG160-64) showed high activity based on high FAM fluorescence levels, they had poor processivity based on the much lower HEX fluorescence levels (FIG. 47C).

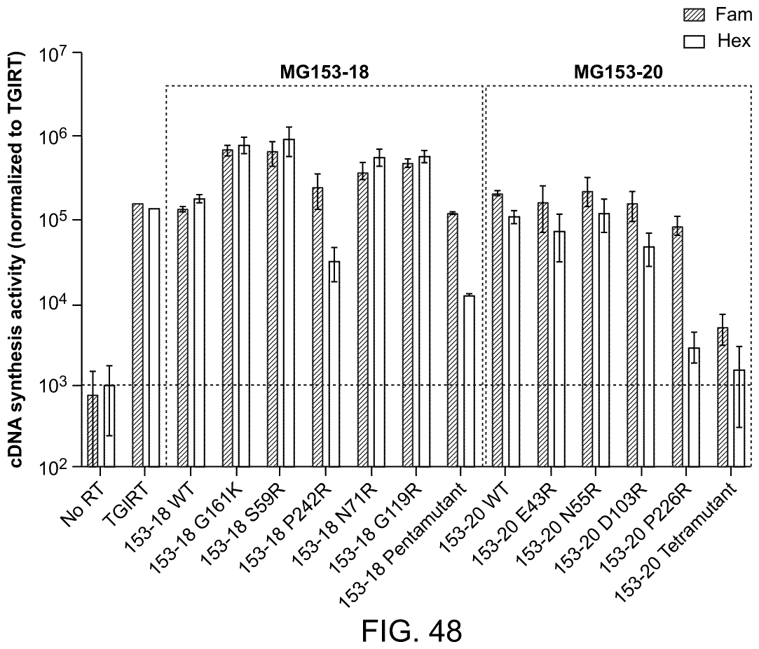

Two GII intron RTs, MG153-18 and MG153-20, were previously selected candidates owing to their high activity and processivity. Rationally engineered mutants were screened for both candidates using the above-mentioned cDNA synthesis assay in mammalian cells. Five individual point mutants and one pentamutant was screened for MG153-18 (FIG. 48). Four of the five point mutations increased RT activity without compromising processivity. MG153-18 G161K and MG153-18 S59R mutants increased RT activity by ~5 fold over WT. MG153-18 N71R and MG153-18 G119R increased activity by ~2.8 fold and ~3.6 fold over WT respectively. MG153-18 P242R led to a decrease in processivity although it marginally increased RT activity. This is likely reflected in the pentamutant as well which displays a low processivity over WT MG153-18. For MG153-20, four individual point mutants and one tetramutant was screened (FIG. 48). None of the MG153-20 variants improved activity over WT. Notably MG153-20 P226R, analogous to MG153-18 P242R, displayed low processivity likely reflected in the MG153-20 tetramutant.

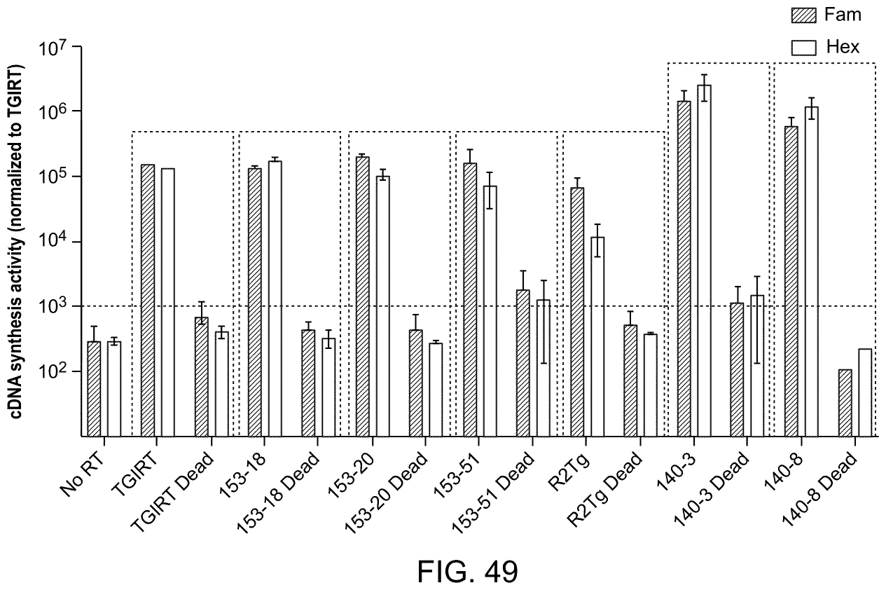

Inactivating mutants for control RTs TGIRT and R2Tg, as well as previously identified selected RTs with high activity and processivity were also screened for their use as negative controls using the cDNA synthesis assay in mammalian cells (FIG. 49). All screened RT dead mutants exhibited activity at or below background (FIG. 49, indicated by a dashed horizontal line), much lower than their WT counterparts.

### Example 30—Non-Specific In Vitro Activity of MG173 and MG192 Family of Retron RTs

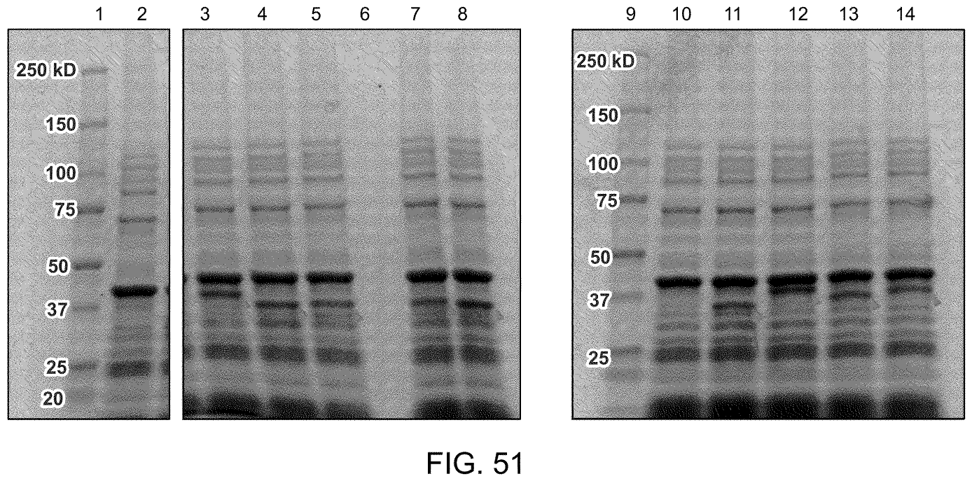

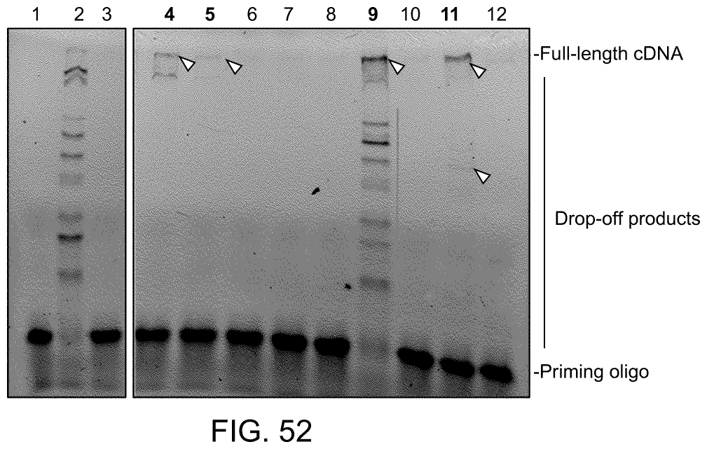

The in vitro activity of retron RTs on a general RNA template was assessed by a primer extension reaction containing RT enzyme derived from a cell-free expression system. Expression constructs were codon-optimized for E. coli and contained an N-terminal single Strep tag. The expression of MG173 and MG192 family of RTs from the cell-free expression system was confirmed by SDS-PAGE analysis (FIG. 51). The substrate for the reaction was 100 nM of RNA template (202 nt) annealed to a 5′-FAM labeled primer. The reaction buffer contained the following components: 50 mM Tris-HCl (pH 8.0), 75 mM KCl, 3 mM MgCl₂, 10 mM DTT, and 0.5 mM dNTPs. Following incubation at 37° C. for 1 h, the reaction was quenched via incubation with RNaseH followed by the addition of 2×RNA loading dye. The resulting cDNA product(s) were separated on a 10% denaturing polyacrylamide gel and visualized. Based on these results (FIG. 52), the following retron RTs are capable of performing primer extension on a general RNA template that is not their own ncRNA: MG173-3 (SEQ ID NO: 1546), 173-4 (SEQ ID NO: 1547), 173-8 (SEQ ID NO: 1551), and 173-10 (SEQ ID NO: 1553).

### Example 31—In Vitro Activity and Processivity of Retron RTs on 4.1 kb RNA Template

The ability for retron RTs to reverse transcribe a 4.1 kb RNA template was assessed by a primer extension reaction containing RT enzyme derived from a cell-free expression system. Expression constructs were codon-optimized for E. coli and contained an N-terminal single Strep tag. The substrate for the reaction was 100 nM of RNA template annealed to a DNA priming oligo. The reaction buffer contained the following components: 50 mM Tris-HCl (pH 8.0), 75 mM KCl, 3 mM MgCl₂, 10 mM DTT, and 0.5 mM dNTPs. Following incubation at 37° C. for 1 h, cDNA products were detected by Taqman qPCR using Taqman probes and primers that amplify 100 bp amplicons corresponding to the beginning (FAM signal) and end (HEX signal) of the resulting cDNA product (4.1 kb). The cDNA products were quantified by extrapolating against a standard curve. The calculated % HEX/FAM represents the percentage of cDNA that corresponds to a full-length, 4.1 kb product.

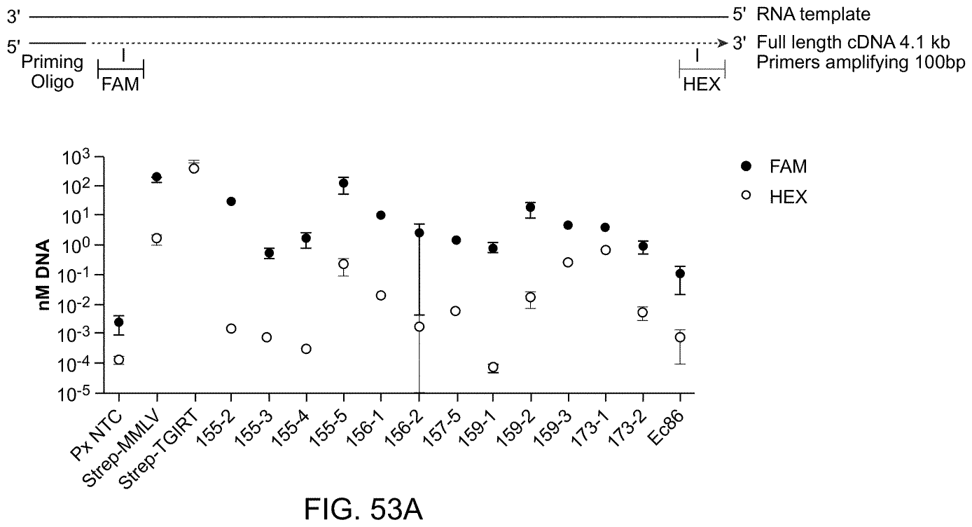

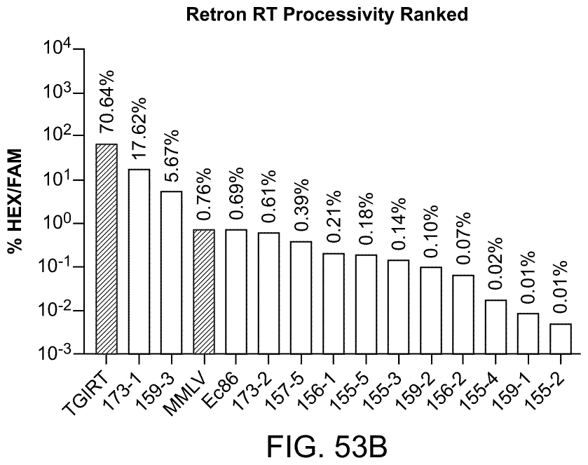

Based on these results (FIGS. 53A-53B), MG173-1 (SEQ ID NO: 734) and MG159-3 (SEQ ID NO: 626) have higher processivity than the control retroviral RT MMLV and positive control retron RT Ec86, where MMLV and Ec86 produce 0.8% and 0.7% full-length cDNA, respectively, and MG173-1 and MG159-3 produce 17.6% and 5.7% full-length cDNA, respectively. Several retron RTs exhibit processivity profiles similar to that of MMLV and Ec86, namely MG173-2 (SEQ ID NO: 735), MG157-5 (SEQ ID NO: 622), and MG156-1 (SEQ ID NO: 616).

### Example 32—Fidelity of Processive Reverse Transcriptases

Targetable integration of large cargo into human genomic DNA has been a long sought goal for gene editing. To date, the most efficient way to achieve large cargo integration is by using lentiviruses. However, lentiviral-mediated integration lacks the targetability feature, as integration occurs mostly randomly in open chromatin. The use of reverse transcriptases (RTs) with high processivity and high fidelity in conjunction with Cas nickases may be a viable rout to achieve large cargo integration. The Cas nickase provides targetability, whereas the RT, via a target-primed reverse transcription mechanism, integrates the large RNA cargo into mammalian gDNA. Overall, these RNA-templated Reverse Transcriptase systems composed of a Cas nickase and a highly active and processive reverse transcriptase (RT) may facilitate integration of large DNA sequences into therapeutic genomic sites of interest. To be successful, RTs must be identified that are able to synthesize cDNA with high fidelity.

The fidelity of RTs was evaluated by NGS of cDNA products generated by primer extension on a standard and modified RNA template of 202 nt in length (SEQ ID NO: 55). The standard RNA was prepared using standard in vitro transcription conditions, while the modified RNA template was prepared with 100% replacement of uridine with N1-methyl pseudouridine (m1Ψ). The primer extension reaction contained an RT enzyme derived from a cell-free expression system. Expression constructs (SEQ ID NOs: 70, 83, 85, 113, and 115) were codon-optimized for E. coli and contained an N-terminal single Strep tag. The substrate for the reaction was 100 nM of RNA template annealed to a 5′-FAM labeled primer (SEQ ID NO: 56). The reaction buffer contained the following components: 50 mM Tris-HCl (pH 8.0), 75 mM KCl, 3 mM MgCl₂, 10 mM DTT, and 0.5 mM dNTPs. Following incubation at 37° C. for 1 h, the reaction was quenched via the addition of RNAse A. The resulting cDNA products were then cleaned up via SPRI beads and ligated on the 3′ end using an adapter oligo containing a 14-nt unique molecular identifier (UMI, SEQ ID NO: 1595). The background control sample was generated by performing a 5-cycle PCR with Q5 polymerase using a plasmid template, reverse primer (SEQ ID NO: 1596), and forward primer encoding a 14-nt UMI SEQ ID NO: 1597). Ligated cDNAs or PCR products (for background samples) were then diluted to the same concentration across samples prior to performing subsequent PCR reactions with primers for Illumina library preparation (SEQ ID NO: 1598 forward primer for RT samples, SEQ ID NO: 1599 forward primer for background samples, SEQ ID NO 57 reverse primer). PCR triplicate samples were sequenced in paired-end mode for 150 cycles with a read depth of 25M. Sequencing reads were then sorted by UMI barcode, and reads that contained identical UMIs were grouped as unique molecules. Only UMI groups that contained at least 5 reads were used in downstream analysis. A consensus sequence was then generated from the reads within an UMI group. If less than 60% of the reads agreed on the identity of any individual base, the consensus was discarded. Errors in consensus sequences passing this threshold were tabulated by aligning to the expected sequence. An error rate was calculated across all consensus sequences as the frequency of base substitutions relative to the expected sequence. Other measures of RT error calculated include frequencies of other RT error types (substitution, insertion, deletion) at each position along the RNA template, base substitution preference, indel size distribution, and base incorporation preference of non-templated addition events.

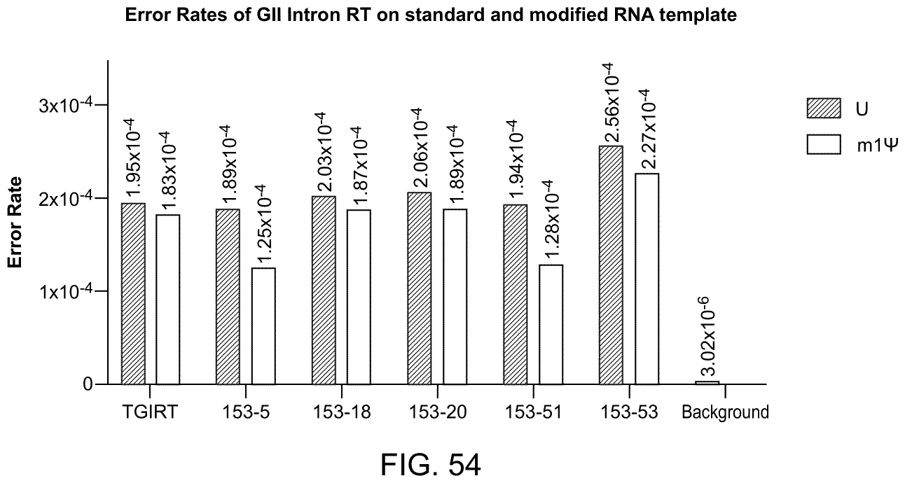

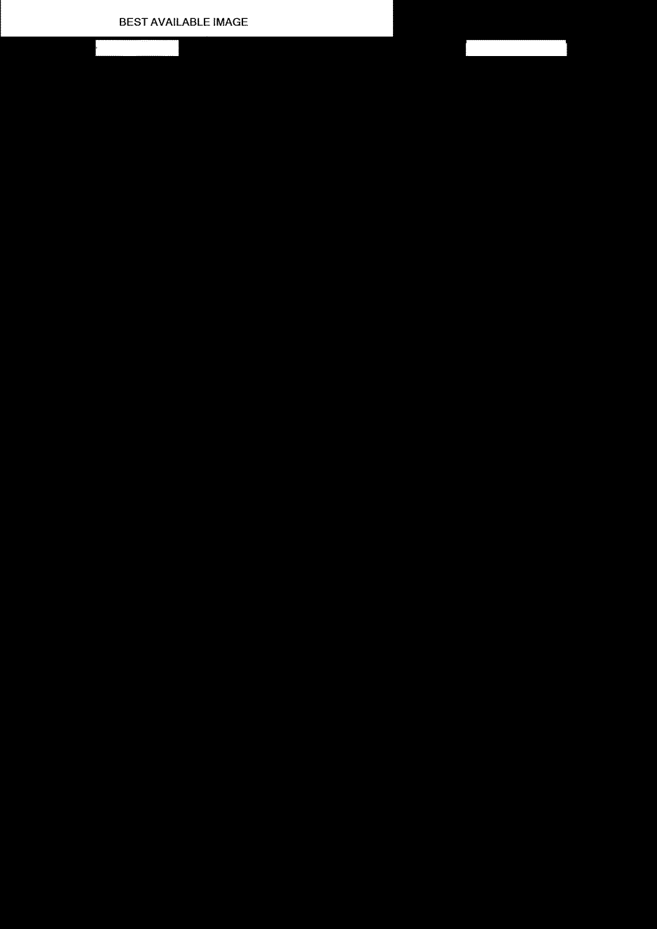

Error rate analysis (FIG. 54) revealed that the positive control GII intron RT TGIRT has an error rate similar to that which has been previously determined also using an UMI-based NGS method (Zhao et al., 2018). GII intron RTs have similarly low substitution rates to that of TGIRT on the standard RNA template (U). Importantly, since the mRNA delivery platform for this system may require RNA templates to be modified, RTs retain high fidelity on the modified (m1Ψ) RNA template. Of note, the data presented are representative of two independent experiments, each of which were sequenced in PCR triplicate, and the data is reproducible.

### Example 33—cDNA Synthesis by Group II Intron RTs, Non-LTR Retrotransposon RTs, and Retron RTs

Group II introns and non-LTR retrotransposases are capable of integrating large cargo into a target site via reverse transcription of an RNA template. These reverse transcriptases (RTs) integrate an RNA template via target primed reverse transcription (TPRT), a mechanism in which cDNA synthesis is primed by the free 3′ hydroxyl group at the target DNA nick. These enzymes are predicted to be active based on the presence of expected RT catalytic residues [F/Y]XDD. Another family of RTs that can produce DNA from RNA for gene editing are retrons. These are compact retroelements that have specific sites of initiation and termination of reverse transcription that make them compelling tools for biotechnology applications (Lopez et al., 2022).

Results: Human Cells cDNA Synthesis by RTs

The ability of RTs to produce cDNA in a mammalian environment is tested by expressing them in mammalian cells and detecting cDNA synthesis by qPCR. Reverse transcriptases are cloned in a plasmid for mammalian expression under the CMV promoter as fusion proteins having MS2 coat protein (MCP) at the N terminus, in addition to a flag-HA tag (FH). MCP is a protein derived from the MS2 bacteriophage that recognizes a 20 nucleotide RNA stem loop with high affinity-subnanomolar Kd. By fusing the RTs with MCP and having the MS2 loops in the RNA template, once the RT is translated it finds the RNA template and starts cDNA synthesis from the DNA primer hybridized to the RNA template was ensured.

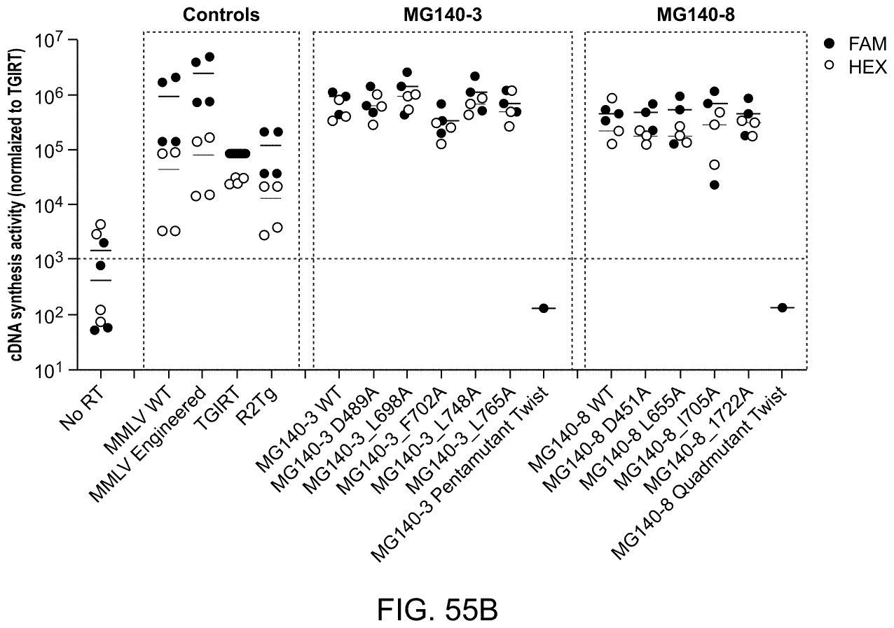

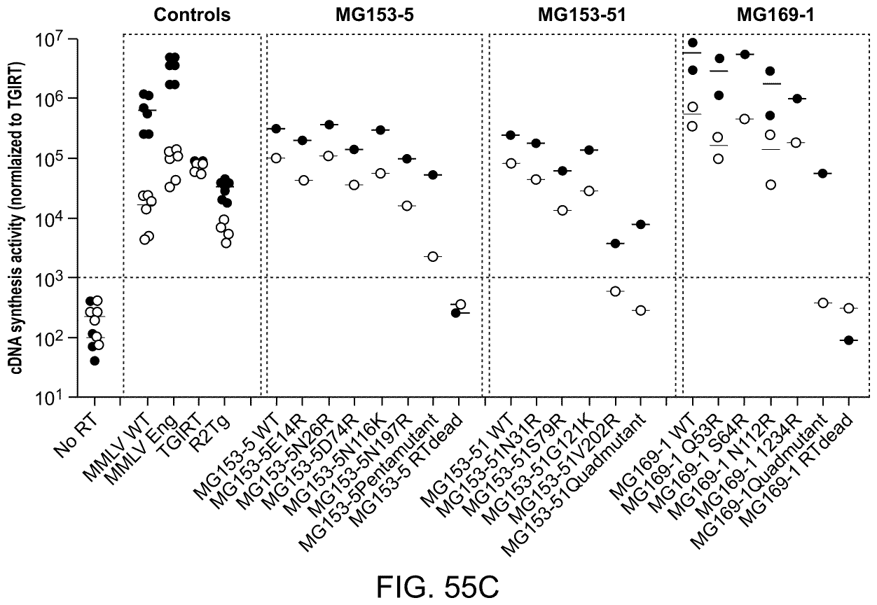

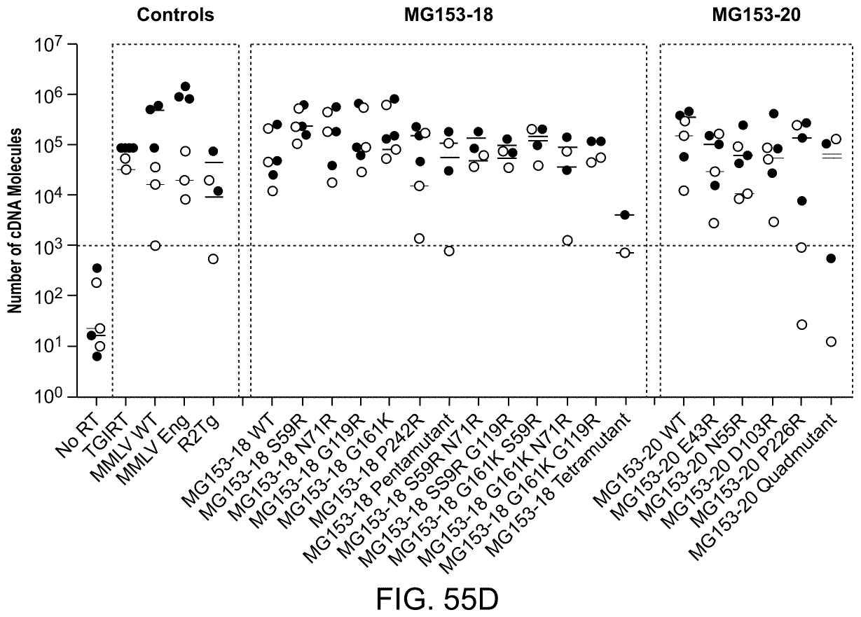

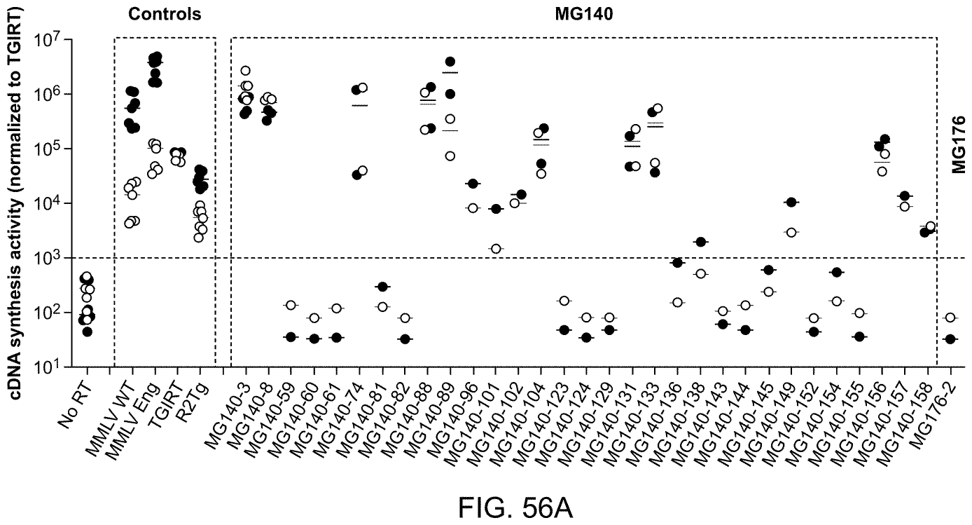

A plasmid containing MCP fused to the RT candidate under CMV promoter is cloned and isolated for transfection in HEK293T cells. Transfection is performed using lipofectamine 2000. mRNA (SEQ ID NO: 1600) encoding dCas9 fused to nanoluciferase is made. To degrade any DNA template left in the mRNA preparation the reaction is treated with DNase for 1.5 hours and the mRNA is cleaned up. The mRNA is hybridized to a complementary DNA primer (SEQ ID NO: 1601) in 10 mM Tris pH 7.5, 50 mM NaCl at 95° C. for 2 min and cooled to 4° C. at the rate of 0.1° C./s. The mRNA/DNA hybrid is transfected into HEK293T cells 6 hours after the plasmid containing the MCP-RT fusion was transfected. 18 hours post mRNA/DNA transfection, cells are lysed. 100 μL of quick extract is added per well in a 24 well plate. The RNA template is ~4247 nt. Primers to amplify first and last 100 bp products from the newly synthesized cDNA (4100 bp) were designed (SEQ ID NOs: 1601-1604), along with taqman probes (SEQ ID NOs: 1605-1606) to quantify their amplification (FIG. 55A).

Activity for the control retroviral MMLV (penta-mutant, SEQ ID NO: 1607 and WT, SEQ ID NO: 1608), control GII intron RT TGIRT (SEQ ID NO: 1609), as well as a positive control for R2 RTs, R2Tg (SEQ ID NO: 1610), was detected (FIGS. 55B-55D), as shown by an early amplification of the first and last 100 bp products. As expected for a low processivity RT, the retroviral RTs (MML Vs) show high amplification levels of the first 100 bps (FAM signal) but the levels at which they complete cDNA synthesis (the last 100 bps) is lower (20-fold lower than first 100 bp, as observed by the FAM/HEX ratio signal). Control group II intron RT TGIRT and control R2 non-LTR retrotransposon RT R2Tg show a closer FAM/HEX ratio, demonstrating their high processivity (FIGS. 55B-55D). Two R2 non-LTR retrotransposon RTs MG140-3 (SEQ ID NO: 1611) and MG140-8 (SEQ ID NO: 1618) as well as five GII intron RTs MG153-5 (SEQ ID NO: 1624), MG153-51 (SEQ ID NO: 1632), MG169-1 (SEQ ID NO: 1638), MG153-18 (SEQ ID NO: 1645), and MG153-20 (SEQ ID NO: 1657) were previously selected candidates owing to their high activity and processivity. Rationally engineered mutants were screened for all candidates using the above-mentioned cDNA synthesis assay in mammalian cells. Six engineered variants of MG140-3 and five engineered variants of MG140-8, belonging to the MG140 family of non-LTR retrotransposon RTs were tested in mammalian cells (FIG. 55B). All tested MG140-3 (SEQ ID NOs: 1612-1616) and MG140-8 single mutants (SEQ ID NOs: 1619-1622) showed comparable activity to the WT (MG140-3 WT and MG140-8 WT, SEQ ID NOs: 1611 and 1618) whereas the combination of all mutations (MG140-3 pentamutant and MG140-8 quadmutant, SEQ ID NOs: 1617 and 1623) was inactive based on low FAM and HEX fluorescence levels. Similarly, six engineered variants of MG153-5 were tested of which the single mutants (SEQ ID NOs: 1625-1629) had comparable activity to the WT (SEQ ID NO: 1624) while the combined pentamutant (SEQ ID NO: 1630) had low processivity (FIG. 55C). RT-domain inactivating mutant for MG153-5, MG153-5 RTdead (SEQ ID NO: 1631), exhibited activity below background (indicated by a dashed horizontal line) demonstrating its use as a negative control for comparison of RT activity (FIG. 55C). Five engineered variants of MG153-51 were tested of which three single mutants MG153-51 N31R (SEQ ID NO: 1633), MG153-51 S79R (SEQ ID NO: 1634), and MG153-51 G121K (SEQ ID NO: 1635) showed comparable activity to the WT (SEQ ID NO: 1632) whereas MG153-51 V202R (SEQ ID NO: 1636) and MG153-51 combined quadmutant (SEQ ID NO: 1637) showed low processivity (FIG. 55C). Likewise, five engineered variants of MG169-1 were tested where all single mutants (SEQ ID NOs: 1639-1642) had comparable activity to the WT (SEQ ID NO: 1638) while the combined quadmutant (SEQ ID NO: 1643) had low processivity (FIG. 55C). RT-domain inactivating mutant for MG169-1, MG169-1 RTdead (SEQ ID NO: 1644), exhibited activity below background (indicated by a dashed horizontal line) demonstrating its use as a negative control for comparison of RT activity (FIG. 55C). Additionally, five single mutants (SEQ ID NOs: 1646-1650) and six combinatorial mutants of MG153-18 (SEQ ID NOs: 1651-1656) were tested. Of note, the tetramutant (MG153-18 N71R S59R G119R G161K, SEQ ID NO: 1656) showed lower activity and processivity compared to the WT (SEQ ID NO: 1645). All other mutants showed comparable activity to the WT with MG153-18 S59R (SEQ ID NO: 1646), MG153-18 N71R (SEQ ID NO: 1647), MG153-18 G119R (SEQ ID NO: 1648), and MG153-18 G161K (SEQ ID NO: 1649) showing marginally higher activities on average than their WT counterpart (FIG. 55D). Five mutants of MG153-20 (SEQ ID NOs: 1658-1662) were also tested, and all five mutants showed comparable activity to the WT (SEQ ID NO: 1657) although more variable activity was noted for MG153-20 P226R (SEQ ID NO: 1661) and the combined quadmutant (SEQ ID NO: 1662) (FIG. 55D).

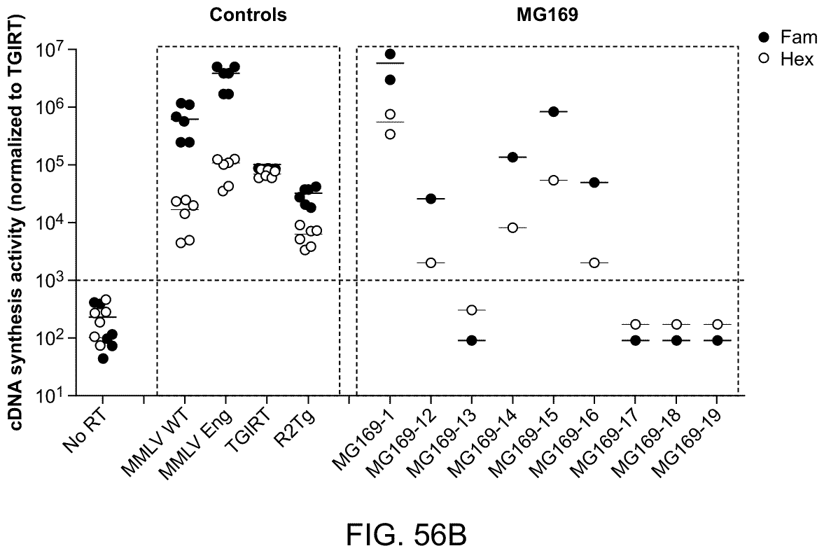

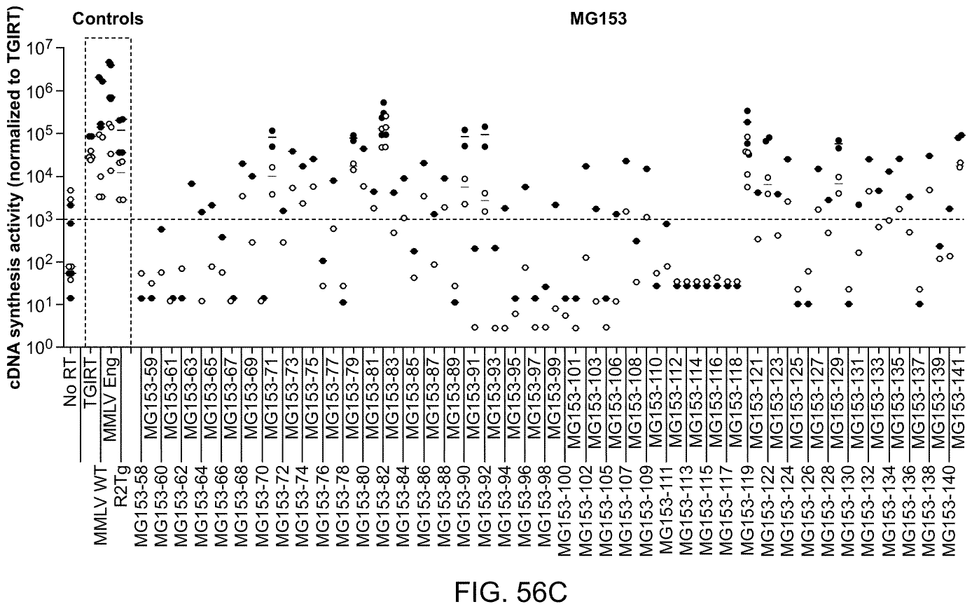

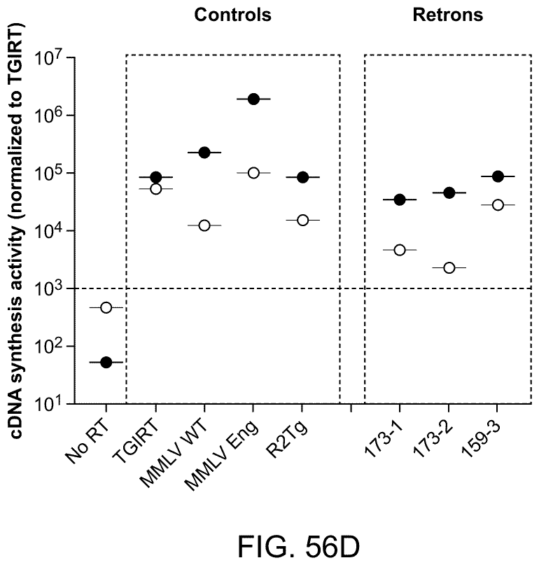

Owing to the identification of several active and processive RT candidates from the GII intron family and non-LTR retrotransposon family of RTs, more RT candidates of each of these families were screened through the mammalian cDNA synthesis assay (FIG. 56). Twenty-nine MG140 family RTs (MG140-59 to -61, -74, -81, -82, -88, -89, -96, -101, -102, -104, -123, -124, -129, -131, -133, -136, -138, -143 through -145, -149, -152, -154 through -158, SEQ ID NOs: 1663-1691) and one RT belonging to the MG176 family, MG176-2 (SEQ ID NO: 1692), were screened. The tested candidates showed a wide range of RT activity in mammalian cells. Candidates with high cDNA synthesis efficiency include MG140-74 (SEQ ID NO: 1666), MG140-88 (SEQ ID NO: 1669), MG140-89 (SEQ ID NO: 1670), MG140-104 (SEQ ID NO: 1674), MG140-131 (SEQ ID NO: 1678), MG140-133 (SEQ ID NO: 1679), and MG140-156 (SEQ ID NO: 1689) (FIG. 56A). Of these, MG140-74 and MG140-88 showed high processivity as evidenced by similar FAM and HEX fluorescence values making them promising candidates for further testing. Eight candidates of the MG169 family (MG169-12 through-19, SEQ ID NOs: 1693-1700) were tested and their activities and processivities were compared to MG169-1, the most active candidate identified from this family (FIG. 56B). However, no candidates of comparable or higher activity than MG169-1 family were identified (FIG. 56B). Along the same lines, eighty-two RT candidates of the MG153 family of GII intron derived RTs were tested (MG153-58 through -103, -105 through -119, -121 through -141, SEQ ID NO: 1701-1782) of which 1 candidate, MG153-82 (SEQ ID NO: 1725), with high cDNA synthesis activity and processivity was identified (FIG. 56C). Additionally, three candidates of the retron family of RTs were tested of which one candidate, MG159-3 (SEQ ID NO: 1785), with comparable activity and processivity to TGIRT was identified while the other two retron candidates, MG173-1 (SEQ ID NO: 1783), and MG173-2 (SEQ ID NO: 1784), had lower processivity (FIG. 56D).

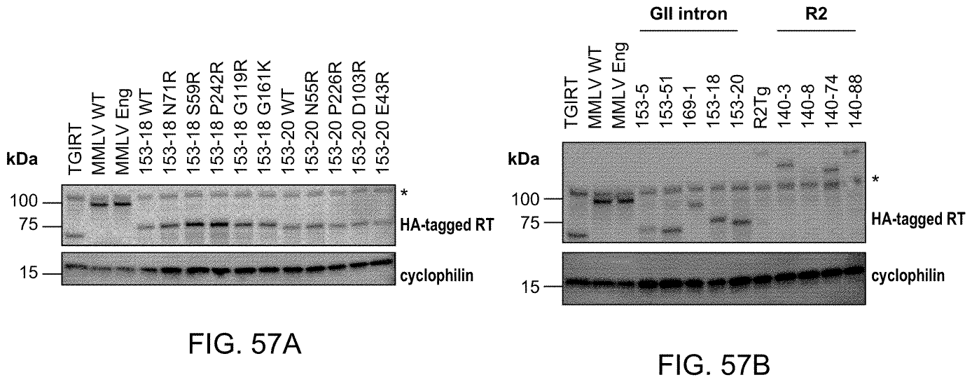

Due to the slight increase in activity noted for four of five MG153-18 single mutant variants, their protein expression was compared against that of MG153-18 WT as well as MG153-20 WT and single mutants (FIG. 57A). No differences in protein expression were observed between MG153-18 WT, the more active MG153-18 single mutants and between MG153-20 WT and mutants suggesting that differences in cDNA synthesis activity are not a consequence of variation in protein expression of the RT candidates. Additionally, protein expression of promising RT candidates with high cDNA synthesis activity and processivity was tested (FIG. 57B). Protein expression of GII intron candidates, except for MG153-5, was observed to be stronger than that of the R2 family of non-LTR retrotransposons. Within the R2 candidates, the expression of MG140-3, MG140-74, and MG140-88 was observed to be stronger than that of MG140-8 (FIG. 57B).

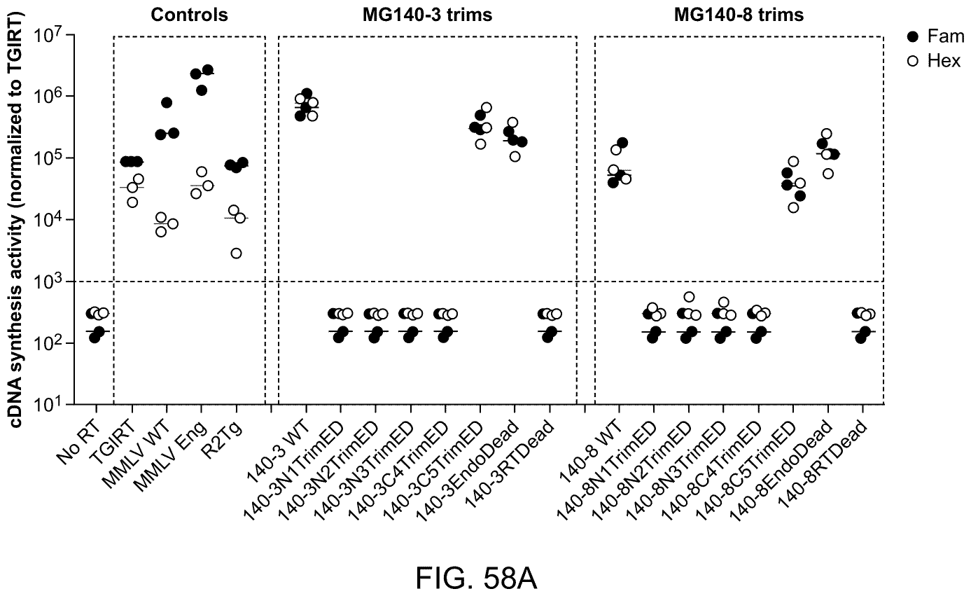

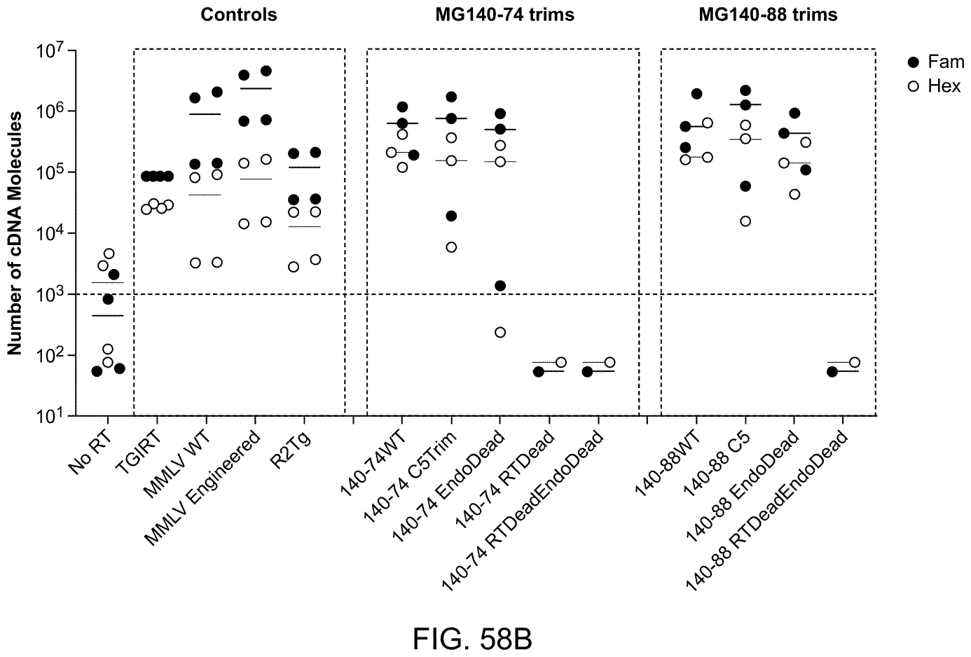

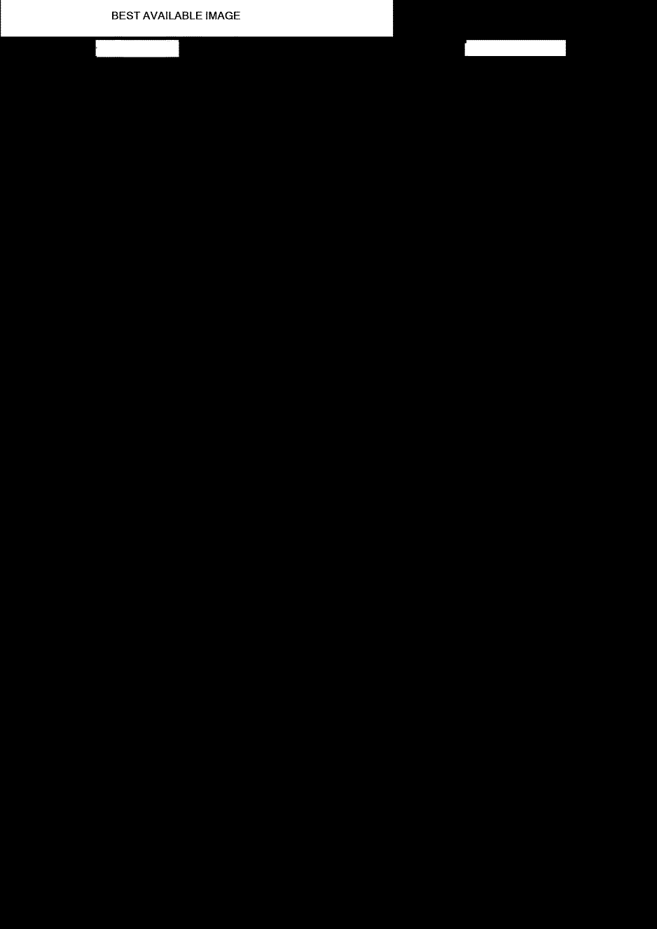

In comparison to the GII intron RTs which are about 450 amino acids in length, the MG140 family of non-LTR retrotransposon RTs are much bigger in size at about 1200 amino acids in length on average. Shorter, trimmed variants of previously identified MG140 candidates MG140-3, MG140-3, MG140-74, and MG140-88 with high activity and processivity were assessed. Five trimmed variants of MG140-3 (SEQ ID NOs: 1786-1790) and MG140-8 (SEQ ID NO: 1793-1797) were tested, alongside their endonuclease domain inactivated (SEQ ID NOs: 1791 and 1798, respectively) and RT domain inactivated mutants (SEQ ID NO: 1792 and 1799). The C5 trims of MG140-3 (SEQ ID NO: 1789) and MG140-8 (SEQ ID NO: 1796), wherein about 200 amino acids were trimmed off the C-terminus, including the catalytic residue of the endonuclease domain D994 and D938 respectively, retained activity comparable to the WT and endonuclease domain inactivated variants. The other four trims-three N-terminus trims N1, N2, and N3 and one C-terminus trim, C4-were as inactive as the RT domain inactivated variant (FIG. 58A). This prompted the testing of exclusively the C5 trim of MG140-74 and MG140-88 (SEQ ID NOs: 1800 and 1804), which was also found to be as active as their respective WT and endonuclease domain (SEQ ID NOs: 1801 and 1805) inactivated counterparts as opposed to the inactive RT domain inactivated mutants (SEQ ID NOs: 1802, 1803, and 1806) (FIG. 58B).

### Results: CDNA Synthesis In Vitro by Retron RTs on a Generic, Short RNA Template

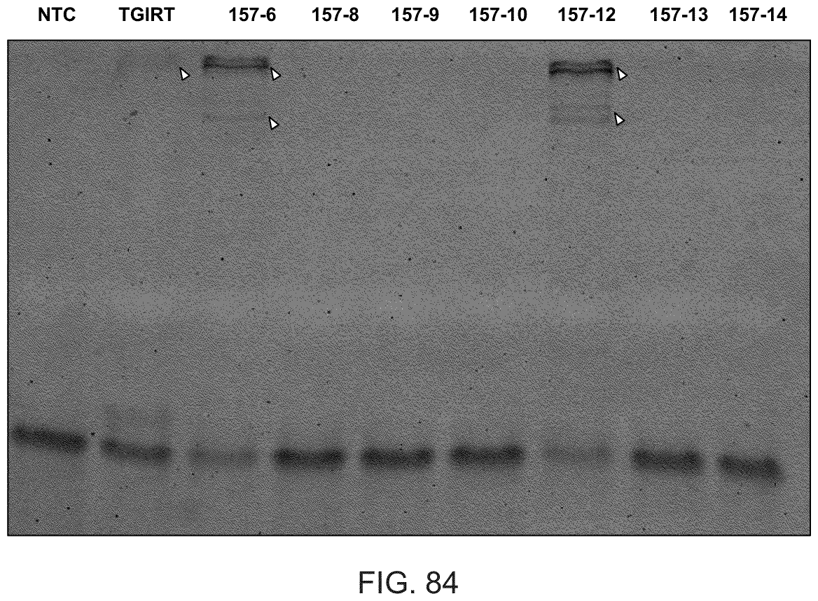

The in vitro activity of newly identified retron RTs (SEQ ID NOs: 2258-2266) on a general RNA template was assessed by a primer extension reaction containing RT enzyme derived from a cell-free expression system. Expression constructs were codon-optimized for E. coli with an N-terminal single Strep tag, and expression reactions were added to a primer extension reaction with 100 nM of substrate RNA template (202 nt) annealed to a 5′-FAM labeled primer. Following incubation, the reaction was quenched, the resulting cDNA product(s) were separated on a 10% denaturing polyacrylamide gel and visualized. Active retron RTs capable of performing primer extension on a generic RNA template that is not their specific ncRNA were MG157-6 and MG157-12 (FIG. 84). Given that retron RTs are predicted to be active based on the presence of key catalytic residues, results do not rule out the possibility that these RTs are active on a different RNA template.

### Results: CDNA Synthesis by Group II Intron and Rationally Engineered R2 Retrotransposon RTs in Human Cells

The ability of RTs to produce cDNA in a mammalian environment was tested by expressing them in mammalian cells and detecting cDNA synthesis by qPCR. Reverse transcriptases were cloned in a plasmid for mammalian expression under the CMV promoter as fusion proteins having MS2 coat protein (MCP) at the N terminus, in addition to a flag-HA tag (FH). A plasmid containing MCP-RT fusion candidate was cloned and isolated for transfection in HEK293T cells. mRNA encoding a cargo fused to nanoluciferase was made, the reaction was treated with DNase for 1.5 hours and the mRNA was cleaned up. The mRNA was hybridized to a complementary DNA primer and the mRNA/DNA hybrid was transfected into HEK293T cells 6 hours after the plasmid containing the MCP-RT fusion was transfected. After transfection, cells were lysed and 100 μl of the quick extract is added per well in a 24 well plate for cDNA synthesis evaluation. Primers amplify the first and last 100 bp products from the newly synthesized full-length cDNA (4100 bp in length).

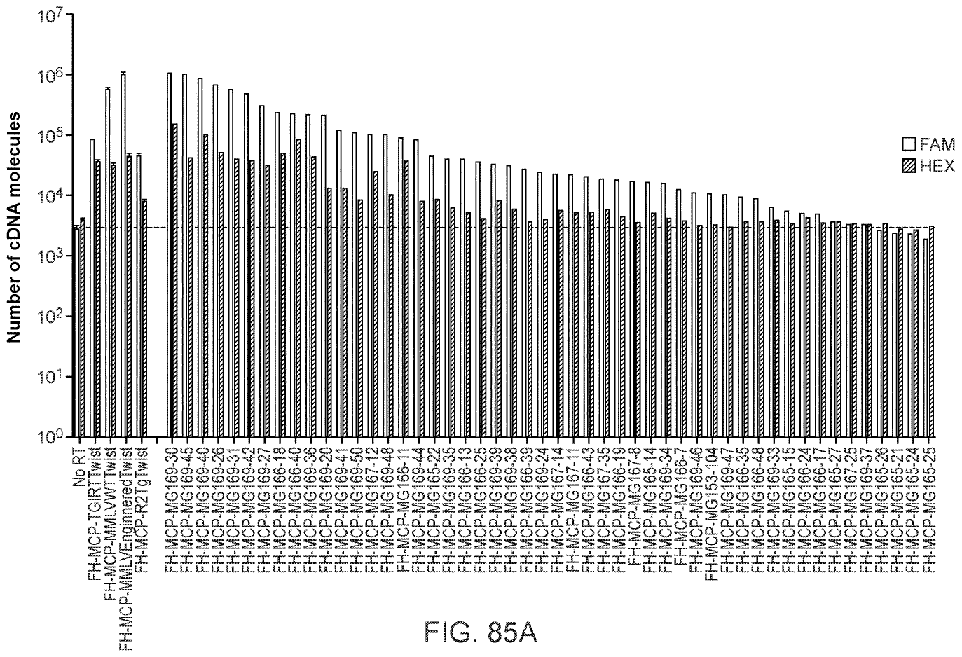

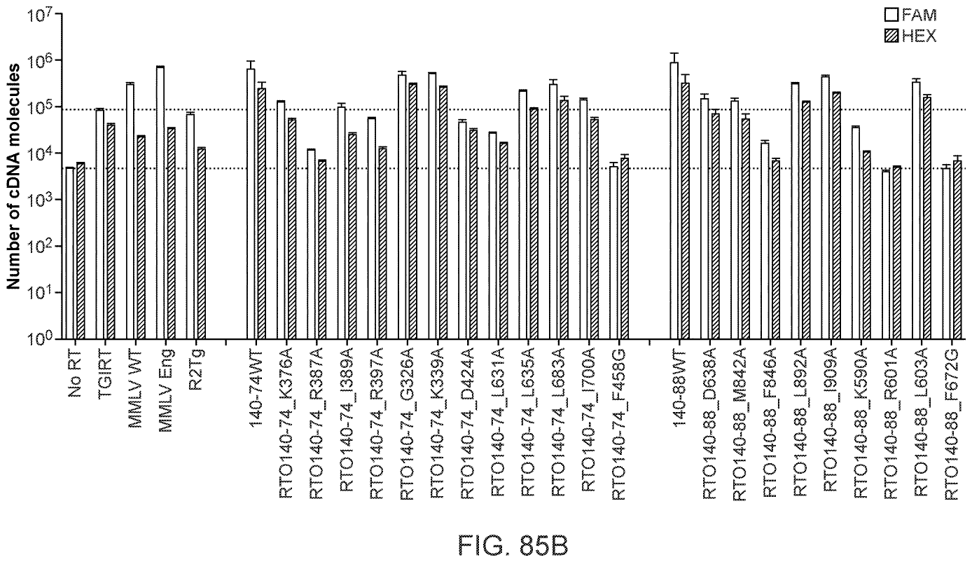

cDNA synthesis activity in HEK293T cells was confirmed for many group II intron RTs (FIG. 85A). In addition, several engineered variants of non-LTR retrotransposon RTs MG140-74 and MG140-88 showed comparable cDNA synthesis activity and processivity to their WT counterparts (FIG. 85B). Results suggest that selected mutations do not alter the mechanistic properties of these RTs when tested in human cells.

### Example 34—Fidelity of cDNA Synthesis of Group II Intron RTs

### Substitution Error Rate Analysis

The fidelity of RTs was evaluated by NGS of cDNA products using either a standard or modified RNA template as described previously. The standard RNA was prepared using an in vitro transcription reaction containing an equimolar mixture of ATP, UTP, GTP, and CTP while the modified RNA template was prepared with 100% replacement of UTP with m1TP (N1-methyl-pseudouridine-5′-triphosphate). Improvements in the quality of the RNA template (SEQ ID NO: 55) were made to improve assay sensitivity and the RT substitution error rates were re-measured. Of note, the fidelity data presented are representative of two independent experiments, each of which were library prepped and sequenced in triplicate, and the data is reproducible. Control RT 1 is a retroviral RT MMLV, Control RT 2 is the GII intron RT TGIRT, and Control RT 3 is the GII intron RT MarathonRT. Analysis of substitution error rate (FIG. 59A) reveals that both positive control GII intron RTs TGIRT (Control 2) and MarathonRT (Control 3) have error rates similar to those that have been published previously which also used an UMI-based NGS method. MMLV (Control 1) has a higher error rate on the standard template compared to GII intron RTs, however is significantly more accurate on the modified (m1Ψ) RNA template. GII intron RTs (MG153-5, MG153-18, MG153-20, MG153-51; SEQ ID NOs: 70, 83, 85, and 113) have similarly low substitution error rates to that of Control 2 and Control 3 on both the standard (U) and modified (m1Ψ) RNA templates.

Analysis of the inverse of the substitution error rate indicates the theoretical length of substitution error-free cDNA that could be synthesized by the RT. Based on these results, MG153-5 has a similar cDNA synthesis accuracy to that of the positive control enzyme MarathonRT, generating a theoretical error-free cDNA molecule of ~8,000 nucleotides in length (FIG. 59B). MG153-5 outperforms the positive control enzyme TGIRT and the other tested GII intron RTs by this fidelity metric.

### Error Type by Position

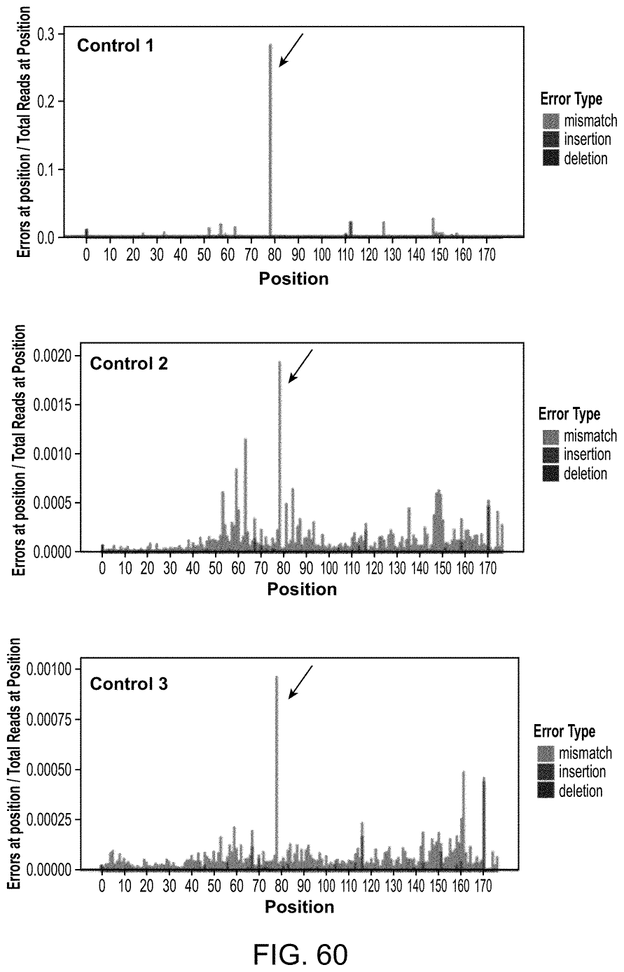

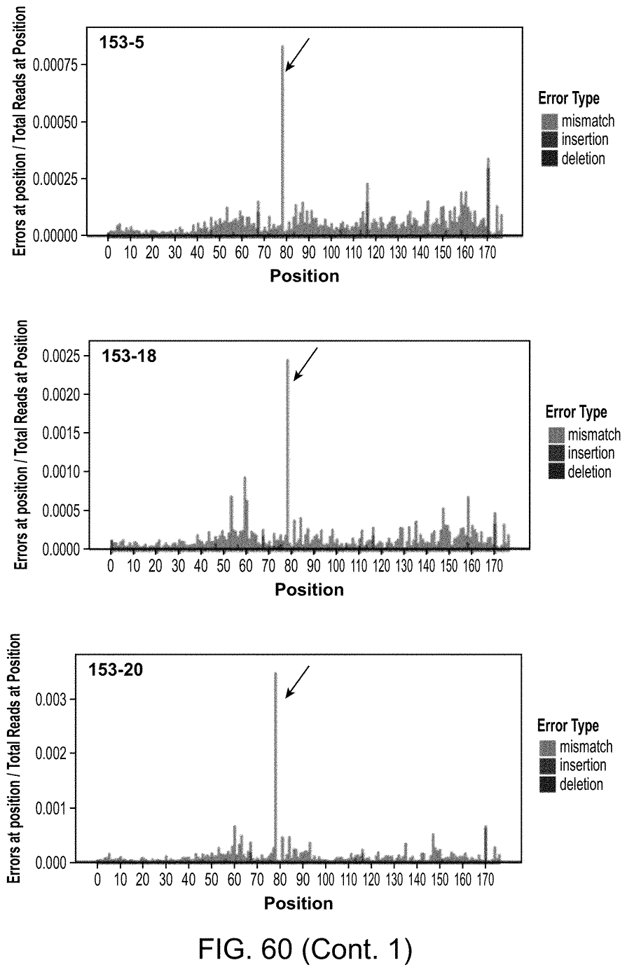

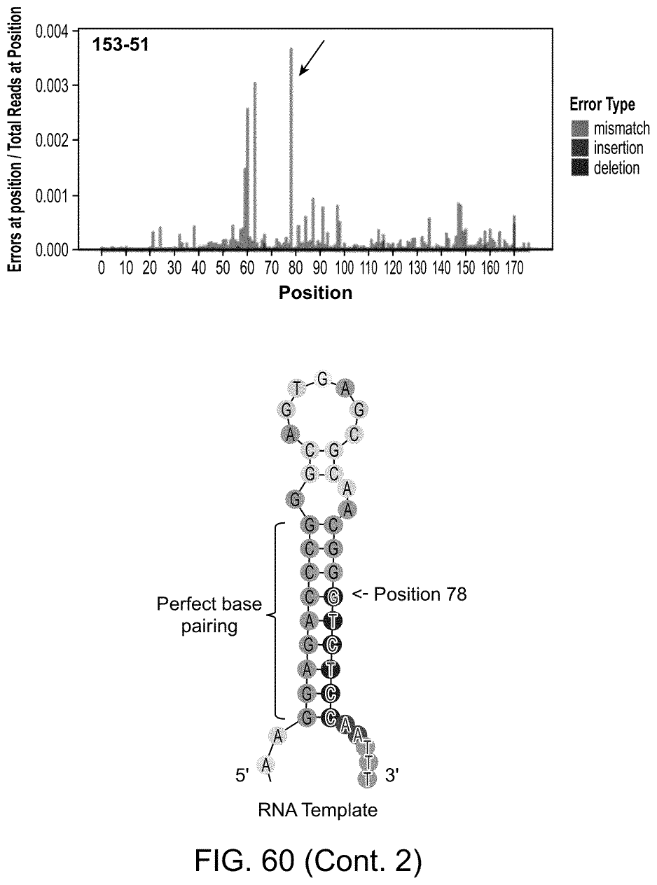

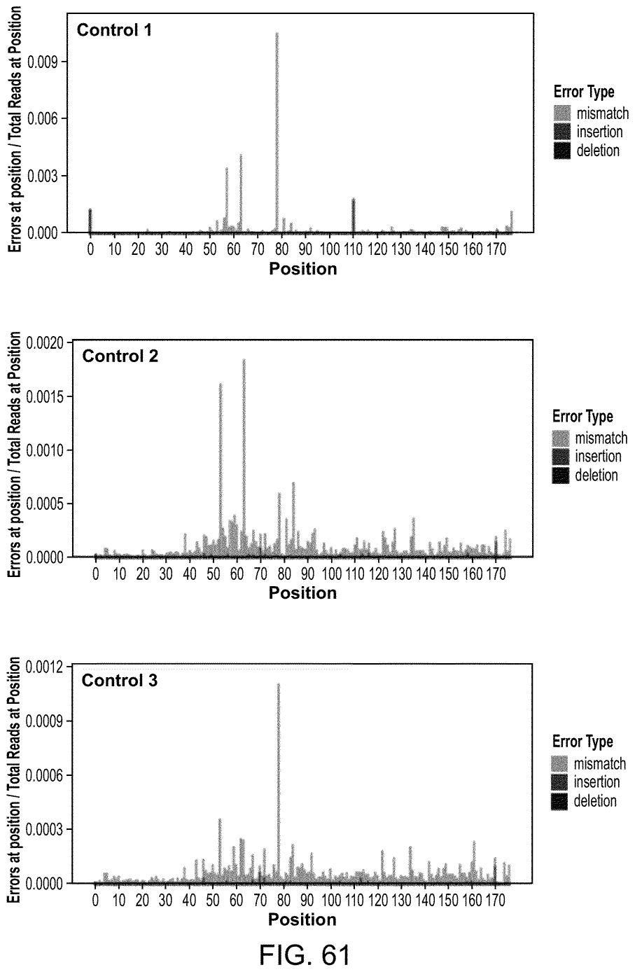

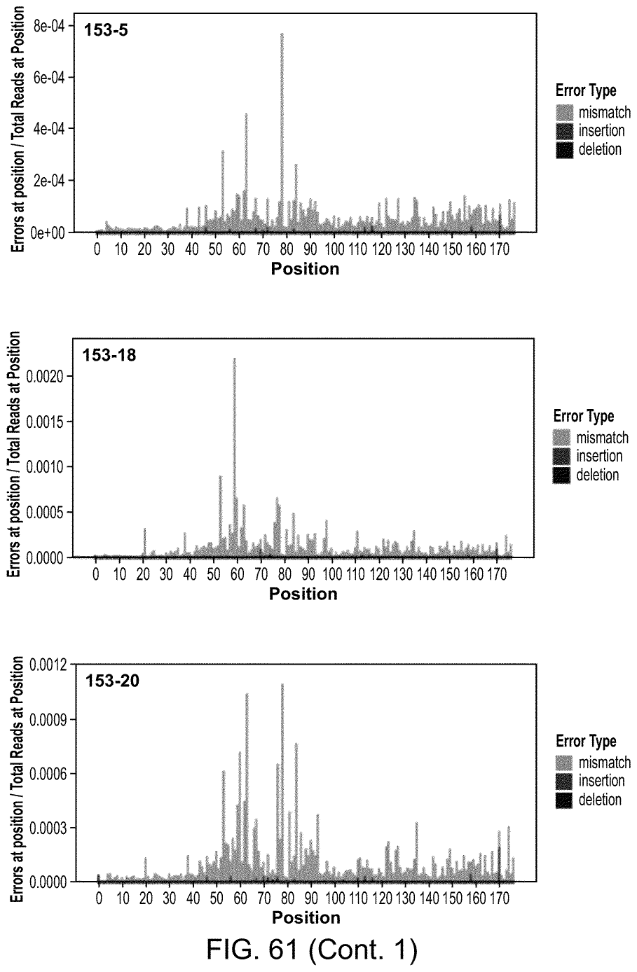

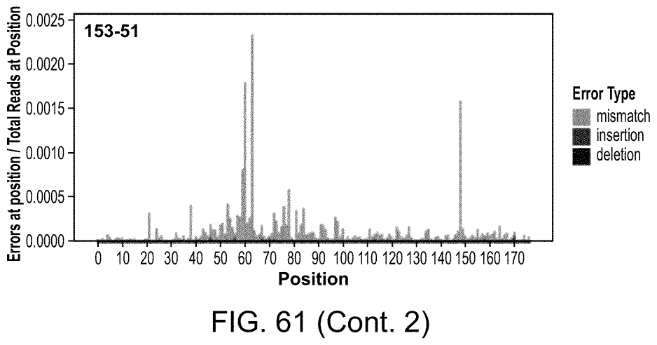

Analysis of error type by position along the RNA template was calculated by dividing the count of error at the position (substitution, insertion, or deletion) by the total consensus cDNA sequences aligned at the position. The analysis reveals that for all tested RTs, substitution (referred to in the figure as a mismatch), as opposed to insertion or deletion, dominates the error profile on both standard and modified templates (FIGS. 60-61). All tested RTs have a propensity to misincorporate at certain positions along the RNA template. A notable substitution hotspot identified on the standard template located at position 78 corresponds to a nucleotide mid-way through a putative hairpin structure innate to the RNA template (FIG. 60, denoted by arrows).

### Substitution Preference

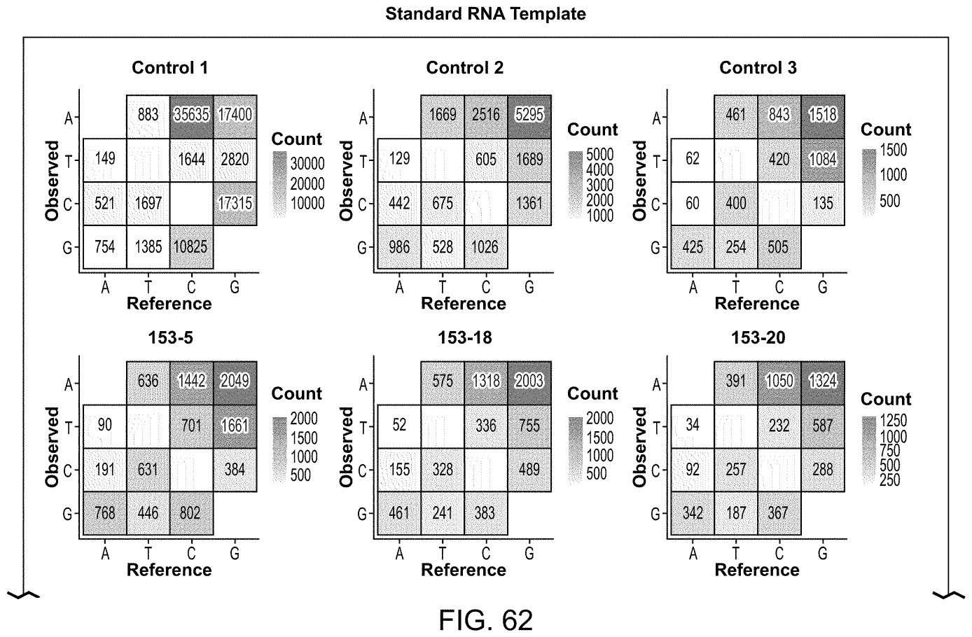

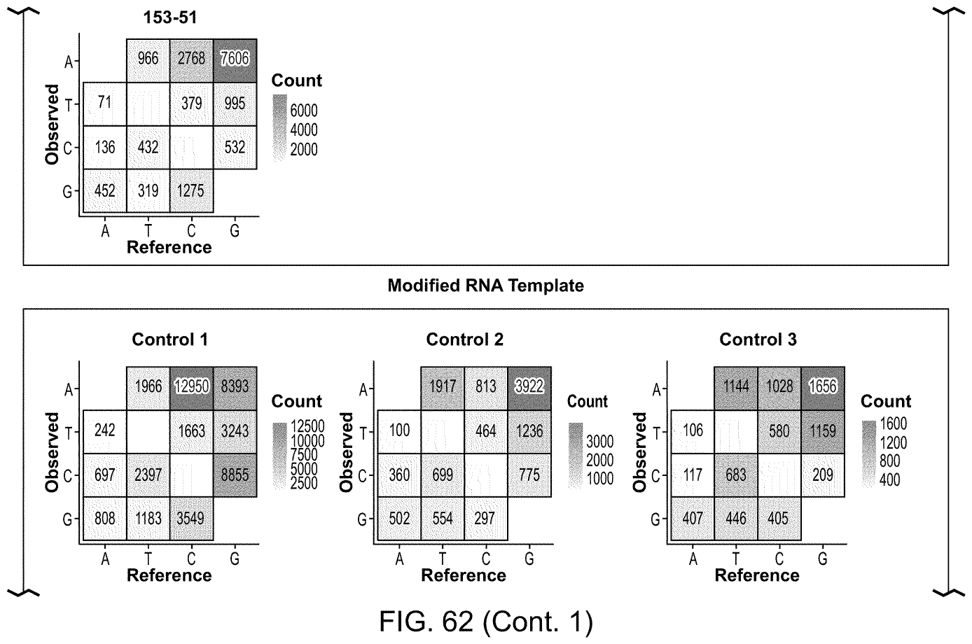

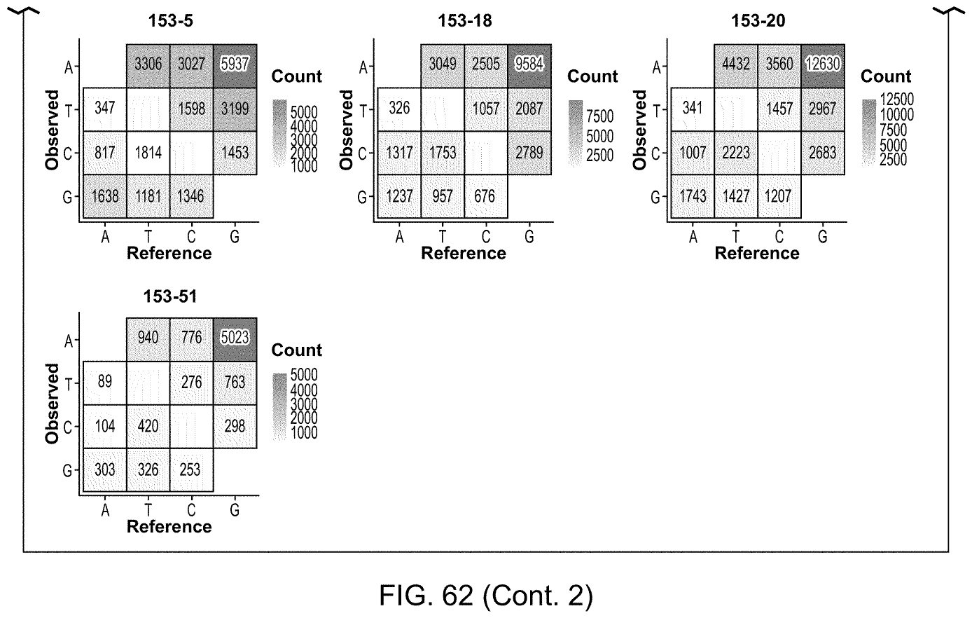

For every substitution error, the nucleotide misincorporated (observed) was compared to the reference nucleotide and tabulated. Counts are displayed as a confusion matrix (FIG. 62). Based on these results, all tested GII intron RTs (including Control 2 and Control 3) tend to misincorporate an A where a G should have been incorporated whereas retroviral RT MMLV (Control 1) has a tendency to misincorporate an A where a C should have been incorporated. Substitution preference is similar between the standard and modified RNA template for all tested RTs, indicating that misincorporation did not occur at a disproportionately higher frequency at modified m1Ψ sites on the RNA template.

The distinct substitution preference between the retroviral control and GII intron RTs can, in part, be explained by the substitution hotspot at position 78 (FIGS. 60-61). At position 78 on the RNA template, the RT should incorporate a C and MMLV (Control 1) exhibits the strongest C->A substitution hotspot at this position.

### Insertion and Deletion Analysis

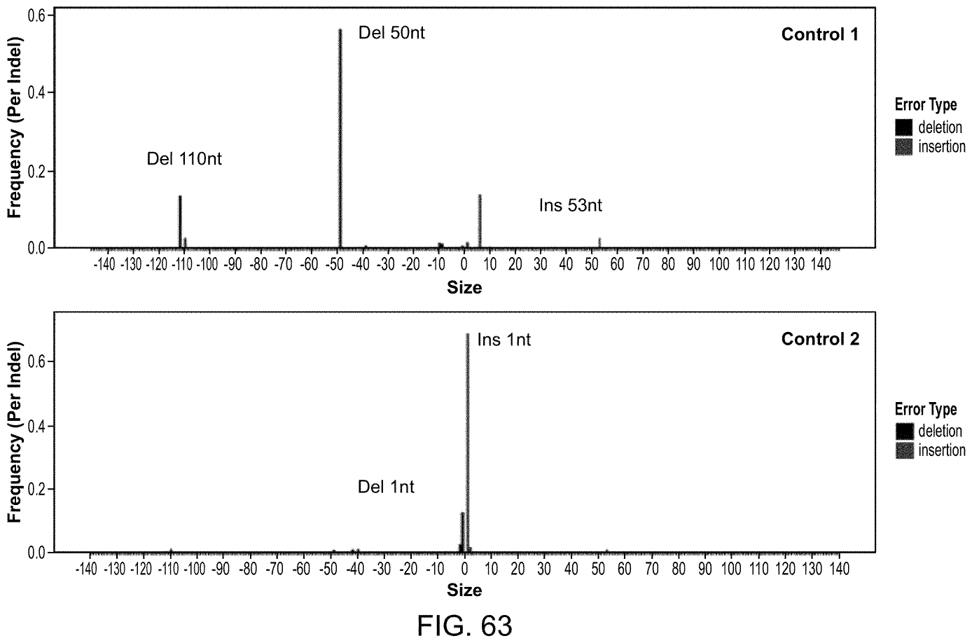

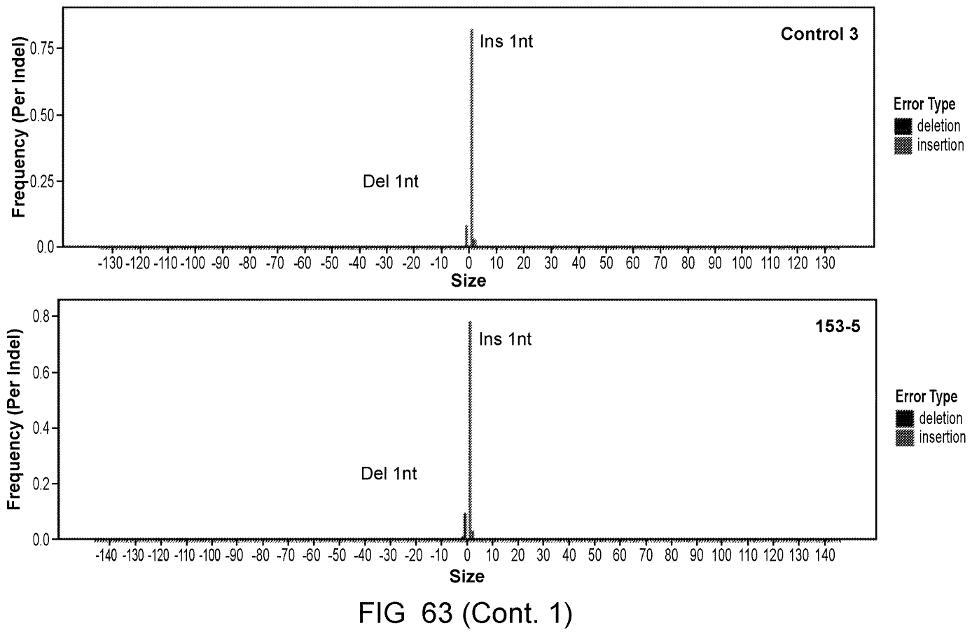

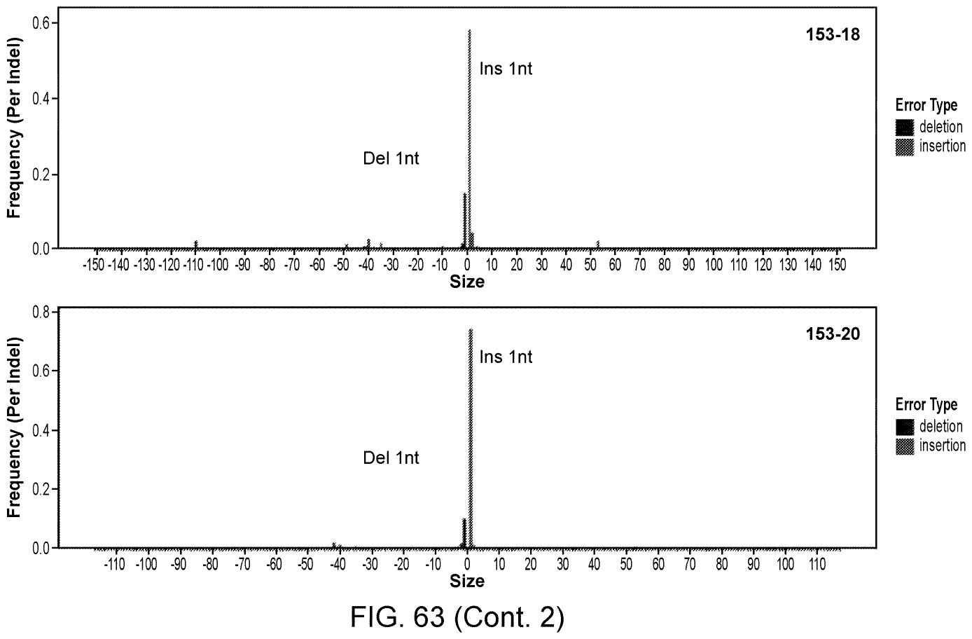

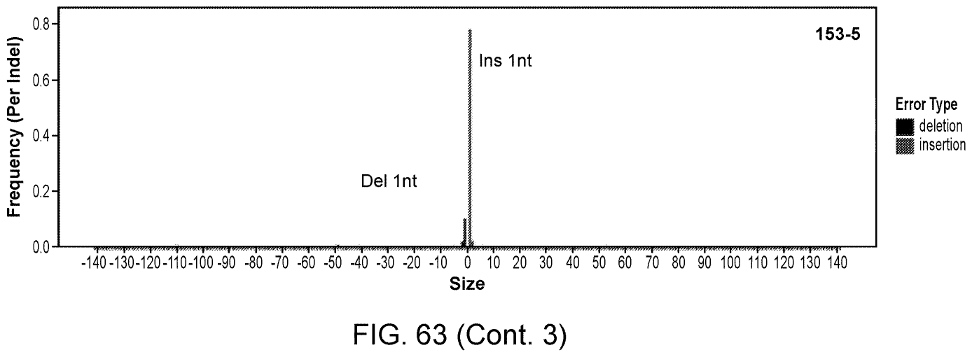

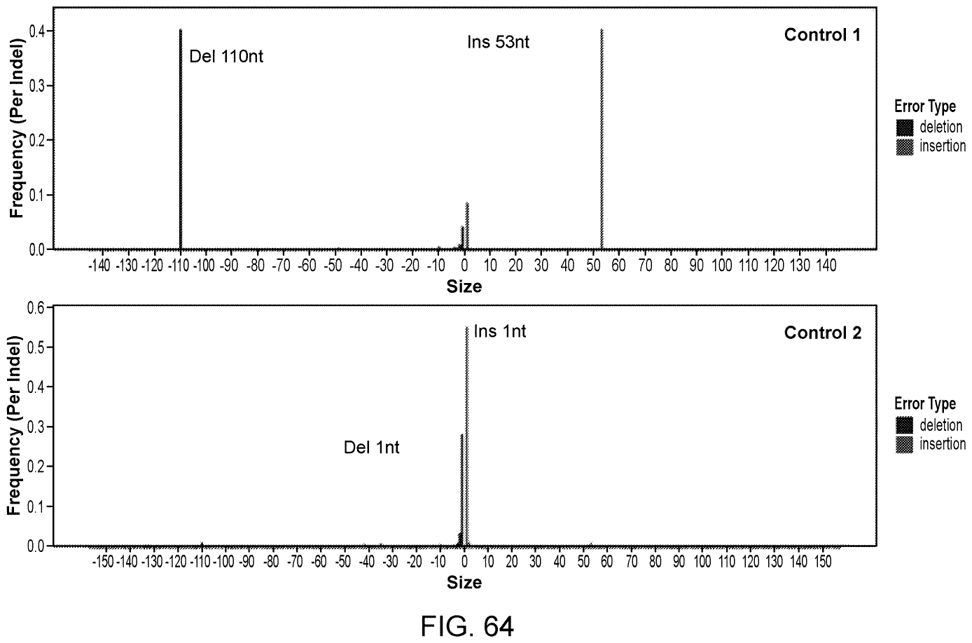

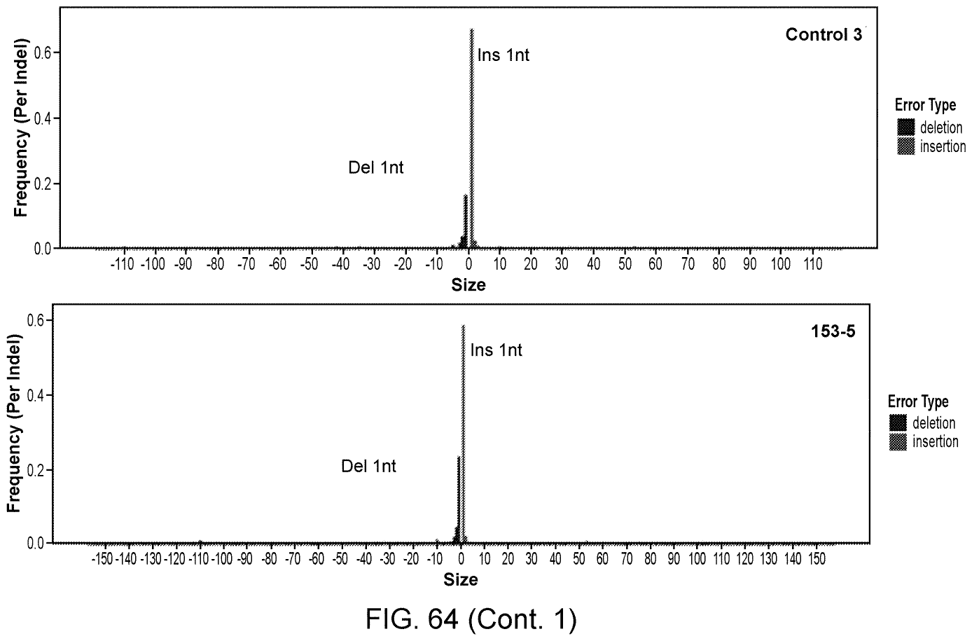

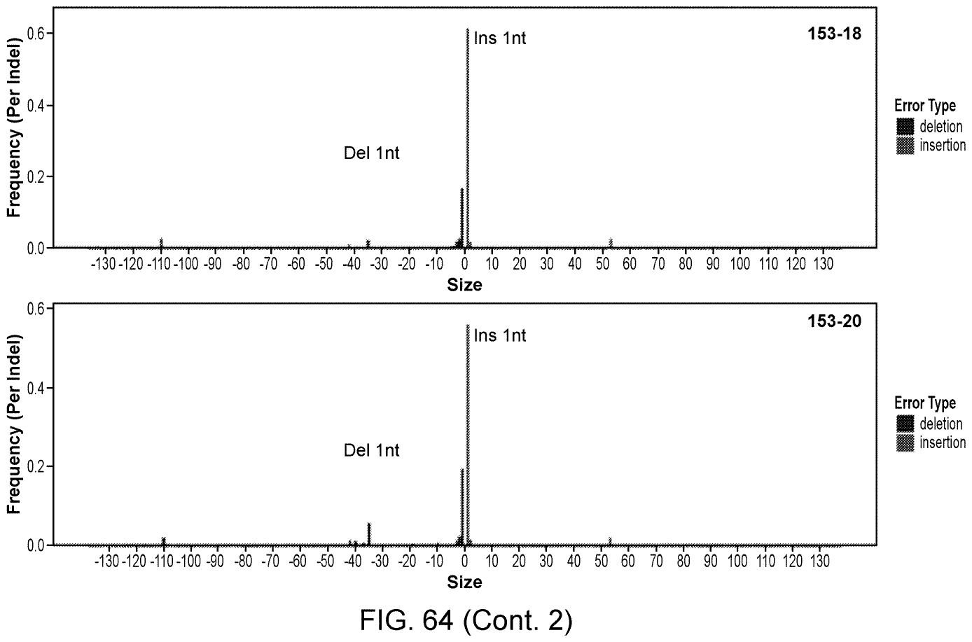

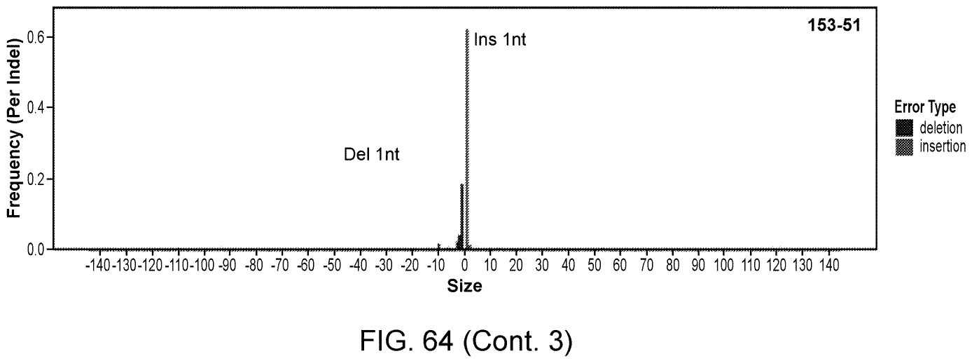

The size and frequency of insertion/deletion (indel) errors was also tabulated. Insertion sizes are displayed as positive values on the X-axis, whereas deletions are displayed as negative values. Frequency is calculated as the count of the indel of the particular size divided by the total number of indel errors. The most prevalent indel error for GII intron RTs (including Control 2 and Control 3) are insertions of 1 nucleotide and, to a lesser extent, deletions of 1 nucleotide (FIGS. 63-64). In contrast, MMLV (Control 1) results in large insertions (53 nucleotides) and large deletions (50 and 110 nucleotides). Of note, indel profiles are similar for GII intron RTs on the standard and modified template.

cDNA Length Distribution: CDNA Drop-Off and Non-Templated Additions

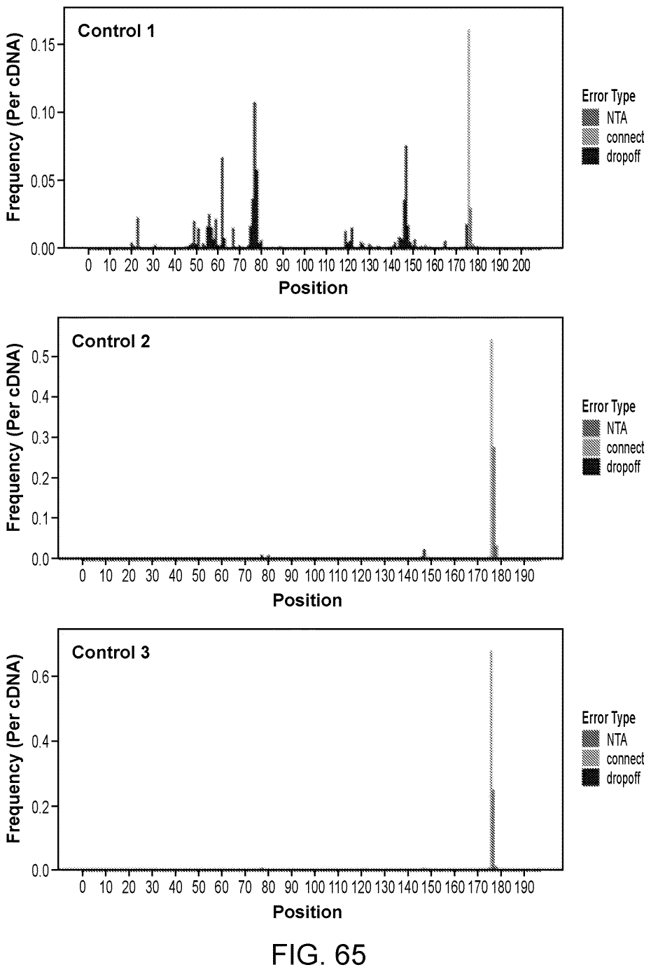

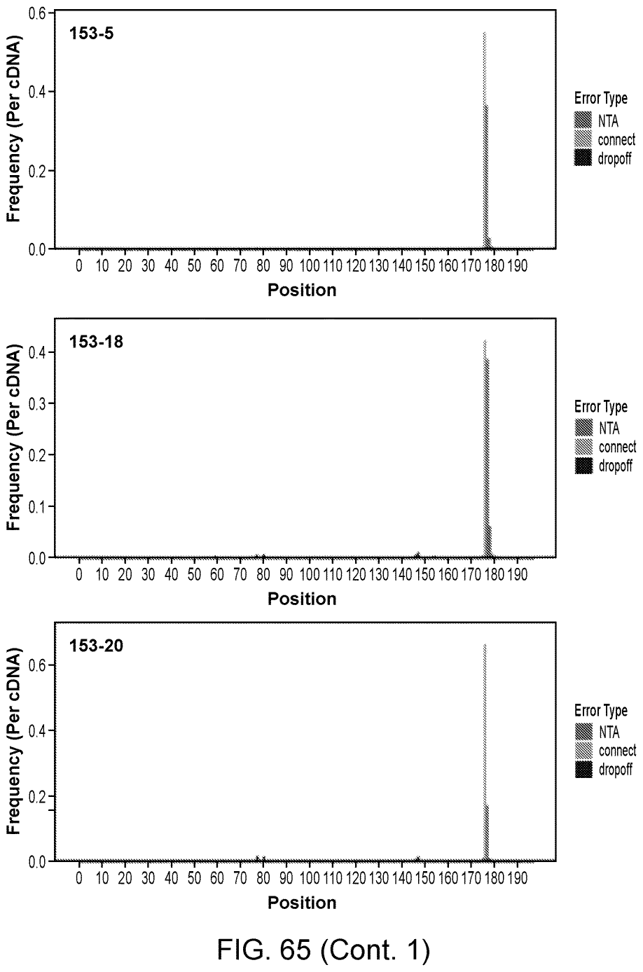

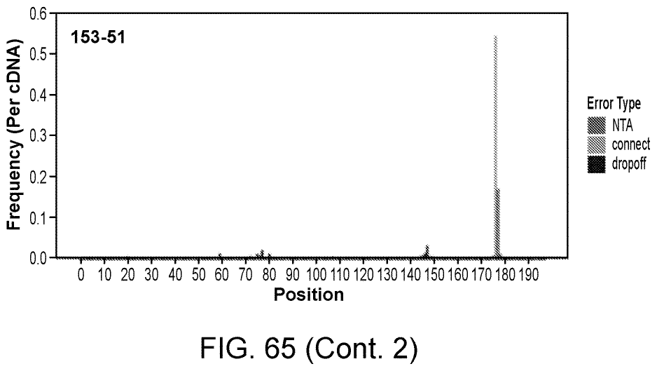

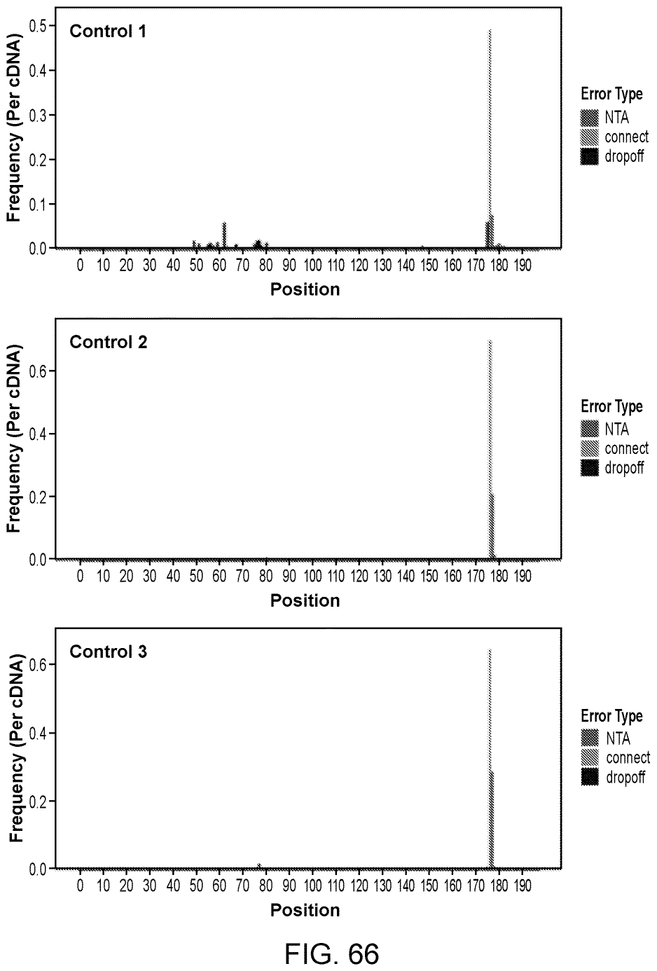

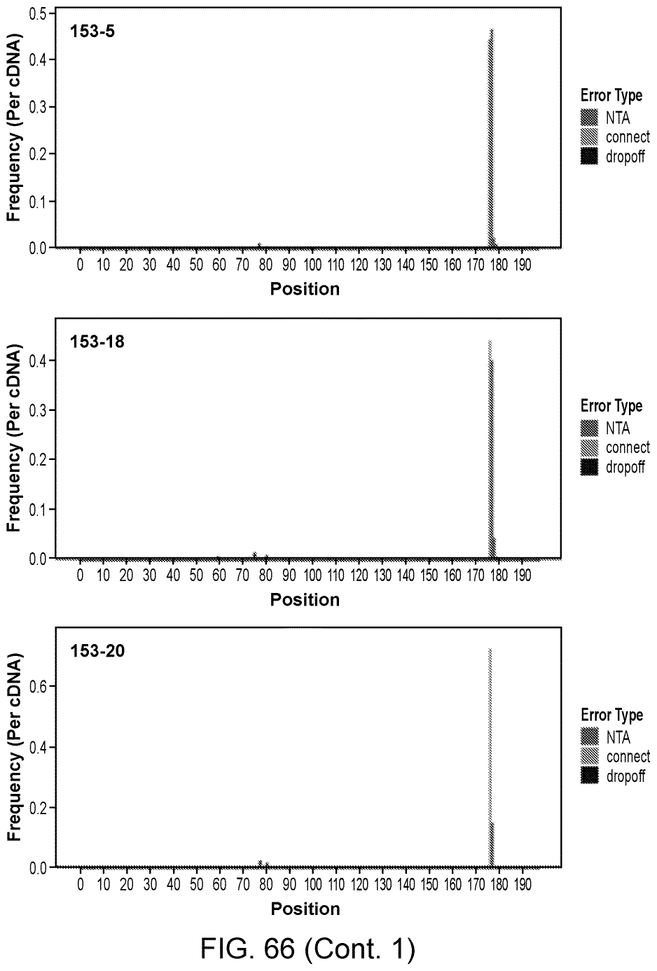

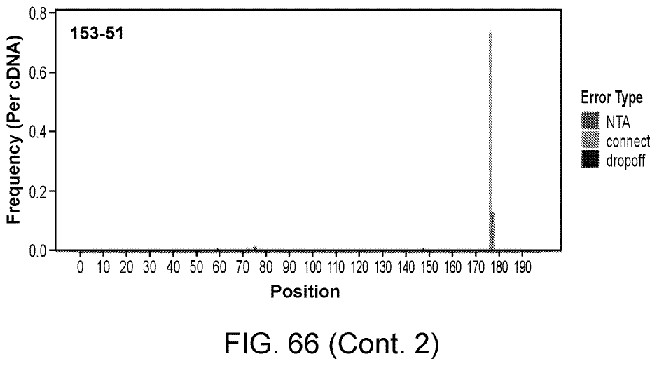

Since the NGS library prep methodology for fidelity analysis relies on 3′ cDNA adapter ligation, cDNA products that are smaller or larger than the expected full-length cDNA product can also be analyzed. These include cDNA drop-off products resulting from the RT falling off the RNA template and RT incorporation of extra nucleotides at the 3′ end of the cDNA past the RNA template, also referred to as non-templated additions (NTA). Frequency of cDNA drop-off, correct length, and NTA was calculated as the frequency per consensus cDNA sequence. For all tested GII intron RTs (including Control 2 and Control 3), minimal cDNA drop-off products are observed (FIGS. 65-66). However, all GII intron RTs have appreciable NTA activity, with the dominant product being an incorporation of 1 extra nucleotide at the 3′ end of the cDNA. These results corroborate previous studies of Control 2 (TGIRT) and Control 3 (MarathonRT), which indicate these GII intron RTs are highly processive and, in the case of Control 2 (TGIRT), have prominent NTA activity where the incorporation of 1 non-templated nucleotide is kinetically preferred.

The less processive retroviral RT MMLV (Control 1) produces prominent drop-off cDNA products on the standard template (FIG. 65), with one drop-off hotspot corresponding at and nearby position 78, the same position as the substitution hotspot correlated to a predicted hairpin in the RNA template. In contrast, MMLV (Control 1) produces mostly full-length cDNA products on the modified template (FIG. 66), which likely explains the improved fidelity of MMLV on the modified RNA substrate. Like the GII intron RTs, MMLV (Control 1) also exhibits NTA activity under these reaction conditions as expected.

### Non-Templated Addition (NTA) Analysis

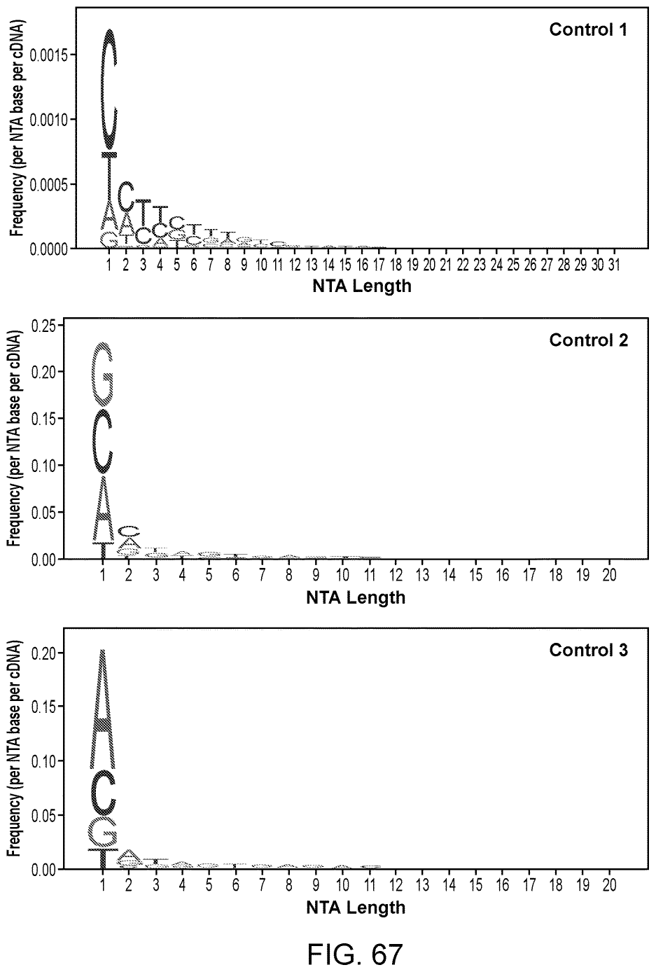

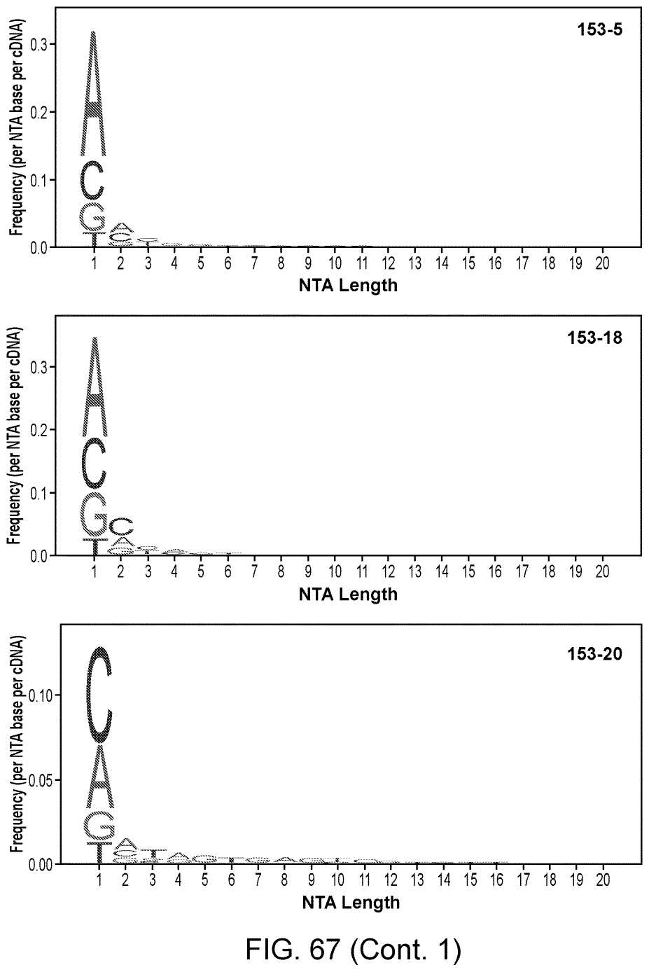

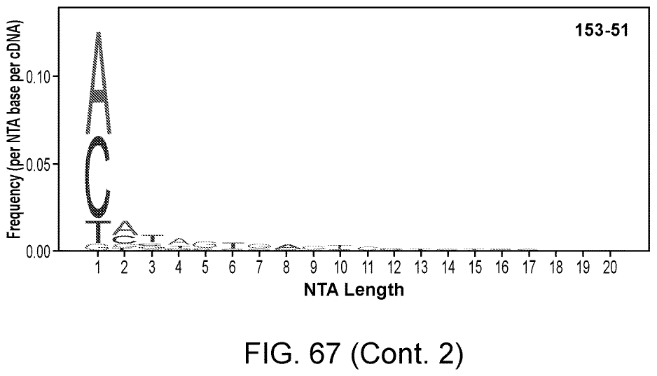

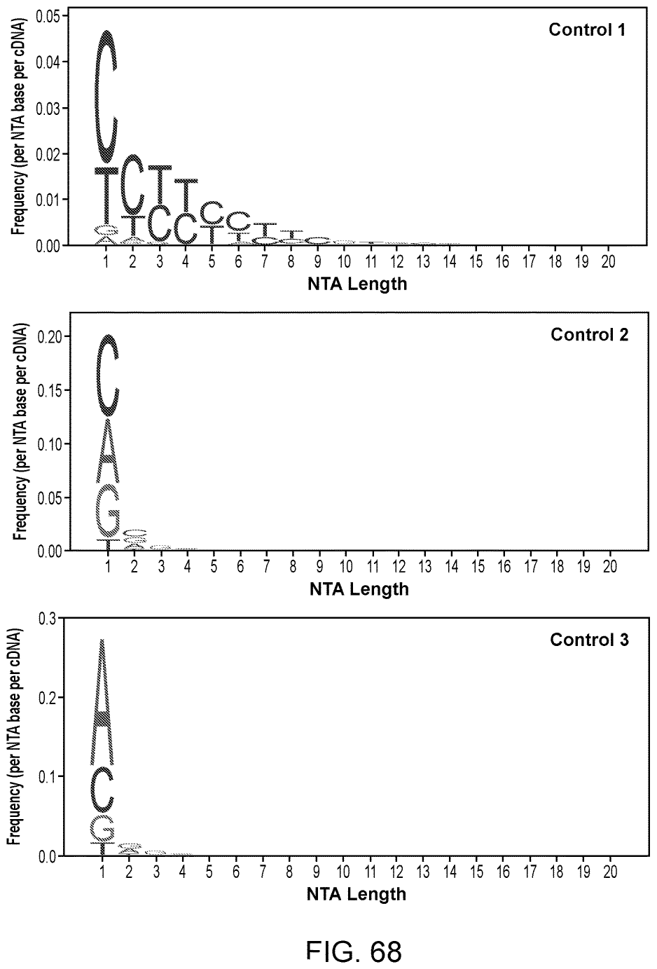

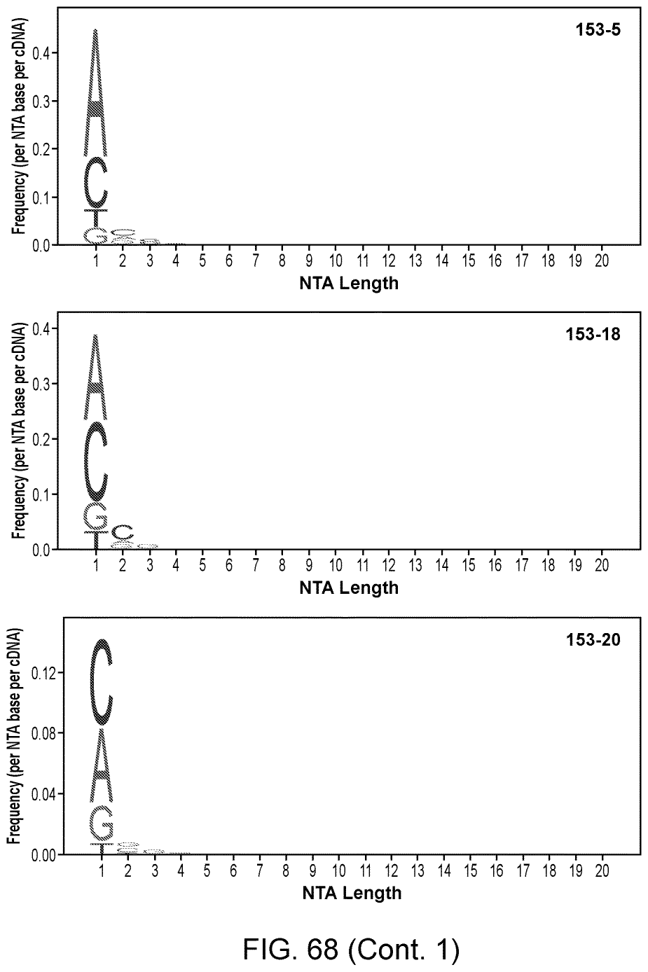

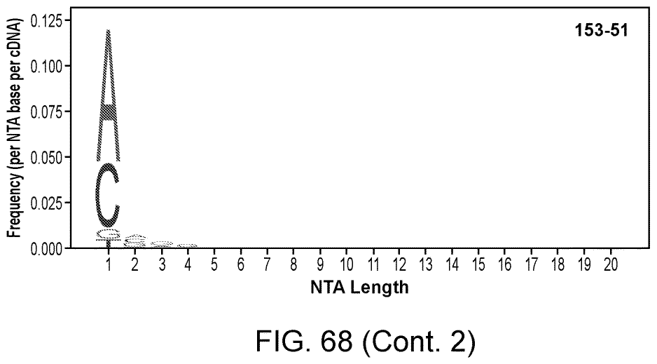

For the cDNA molecules that contain a non-templated addition, the type of nucleotide incorporated was also analyzed. The count of each NTA base identity is divided by the total number of NTA bases. The calculated frequencies are then multiplied to the frequency of cDNAs with NTAs relative to all other cDNAs. NTA analysis for MMLV (Control 1) corroborates previous findings that demonstrated that MMLV tends to incorporate 2-3 cytosines (FIGS. 67-68). Previous results have shown that NTA by TGIRT has a strong preference for incorporating purines, specifically an A (A>G>>C≈T). However, under these reaction conditions, the first NTA nucleotides observed are C, G, and A in almost equivalent abundance, with T being strongly disfavored. The discrepancy may be explained by the fact that the first NTA nucleotide identity was previously determined from a blunt-end duplex, whereas this data was obtained from a primer extension reaction. Additionally, it is unclear whether the identity of the 5′ terminal nucleotide on the RNA template can impact the preference of NTA nucleotide incorporation. Similar to TGIRT (Control 2), MarathonRT (Control 3) and the MG GII intron RTs (MG153 family), T is also strongly disfavored for NTA. MarathonRT (Control 3), MG153-5, and MG153-51 have a strong A preference for the first NTA nucleotide, whereas MG153-18 and MG153-20 have an NTA preference profile more similar to TGIRT (Control 2). Of note, the NTA base incorporation profile is similar for all RTs on the standard and modified RNA template.

### Example 35—Expression and Purification of a R2 Retrotransposon RT

### MTP Screening MG140-8c4 and MG140-8c5 Constructs

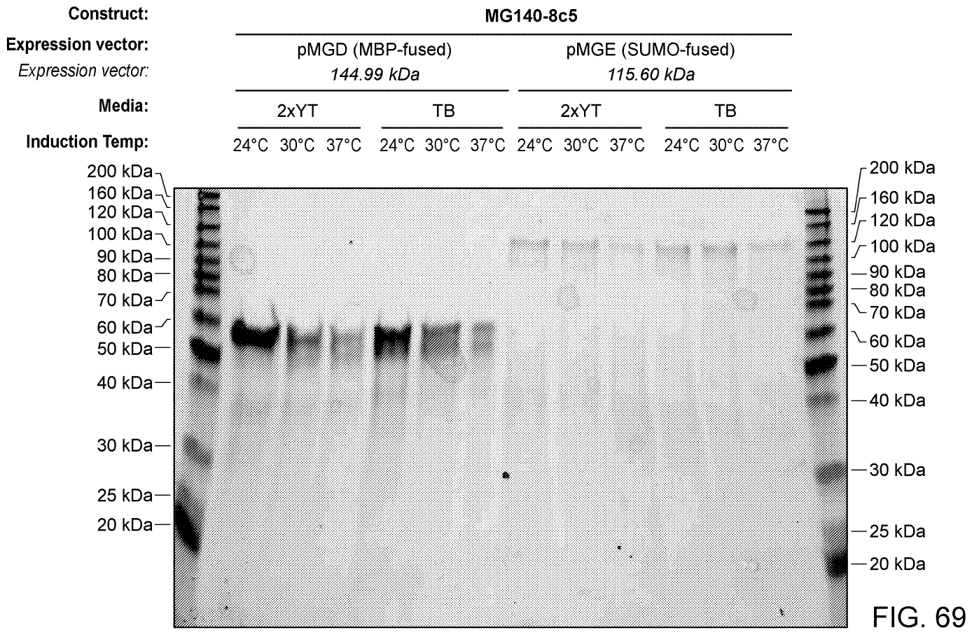

Expression of full-length WT MG140-3 and MG140-8 proteins was unsuccessful due to possible toxicity from the active endonuclease domain of these constructs. Two truncations were designed with the goal of removing the endonuclease domain and leaving the rest of the protein intact. These two truncations (MG140-8c4 and MG140-8c5, SEQ ID NOs: 1807-1808) were tested for expression and purifiability in a small screen which evaluated expression in two different expression vectors (pMGD, pMGE), two different growth media (2×YT, TB), and three different induction temperatures (24° C., 30° C., 37° C.). The final expression construct was 6×His-GS-SUMO-(GS)₂(SG)₂-PSP-nucleoplasmin bipartite NLS-MG140-8c5-SV40 NLS (Table 3). All expressions were performed in the Iq cell strain. Five mL cultures of each construct were grown overnight, shaking at 37° C., in 2×YT media. The following morning, cultures were diluted to 0.1 OD₆₀₀ in 50 mL pre-warmed 2×YT or TB with 100 μg/mL Carbenicillin and grown, shaking at 37° C. Cultures were induced at OD₆₀₀=0.6-0.8 with 0.5 mM IPTG. Following induction, cultures were incubated at 24° C., 30° C., or 37° C. for 4 hrs before harvesting via centrifugation (2,272×g, 10 min) in a 24 deep-well plate. The supernatant was decanted, and all pellets were resuspended in 500 μL resuspension buffer (50 mM HEPES pH 7.5, 1000 mM NaCl, 10 mM MgCl₂, 0.5 mM EDTA, 25 mM imidazole, 10% glycerol)+protease inhibitors+2 mg/mL lysozyme. Resuspended pellets were placed at −80° C. for storage until ready for use. Upon thawing, each well was supplemented with 4.5 mL resuspension buffer and sonicated to lyse (2 s on, 8 s off, 65% amplitude, 2 min total process time). Lysed samples were then clarified via centrifugation (5,000×g, 20 min), and 4.8 mL supernatant was transferred to a new 24 deep well plate. HisPur magnetic Ni-NTA resin was washed twice with Eq buffer (50 mM HEPES pH 7.5, 1000 mM NaCl, 10 mM MgCl₂, 30 mM imidazole, 0.1% Tween-20), and added to each individual sample well (approximately 950 μg resin per well in a volume of 200 μL). A KingFisher Flex was used to conduct purification in a 24 well format. Samples were allowed to bind resin with gentle mixing, and were then washed twice in 3 mL wash buffer (50 mM HEPES pH 7.5, 1000 mM NaCl, 10 mM MgCl₂, 0.5 mM EDTA, 50 mM imidazole, 0.1% Tween-20) and eluted in elution buffer (50 mM HEPES pH 7.5, 1000 mM NaCl, 10 mM MgCl₂, 0.5 mM EDTA, 500 mM imidazole, 5% glycerol, 0.5 mM TCEP). Samples of eluates were mixed with equal volume 2× Laemmli Sample Buffer+10% 2-mercaptoethanol and run on a gel for analysis (FIG. 69). The gels were evaluated by presence or absence of a protein band near the expected molecular weight of each protein construct. Expression of MG140-8c4 in either the pMGD or pMGE vector failed to produce a significant band at the expected molecular weight (132 kDa in pMGD, 102 kDa in pMGE). Expression of MG140-8c5 produced a similar failed result when expressed in the pMGD vector (expected molecular weight of 145 kDa), while expression of this construct in the pMGE vector produced a band of the expected molecular weight (115.6 kDa). Yield was evaluated by assessing protein-of-interest band intensity relative to contaminants. Overall yields of proteins expressed in 2×YT and TB media are comparable, and expressions in both types of media show slightly higher yield at lower temperatures (24° C. and 30° C.) compared to 37° C.

TABLE 3

| Element | Element Sequence (AA) | Description |
|---|---|---|
| 6xHis | Affinity purification | HHHHHH |
| | tag | (SEQ ID |
| | | NO: 2271) |
| GS | GS | linker |
| SUMO | TCGGACTCAGAAGTCAATCAA | Small |
| | GAAGCTAAGCCAGAGGTCAAG | ubiquitin- |
| | CCAGAAGTCAAGCCTGAGACT | like |
| | CACATCAATTTAAAGGTGTCC | modifier |
| | GATGGATCTTCAGAGATCTTC | fusion |
| | TTCAAGATCAAAAAGACCACT | protein |
| | GCCTTTAAGAAGGCTATGGAA | to aid |
| | GCGTTCGCTAAAAGACAGGGT | expression |
| | AAGGAAATGGACTCCTTAAGA | and |
| | TTCTTGTACGACGGTATTAGA | solubility |
| | ATTCAAGCTGATCAGACCCCT | |
| | GAAGATTTGGACATGGAGGAT | |
| | AACGATATTATTGAGGCTCAC | |
| | AGAGAACAGATTGGTGGA | |
| | (SEQ ID NO: 2272) | |
| (SG)₂(GS)₂ | SGSGGSGS | linker |
| | (SEQ ID NO: 2273) | |
| PSP | LEVLFQGP | PreScission |
| | (SEQ ID NO: 2274) | Protease |
| | | cut site |
| | | motif |
| Nucleoplasmin | KRPAATKKAGQAKKKK | Nuclear |
| bipartite NLS | (SEQ ID NO: 1478) | localization |
| | | sequence |
| SV40 NLS | PKKKRKV | Nuclear |
| | (SEQ ID NO: 1477) | localization |
| | | sequence |

### Scaled-Up Expression/Purification of MG140-8c5

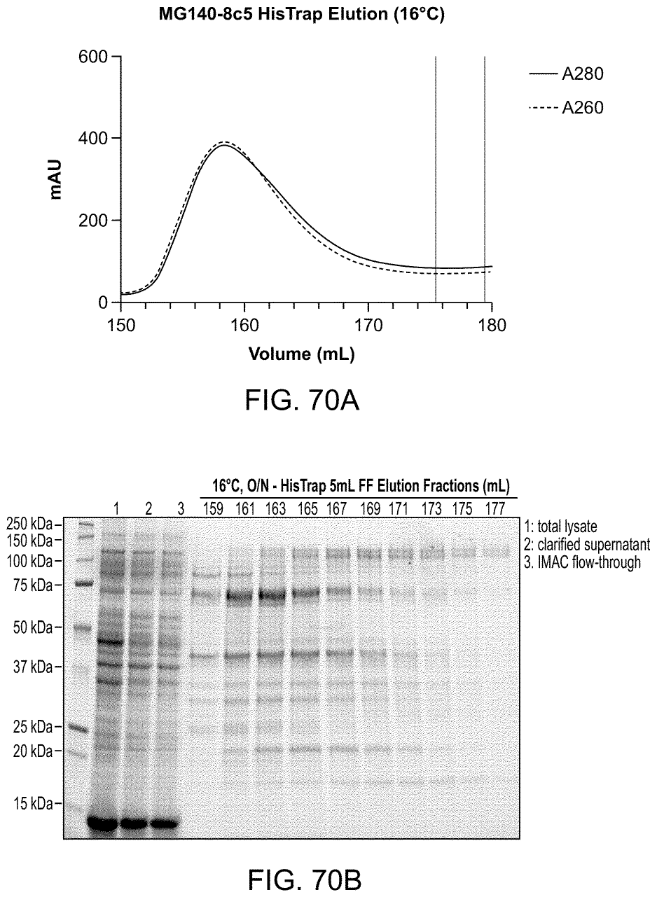

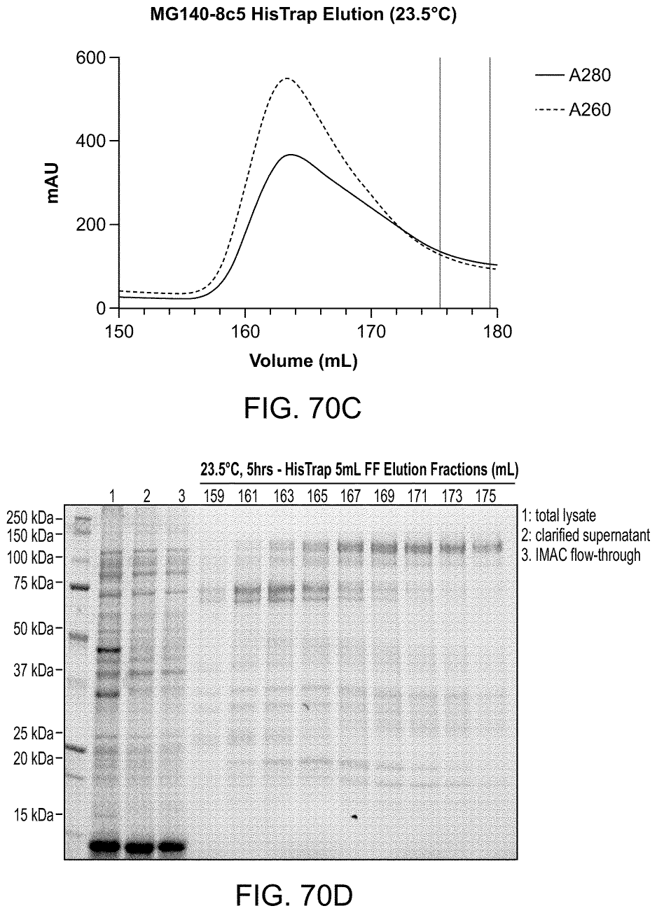

An expression screen of MG140-8c5 revealed the best expression conditions to be at lower temperatures (24° C. and 30° C.), but an overnight expression of MG140-8c5 at 16° C. had yet to be tested. The final expression construct was 6×His-GS-SUMO-(GS) 2 (SG) 2-PSP-nucleoplasmin bipartite NLS-MG140-8c5-SV40 NLS (Table 1, SEQ ID NOs: 1807-1808). All expressions were performed in the Iq cell strain. A 50 mL culture of MG140-8c5 in the pMGE expression vector was grown overnight, shaking at 37° C., in 2×YT media. The following morning, 10 mL of the overnight culture were used to inoculate two 1 L cultures of TB with 100 μg/mL Carbenicillin, which were grown, shaking at 37° C. Prior to induction, cultures were cooled to 20-25° C. in an ice-water bath. Cultures were induced at OD₆₀₀=0.6-0.8 with 0.5 mM IPTG; following, one 1 L culture was incubated overnight at 16° C., shaking, while the other 1 L culture was incubated at 23.5° C., shaking, for 5 hrs. At the end of the induction period, cultures were harvested by centrifugation (6,000×g, 4° C., 10 min) and the pellets were resuspended in resuspension buffer (50 mM HEPES pH 7.5, 1000 mM NaCl, 10 mM MgCl₂, 0.5 mM EDTA, 25 mM imidazole, 10% glycerol)+protease inhibitors+2 mg/mL lysozyme and stored at −80° C. until purified. Upon thawing, resuspended cells were sonicated with a one-half inch sonicator tip at 75% amplitude, 5 s on, 15 s off, for a total process time of 2-3 min in the presence of 0.5% β-octylglucoside detergent. Cell lysates were then clarified via centrifugation (25,000×g, 4° C., 30 min). The supernatants were filtered through a 0.2 μm PES membrane filter and passed over a 5 mL HisTrap using an AKTA Pure FPLC. After sample application, the HisTrap was washed with 6 CV wash buffer A1 (50 mM HEPES pH 7.5, 1000 mM NaCl, 10 mM MgCl₂, 0.5 mM EDTA, 25 mM imidazole, 0.01% Tween-20, 5% glycerol) and 2 CV wash buffer A2 (50 mM HEPES pH 7.5, 1000 mM NaCl, 10 mM MgCl₂, 0.5 mM EDTA, 25 mM imidazole, 5% glycerol) before elution with a 10 CV gradient into elution buffer (50 mM HEPES pH 7.5, 1000 mM NaCl, 10 mM MgCl₂, 0.5 mM EDTA, 500 mM imidazole, 5% glycerol) and collected in 0.5 mL fractions (FIGS. 70A and 70C). Samples of select fractions were mixed with equal volume 2× Laemmli Sample Buffer+10% 2-mercaptoethanol and run on a gel for analysis (FIGS. 70B and 70D). In contrast to the analysis gels from the expression screening performed on the KingFisher Flex, these purifications were very impure with a lot of contaminants. Interestingly, a band at the expected molecular weight eluted off the HisTrap later (at a higher imidazole concentration) than most contaminants, and these fractions (175-179 mL) were able to be separately combined and concentrated. A comparison of the MG140-8c5 constructs expressed at 16° C. overnight versus expression at 23.5° C. for 5 hrs shows little difference in the elution profile off the HisTrap; however, protein purified from the 23.5° C. expression had considerably more nucleic acid contamination, as determined by the absorbance at 260 nm (A260) monitored during elution off the HisTrap (FIGS. 70A and 70C).

### Example 36—cDNA Synthesis Activity of R2 Retrotransposon RT on Standard and Modified RNA Templates

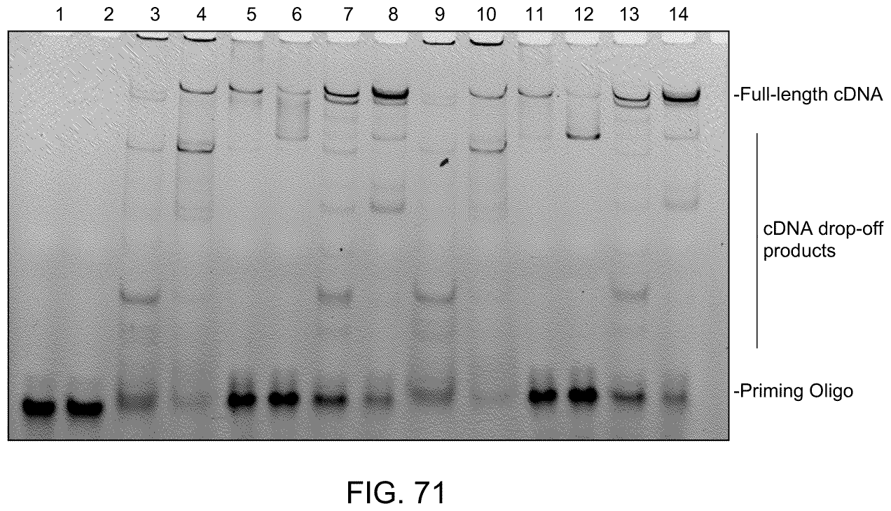

The in vitro activity of the purified R2 retrotransposon RT MG140-8c5 (SEQ ID NOS: 1807-1808) on a standard and modified RNA sequence (SEQ ID NO: 55) was assessed by a primer extension reaction. Two control RTs, MMLV (Control 1) and AccupScript (Control 4) were tested as control enzymes. The standard RNA was prepared using an in vitro transcription reaction containing an equimolar mixture of ATP, UTP, GTP, and CTP while the modified RNA template was prepared with 100% replacement of UTP with m1ΨTP (N1-methyl-pseudouridine-5′-triphosphate). The substrate for the reaction was 100 nM of either standard or modified RNA template (202 nt) annealed to a 5′-FAM labeled primer (SEQ ID NO: 56), and the enzyme was used at a final concentration of 100 nM. The reaction buffer contained the following components: 40 mM Tris-HCl (pH 7.5), 0.2 M NaCl, 10 mM MgCl₂, 1 mM TCEP, RNase inhibitor (murine), and 0.5 mM dNTPs. Following incubation at 37° C. for 1 h, the reaction was quenched via incubation with RNaseH, followed by the addition of 2×RNA loading dye. The resulting cDNA product(s) were separated on a 10% denaturing polyacrylamide gel and were visualized. Based on these results (FIG. 71), purified MG140-8c5 is active and retains appreciable cDNA synthesis activity on the modified RNA template. Of note, data is the result of two independent experimenters and the data is reproducible.

### Example 37—cDNA Synthesis Strand Displacement Activity of R2 Retrotransposon RT

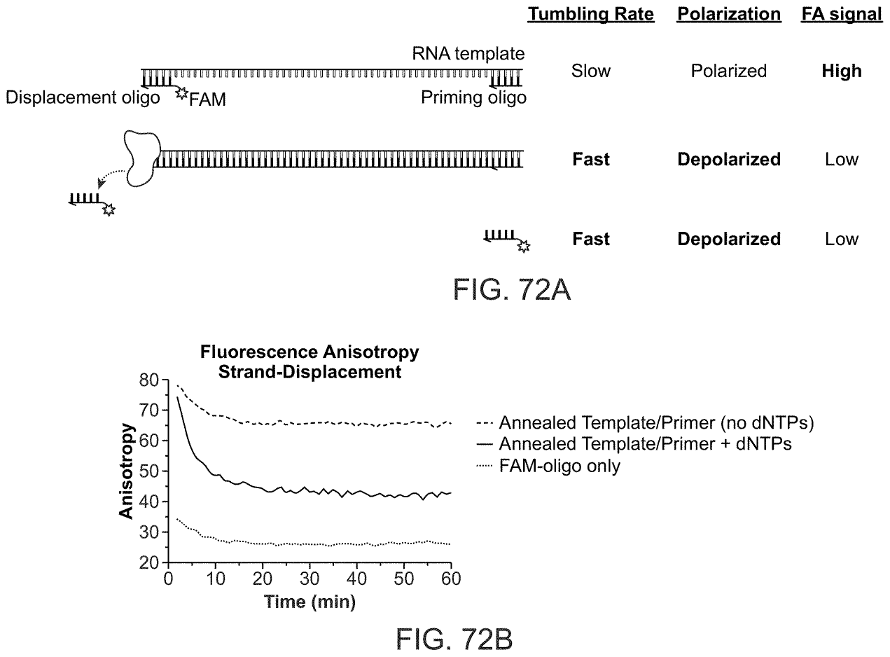

Some reverse transcriptases possess strand displacement activity and are able to displace segments of nucleic acids annealed to the single-stranded RNA template on which they are synthesizing cDNA. To test this, a fluorescence anisotropy-based assay was developed to detect strand displacement. In this assay, a ssDNA priming oligo (SEQ ID NO: 1809) is annealed to the 3′ end of the template RNA (SEQ ID NO: 55) strand and a ssDNA displacement oligo with a 5′ FAM (SEQ ID NO: 1810) is annealed to the 5′ end of the same template RNA (FIG. 72A). Priming oligo is annealed in a 1:1 molar ratio with the template RNA, while the displacement oligo is annealed at a slightly sub-stoichiometric ratio of 0.9:1 with the template RNA. In the annealed state, the molecule—and therefore the fluorophore—tumble slowly in solution and therefore fluorescence polarization remains high. In the event of strand displacement, however, the displaced oligo-conjugated FAM molecule will tumble faster and will therefore depolarize light to a higher degree, which is measured as a decrease in polarization, which is then used to calculate anisotropy. Reactions consisted of 100 nM RNA template annealed to priming and displacement oligos, 1000 nM purified MG140-8c5 protein, 1×RT buffer (40 mM Tris-HCl (pH 7.5), 0.2 M NaCl, 10 mM MgCl₂, 1 mM TCEP), and 1 U/μL RNase inhibitor, murine. Experimental samples were then initiated with the addition of Cf=0.5 mM dNTPs in 1×RT buffer, while negative controls instead received an equal volume of 1×RT buffer. FAM polarization was then monitored using a plate reader. The data show a rapid depolarization of FAM in the presence of dNTPs but not in the absence of dNTPs, suggesting that the FAM-labeled oligo has been displaced by MG140-8c5 cDNA synthesis activity (FIG. 72B).

### Example 38—Second-Strand Synthesis Strand Displacement Activity of R2 Retrotransposon RT

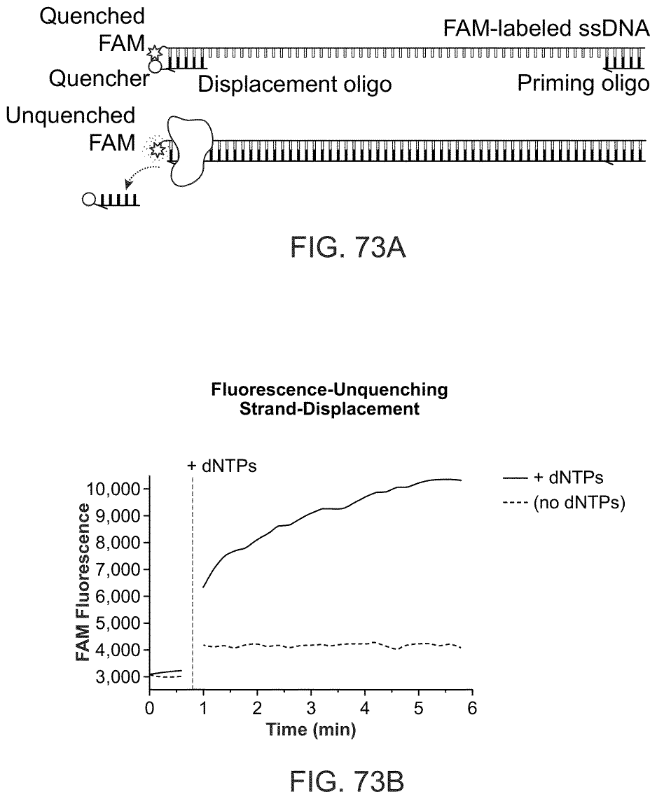

Having already developed an assay to detect strand displacement during cDNA synthesis, an assay was developed to detect strand displacement during second-strand synthesis, where a reverse transcriptase polymerizes complementary DNA on a ssDNA template. To test end, a fluorescence-based assay was developed to detect strand displacement. In this assay, a 100-nt ssDNA oligo (SEQ ID NO: 1811) is synthesized with a 5′ FAM. A ssDNA priming oligo (SEQ ID NO: 1812) is annealed to the 3′ end of the template DNA strand and a ssDNA displacement oligo with a 3′ quencher moiety (SEQ ID NO: 1813) is annealed to the 5′ end of the same template DNA (FIG. 73A). Priming oligo is annealed in a 1:1 molar ratio with the template RNA, while the displacement oligo is annealed at a slightly super-stoichiometric ratio of 1.05:1 with the template DNA. In the annealed state, the quenching moiety is in close proximity to the template FAM, and the FAM fluorescence is quenched. In the event of strand displacement, however, the quencher-conjugated molecule will no longer be close enough to FAM to quench fluorescence, and an increase in fluorescence is observed (FIG. 73A). Reactions consisted of 25 nM DNA template annealed to priming and displacement oligos, 1000 nM purified MG140-8c5 protein, and 1×RT buffer (40 mM Tris-HCl (pH 7.5), 0.2 M NaCl, 10 mM MgCl₂, 1 mM TCEP). Baseline measurements were taken before experimental samples were initiated with the addition of Cf=0.5 mM dNTPs in 1×RT buffer at the 48 s mark, while negative controls instead received an equal volume of 1×RT buffer. FAM fluorescence was then monitored using a plate reader. The data show an increase in FAM fluorescence in the presence of dNTPs but not in the absence of dNTPs, suggesting that the quenching oligo has been displaced by MG140-8c5 second-strand synthesis activity (FIG. 73B).

### Example 39—Second Strand Synthesis Activity and Strand Displacement Activity of GII Intron RTs

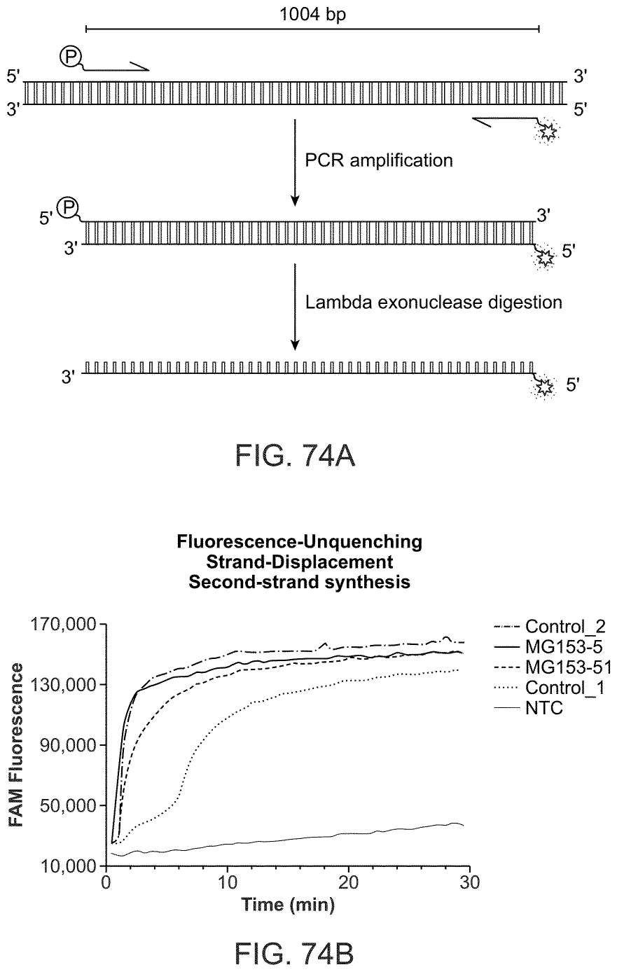

Having demonstrated the feasibility of detecting strand displacement activity during second-strand synthesis using fluorescence-unquenching on a 100-nt template, the use of this assay design was expanded to assess second-strand synthesis processivity by using a 1004-nt FAM-labeled ssDNA template. This 1004-nt FAM-labeled template was developed by first PCR-amplifying a 1004-bp sequence using a primer pair where one primer was synthesized with a 5′ phosphate (SEQ ID NO: 1814), and the other primer was synthesized with a 5′ FAM label (SEQ ID NO: 1815). The PCR products were purified using a 1.5×volume excess of SPRI beads following manufacturer-recommended protocols, and the eluate was concentrated using a 100 k MWCO concentrator. The resulting dsDNA was used as substrate in a reaction with Lambda Exonuclease to produce ssDNA with a 5′ FAM label (FIG. 74A; SEQ ID NO: 1816).

GII intron RT enzymes TGIRT (Control 2), MarathonRT, (Control 3), MG153-5 (SEQ ID NO: 70), and MG153-51 (SEQ ID NO: 113), were generated by a cell-free expression system. Expression constructs were codon-optimized for E. coli and contain an N-terminal single Strep tag. To evaluate if these RTs are capable of performing second strand synthesis and strand displacement on a 1004-nt ssDNA template, expression reactions were diluted to a final 10% v/v in a reaction containing 40 mM Tris-HCl (pH 7.5), 0.2 M NaCl, 10 mM MgCl₂, 1 mM TCEP, 0.5 mM dNTPs, and 33 nM substrate. The substrate for the reaction was prepared by annealing the 5′ FAM-labeled ssDNA template in a 1:1 molar ratio to a DNA priming oligo and 0.95:1 (oligo: template) molar ratio of displacement oligo labeled with a 3′ quencher (SEQ ID NO: 1813). Reactions were initiated by the addition of dNTPs to a final concentration of 0.5 mM dNTPs and FAM fluorescence was monitored over time using a plate. The data show a rapid increase in fluorescence after the addition of dNTPs for Control 2, MG153-5, and MG153-51, consistent with strand displacement via second-strand synthesis (FIG. 74B). Control_1 demonstrates a short lag phase before a similar increase in FAM fluorescence. Contrastingly, reactions that lacked a DNA expression template for a reverse transcriptase (NTC) showed only gradual and modest increase in FAM fluorescence over the course of the reaction. These data show that strand displacement, and therefore second-strand synthesis activity, is measurable from reaction products, without the need to purify heterologously-expressed proteins.

### Example 40—Template Switching Activity of GII Intron and R2 Retrotransposon RTs

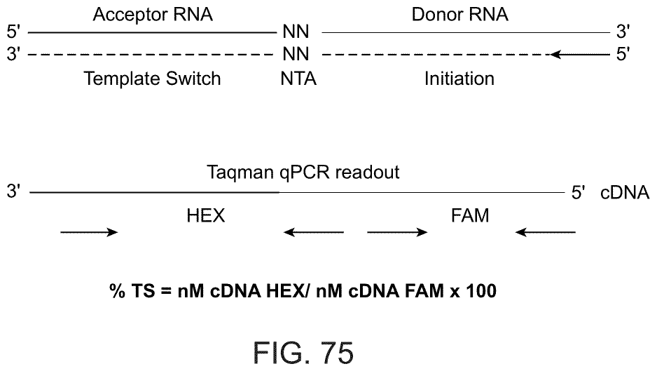

GII intron and R2 retrotransposon RTs possess the ability to perform template switching from the 5′ end of one RNA template (herein referred to as “Donor”) to the 3′ end of another RNA template (herein referred to as “Acceptor”) (FIG. 75). This is facilitated by the RT's NTA activity whereby the RT adds extra nucleotides to the 3′ end of the cDNA molecule in a non-templated manner, creating a small overlap sequence with the Acceptor RNA. To quantify the template switching activity of RTs, a multiplexed Taqman qPCR assay was developed (FIG. 75). The FAM Taqman probe (SEQ ID NO: 1605) and primer set (SEQ ID NOs: 35-36) were designed to detect cDNA resulting from initiation from the priming oligo. The HEX Taqman probe/5HEX/CACTAGTTC/ZEN/TAGAGCGGCCG/3IABKFQ/with TAGAGCGGCCG corresponding to SEQ ID NO: 1817 and primer set (SEQ ID NOs: 1818-1819) were designed to detect cDNA resulting from a template switch, with the primers designed to amplify the junction between Acceptor and Donor cDNA. Taqman primers and probes were validated using a standard curve prepared using a serial dilution of DNA template with known concentrations.

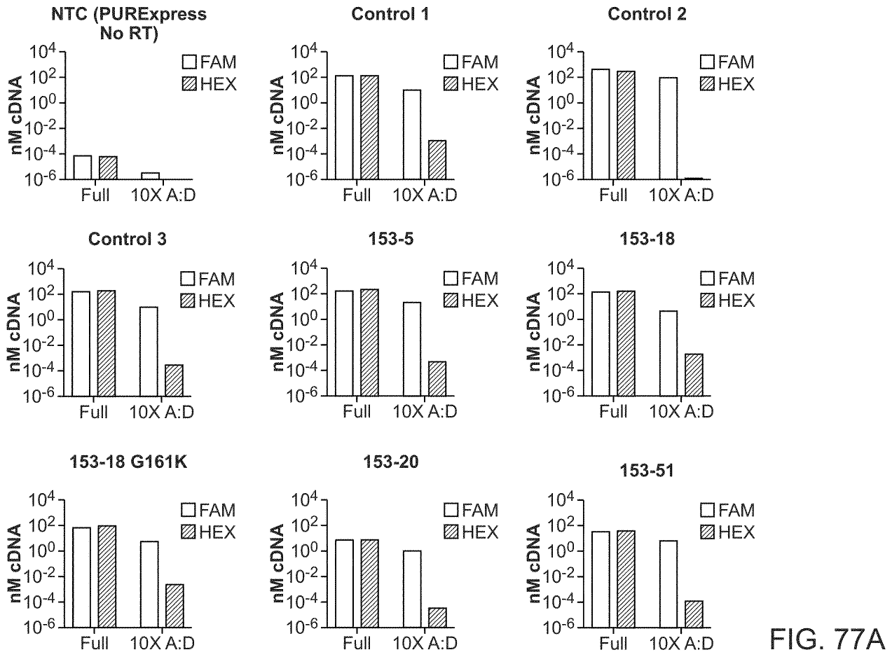

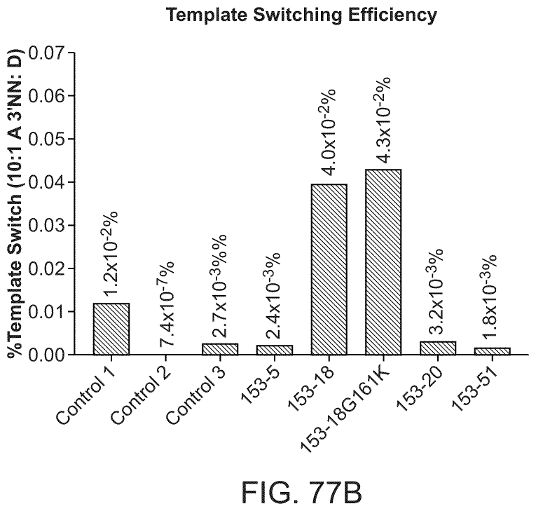

TGIRT (Control 1), MMLV (Control 2), MarathonRT (Control 3), and MG153 family of GII intron enzymes were derived from a cell-free expression system. Expression constructs were codon-optimized for E. coli and contain an N-terminal single Strep tag, except for MG153-18 (SEQ ID NOs: 1820-1821) and MG153-18_G161K (SEQ ID NOs: 1822-1823) in FIG. 77 which were expressed as an N-terminal fusion of 6×His-GS-SUMO-(GS)₂ (SG)₂-PSP (Table 1). R2 retrotransposon RT MG140-8c5 was tested as a purified protein as described above (SEQ ID NOs: 1807-1808).

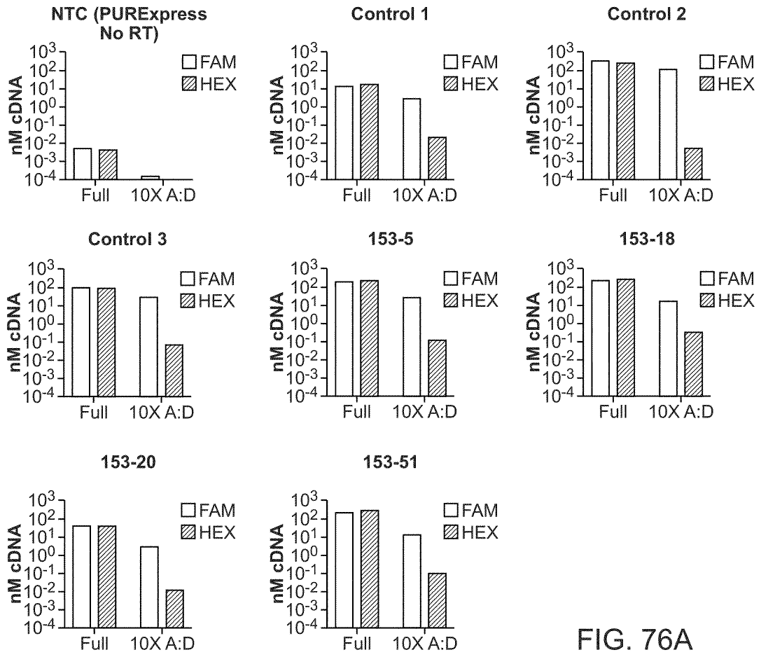

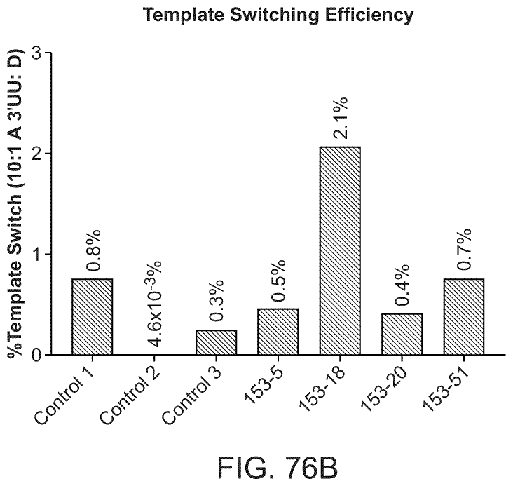

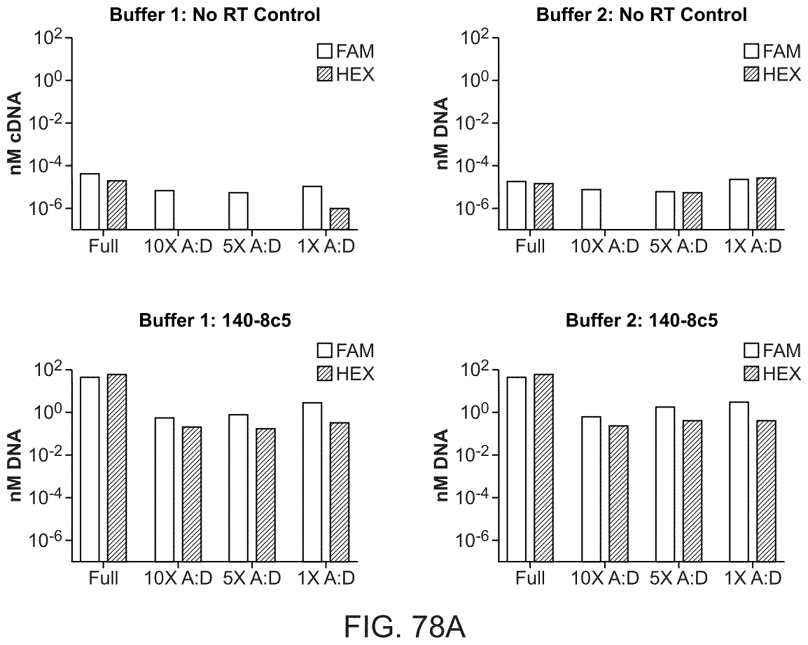

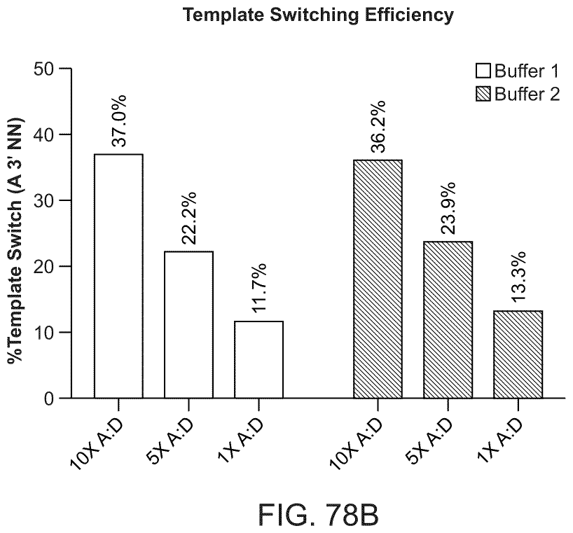

Template switching reactions were prepared by combining each RT with primed Donor RNA template (SEQ ID NO: 1824, IDT) and Acceptor RNA template (SEQ ID NO: 1825 or 1826, IDT). Each RT was also tested against a primed control RNA template, referred to as “Full template” where the Acceptor and Donor RNA sequences are concatenated (SEQ ID NO: 1827). RTs produced by a cell-free expression system were used at a final 10% v/v in the reaction, while MG140-8c5 was used at a final concentration of 200 nM. The primed Donor RNA template and primed Full template were prepared by annealing each template to a ssDNA priming oligo in a 1:1 molar ratio. The template switching reactions contained 100 nM of primed Donor and 100 nM (1×), 500 nM (5×), or 1 μM (10×) of unprimed Acceptor RNA template. The control template was tested at a final concentration of 100 nM. Unless otherwise specified, the reaction buffer used for the reactions was 50 mM Tris-HCl (pH 8.0), 75 mM KCl, 3 mM MgCl₂, 1 mM TCEP, RNase inhibitor, and 0.5 mM dNTPs. Following incubation at 37° C. for 1 h, the reaction was quenched via heat inactivation at 95° C. for 2 minutes. The cDNA products were detected by the Taqman FAM and HEX primers and probes as described above and quantified by extrapolating against a standard curve. Efficiency of template switching was calculated by dividing the quantity of cDNA resulting from a template switch (HEX signal) by the quantity of cDNA resulting from initiation (FAM signal). Results demonstrate that both the GII intron MG153 family of RTs (FIGS. 76A-77B) and the R2 retrotransposon RT MG140-8c5 (FIG. 78) possess template switching activity, albeit to varying degrees.

To test how the identity of the 3′ terminal nucleotides on the Acceptor RNA impact the template switching efficiency of GII intron RTs, an Acceptor RNA with 3′UU nucleotides was compared to a mixed Acceptor substrate prepared by combining 4 templates in equimolar ratio whose 3′ terminal nucleotides are UU, AA, GG, and CC. Template switching efficiency is reduced, albeit still detectable, for all RTs when using an Acceptor template with mixed 3′ ends (denoted as NN, FIG. 77) compared to the 3′UU Acceptor (FIG. 76). For example, TGIRT (Control 1) template switching drops from 0.76% to 0.012% with the mixed Acceptor. This result is likely explained by the fact that GII intron RTs prefer to incorporate A nucleotides during NTA activity, which would facilitate template switching preferentially to acceptors that contain terminal U nucleotides. Interestingly, even with the lowered template switching activity with mixed acceptor, the overall efficiency trends remain relatively the same when comparing between RTs, with MG153-18 and its accompanying variant having the highest efficiency (FIGS. 76-77) across datasets.

To evaluate how different ratios of Acceptor to Donor RNA may impact template switching efficiency of the R2 retrotransposon RT MG140-8c5, Acceptor RNA was added in 10×, 5×, or 1× molar ratio to the Donor RNA (FIG. 76). The template switching efficiency is correlated to the quantity of Acceptor RNA added to the reaction, with a highest determined efficiency of ~37% when Acceptor is present in 10× molar excess to Donor. Slight changes in the reaction buffer composition do not appear to impact template switching.

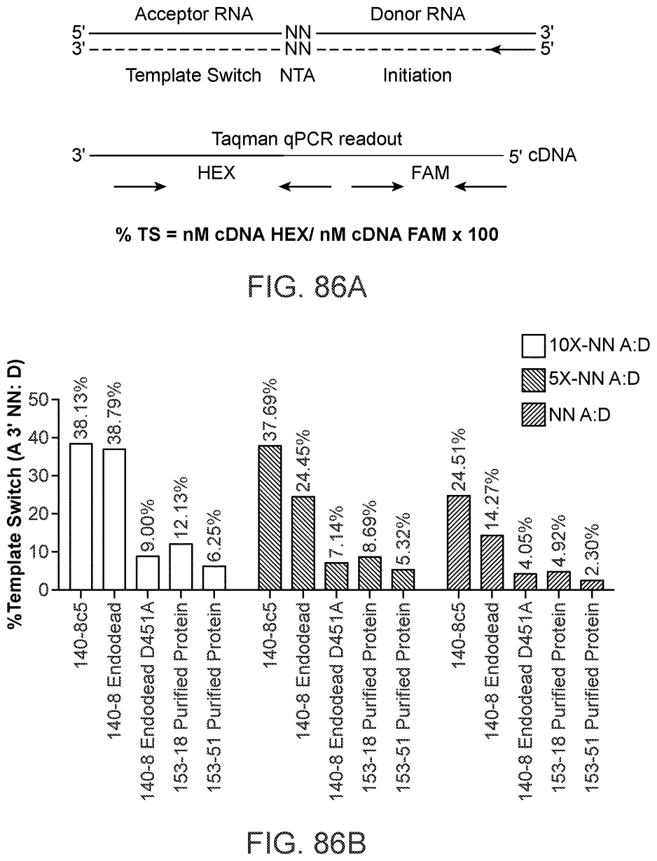

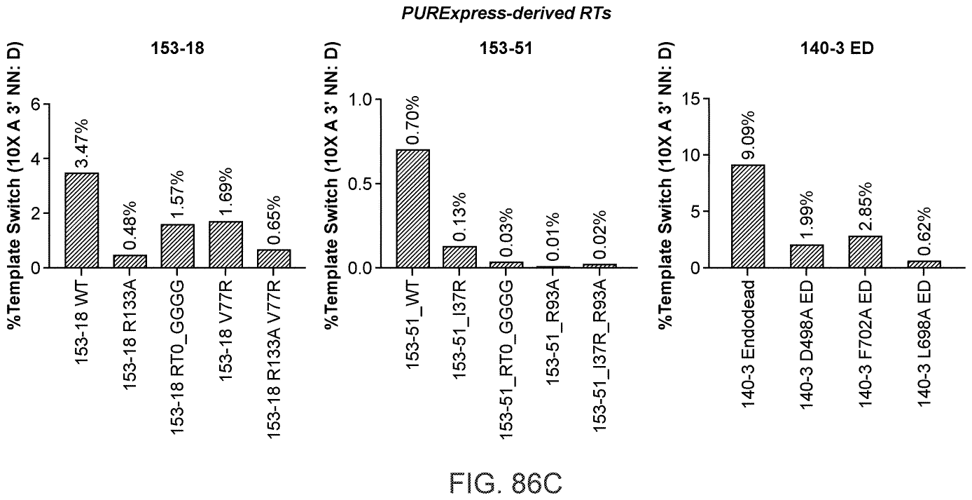

In order to evaluate some of the biochemical properties of rationally engineered RTs vs. their WT variants, the in vitro template switching activities for rationally engineered group II intron and non-LTR R2 retrotransposon RTs was determined. A multiplexed Taqman qPCR assay was developed, in which the FAM Taqman probe and primer set were designed to detect cDNA resulting from initiation from the priming oligo, whereas the HEX Taqman probe and second primer set were designed to detect cDNA resulting from a template switch (FIG. 86A). Taqman primers and probes were validated using a standard curve prepared using a serial dilution of DNA template with known concentrations. RT variants tested were either purified or expressed in cell-free expression systems.

Template switching reactions were prepared by combining each RT preparation (purified or cell-free extract) with primed Donor RNA template and Acceptor RNA template. Each RT was also tested against a primed control RNA template, referred to as “Full template” where the Acceptor and Donor RNA sequences are concatenated. RTs produced by a cell-free expression system were used at a final 10% v/v in the reaction, while purified RTs were used at a final concentration of 200 nM. The primed Donor RNA template and primed “Full template” were prepared by annealing each template to a ssDNA priming oligo in a 1:1 molar ratio. The template switching reactions contained 100 nM of primed Donor and 100 nM (1×), 500 nM (5×), or 1 μM (10×) of unprimed Acceptor RNA template. The control template was tested at a final concentration of 100 nM. Unless otherwise specified, the reaction buffer used for the reactions was 50 mM Tris-HCl (pH 8.0), 75 mM KCl, 3 mM MgCl₂, 1 mM TCEP, RNase inhibitor, and 0.5 mM dNTPs. Following incubation at 37° C. for 1 h, the reaction was quenched via heat inactivation at 95° C. for 2 minutes. The cDNA products were detected by the Taqman FAM and HEX primers and probes as described above and quantified by extrapolating against a standard curve. Efficiency of template switching was calculated by dividing the quantity of cDNA resulting from a template switch (HEX signal) by the quantity of cDNA resulting from initiation (FAM signal). Results indicate that purified group II intron RTs, MG153-18 and MG153-51 exhibit much lower levels of template switching activity than the purified non-LTR retrotransposon RT MG140-8 with the endonuclease domain deletion (140-8c5) or with a dead endonuclease domain (FIG. 86B). Mutations on group II intron RTs MG153-18 and MG153-51, and on non-LTR retrotransposon RTs MG140-3 and MG140-8 with a dead endonuclease domain further reduce the levels of template switching activity in vitro (FIG. 86B and FIG. 86C). Results indicate that rational engineering of RT reduces unwanted activities such as template switching.

### Example 41—Unprimed Activity of GII Intron and R2 Retrotransposon RTs

Evaluating Unprimed cDNA Synthesis Activity by qPCR

To evaluate the impact of 3′ RNA template structure/sequence on the ability of RTs to perform unprimed cDNA synthesis activity, RNA templates were designed to contain either a 3′ polyA (SEQ ID NO: 1828) or 3′ hairpin (MS2 loop) (SEQ ID NO: 1829). To evaluate whether the 3′OH of the RNA template could be used to initiate cDNA synthesis, each template was also tested with the 3′OH masked by a blocking group (C3 spacer, /3SpC3/) (SEQ ID NOs: 1830-1831). GII intron and R2 RTs were tested for their ability to perform cDNA synthesis using a 100 nM RNA template that was either annealed or not annealed to a priming DNA oligo (SEQ ID NO: 34 or 35). For these reactions, the RT enzymes MMLV (Control 1), TGIRT (Control 2), MG153-5 (SEQ ID NO: 70), MG153-18 (SEQ ID NOs: 1820-1821), MG153-18_G161K (SEQ ID NOs: 1822-1823), MG153-20 (SEQ ID NO: 85) and MG153-51 (SEQ ID NO: 113) were derived from a cell-free expression system and used 10% final v/v % in the cDNA synthesis reaction. Expression constructs were codon-optimized for E. coli and contain an N-terminal single Strep tag, except for MG153-18 and MG153-18_G161K which were expressed as an N-terminal 6×His-GS-SUMO-(GS)₂ (SG)₂-PSP (Table 1) fusion. Expression for all PURExpress-derived RTs were confirmed by SDS-PAGE analysis. MG140-8c5 trim (SEQ ID NOs: 1807-1808) was used as purified protein as described above at a final concentration of 200 nM. For MMLV (Control 1), TGIRT (Control 2), and the MG153 family, the reaction buffer contained the following components: 50 mM Tris-HCl (pH 8.0), 75 mM KCl, 3 mM MgCl₂, 1 mM TCEP, RNase inhibitor and 0.5 mM dNTPs. For 140-8c5 trim, the reaction buffer contained the following: 40 mM Tris-HCl (pH 7.5), 0.2 M NaCl, 10 mM MgCl₂, 1 mM TCEP, RNase inhibitor, and 0.5 mM dNTPs. Following incubation at 37° C. for 1 h, cDNA products were detected by Taqman qPCR using a taqman probe (FAM) (SEQ ID NO: 1605) and primers (SEQ ID NOs: 35-36) specific to the expected cDNA product.

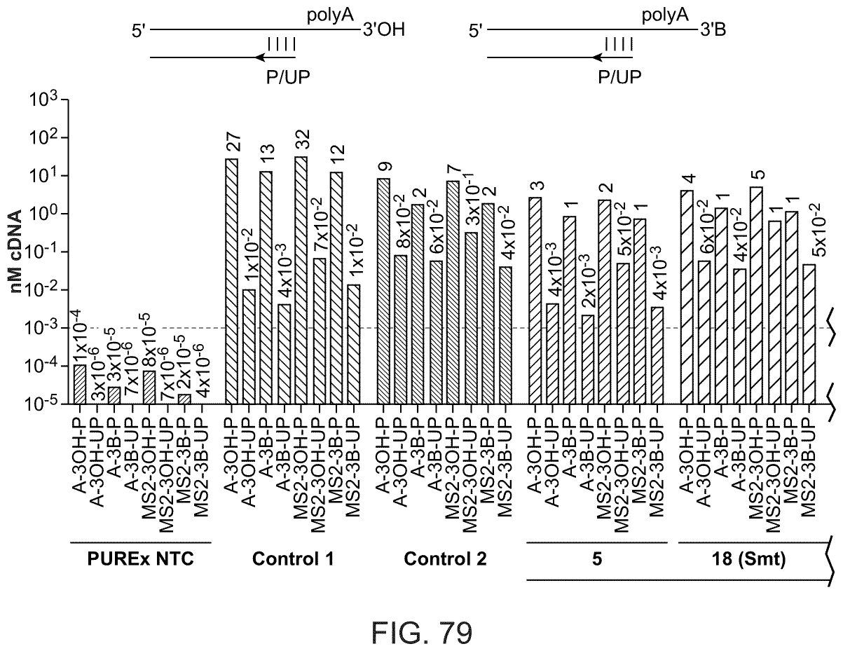

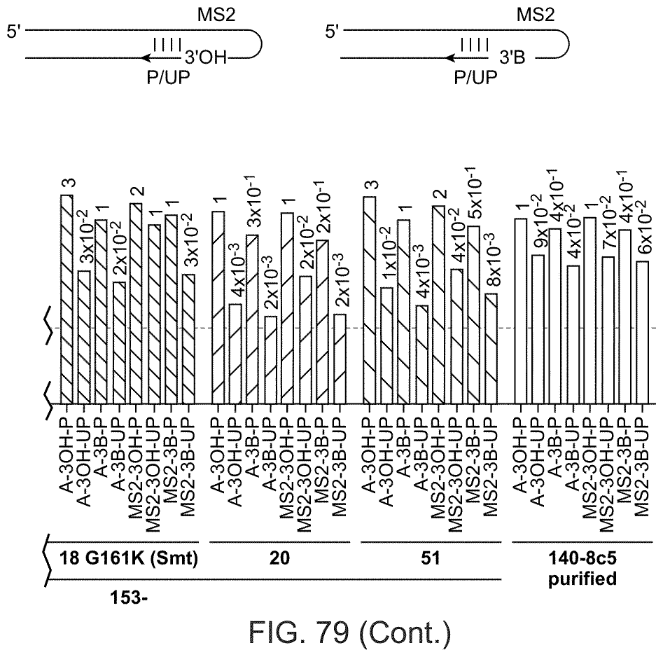

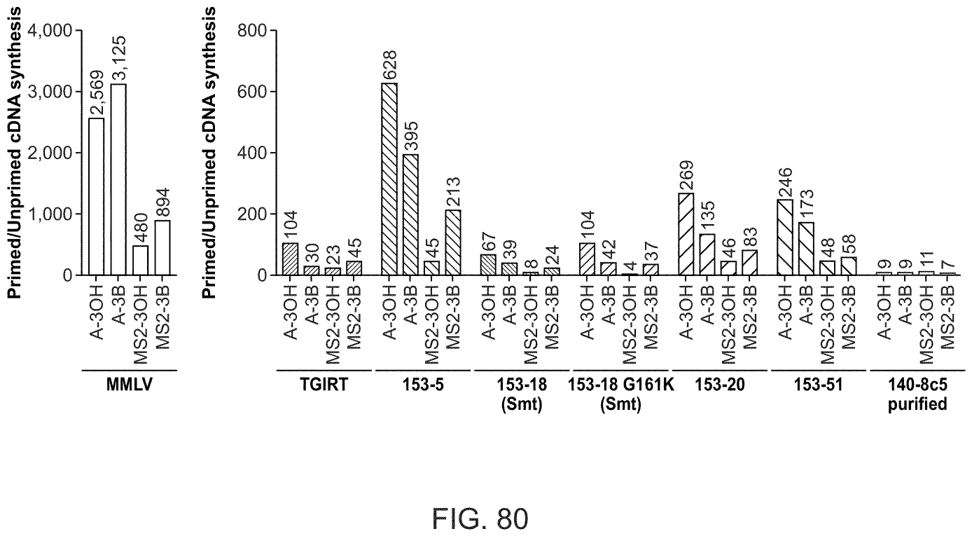

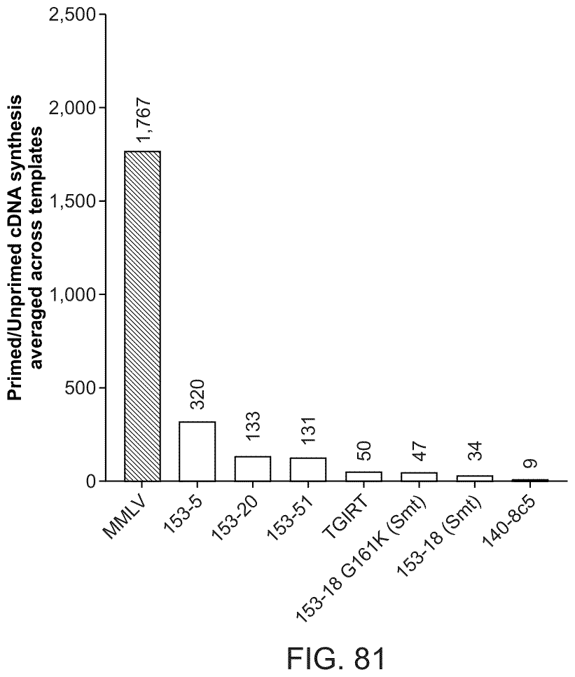

Based on these results, all tested RTs have some extent of unprimed cDNA synthesis activity, with detectable cDNA product levels at least 10-fold above the PURExpress NTC (no expression template control) background (FIG. 79, dashed line). However, all RTs do preferentially perform cDNA synthesis on primed RNA templates, albeit to different extents. To compare RT preference for primed vs. unprimed templates, the quantity (nM) of cDNA produced from a primed RNA template was divided by the quantity unprimed. Strikingly, MMLV (Control 1) has the strongest preference for primed RNA templates (~480-2,570-fold, FIG. 80). GII intron RTs (Control 2 and MG153 family) have more unprimed cDNA synthesis if the 3′ end of the RNA template is structured via an MS2 loop. Some, but not all, of the unprimed cDNA synthesis activity appears to be facilitated by the 3′OH, as some cDNA synthesis is still observed when the 3′OH is masked. In contrast, the R2 retrotransposon RT MG140-8c5 has prevalent unprimed activity when the 3′ end of the RNA template is both structured or unstructured. Unprimed cDNA synthesis, in this case, does not seem to be facilitated by the 3′OH since significant activity remains if the 3′OH is blocked. When the preference for primed substrates across all templates was averaged, the results indicate MMLV (retroviral Control) has the strongest preference for primed (~1,800-fold preference), while the R2 retrotransposon MG140-8c5 has the weakest (~9-fold preference) (FIG. 81).

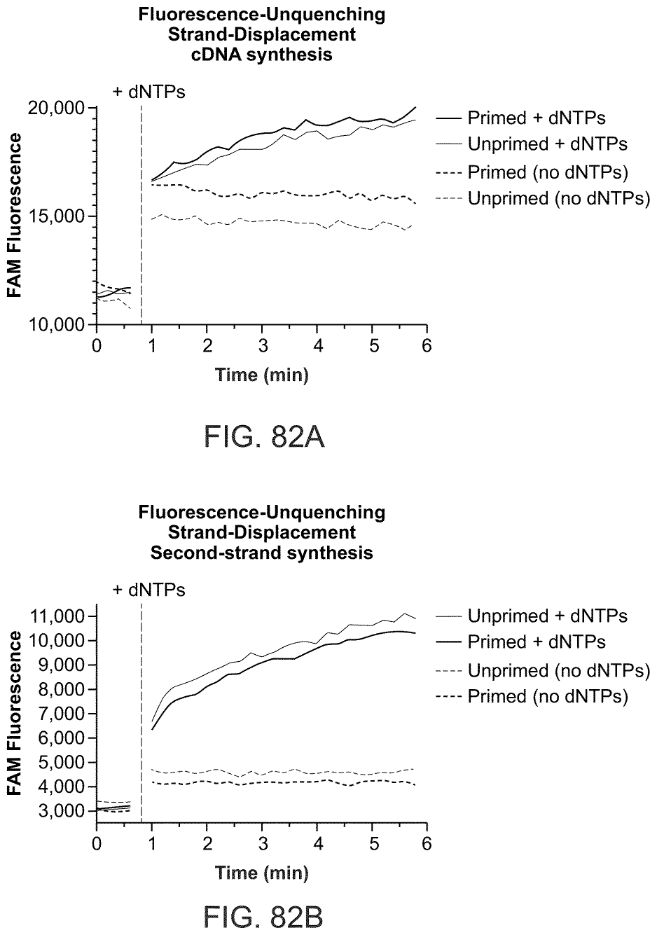

Evaluating unprimed cDNA synthesis and S3 activity by quencher displacement assay Up to this point, negative controls in strand-displacement assays were performed by omitting dNTPs, without which reverse transcriptases cannot polymerize either cDNA or second-strand synthesis. To test whether the cDNA and second-strand synthesis in these reactions are initiating at the desired priming site, control experiments comparing displacement of quenching oligos from both primed and unprimed template strands were conducted. RNA templates (SEQ ID NO: 1832) or ssDNA templates (SEQ ID NO: 1811), both 100 nt long, were ordered from IDT with 5′ FAM modifications. Primed templates were annealed to both an equimolar ratio priming oligo (SEQ ID NO: 1812) and a slight excess (1.05:1) of quenching displacement oligo (SEQ ID NO: 1813); unprimed templates were annealed only to the quenching displacement oligo (also at a 1.05× molar excess over template). Reactions were set up with 25 nM primed or unprimed template, 1000 nM MG140-8c5 enzyme, 1× buffer (40 mM Tris-HCl (pH 7.5), 0.2 M NaCl, 10 mM MgCl₂, 1 mM TCEP), and 1 U/μL RNase inhibitor, murine. Baseline measurements were taken before experimental samples were initiated with the addition of Cf=0.5 mM dNTPs in 1×RT buffer at the 48 s mark, while negative controls instead received an equal volume of 1×RT buffer. FAM fluorescence was then monitored using a plate reader. The data show an increase in fluorescence in all reactions where dNTPs were added, regardless of whether the template was primed or unprimed. This is true for reactions set up to detect strand displacement from both cDNA synthesis (FIG. 82A) and second-strand synthesis (FIG. 82B). Collectively, these data suggest that MG140-8c5 has significant activity on unprimed templates and that it is likely to perform both cDNA synthesis and second-strand synthesis on single-stranded nucleic acid templates.

### Example 42—Diversification of Reverse Transcriptases Generates Active Retron-Like and R2 Retrotransposon RTs

### Computational Reconstruction of RT Ancestral Intermediate Sequences

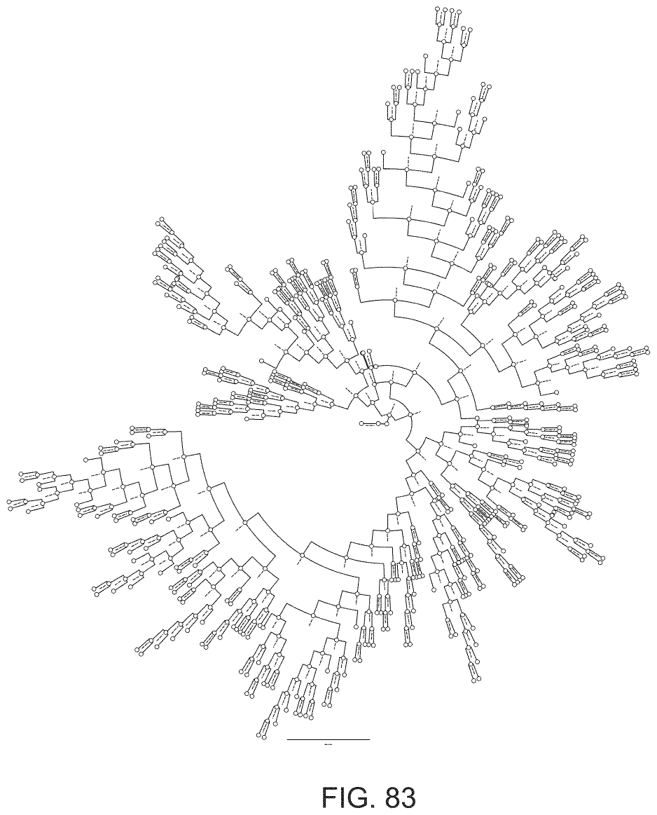

In an effort to generate further diversity and improve the biochemical properties of R2 retrotransposons and retron-like RT families, ancestral sequence reconstruction (ASR) algorithms were used. ASR is a computational technique that uses existing protein sequences and the relationships inferred between them to reconstruct putative sequences of ancient, now extinct, proteins. This technique was used to computationally reconstruct sequences of the MG160 and MG140 RT families. For the analysis, 367 MG160 protein sequences, 351 MG140 protein sequences, as well as subsets of MG140 protein sequences were separately aligned. Phylogenetic trees were built (FIG. 83) and the trees were rooted using distant RT sequences as outgroups. Ancestral sequence reconstruction was done. Insertions and deletions were identified manually for each reconstructed node. Reconstructed RT sequences (SEQ ID NOs: 1894-1926 and 2011-2026) are evaluated for cDNA synthesis activity in vitro and in human cell lines.

### Example 43—Integrations of Large Cargo Templates by Non-LTR Retrotransposon RTs and GII Intron RTs (Prophetic)

Group II introns and non-LTR retrotransposases are capable of integrating large cargo into a target site via target primed reverse transcription of an RNA template. To determine the most efficient cargo designs for integration by each RT, diverse RNA templates containing various combinations of 5′ and 3′ UTRs (SEQ ID NOs: 2211-2257) are designed. The ability of RTs to reverse transcribe and integrate cDNA from an RNA cargo into a target site is tested by expressing RTs in the presence of the RNA cargo.

Reverse transcriptases are cloned under a CMV or alternative promoter, and a Flag-HA-SV40 NLS tag is added at the N-terminus and another SV40-NLS is added at the C-terminus to ensure localization to the nucleus upon expression. Optionally, an MS2 coat protein (MCP) tag is fused to the RT to facilitate recognition of alternative MS2 tagged RNA template cargoes. Different RNA templates are designed for testing each RT for integration. For example, some templates can contain MS2 loops for recognition by the MCP-tagged RT, some template designs contain endogenous UTR elements, while some cargo designs contain additional homology arms flanking the desired cargo. Cargo for integration by each RT can encompass an antisense-mCherry open reading frame (ORF) driven by an EF1 alpha promoter, other reporter cargos, or any other desired cargo. The DNA sequence corresponding to each template with an additional T7 promoter is generated and PCR amplified by phusion polymerase according to the manufacturer's instructions. The PCR reaction is cleaned and 200-500 ng of cleaned PCR product is used for in vitro transcription reaction (IVT). The IVT reaction buffer contains 1×T7 buffer (40 mM Tris HCl, pH 7.5, 16.5 mM MgCl₂, 50 mM NaCl, 2.5 mM Spermidine and 1 mM DTT), 5 mM rATP, 5 mM rUTP, 5 mM rGTP, 4 mM CleanCap-AG, 0.1 unit IPPase (inorganic pyrophosphatase), 40 units RNase inhibitor and 750 units high concentration Hi-T7 RNA polymerase. The IVT reaction is incubated at 50° C. for 1 hr, followed by DNase I treatment with 10 units of DNaseI for 10 minutes at 37° C. The reactions are then cleaned using the MEGAclear transcription clean up kit following the manufacturer's instructions. The purity of RNA templates is confirmed by Tapestation and their quantities determined.

Integration assays are set up in a 6-well format with 1 million engineered cells plated per 6-well in 2 ml media. Each well is transfected with 2500 ng plasmid encoding the RT protein and 2400 ng of RNA cargoes. 24 hours later cells are split into puromycin containing media (2 ug/ml) to select for cells transfected with the RT plasmid, which contains a puromycin resistance cassette. Cells are switched to media without puromycin 3 days post-transfection and split every 2-3 days until 10 days post-transfection. Cells are collected at 4-10 days post transfection and lysed in 100 μL DNA Extraction Solution. Integration of cargo is detected by nested PCR at the left end junction (LE) and the right end junction (RE) using primers designed to anneal to the target and donor regions. PCR products are run on a tapestation and LE and RE PCR products are sequenced. Sequencing reads are analyzed to determine successful integration of cargo at the target site.

### REFERENCES

Price M N, Dehal P S, Arkin A P. FastTree 2—approximately maximum-likelihood trees for large alignments. Plos One 2010, 5, e9490.
Yamada K D, Tomii K, Katoh K. Application of the MAFFT sequence alignment program to large data—reexamination of the usefulness of chained guide trees. Bioinformatics 2016, 32, 3246-3251.
Jumper J, Evans R, Pritzel A, Green T, Figurnov M, Ronneberger O, et al. Highly accurate protein structure prediction with AlphaFold. Nature 2021, 596, 583-589.
Varadi M, Anyango S, Deshpande M, Nair S, Natassia C, Yordanova G, et al. AlphaFold Protein Structure Database: massively expanding the structural coverage of protein-sequence space with high-accuracy models. Nucleic Acids Res 2022, 50, D439-D444.
Wang Y, Guan Z, Wang C, Nie Y, Chen Y, Qian Z, Cui Y, Xu H, Wang Q, Zhao F, Zhang D, Tao P, Sun M, Yin P, Jin S, Wu S, Zou T. Cryo-EM structures of Escherichia coli Ec86 retron complexes reveal architecture and defense mechanism. Nat Microbiol. 2022, 7 (9), 1480-1489.
Mestre M R, González-Delgado A, Gutiérrez-Rus L I, Martínez-Abarca F, Toro N. Systematic prediction of genes functionally associated with bacterial retrons and classification of the encoded tripartite systems. Nucleic Acids Res. 2020, 48 (22), 12632-12647.
Kapitonov V V, Tempel S, Jurka J. Simple and fast classification of non-LTR retrotransposons based on phylogeny of their RT domain protein sequences. Gene 2009, 448 (2), 207-13.
Shimamoto, T., Shimada, M., Inouye, M. and Inouye, S. (1995) The role of ribonuclease H in multicopy single-stranded DNA synthesis in retron-Ec73 and retron-Ec107 of Escherichia coli. J. Bacteriol., 177, 264-267.
Simon A J, Ellington A D, Finkelstein I J. Retrons and their applications in genome engineering. Nucleic Acids Res 2019; 47:11007-11019. DOI: 10.1093/nar/gkz865.
Shimamoto, T., Hsu, M. Y., Inouye, S. and Inouye, M. (1993) Reverse transcriptases from bacterial retrons require specific secondary structures at the 5′-end of the template for the cdna priming reaction. J. Biol. Chem., 268, 2684-2692.
Zhao C, Liu F, Pyle A M. An ultraprocessive, accurate reverse transcriptase encoded by a metazoan group II intron. RNA. 2018 February; 24 (2): 183-195. doi: 10.1261/rna.063479.117. Epub 2017 Nov. 6. PMID: 29109157; PMCID: PMC5769746.
Lopez S C, Crawford K D, Lear S K, Bhattarai-Kline S, Shipman S L. Precide genome editing across kingdoms of life using retron-derived DNA. Nat Chem Biol. 2022 February; 18 (2): 199-206.
Lentzsch A M, Yao J, Russell R, Lambowitz A M. Template-switching mechanism of a group II intron-encoded reverse transcriptase and its implications for biological function and RNA-Seq. J Biol Chem. 2019 Dec. 20; 294 (51): 19764-19784. doi: 10.1074/jbc.RA119.011337. Epub 2019 Nov. 11. PMID: 31712313; PMCID: PMC6926447.
Wellenreuther R, Schupp I, Poustka A, Wiemann S; German cDNA Consortium. SMART amplification combined with cDNA size fractionation in order to obtain large full-length clones. BMC Genomics. 2004 Jun. 15; 5 (1): 36. doi: 10.1186/1471-2164-5-36. PMID: 15198809; PMCID: PMC436056.
Park S K, Mohr G, Yao J, Russell R, Lambowitz A M. Group II intron-like reverse transcriptases function in double-strand break repair. Cell. 2022 Sep. 29; 185 (20): 3671-3688.e23. doi: 10.1016/j.cell.2022.08.014. Epub 2022 Sep. 15. PMID: 36113466; PMCID: PMC9530004.
Harms, M. & Thornton J. W. Analyzing protein structure and function using ancestral gene reconstruction. Current Opinion in Structural Biology, 20:360-366 (2010).
Katoh K, Standley D M. MAFFT multiple sequence alignment software version 7: improvements in performance and usability. Mol Biol Evol. 2013; 30 (4): 772-780. doi: 10.1093/molbev/mst010
Stamatakis, A. RAXML version 8: a tool for phylogenetic analysis and post-analysis of large phylogenies. Bioinformatics 30 (9), 1312-1313 (2014).
Yang, Z. PAML 4: a program package for phylogenetic analysis by maximum likelihood. Molecular Biology and Evolution 24:1586-1591 (2007).

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations, or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations, or equivalents. It is intended that the following claims define the scope of the disclosure and that methods and structures within the scope of these claims and their equivalents be covered thereby.

What is claimed is:

1. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 1-29, 393-735, 799-895, 1020-1476, 1544-1554, 1850-2160, 2165-2210, and 2258-2266.

2. The engineered retrotransposase system of claim 1, wherein the retrotransposase comprises an amino acid sequence having at least 80% sequence identity to any one of SEQ ID NOs: 1-29, 393-735, 799-895, 1020-1476, 1544-1554, 1850-2160, 2165-2210, and 2258-2266.

3. The engineered retrotransposase system of claim 1, wherein the retrotransposase comprises an amino acid sequence having at least 90% sequence identity to any one of SEQ ID NOs: 1-29, 393-735, 799-895, 1020-1476, 1544-1554, 1850-2160, 2165-2210, and 2258-2266.

4. The engineered retrotransposase system of claim 1, wherein the retrotransposase comprises an amino acid sequence having at least 95% sequence identity to any one of SEQ ID NOs: 1-29, 393-735, 799-895, 1020-1476, 1544-1554, 1850-2160, 2165-2210, and 2258-2266.

5. The engineered retrotransposase system of claim 1, wherein the retrotransposase is encoded by a nucleic acid having at least 75% sequence identity to any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806.

6. The engineered retrotransposase system of claim 1, wherein the retrotransposase is encoded by a nucleic acid sequence having at least 80% sequence identity to any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806.

7. The engineered retrotransposase system of claim 1, wherein retrotransposase is encoded by a nucleic acid sequence having at least 90% sequence identity to any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806.

8. The engineered retrotransposase system of claim 1, wherein retrotransposase is encoded by a nucleic acid sequence having at least 95% sequence identity to any one of SEQ ID NOs: 120-173, 181-187, 193-197, 203-207, 217-225, 231-235, 241-245, 251-255, 267-277, 288-297, 303-307, 324-339, 964-981, 1003-1019, 1504-1520, 1521-1536, 1539-1543, 1556-1568, and 1611-1806.

9. The engineered retrotransposase system of any one of claims 1-8, wherein the double-stranded nucleic acid comprises a 5′ recognition sequence comprising a GG nucleotide sequence and a 3′ recognition sequence comprising a TGAC nucleotide sequence.

10. The engineered retrotransposase system of claim 9, wherein the 5′ recognition sequence and the 3′ recognition sequence are configured to interact with the retrotransposase.

11. The engineered retrotransposase system of any one of claims 1-10, wherein the double-stranded nucleic acid comprising a cargo nucleotide sequence is RNA.

12. The engineered retrotransposase system of claim 11, wherein the RNA is an in vitro transcribed RNA.

13. The engineered retrotransposase system of any one of claims 11-12, wherein the RNA comprises a sequence 5′ to the cargo sequence or a sequence 3′ to the cargo sequence that has at least 80% sequence identity to an RNA cognate of any one of SEQ ID NOs: 761-798, 2161-2164, and 2211-2257, a complement thereof, or a reverse complement thereof.

14. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 1-29, 393-401, 799-894, 1476, 1850-1926, and 2165-2210.

15. The engineered retrotransposase system of claim 14, wherein the retrotransposase is encoded by a nucleic acid sequence having at least 80% sequence identity to any one of SEQ ID NOs: SEQ ID NOs: 1535-1536, 1542-1543, 1611-1623, 1663-1691, and 1786-1806.

16. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to SEQ ID NO: 402 or SEQ ID NO: 895.

17. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to SEQ ID NO: 388.

18. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 403-426.

19. The engineered retrotransposase system of claim 18, wherein the retrotransposase is encoded by a nucleic acid sequence having at least 80% sequence identity to any one of SEQ ID NOs: 389-392 and 1504-1507.

20. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 427-439.

21. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 440-554 and 1020-1037.

22. The engineered retrotransposase system of claim 21, wherein the retrotransposase is encoded by a nucleic acid sequence having at least 80% sequence identity to any one of SEQ ID NOs: 356-373, 964-981, and 1003-1019.

23. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 555-608 and 1927-2010.

24. The engineered retrotransposase system of claim 23, wherein the retrotransposase is encoded by a nucleic acid sequence having at least 80% sequence identity to any one of SEQ ID NOs: 66-173, 740-756, 1521-1534, 1539-1541, 1624-1637, 1645-1662, and 1701-1782.

25. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 609-610 and 1555.

26. The engineered retrotransposase system of claim 25, wherein the retrotransposase is encoded by a nucleic acid sequence having at least 80% sequence identity to any one of SEQ ID NOs: 308-309 and 324-325.

27. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 611-615 and 1544-1545.

28. The engineered retrotransposase system of claim 27, wherein the retrotransposase is encoded by a nucleic acid sequence having at least 80% sequence identity to any one of SEQ ID NOs: 310-312, 326-328, 1556-1557, and 1569-1570.

29. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to SEQ ID NO: 616 or SEQ ID NO: 617.

30. The engineered retrotransposase system of claim 29, wherein the retrotransposase is encoded by a nucleic acid sequence having at least 80% sequence identity to any one of SEQ ID NOs: 313-314 and 329-330.

31. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 618-622 and 2258-2266.

32. The engineered retrotransposase system of claim 31, wherein the retrotransposase is encoded by a nucleic acid sequence having at least 80% sequence identity to any one of SEQ ID NOs: 315-319 and 331-335.

33. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to SEQ ID NO: 623.

34. The engineered retrotransposase system of claim 33, wherein the retrotransposase is encoded by a nucleic acid sequence having at least 80% sequence identity to SEQ ID NO: 320 or SEQ ID NO: 336.

35. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 624-626.

36. The engineered retrotransposase system of claim 35, wherein the retrotransposase is encoded by a nucleic acid sequence having at least 80% sequence identity to any one of SEQ ID NOs: 321-323, 337-339, and 1785.

37. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 624-626.

38. The engineered retrotransposase system of claim 35, wherein the retrotransposase is encoded by a nucleic acid sequence having at least 80% sequence identity to any one of SEQ ID NOs: 321-323, 337-339, and 1785.

39. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 627-673, 1039-1475, and 2011-2026.

40. The engineered retrotransposase system of claim 39, wherein the retrotransposase is encoded by a nucleic acid sequence having at least 80% sequence identity to any one of SEQ ID NOs: 174-187 and 1508-1520.

41. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 674-678.

42. The engineered retrotransposase system of claim 41, wherein the retrotransposase is encoded by a nucleic acid sequence having at least 80% sequence identity to any one of SEQ ID NOs: 188-197.

43. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 679-683.

44. The engineered retrotransposase system of claim 43, wherein the retrotransposase is encoded by a nucleic acid sequence having at least 80% sequence identity to any one of SEQ ID NOs: 198-207.

45. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 684-692 and 2027-2046.

46. The engineered retrotransposase system of claim 45, wherein the retrotransposase is encoded by a nucleic acid sequence having at least 80% sequence identity to any one of SEQ ID NOs: 208-225 and 757-759.

47. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 693-697 and 2047-2090.

48. The engineered retrotransposase system of claim 47, wherein the retrotransposase is encoded by a nucleic acid sequence having at least 80% sequence identity to any one of SEQ ID NOs: 226-235.

49. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 698-702 and 2091-2119.

50. The engineered retrotransposase system of claim 49, wherein the retrotransposase is encoded by a nucleic acid sequence having at least 80% sequence identity to any one of SEQ ID NOs: 236-245 and 759-760.

51. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 703-707.

52. The engineered retrotransposase system of claim 51, wherein the retrotransposase is encoded by a nucleic acid sequence having at least 80% sequence identity to any one of SEQ ID NOs: 246-255.

53. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 708-718 and 2121-2159.

54. The engineered retrotransposase system of claim 53, wherein the retrotransposase is encoded by a nucleic acid sequence having at least 80% sequence identity to any one of SEQ ID NOs: 256-277, 1638-1644, and 1693-1700.

55. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 719-728.

56. The engineered retrotransposase system of claim 55, wherein the retrotransposase is encoded by a nucleic acid sequence having at least 80% sequence identity to any one of SEQ ID NOs: 278-297.

57. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 729-733.

58. The engineered retrotransposase system of claim 57, wherein the retrotransposase is encoded by a nucleic acid sequence having at least 80% sequence identity to any one of SEQ ID NOs: 298-307.

59. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to any one of SEQ ID NOs: 734-735 and 1546-1553.

60. The engineered retrotransposase system of claim 59, wherein the retrotransposase is encoded by a nucleic acid sequence having at least 80% sequence identity to any one of SEQ ID NOs: 1558-1567, 1571-1580, and 1783-1784.

61. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to SEQ ID NO: 1038 or SEQ ID NO: 2160.

62. The engineered retrotransposase system of claim 61, wherein the retrotransposase is encoded by a nucleic acid sequence having at least 80% sequence identity to SEQ ID NO: 1692.

63. An engineered retrotransposase system, comprising:
(a) a double-stranded nucleic acid comprising a cargo nucleotide sequence configured to form a complex with a retrotransposase; and
(b) a retrotransposase configured to transpose the cargo nucleotide sequence to a target nucleic acid sequence and comprising an amino acid sequence having at least 75% sequence identity to SEQ ID NO: 1554.

64. The engineered retrotransposase system of claim 63, wherein the retrotransposase is encoded by a nucleic acid sequence having at least 80% sequence identity to SEQ ID NO: 1568 or SEQ ID NO: 1594.

65. The engineered retrotransposase system of any one of claims 1-64, wherein the retrotransposase comprises one or more nuclear localization sequences (NLSs) proximal to an N- or C-terminus of the retrotransposase.

66. The engineered retrotransposase system of claim 65, wherein the NLS comprises a sequence at least 80% identical to a sequence from the group consisting of SEQ ID NO: 1477-1492.

67. The engineered retrotransposase system of claim 65, wherein the NLS comprises SEQ ID NO: 1478.

68. The engineered retrotransposase system of claim 65, wherein the NLS is proximal to the N-terminus of the retrotransposase.

69. The engineered retrotransposase system of claim 65, wherein the NLS comprises SEQ ID NO: 1477.

70. The engineered retrotransposase system of claim 65, wherein the NLS is proximal to the C-terminus of the retrotransposase.

71. A polypeptide comprising a reverse transcriptase comprising an amino acid sequence having at least 75% sequence identity to any one of SEQ ID NOs: 1-29, 393-735, 799-895, 1020-1476, 1544-1554, 1850-2160, 2165-2210, and 2258-2266 fused N- or C-terminally to a non-retrotransposase domain or an affinity tag.

72. The polypeptide of claim 71, wherein the non-retrotransposase domain is an RNA-binding protein domain.

73. The polypeptide of claim 72, wherein the RNA binding protein domain comprises a bacteriophage MS2 coat protein (MCP) domain.

74. A nucleic acid encoding the engineered retrotransposase system of any one of claims 1-64 or the polypeptide of any one of claims 71-73.

75. A method for modifying a target nucleic acid sequence comprising contacting the target nucleic acid sequence using the engineered nuclease system of any one of claims 1-64.

76. The method of claim 75, wherein modifying the target nucleic acid sequence comprises binding, nicking, or cleaving, the target nucleic acid sequence.

77. The method of any one of claims 75-76, wherein the target nucleic acid sequence comprises genomic DNA, viral DNA, viral RNA, or bacterial DNA.

78. The method of any one of claims 75-76, wherein the target nucleic acid sequence comprises deoxyribonucleic acid (DNA).

79. The method of any one of claims 75-78, wherein the modification is in vitro.

80. The method of any one of claims 75-78, wherein the modification is in vivo.

81. The method of any one of claims 75-78, wherein the modification is ex vivo.

82. A method of modifying a target nucleic acid sequence in a mammalian cell comprising contacting the mammalian cell using the engineered nuclease system of any one of claims 1-64.

83. A method for synthesizing complementary DNA (cDNA), comprising:
(a) providing an RNA molecule as a template for cDNA synthesis,
(b) providing a primer oligonucleotide to initiate cDNA synthesis from the RNA molecule; and
(c) synthesizing cDNA initiated by the primer oligonucleotide from the template using a reverse transcriptase comprising a sequence having at least 80% sequence identity to a reverse transcriptase domain of any one of SEQ ID NOs: 1-29, 393-735, 799-895, 1020-1476, 1544-1554, 1850-2160, 2165-2210, and 2258-2266.

84. The method of claim 83, wherein the primer oligonucleotide comprises an oligo (dT) sequence or a degenerate sequence of at least six oligonucleotides.

85. A vector comprising the nucleic acid of claim 74.

86. The vector of claim 85, wherein the vector is a plasmid, a minicircle, a CELiD, an adeno-associated virus (AAV) derived virion, or a lentivirus.

87. A cell comprising the engineered nuclease system of any one of claims 1-64 or the polypeptide of any one of claims 71-73.

88. The cell of claim 87, wherein the cell is a eukaryotic cell.

89. The cell of claim 87, wherein the cell is a mammalian cell.

90. The cell of claim 87, wherein the cell is an immortalized cell.

91. The cell of claim 87, wherein the cell is an insect cell.

92. The cell of claim 87, wherein the cell is a yeast cell.

93. The cell of claim 87, wherein the cell is a plant cell.

94. The cell of claim 87, wherein the cell is a fungal cell.

95. The cell of claim 87, wherein the cell is a prokaryotic cell.

96. The cell of claim 87, wherein the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, primary cell, or a derivative thereof.

97. The cell of claim 87, wherein the cell is an engineered cell.

98. The cell of claim 87, wherein the cell is a stable cell.
